SEQUENCE LISTING (437 sequences)

SEQ ID NO: 1 - protein, 120 aa, Artificial Sequence
  Derived from a Human germline sequence
EVQLLESGGGLVQPGGSLRLSCAASGFTFSQYRMHWVRQAPGKSLEWVSSIDTRGSSTYYADPVKGRFTISRDNSKNTLYLQMNSLRAEDTAVYYCAKAVTMFSPFFDYWGQGTLVTVSS

SEQ ID NO: 2 - protein, 123 aa, Artificial Sequence
  Derived from a Human germline sequence
EVQLLESGGGLVQPGGSLRLSCAASGFTFADYGMRWVRQAPGKGLEWVSSITRTGRVTYYADSVKGRFTISRDNSKNTLYLQMNSLRAEDTAVYYCAKWRNRHGEYLADFDYWGQGTLVTVSS

SEQ ID NO: 3 - protein, 120 aa, Artificial Sequence
  Derived from a Human germline sequence
EVQLLESGGGLVQPGGSLRLSCAASGFTFMRYRMHWVRQAPGKGLEWVSSIDSNGSSTYYADSVKGRFTISRDNSKNTLYLQMNSLRAEDTAVYYCAKDRTERSPVFDYWGQGTLVTVSS

SEQ ID NO: 4 - protein, 119 aa, Artificial Sequence
  Derived from a Human germline sequence
EVQLLESGGGLVQPGGSLRLSCAASGFTFVDYEMHWVRQAPGKGLEWVSSISESGTTTYYADSVKGRFTISRDNSKNTLYLQMNSLRAEDTAVYYCAKRRFSASTFDYWGQGTLVTVSS

SEQ ID NO: 5 - protein, 119 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAKYTGHWEPFDYWGQGTLVTVSS

SEQ ID NO: 6 - protein, 119 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAKYTGRWEPYDYWGQGTLVTVSS

SEQ ID NO: 7 - protein, 119 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAKYTGRWEPFDYWGQGTLVTVSS

SEQ ID NO: 8 - protein, 119 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAKYTGRWEPFEYWGQGTLVTVSS

SEQ ID NO: 9 - protein, 119 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAIYTGRWEPFDYWGQGTLVTVSS

SEQ ID NO: 10 - protein, 119 aa, Artificial Sequence
  Derived from a Human germline sequence
EVQLLESGGGLVQPGGSLRLSCAASGFTFVKYSMGWVRQAPGKGPEWVSQISNTGGHTYYADSVKGRFTISRDNSKNTLYLQMNSLRAEDTAVYYCAIYTGRWEPFDYWGQGTLVTVSS

SEQ ID NO: 11 - protein, 119 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYMQMNSLRAEDTAVYYCAIYTGRWEPFDYWGQGTLVTVSS

SEQ ID NO: 12 - protein, 119 aa, Artificial Sequence
  Derived from a Human germline sequence
EVQLLESGGGLVQPGGSLRLSCAASGFTFGKYSMGWVRQAPGKDLEWVSQISNTGGHTYYADSVKGRFTISRDNSKNTLYLQMNSLRAEDTAVYYCAIYTGRWEPFDYWGQGTLVTVSS

SEQ ID NO: 13 - protein, 119 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAKYTGRWEPFDHWGQGTLVTVSS

SEQ ID NO: 14 - protein, 119 aa, Artificial Sequence
  Derived from a Human germline sequence
EVQLLESGGGLVQPGGSLRLSCAASGFTFVKYSMGWVRQAPGKGLEWVSQISNTGDHTYYADSVKGRFTISRDNSKNTLYLQMNSLRAEDTAVYYCAKYTGRWEPFDYWGQGTLVTVSS

SEQ ID NO: 15 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAKYTGRWEPFDYWGQGTLVTVSS

SEQ ID NO: 16 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWEPFDYWGQGTLVTVSS

SEQ ID NO: 17 - protein, 119 aa, Artificial Sequence
  Derived from a Human germline sequence
EVQLLESGGGLVQPGGSLRLSCAASGFTFVKYSMGWVRQAPGKGLEWVSQISNTGDHTYYADSVKGRFTISRDNSKNTLYLQMNSLRAEDTAVYYCAIYTGRWEPFDYWGQGTLVTVSS

SEQ ID NO: 18 - protein, 119 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSS

SEQ ID NO: 19 - protein, 119 aa, Artificial Sequence
  Derived from a Human germline sequence
EVQLLESGGGLVQPGGSLRLSCAASGFTFVKYSMGWVRQAPGKGPEWVSQISNTGDHTYYADSVKGRFTISRDNSKNTLYLQMNSLRAEDTAVYYCAIYTGRWEPFDYWGQGTLVTVSS

SEQ ID NO: 20 - protein, 119 aa, Artificial Sequence
  Derived from a Human germline sequence
EVQLLESGGGLVQPGGSLRLSCAASGFTFGKYSMGWVRQAPGKDLEWVSQISNTGDRTYYADSVKGRFTISRDNSKNTLYLQMNSLRAEDTAVYYCAIYTGRWEPFDYWGQGTLVTVSS

SEQ ID NO: 21 - protein, 119 aa, Artificial Sequence
  Derived from a Human germline sequence
EVQLLESGGGLVQPGGSLRLSCAASGFTFGKYSMGWVRQAPGKDLEWVSQISNTGDHTYYADSVKGRFTISRDNSKNTLYLQMNSLRAEDTAVYYCAIYTGRWEPFDYWGQGTLVTVSS

SEQ ID NO: 22 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWEPFVYWGQGTLVTVSS

SEQ ID NO: 23 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWEPFEYWGQGTLVTVSS

SEQ ID NO: 24 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWKPFEYWGQGTLVTVSS

SEQ ID NO: 25 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWVPFEYWGQGTLVTVSS

SEQ ID NO: 26 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWRPFEYWGQGTLVTVSS

SEQ ID NO: 27 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAAYYCAIYTGRWEPFDYWGQGTLVTVSS

SEQ ID NO: 28 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWEPFNYWGQGTLVTVSS

SEQ ID NO: 29 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWAPFEYWGQGTLVTVSS

SEQ ID NO: 30 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNSLYLQMNSLRAEDTAVYYCAIYTGRWVPFDNWGQGTLVTVSS

SEQ ID NO: 31 - protein, 119 aa, Artificial Sequence
  Derived from a Human germline sequence
EVQLLESGGGLVQPGGSLRLSCAASGFTFITYSMGWVRQAPGKGLEWVSQISNTGDRTYYADSVKGRFTISRDNSKNTLYLQMNSLRAEDTAVYYCAIYTGRWEPFQYWGQGTLVTVSS

SEQ ID NO: 32 - protein, 119 aa, Artificial Sequence
  Derived from a Human germline sequence
EVQLLESGGGLVQPGGSLRLSCAASGFTFGKYSMGWVRQAPGKGLEWVSQISNTGDRTYYADSVKGRFTISRDNSKNTLYLQMNSLRAEDTAVYYCAIYTGRWEPFDYWGQGTLVTVSS

SEQ ID NO: 33 - protein, 119 aa, Artificial Sequence
  Derived from a Human germline sequence
EVQLLESGGGLVQPGGSLRLSCAASGFTFFKYSMGWVRQAPGKGLEWVSQISNTGDRTYYADSVKGRFTISRDNSKNTLYLQMNSLRAEDTAVYYCAIYTGRWEPFDYWGQGTLVTVSS

SEQ ID NO: 34 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWEPFDYWGQGTLVTVSS

SEQ ID NO: 35 - protein, 119 aa, Artificial Sequence
  Derived from a Human germline sequence
EVQLLESGGGLVQPGGSLRLSCAASGFTFVKYSMGWVRQAPGKGLEWVSQISNTGDRRYYADAVKGRFTISRDNSKNTLYLQMNSLRAEDTAVYYCAIYTGRWEPFDYWGQGTLVTVSS

SEQ ID NO: 36 - protein, 119 aa, Artificial Sequence
  Derived from a Human germline sequence
EVQLLESGGGLVQPGGSLRLSCAASGFTFVKYSMGWVRQAPGKGLEWVSQISNTGDRTYYADSVKGRFTISRDNSKNTLYLQMNSLRAEDTAVYYCAIYTGRWEPFKYWGQGTLVTVSS

SEQ ID NO: 37 - protein, 119 aa, Artificial Sequence
  Derived from a Human germline sequence
EVQLLESGGGLVQPGGSLRLSCAASGFTFSKYSMGWVRQAPGKGLEWVSQISNTGERRYYADSVKGRFTISRDNPKNTLYLQMNSLRAEDTAVYYCAIYTGRWEPFEYWGQGTLVTVSS

SEQ ID NO: 38 - protein, 119 aa, Artificial Sequence
  Derived from a Human germline sequence
EVQLLESGGGLVQPGGSLRLSCAASGFTFVNYSMGWVRQAPGKGLEWVSQISNTGDRTYYADSVKGRFTISRDNSKNTLYLQMNSLRAEDTAVYYCAIYTGRWEPYEYWGQGTLVTVTS

SEQ ID NO: 39 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAVYYCAIYTGRWEPFVYWGQGTLVTVSS

SEQ ID NO: 40 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAVYYCAIYTGRWKPFEYWGQGTLVTVSS

SEQ ID NO: 41 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAVYYCAIYTGRWVPFEYWGQGTLVTVSS

SEQ ID NO: 42 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAVYYCAIYTGRWRPFEYWGQGTLVTVSS

SEQ ID NO: 43 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAVYYCAIYTGRWAPFEYWGQGTLVTVSS

SEQ ID NO: 44 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAVYTGRWEPFVYWGQGTLVTVSS

SEQ ID NO: 45 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWKPFEYWGQGTLVTVSS

SEQ ID NO: 46 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSS

SEQ ID NO: 47 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWRPFEYWGQGTLVTVSS

SEQ ID NO: 48 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWAPFEYWGQGTLVTVSS

SEQ ID NO: 49 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWEPFVYWGQGTLVTVSS

SEQ ID NO: 50 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWKPFEYWGQGTLVTVSS

SEQ ID NO: 51 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWVPFEYWGQGTLVTVSS

SEQ ID NO: 52 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWRPFEYWGQGTLVTVSS

SEQ ID NO: 53 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWAPFEYWGQGTLVTVSS

SEQ ID NO: 54 - protein, 119 aa, Artificial Sequence
  Derived from a Human germline sequence
EVQLLESGGGLVQPGGSLRLSCAASGFTFVKYSMGWVRQAPGKGLEWVSQISNTGDRRYYADAVKGRFTISRDNSKNTLYLQMNSLRAEDTAVYYCAIYTGRWEPFVYWGQGTLVTVSS

SEQ ID NO: 55 - protein, 119 aa, Artificial Sequence
  Derived from a Human germline sequence
EVQLLESGGGLVQPGGSLRLSCAASGFTFVKYSMGWVRQAPGKGLEWVSQISNTGDRRYYADAVKGRFTISRDNSKNTLYLQMNSLRAEDTAVYYCAIYTGRWKPFEYWGQGTLVTVSS

SEQ ID NO: 56 - protein, 119 aa, Artificial Sequence
  Derived from a Human germline sequence
EVQLLESGGGLVQPGGSLRLSCAASGFTFVKYSMGWVRQAPGKGLEWVSQISNTGDRRYYADAVKGRFTISRDNSKNTLYLQMNSLRAEDTAVYYCAIYTGRWVPFEYWGQGTLVTVSS

SEQ ID NO: 57 - protein, 119 aa, Artificial Sequence
  Derived from a Human germline sequence
EVQLLESGGGLVQPGGSLRLSCAASGFTFVKYSMGWVRQAPGKGLEWVSQISNTGDRRYYADAVKGRFTISRDNSKNTLYLQMNSLRAEDTAVYYCAIYTGRWRPFEYWGQGTLVTVSS

SEQ ID NO: 58 - protein, 119 aa, Artificial Sequence
  Derived from a Human germline sequence
EVQLLESGGGLVQPGGSLRLSCAASGFTFVKYSMGWVRQAPGKGLEWVSQISNTGDRRYYADAVKGRFTISRDNSKNTLYLQMNSLRAEDTAVYYCAIYTGRWAPFEYWGQGTLVTVSS

SEQ ID NO: 59 - protein, 119 aa, Artificial Sequence
  Derived from a Human germline sequence
EVQLLESGGGLVQPGGSLRLSCAASGFTFLKFSMGWVRQAPGKGLEWVSQIANTGDRRYYADSVKGRFTISRDNSKNTLYLQMNSLRAEDTAVYYCAIYTGRWAPFEYWGQGTLVTVSS

SEQ ID NO: 60 - protein, 119 aa, Artificial Sequence
  Derived from a Human germline sequence
EVQLLESGGGLVQPGGSLRLSCAASGFTFLKYSMGWVRQAPGKGLEWVSQISNTADRTYYAHSVKGRFTISRDNSKNTLYLQMNSLRAEDTAVYYCAIYTGRWAPFEYWGQGTLVTVSS

SEQ ID NO: 61 - protein, 119 aa, Artificial Sequence
  Derived from a Human germline sequence
EVQLLESGGGLVQPGGSLRLSCAASGFTFFKYSMGWVRQAPGKGLEWVSQISDTGDRRYYDDSVKGRFTISRDNSKNTLYLQMNSLRAEDTAVYYCAIYTGRWEPFVYWGQGTLVTVSS

SEQ ID NO: 62 - protein, 119 aa, Artificial Sequence
  Derived from a Human germline sequence
EVQLLESGGGLVQPGGSLRLSCAASGFTFLKYSMGWVRQAPGKGLEWVSQISDTGDRRYYDDSVKGRFTISRDNSKNTLYLQMNSLRAEDTAVYYCAIYTGRWEPFVYWGQGTLVTVSS

SEQ ID NO: 63 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAAYYCAIYTGRWPDFDYWGQGTLVTVSS

SEQ ID NO: 64 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADSVKGRFTISRDNSKNTLYLQMNSLRAEDTAAYYCAIYTGRWPDFEYWGQGTLVTVSS

SEQ ID NO: 65 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWPDFDYWGQGTLVTVSS

SEQ ID NO: 66 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWPDFEYWGQGTLVTVSS

SEQ ID NO: 67 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWPDFDYWGQGTLVTVSS

SEQ ID NO: 68 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDDSVKGRFTISRDNSKNTLYLQMNSLRAEDTAVYYCAIYTGRWPDFEYWGQGTLVTVSS

SEQ ID NO: 69 - protein, 119 aa, Artificial Sequence
  Derived from a Human germline sequence
EVQLLESGGGLVQPGGSLRLSCAASGFTFVKYSMGWVRQAPGKGPEWVSQISAWGDRTYYADSVKGRFTISRDNSKNTLYLQMNSLRAEDTAVYYCAIYTGRWEPFDYWGQGTLVTVSS

SEQ ID NO: 70 - protein, 119 aa, Artificial Sequence
  Derived from a Human germline sequence
EVQLLESGGGLVQPGGSLRLSCAASGFTFVKYSMGWVRQAPGKGPEWVSQISDGGQRTYYADSVKGRFTISRDNSKNTLYLQMNSLRAEDTAVYYCAIYTGRWEPFDYWGQGTLVTVSS

SEQ ID NO: 71 - protein, 119 aa, Artificial Sequence
  Derived from a Human germline sequence
EVQLLESGGGLVQPGGSLRLSCAASGFTFVKYSMGWVRQAPGKGPEWVSQISDSGYRTYYADSVKGRFTISRDNSKNTLYLQMNSLRAEDTAVYYCAIYTGRWEPFDYWGQGTLVTVSS

SEQ ID NO: 72 - protein, 119 aa, Artificial Sequence
  Derived from a Human germline sequence
EVQLLESGGGLVQPGGSLRLSCAASGFTFVKYSMGWVRQAPGKGPEWVSQISDGGTRTYYADSVKGRFTISRDNSKNTLYLQMNSLRAEDTAVYYCAIYTGRWEPFDYWGQGTLVTVSS

SEQ ID NO: 73 - protein, 119 aa, Artificial Sequence
  Derived from a Human germline sequence
EVQLLESGGGLVQPGGSLRLSCAASGFTFVKYSMGWVRQAPGKGPEWVSQISDKGTRTYYADSVKGRFTISRDNSKNTLYLQMNSLRAEDTAVYYCAIYTGRWEPFDYWGQGTLVTVSS

SEQ ID NO: 74 - protein, 119 aa, Artificial Sequence
  Derived from a Human germline sequence
EVQLLESGGGLVQPGGSLRLSCAASGFTFVKYSMGWVRQAPGKGPEWVSQISETGRRTYYADSVKGRFTISRDNSKNTLYLQMNSLRAEDTAVYYCAIYTGRWEPFDYWGQGTLVTVSS

SEQ ID NO: 75 - protein, 119 aa, Artificial Sequence
  Derived from a Human germline sequence
EVQLLESGGGLVQPGGSLRLSCAASGFTFVKYSMGWVRQAPGKGLEWVSQINNTGSTTYYADSVKGRFTISRDNSKNTLYLQMNSLRAEDTAVYYCAIYTGRWEPFDYWGQGTLVTVSS

SEQ ID NO: 76 - protein, 117 aa, Artificial Sequence
  Derived from a Human germline sequence
QLLESGGGLVQPGGSLRLSCAASGFTFVKYSMGWVRQAPGKGPEWVSQISNTADRTYYAHSVKGRFTISRDNSKNTLYLQMNSLRAEDTAVYYCAIYTGRWVPFEYWGQGTLVTVSS

SEQ ID NO: 77 - protein, 119 aa, Artificial Sequence
  Derived from a Human germline sequence
EVQLLESGGGLVQPGGSLRLSCAASGFTFVKYSMGWVRQAPGKGPEWVSQISNTADRTYYAHSVKGRFTISRDNSKNTLYLQMNSLRAEDTAVYYCAIYTGRWAPFEYWGQGTLVTVSS

SEQ ID NO: 78 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWVPFEYWGQGTLVTVSS

SEQ ID NO: 79 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWAPFEYWGQGTLVTVSS

SEQ ID NO: 80 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYDDSVKGRFTISRDNSKNTLYLQMNSLRAEDTAVYYCAIYTGRWRPFEYWGQGTLVTVSS

SEQ ID NO: 81 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYTHSVKGRFTISRDNSKNTLYLQMNSLRAEDTAVYYCAIYTGRWAPFEYWGQGTLVTVSS

SEQ ID NO: 82 - protein, 119 aa, Artificial Sequence
  Derived from a Human germline sequence
EVQLLESGGGLVQPGGSLRLSCAASGFTFVKYSMGWVRQAPGKGLEWVSQISNTADRRYYAHSVKGRFTISRDNSKNTLYLQMNSLRAEDTAVYYCAIYTGRWAPFEYWGQGTLVTVSS

SEQ ID NO: 83 - protein, 119 aa, Artificial Sequence
  Derived from a Human germline sequence
EVQLLESGGGLVQPGGSLRLSCAASGFTFVKYSMGWVRQAPGKGLEWVSQILNTADRTYYDHSVKGRFTISRDNSKNTLYLQMNSLRAEDTAVYYCAIYTGRWAPFEYWGQGTLVTVSS

SEQ ID NO: 84 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYDHSVKGRFTISRDNSKNTLYLQMNSLRAEDTAVYYCAIYTGRWAPFEYWGQGTLVTVSS

SEQ ID NO: 85 - protein, 119 aa, Artificial Sequence
  Derived from a Human germline sequence
EVQLLESGGGLVQPGGSLRLSCAASGFTFVKYSMGWVRQAPGKGLEWVSQISDTADRRYYAHSVKGRFTISRDNSKNTLYLQMNSLRAEDTAVYYCAIYTGRWAPFEYWGQGTLVTVSS

SEQ ID NO: 86 - protein, 119 aa, Artificial Sequence
  Derived from a Human germline sequence
EVQLLESGGGLVQPGGSLRLSCAASGFTFVKYSMGWVRQAPGKGLEWVSQISDTADRRYYDHSVKGRFTISRDNSKNTLYLQMNSLRAEDTAVYYCAIYTGRWAPFEYWGQGTLVTVSS

SEQ ID NO: 87 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAVYTGRWVSFEYWGQGTLVTVSS

SEQ ID NO: 88 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCALYTGRWVSFEYWGQGTLVTVSS

SEQ ID NO: 89 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAVYTGRWVPFEYWGQGTLVTVSS

SEQ ID NO: 90 - protein, 119 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCALYTGRWVPFEYWGQGTLVTVSS

SEQ ID NO: 91 - protein, 119 aa, Artificial Sequence
  Derived from a Human germline sequence
EVQLLESGGGLVQPGGSLRLSCAASGFTFVKYSMGWVRQAPGKGLEWVSQIANTADRRYYAHSVKGRFTISRDNSKNTLYLQMNSLRAEDTAVYYCAIYTGRWAPFEYWGQGTLVTVSS

SEQ ID NO: 92 - protein, 119 aa, Artificial Sequence
  Derived from a Human germline sequence
EVQLLESGGGLVQPGGSLRLSCAASGFTFVKYSMGWVRQAPGKGLEWVSQISNTADRRYYADAVKGRFTISRDNSKNTLYLQMNSLRAEDTAVYYCAIYTGRWEPFVYWGQGTLVTVSS

SEQ ID NO: 93 - protein, 119 aa, Artificial Sequence
  Derived from a Human germline sequence
EVQLLESGGGLVQPGGSLRLSCAASGFTFVKYSMGWVRQAPGKGLEWVSQISNTGDRRYYAHAVKGRFTISRDNSKNTLYLQMNSLRAEDTAVYYCAIYTGRWEPFVYWGQGTLVTVSS

SEQ ID NO: 94 - protein, 119 aa, Artificial Sequence
  Derived from a Human germline sequence
EVQLLESGGGLVQPGGSLRLSCAASGFTFVKYSMGWVRQAPGKGLEWVSQIANTADRRYYADAVKGRFTISRDNSKNTLYLQMNSLRAEDTAVYYCAIYTGRWEPFVYWGQGTLVTVSS

SEQ ID NO: 95 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYAHAVKGRFTISRDNSKNTLYLQMNSLRAEDTAVYYCAIYTGRWEPFVYWGQGTLVTVSS

SEQ ID NO: 96 - protein, 119 aa, Artificial Sequence
  Derived from a Human germline sequence
EVQLLESGGGLVQPGGSLRLSCAASGFTFVKYSMGWVRQAPGKGLEWVSQISNTADRRYYAHAVKGRFTISRDNSKNTLYLQMNSLRAEDTAVYYCAIYTGRWEPFVYWGQGTLVTVSS

SEQ ID NO: 97 - protein, 119 aa, Artificial Sequence
  Derived from a Human germline sequence
EVQLLESGGGLVQPGGSLRLSCAASGFTFVKYSMGWVRQAPGKGLEWVSQIANTADRRYYAHAVKGRFTISRDNSKNTLYLQMNSLRAEDTAVYYCAIYTGRWEPFVYWGQGTLVTVSS

SEQ ID NO: 98 - protein, 119 aa, Artificial Sequence
  Derived from a Human germline sequence
EVQLLESGGGLVQPGGSLRLSCAASGFTFVKYSMGWVRQAPGKGLEWVSQIVNTGDRRYYADAVKGRFTISRDNSKNTLYLQMNSLRAEDTAVYYCAIYTGRWEPFVYWGQGTLVTVSS

SEQ ID NO: 99 - protein, 119 aa, Artificial Sequence
  Derived from a Human germline sequence
EVQLLESGGGLVQPGGSLRLSCAASGFTFVKYSMGWVRQAPGKGLEWVSQIANTGDRRYYADAVKGRFTISRDNSKNTLYLQMNSLRAEDTAVYYCAIYTGRWEPFVYWGQGTLVTVSS

SEQ ID NO: 100 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYDHSVKGRFTISRDNSKNTLYLQMNSLRAEDTAVYYCAIYTGRWAPFEYWGQGTLVTVSS

SEQ ID NO: 101 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYDHSVKGRFTISRDNSKNTLYLQMNSLRAEDTAVYYCAIYTGRWRPFEYWGQGTLVTVSS

SEQ ID NO: 102 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYDHSVKGRFTISRDNSKNTLYLQMNSLRAEDTAVYYCAIYTGRWEPFVYWGQGTLVTVSS

SEQ ID NO: 103 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYSHSVKGRFTISRDNSKNTLYLQMNSLRAEDTAVYYCAIYTGRWVPFEYWGQGTLVTVSS

SEQ ID NO: 104 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYTHSVKGRFTISRDNSKNTLYLQMNSLRAEDTAVYYCAIYTGRWVPFEYWGQGTLVTVSS

SEQ ID NO: 105 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYTDAVKGRFTISRDNSKNTLYLQMNSLRAEDTAVYYCAIYTGRWEPFVYWGQGTLVTVSS

SEQ ID NO: 106 - protein, 119 aa, Artificial Sequence
  Derived from a Human germline sequence
EVQLLESGGGLVQPGGSLRLSCAASGFTFFKYSMGWVRQAPGKGLEWVSQISDTADRTYYAHSVKGRFTISRDNSKNTLYLQMNSLRAEDTAVYYCAIYTGRWAPFEYWGQGTLVTVSS

SEQ ID NO: 107 - protein, 119 aa, Artificial Sequence
  Derived from a Human germline sequence
EVQLLESGGGLVQPGGSLRLSCAASGFTFLKYSMGWVRQAPGKGLEWVSQISDTADRTYYAHSVKGRFTISRDNSKNTLYLQMNSLRAEDTAVYYCAIYTGRWAPFEYWGQGTLVTVSS

SEQ ID NO: 108 - protein, 119 aa, Artificial Sequence
  Derived from a Human germline sequence
EVQLLESGGGLVQPGGSLRLSCAASGFTFFKYSMGWVRQAPGKGLEWVSQIADTGDRRYYDDSVKGRFTISRDNSKNTLYLQMNSLRAEDTAVYYCAIYTGRWEPFVYWGQGTLVTVSS

SEQ ID NO: 109 - protein, 119 aa, Artificial Sequence
  Derived from a Human germline sequence
EVQLLESGGGLVQPGGSLRLSCAASGFTFFKYSMGWVRQAPGKGLEWVSQISDTADRRYYDDSVKGRFTISRDNSKNTLYLQMNSLRAEDTAVYYCAIYTGRWEPFVYWGQGTLVTVSS

SEQ ID NO: 110 - protein, 119 aa, Artificial Sequence
  Derived from a Human germline sequence
EVQLLESGGGLVQPGGSLRLSCAASGFTFFKYSMGWVRQAPGKGLEWVSQISDTGDRRYYDHSVKGRFTISRDNSKNTLYLQMNSLRAEDTAVYYCAIYTGRWEPFVYWGQGTLVTVSS

SEQ ID NO: 111 - protein, 119 aa, Artificial Sequence
  Derived from a Human germline sequence
EVQLLESGGGLVQPGGSLRLSCAASGFTFFKYSMGWVRQAPGKGLEWVSQISDTGDRRYYDDAVKGRFTISRDNSKNTLYLQMNSLRAEDTAVYYCAIYTGRWEPFVYWGQGTLVTVSS

SEQ ID NO: 112 - protein, 119 aa, Artificial Sequence
  Derived from a Human germline sequence
EVQLLESGGGLVQPGGSLRLSCAASGFTFFKYSMGWVRQAPGKGLEWVSQIADTADRRYYDDSVKGRFTISRDNSKNTLYLQMNSLRAEDTAVYYCAIYTGRWEPFVYWGQGTLVTVSS

SEQ ID NO: 113 - protein, 119 aa, Artificial Sequence
  Derived from a Human germline sequence
EVQLLESGGGLVQPGGSLRLSCAASGFTFFKYSMGWVRQAPGKGLEWVSQIADTGDRRYYDHSVKGRFTISRDNSKNTLYLQMNSLRAEDTAVYYCAIYTGRWEPFVYWGQGTLVTVSS

SEQ ID NO: 114 - protein, 119 aa, Artificial Sequence
  Derived from a Human germline sequence
EVQLLESGGGLVQPGGSLRLSCAASGFTFFKYSMGWVRQAPGKGLEWVSQIADTGDRRYYDDAVKGRFTISRDNSKNTLYLQMNSLRAEDTAVYYCAIYTGRWEPFVYWGQGTLVTVSS

SEQ ID NO: 115 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWGPFVYWGQGTLVTVSS

SEQ ID NO: 116 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWVPFAYWGQGTLVTVSS

SEQ ID NO: 117 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWGPFQYWGQGTLVTVSS

SEQ ID NO: 118 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWEPFQYWGQGTLVTVSS

SEQ ID NO: 119 - protein, 119 aa, Artificial Sequence; identical to SEQ ID NO: 79
  Derived from a Human germline sequence

SEQ ID NO: 120 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWAPFQYWGQGTLVTVSS

SEQ ID NO: 121 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWVPFQYWGQGTLVTVSS

SEQ ID NO: 122 - protein, 119 aa, Artificial Sequence
  Derived from a Human germline sequence
EVQLLESGGGLVQPGGSLRLSCAASGFTFVKYSMGWVRQAPGKGLEWVSQISDTGDRRYYDHSVKGRFTISRDNSKNTLYLQMNSLRAEDTAVYYCAIYTGRWAPFEYWGQGTLVTVSS

SEQ ID NO: 123 - protein, 119 aa, Artificial Sequence
  Derived from a Human germline sequence
EVQLLESGGGLVQPGGSLRLSCAASGFTFLKYSMGWVRQAPGKGLEWVSQISDTADRTYYAHSVKGRFTISRDNSKNTLYLQMNSLRAEDTAVYYCAIYTGRWVPFEYWGQGTLVTVSS

SEQ ID NO: 124 - protein, 119 aa, Artificial Sequence
  Derived from a Human germline sequence
EVQLLESGGGLVQPGGSLRLSCAASGFTFFKYSMGWVRQAPGKGLEWVSQISDTADRTYYAHSVKGRFTISRDNSKNTLYLQMNSLRAEDTAVYYCAIYTGRWVPFEYWGQGTLVTVSS

SEQ ID NO: 125 - protein, 119 aa, Artificial Sequence
  Derived from a Human germline sequence
EVQLLESGGGLVQPGGSLRLSCAASGFTFLKYSMGWVRQAPGKGLEWVSQISDTADRTYYDHSVKGRFTISRDNSKNTLYLQMNSLRAEDTAVYYCAIYTGRWRPFEYWGQGTLVTVSS

SEQ ID NO: 126 - protein, 119 aa, Artificial Sequence
  Derived from a Human germline sequence
EVQLLESGGGLVQPGGSLRLSCAASGFTFFKYSMGWVRQAPGKGLEWVSQISDTADRTYYDHSVKGRFTISRDNSKNTLYLQMNSLRAEDTAVYYCAIYTGRWRPFEYWGQGTLVTVSS

SEQ ID NO: 127 - protein, 119 aa, Artificial Sequence
  Derived from a Human germline sequence
EVQLLESGGGLVQPGGSLRLSCAASGFTFFKYSMGWVRQAPGKGLEWVSQISDTADRTYYDHSVKGRFTISRDNSKNTLYLQMNSLRAEDTAVYYCAIYTGRWEPFVYWGQGTLVTVSS

SEQ ID NO: 128 - protein, 119 aa, Artificial Sequence
  Derived from a Human germline sequence
EVQLLESGGGLVQPGGSLRLSCAASGFTFLKYSMGWVRQAPGKGLEWVSQISDTADRTYYDHSVKGRFTISRDNSKNTLYLQMNSLRAEDTAVYYCAIYTGRWEPFVYWGQGTLVTVSS

SEQ ID NO: 129 - protein, 119 aa, Artificial Sequence
  Derived from a Human germline sequence
EVQLLESGGGLVQPGGSLRLSCAASGFTFLKYSMGWVRQAPGKGLEWVSQISDTADRTYYSHSVKGRFTISRDNSKNTLYLQMNSLRAEDTAVYYCAIYTGRWVPFEYWGQGTLVTVSS

SEQ ID NO: 130 - protein, 119 aa, Artificial Sequence
  Derived from a Human germline sequence
EVQLLESGGGLVQPGGSLRLSCAASGFTFFKYSMGWVRQAPGKGLEWVSQISDTADRTYYSHSVKGRFTISRDNSKNTLYLQMNSLRAEDTAVYYCAIYTGRWVPFEYWGQGTLVTVSS

SEQ ID NO: 131 - protein, 119 aa, Artificial Sequence
  Derived from a Human germline sequence
EVQLLESGGGLVQPGGSLRLSCAASGFTFFKYSMGWVRQAPGKGLEWVSQISDTADRTYYTHSVKGRFTISRDNSKNTLYLQMNSLRAEDTAVYYCAIYTGRWVPFEYWGQGTLVTVSS

SEQ ID NO: 132 - protein, 119 aa, Artificial Sequence
  Derived from a Human germline sequence
EVQLLESGGGLVQPGGSLRLSCAASGFTFLKYSMGWVRQAPGKGLEWVSQISDTADRTYYTHSVKGRFTISRDNSKNTLYLQMNSLRAEDTAVYYCAIYTGRWVPFEYWGQGTLVTVSS

SEQ ID NO: 133 - protein, 119 aa, Artificial Sequence; identical to SEQ ID NO: 106
  Derived from a Human germline sequence

SEQ ID NO: 134 - protein, 119 aa, Artificial Sequence; identical to SEQ ID NO: 107
  Derived from a Human germline sequence

SEQ ID NO: 135 - protein, 119 aa, Artificial Sequence
  Derived from a Human germline sequence
EVQLLESGGGLVQPGGSLRLSCAASGFTFLKYSMGWVRQAPGKGLEWVSQISDTGDRRYYDHSVKGRFTISRDNSKNTLYLQMNSLRAEDTAVYYCAIYTGRWAPFEYWGQGTLVTVSS

SEQ ID NO: 136 - protein, 119 aa, Artificial Sequence
  Derived from a Human germline sequence
EVQLLESGGGLVQPGGSLRLSCAASGFTFFKYSMGWVRQAPGKGLEWVSQISDTGDRRYYDHSVKGRFTISRDNSKNTLYLQMNSLRAEDTAVYYCAIYTGRWAPFEYWGQGTLVTVSS

SEQ ID NO: 137 - protein, 119 aa, Artificial Sequence
  Derived from a Human germline sequence
EVQLLESGGGLVQPGGSLRLSCAASGFTFVKYSMGWVRQAPGKGLEWVSQIADTADRTYYAHSVKGRFTISRDNSKNTLYLQMNSLRAEDTAVYYCAIYTGRWVPFEYWGQGTLVTVSS

SEQ ID NO: 138 - protein, 119 aa, Artificial Sequence
  Derived from a Human germline sequence
EVQLLESGGGLVQPGGSLRLSCAASGFTFFKYSMGWVRQAPGKGLEWVSQISDTADRTYYAHAVKGRFTISRDNSKNTLYLQMNSLRAEDTAVYYCAIYTGRWVPFEYWGQGTLVTVSS

SEQ ID NO: 139 - protein, 119 aa, Artificial Sequence
  Derived from a Human germline sequence
EVQLLESGGGLVQPGGSLRLSCAASGFTFVKYSMGWVRQAPGKGLEWVSQIADTADRTYYDHSVKGRFTISRDNSKNTLYLQMNSLRAEDTAVYYCAIYTGRWVPFEYWGQGTLVTVSS

SEQ ID NO: 140 - protein, 119 aa, Artificial Sequence
  Derived from a Human germline sequence
EVQLLESGGGLVQPGGSLRLSCAASGFTFVKYSMGWVRQAPGKGLEWVSQIADTADRTYYDHAVKGRFTISRDNSKNTLYLQMNSLRAEDTAVYYCAIYTGRWVPFEYWGQGTLVTVSS

SEQ ID NO: 141 - protein, 119 aa, Artificial Sequence
  Derived from a Human germline sequence
EVQLLESGGGLVQPGGSLRLSCAASGFTFVKYSMGWVRQAPGKGLEWVSQIADTADRRYYAHSVKGRFTISRDNSKNTLYLQMNSLRAEDTAVYYCAIYTGRWAPFEYWGQGTLVTVSS

SEQ ID NO: 142 - protein, 119 aa, Artificial Sequence
  Derived from a Human germline sequence
EVQLLESGGGLVQPGGSLRLSCAASGFTFVKYSMGWVRQAPGKGLEWVSQISDTADRRYYAHAVKGRFTISRDNSKNTLYLQMNSLRAEDTAVYYCAIYTGRWAPFEYWGQGTLVTVSS

SEQ ID NO: 143 - protein, 119 aa, Artificial Sequence
  Derived from a Human germline sequence
EVQLLESGGGLVQPGGSLRLSCAASGFTFVKYSMGWVRQAPGKGLEWVSQIADTADRRYYAHAVKGRFTISRDNSKNTLYLQMNSLRAEDTAVYYCAIYTGRWAPFEYWGQGTLVTVSS

SEQ ID NO: 144 - protein, 119 aa, Artificial Sequence
  Derived from a Human germline sequence
EVQLLESGGGLVQPGGSLRLSCAASGFTFVKYSMGWVRQAPGKGLEWVSQISDTADRRYYDHAVKGRFTISRDNSKNTLYLQMNSLRAEDTAVYYCAIYTGRWAPFEYWGQGTLVTVSS

SEQ ID NO: 145 - protein, 119 aa, Artificial Sequence
  Derived from a Human germline sequence
EVQLLESGGGLVQPGGSLRLSCAASGFTFVKYSMGWVRQAPGKGLEWVSQIADTADRRYYDHAVKGRFTISRDNSKNTLYLQMNSLRAEDTAVYYCAIYTGRWAPFEYWGQGTLVTVSS

SEQ ID NO: 146 - protein, 119 aa, Artificial Sequence
  Derived from a Human germline sequence
EVQLLESGGGLVQPGGSLRLSCAASGFTFVKYSMGWVRQAPGKGLEWVSQIADTADRRYYDHSVKGRFTISRDNSKNTLYLQMNSLRAEDTAVYYCAIYTGRWAPFEYWGQGTLVTVSS

SEQ ID NO: 147 - protein, 119 aa, Artificial Sequence
  Derived from a Human germline sequence
EVQLLESGGGLVQPGGSLRLSCAASGFTFFKYSMGWVRQAPGKGLEWVSQISDTADRRYYDDAVKGRFTITRDNSKNTLYLQMNSLRAEDTAVYYCAIYTGRWEPFVYWGQGTLVTVSS

SEQ ID NO: 148 - protein, 119 aa, Artificial Sequence
  Derived from a Human germline sequence
EVQLLESGGGLVQPGGSLRLSCAASGFTFVKYSMGWVRQAPGKGLEWVSQISDTADRTYYAHAVKGRFTISRDNSKNTLYLQMNSLRAEDTAVYYCAIYTGRWVPFEYWGQGTLVTVSS

SEQ ID NO: 149 - protein, 230 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 150 - protein, 230 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKR

SEQ ID NO: 151 - protein, 230 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 152 - protein, 230 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTDIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKR

SEQ ID NO: 153 - protein, 234 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 154 - protein, 234 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKR

SEQ ID NO: 155 - protein, 234 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 156 - protein, 234 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKR

SEQ ID NO: 157 - protein, 244 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASGGGGSGGGGSGGGGSDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 158 - protein, 244 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASGGGGSGGGGSGGGGSDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKR

SEQ ID NO: 159 - protein, 244 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 160 - protein, 244 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKR

SEQ ID NO: 161 - protein, 230 aa, Artificial Sequence
  Derived from a Human germline sequence
EVQLLESGGGLVQPGGSLRLSCAASGFTFVKYSMGWVRQAPGKGLEWVSQISDTADRTYYAHSVKGRFTISRDNSKNTLYLQMNSLRAEDTAVYYCAIYTGRWVPFEYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 162 - protein, 230 aa, Artificial Sequence
  Derived from a Human germline sequence
EVQLLESGGGLVQPGGSLRLSCAASGFTFFKYSMGWVRQAPGKGLEWVSQISDTADRTYYAHSVKGRFTISRDNSKNTLYLQMNSLRAEDTAVYYCAIYTGRWAPFEYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 163 - protein, 230 aa, Artificial Sequence
  Derived from a Human germline sequence
EVQLLESGGGLVQPGGSLRLSCAASGFTFFKYSMGWVRQAPGKGLEWVSQISDTADRTYYAHSVKGRFTISRDNSKNTLYLQMNSLRAEDTAVYYCAIYTGRWVPFEYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 164 - protein, 230 aa, Artificial Sequence
  Derived from a Human germline sequence
EVQLLESGGGLVQPGGSLRLSCAASGFTFFKYSMGWVRQAPGKGLEWVSQISDTADRTYYSHSVKGRFTISRDNSKNTLYLQMNSLRAEDTAVYYCAIYTGRWVPFEYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 165 - protein, 230 aa, Artificial Sequence
  Derived from a Human germline sequence
EVQLLESGGGLVQPGGSLRLSCAASGFTFVKYSMGWVRQAPGKGLEWVSQISDTADRTYYAHAVKGRFTISRDNSKNTLYLQMNSLRAEDTAVYYCAIYTGRWVPFEYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 166 - protein, 119 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSC

SEQ ID NO: 167 - protein, 248 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKRAAAEQKLISEEDLN

SEQ ID NO: 168 - protein, 234 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 169 - protein, 248 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKRAAAEQKLISEEDLN

SEQ ID NO: 170 - protein, 234 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 171 - protein, 248 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKRAAAEQKLISEEDLN

SEQ ID NO: 172 - protein, 234 aa, Artificial Sequence; identical to SEQ ID NO: 154
  Derived from a Human germline sequence

SEQ ID NO: 173 - protein, 227 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSDIQMTQSPSSLSASVGDRVTITCRASRPIGTTLSWYQQKPGKAPKLLIWFGSRLQSGVPSRFSGSGSGTDFTLTISSLQPEDFATYYCAQAGTHPTTFGQGTKVEIKR

SEQ ID NO: 174 - protein, 227 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 175 - protein, 244 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASRPIGTTLSWYQQKPGKAPKLLIWFGSRLQSGVPSRFSGSGSGTDFTLTISSLQPEDFATYYCAQAGTHPTTFGQGTKVEIKRAAAEQKLISEEDLN

SEQ ID NO: 176 - protein, 230 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASRPIGTTLSWYQQKPGKAPKLLIWFGSRLQSGVPSRFSGSGSGTDFTLTISSLQPEDFATYYCAQAGTHPTTFGQGTKVEIKR

SEQ ID NO: 177 - protein, 234 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASRPIGTTLSWYQQKPGKAPKLLIWFGSRLQSGVPSRFSGSGSGTDFTLTISSLQPEDFATYYCAQAGTHPTTFGQGTKVEIKR

SEQ ID NO: 178 - protein, 244 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKRAAAEQKLISEEDLN

SEQ ID NO: 179 - protein, 230 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKR

SEQ ID NO: 180 - protein, 248 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKRAAAEQKLISEEDLN

SEQ ID NO: 181 - protein, 234 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKR

SEQ ID NO: 182 - protein, 244 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKRAAAEQKLISEEDLN

SEQ ID NO: 183 - protein, 230 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 184 - protein, 248 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKRAAAEQKLISEEDLN

SEQ ID NO: 185 - protein, 234 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 186 - protein, 244 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASQSIIKHLKWYQQKPGKAPKLLIYGASRLQSGVPSRFSGSGSGTDFTLTISSLQPEDFATYYCQQGTRWPQTFGQGTKVEIKRAAAEQKLISEEDLN

SEQ ID NO: 187 - protein, 230 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTDIQMTQSPSSLSASVGDRVTITCRASQSIIKHLKWYQQKPGKAPKLLIYGASRLQSGVPSRFSGSGSGTDFTLTISSLQPEDFATYYCQQGTRWPQTFGQGTKVEIKR

SEQ ID NO: 188 - protein, 248 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKRAAAEQKLISEEDLN

SEQ ID NO: 189 - protein, 234 aa, Artificial Sequence
  Derived from a Human germline sequence
EVQLLESGGGLVQPGGSLRLSCAASGFTFVKYSMGWVRQAPGKGPEWVSQISNTGDRTYYADSVKGRFTISRDNSKNTLYLQMNSLRAEDTAVYYCAIYTGRWEPFDYWGQGTLVTVSSASTSGPSDIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 190 - protein, 234 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 191 - protein, 234 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAKYTGHWEPFDYWGQGTLVTVSSASTSGPSDIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 192 - protein, 230 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTDIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 193 - protein, 244 aa, Artificial Sequence
  Derived from a Human germline sequence
EVQLLESGGGLVQPGGSLRLSCAASGFTFVKYSMGWVRQAPGKGLEWVSQISNTADRTYYAHSVKGRFTISRDNSKNTLYLQMNSLRAEDTAVYYCAIYTGRWVPFEYWGQGTLVTVSSASTDIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKRAAAEQKLISEEDLN

SEQ ID NO: 194 - protein, 230 aa, Artificial Sequence; identical to SEQ ID NO: 192
  Derived from a Human germline sequence

SEQ ID NO: 195 - protein, 234 aa, Artificial Sequence
  Derived from a Human germline sequence
EVQLLESGGGLVQPGGSLRLSCAASGFTFVKYSMGWVRQAPGKGLEWVSQISNTGGHTYYADSVKGRFTISRDNSKNTLYLQMNSLRAEDTAVYYCAKYTGHWEPFDYWGQGTLVTVSSASTSGPSDIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 196 - protein, 234 aa, Artificial Sequence
  Derived from a Human germline sequence
EVQLLESGGGLVQPGGSLRLSCAASGFTFFKYSMGWVRQAPGKGLEWVSQISDTADRTYYAHSVKGRFTISRDNSKNTLYLQMNSLRAEDTAVYYCAIYTGRWVPFEYWGQGTLVTVSSASTSGPSDIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKR

SEQ ID NO: 197 - protein, 108 aa, Artificial Sequence
  Derived from a Human germline sequence
DIQMTQSPSSLSASVGDRVTITCRASRPIGTTLSWYQQKPGKAPKLLIWFGSRLQSGVPSRFSGSGSGTDFTLTISSLQPEDFATYYCAQAGTHPTTFGQGTKVEIKR

SEQ ID NO: 198 - protein, 108 aa, Artificial Sequence
  Derived from a Human germline sequence
DIQMTQSPSSLSASVGDRVTITCRASRPIGTTLSWYQQKPGKAPKLLILWNSRLQSGVPSRFSGSGSGTDFTLTISSLQPEDFATYYCAQAGTHPTTFGQGTKVEIKR

SEQ ID NO: 199 - protein, 108 aa, Artificial Sequence
  Derived from a Human germline sequence
DIQMTQSPSSLSASVGDRVTITCRASRPIGTMLSWYQQKPGKAPKLLILFGSRLQSGVPSRFSGSGSGTDFTLTISSLQPEDFATYYCAQAGTHPTTFGQGTKVEIKR

SEQ ID NO: 200 - protein, 108 aa, Artificial Sequence
  Derived from a Human germline sequence
DIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 201 - protein, 108 aa, Artificial Sequence
  Derived from a Human germline sequence
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 202 - protein, 108 aa, Artificial Sequence
  Derived from a Human germline sequence
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 203 - protein, 108 aa, Artificial Sequence
  Derived from a Human germline sequence
DIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKR

SEQ ID NO: 204 - protein, 108 aa, Artificial Sequence
  Derived from a Human germline sequence
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 205 - protein, 119 aa, Artificial Sequence
  Derived from a Human germline sequence
EVQLLESGGGLVQPGGSLRLSCAASGFTFAHETMVWVRQAPGKGLEWVSHIPPVGQDPFYADSVKGRFTISRDNSKNTLYLQMNSLRAEDTAVYYCALLPKRGPWFDYWGQGTLVTVSS

SEQ ID NO: 206 - protein, 120 aa, Artificial Sequence
  Derived from a Human germline sequence
EVQLLESGGGLVQPGGSLRLSCAASGFTFNRYSMGWLRQAPGKGLEWVSRIDSYGRGTYYEDPVKGRFSISRDNSKNTLYLQMNSLRAEDTAVYYCAKISQFGSNAFDYWGQGTQVTVSS

SEQ ID NO: 207 - protein, 108 aa, Artificial Sequence
  Derived from a Human germline sequence
DIQMTQSPSSLSASVGDRVTITCRASQYIHTSVQWYQQKPGKAPKLLIYGSSRLHSGVPSRFSGSGSGTDFTLTISSLQPEDFATYYCQQNHYSPFTYGQGTKVEIKR

SEQ ID NO: 208 - DNA, 360 bp, Artificial Sequence
  Derived from a Human germline sequence
gaggtgcagctgttggagtctgggggaggcttggtacagcctggggggtccctgcgtctctcctgtgcagcctccggattcacctttagtcagtataggatgcattgggtccgccaggctccagggaagagtctagagtgggtctcaagtattgatactaggggttcgtctacatactacgcagaccccgtgaagggccggttcaccatctcccgcgacaattccaagaacacgctgtatctgcaaatgaacagcctgcgtgccgaggacaccgcggtatattactgtgcgaaagctgtgacgatgttttctcctttttttgactactggggtcagggaaccctggtcaccgtctcgagc

SEQ ID NO: 209 - DNA, 369 bp, Artificial Sequence
  Derived from a Human germline sequence
gaggtgcagctgttggagtctgggggaggcttggtacagcctggggggtccctgcgtctctcctgtgcagcctccggattcacctttgctgattatgggatgcgttgggtccgccaggctccagggaagggtctagagtgggtctcatctattacgcggactggtcgtgttacatactacgcagactccgtgaagggccggttcaccatctcccgcgacaattccaagaacacgctgtatctgcaaatgaacagcctgcgtgccgaggacaccgcggtatattactgtgcgaaatggcggaatcggcatggtgagtatcttgctgattttgactactggggtcagggaaccctggtcaccgtctcgagc

SEQ ID NO: 210 - DNA, 360 bp, Artificial Sequence
  Derived from a Human germline sequence
gaggtgcagctgttggagtctgggggaggcttggtacagcctggggggtccctgcgtctctcctgtgcagcctccggattcacctttatgaggtataggatgcattgggtccgccaggctccagggaagggtctagagtgggtctcatcgattgattctaatggttctagtacatactacgcagactccgtgaagggccggttcaccatctcccgcgacaattccaagaacacgctgtatctgcaaatgaacagcctgcgtgccgaggacaccgcggtatattactgtgcgaaagatcgtacggagcgttcgccggtttttgactactggggtcagggaaccctggtcaccgtctcgagc

SEQ ID NO: 211 - DNA, 357 bp, Artificial Sequence
  Derived from a Human germline sequence
gaggtgcagctgttggagtctgggggaggcttggtgcagcctggggggtccctgcgtctctcctgtgcagcctccggattcacctttgttgattatgagatgcattgggtccgccaggctccagggaagggtctagagtgggtctcatctattagtgagagtggtacgacgacatactacgcagactccgtgaagggccggttcaccatctcccgcgacaattccaagaacacgctgtatctgcaaatgaacagcctgcgtgccgaggacaccgcggtatattactgtgcgaaacgtcgtttttctgcttctacgtttgactactggggtcagggaaccctggtcaccgtctcgagc

SEQ ID NO: 212 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgaaatatacgggtcattgggagccttttgactactggggtcagggaaccctggtcaccgtctcgagc

SEQ ID NO: 213 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgaaatatacgggtcgttgggagccttatgactactggggtcagggaaccctggtcaccgtctcgagc

SEQ ID NO: 214 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgaaatatacgggtcgttgggagccttttgactactggggtcagggaaccctggtcaccgtctcgagc

SEQ ID NO: 215 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgaaatatacgggtcgttgggagccttttgagtactggggtcagggaaccctggtcaccgtctcgagc

SEQ ID NO: 216 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 217 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagc

SEQ ID NO: 218 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatatcccgcgacaattccaagaacacgctgtatatgcaaatgaacagcctgcgtgccgaggacaccgcggtatattactgtgcgatatatacgggtcgttgggagccttttgactactggggtcagggaaccctggtcaccgtctcgagc

SEQ ID NO: 219 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttggtaagtattcgatggggtgggtccgccaggctccagggaaggatctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 220 - DNA, 357 bp, Artificial Sequence
  Derived from a Human germline sequence
gaggtgcagctgttggagtcagggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgaaatatacgggtcgttgggagccttttgaccactggggtcaggggaccctggtcaccgtctcgagc

SEQ ID NO: 221 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcatacatactacgcagactccgtgaagggccggttcaccatctcccgcgacaattccaagaacacgctgtatctgcaaatgaacagcctgcgtgccgaggacaccgcggtatattactgtgcgaaatatacgggtcgttgggagccttttgactactggggtcagggaaccctggtcaccgtctcgagc

SEQ ID NO: 222 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgaaatatacgggtcgttgggagccttttgactactggggtcagggaaccctggtcaccgtctcgagc

SEQ ID NO: 223 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 224 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcatacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 225 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagc

SEQ ID NO: 226 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcatacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagc

SEQ ID NO: 227 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttggtaagtattcgatggggtgggtccgccaggctccagggaaggatctagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 228 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttggtaagtattcgatggggtgggtccgccaggctccagggaaggatctagagtgggtctcacagatttcgaatacgggtgatcatacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 229 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 230 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgctgaggacaccgcggtatattactgtgcgatatatacgggtcgttgggagccttttgagtactggggtcagggaaccctggtcaccgtctcgagc

SEQ ID NO: 231 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcggactccgtgaagggccggttcaccatctcccgcgacaattccaagaacacgctgtatctgcaaatgaacagcctgcgtgctgaggacaccgcggtatattactgtgcgatatatacgggtcgttggaagccttttgagtactggggtcagggaaccctggtcaccgtctcgagc

SEQ ID NO: 232 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 233 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 234 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgccgaggacaccgcggcatattactgtgcgatatatacgggtcgttgggagccttttgactactggggtcagggaaccctggtcaccgtctcgagc

SEQ ID NO: 235 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggagccttttaactactggggtcagggaaccctggtcaccgtctcgagc

SEQ ID NO: 236 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 237 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaactcgctgtatctgcaaatgaacagcctgcgtgccgaggacaccgcggtatattactgtgcgatatatacgggtcgttgggtgccttttgacaactggggtcagggaaccctggtcaccgtctcgagc

SEQ ID NO: 238 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttattacgtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttcagtactggggtcagggaaccctggtcaccgtctcgagc

SEQ ID NO: 239 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttggtaagtattcgatggggtgggtccgccaggctccagggaagggtctagagtgggtctcacagatttcgaatacgggtgatcgtacatactacgcggactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 240 - DNA, 357 bp, Artificial Sequence
  Derived from a Human germline sequence
gaggtgcagctgttggagtctgggggaggcttggtacagcctggggggtccctgcgtctctcctgtgcagcctccggattcaccttttttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgccgaagacaccgcggtatattactgtgcgatatatacgggtcgttgggagccttttgactactggggtcagggaaccctggtcaccgtctcgagc

SEQ ID NO: 241 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 242 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 243 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtacatactacgcagactccgtgaagggccggttcaccatctcccgcgacaattccaagaacacgctgtatctgcaaatgaacagcctgcgtgctgaggacaccgcggtatattactgtgcgatatatacgggtcgttgggagccttttaagtactggggtcagggaaccctggtcaccgtctcgagc

SEQ ID NO: 244 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttagtaagtattcgatggggtgggtccgccaggctccagggaagggtctagagtgggtctcacagatttcgaatacgggtgagcgtagatactacgcagactcagtgaagggccggttcaccatctcccgcgacaatcccaagaacacgctgtatctgcaaatgaacagcctgcgtgccgaggacaccgcggtatattactgtgcgatatatacgggtcggtgggagccttttgaatactggggtcagggaaccctggtcaccgtctcgagc

SEQ ID NO: 245 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaactattcgatggggtgggtccgccaggctccagggaagggtctagagtgggtctcacagatttcgaatacgggtgatcgtacatactacgcggactccgtgaagggccggttcaccatctcccgcgacaattccaagaacacgctgtatctgcaaatgaacagcctgcgtgccgaggacaccgcggtatattactgtgcgatatatacgggtcgttgggagccttatgagtactggggtcagggaaccctggtcaccgtcacgagc

SEQ ID NO: 246 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgataattccaagaacacactgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 247 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgccgaggacaccgcggtatattactgtgcgatatatacgggtcgttggaagccttttgagtactggggtcagggaaccctggtcaccgtctcgagc

SEQ ID NO: 248 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 249 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 250 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 251 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcggtatatacgggtcgttgggagccttttgtctactggggtcagggaaccctggtcaccgtctcgagc

SEQ ID NO: 252 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatacgggtcgttggaagccttttgagtactggggtcagggaaccctggtcaccgtctcgagc

SEQ ID NO: 253 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 254 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 255 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 256 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 257 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggccccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgctgaggacaccgcggtatattactgtgcgatatatacgggtcgttggaagccttttgagtactggggtcagggaaccctggtcaccgtctcgagc

SEQ ID NO: 258 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatactgggcgttgggtgccttttgagtactggggtcagggaaccctggtcaccgtctcgagc

SEQ ID NO: 259 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 260 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 261 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 262 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtagatactacgcagacgcggtgaaggggcggttcaccatctcccgcgacaattccaagaacacgctgtatctgcaaatgaacagcctgcgtgctgaggacaccgcggtatattactgtgcgatatatacgggtcgttggaagccttttgagtactggggtcagggaaccctggtcaccgtctcgagc

SEQ ID NO: 263 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggccccagggaagggtctagagtgggtctcacagatttcgaatacgggtgatcgtagatactacgcagacgcggtgaaggggcggttcaccatctcccgcgacaattccaagaacacgctgtatctgcaaatgaacagcctgcgtgccgaagacaccgcggtatattactgtgcgatatatactgggcgttgggtgccttttgagtactggggtcagggaaccctggtcaccgtctcgagc

SEQ ID NO: 264 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 265 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 266 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagttttcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 267 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 268 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 269 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 270 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgccgaggacaccgcggcatattactgtgcgatatatacgggtcggtggcccgactttgactactggggtcagggaaccctggtcaccgtctcgagc

SEQ ID NO: 271 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagactctgtgaagggccggttcaccatctcccgcgacaattccaagaacacgctgtatctgcaaatgaacagcctgcgtgccgaggacaccgcggcatattactgtgcgatatatacgggtcggtggcccgactttgagtactggggtcagggaaccctggtcaccgtctcgagc

SEQ ID NO: 272 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtggcccgactttgactactggggtcagggaaccctggtcaccgtctcgagc

SEQ ID NO: 273 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtggcccgactttgagtactggggtcagggaaccctggtcaccgtctcgagc

SEQ ID NO: 274 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcggtggcccgactttgactactggggtcagggaaccctggtcaccgtctcgagc

SEQ ID NO: 275 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcggtggcccgactttgagtactggggtcagggaaccctggtcaccgtctcgagc

SEQ ID NO: 276 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcggcctggggtgacaggacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 277 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaaaggtccagagtgggtctcacagatttcggacggcggtcagaggacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 278 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcggactccggttaccgcacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 279 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtccagagtgggtctcacagatttcggacgggggtacgcggacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 280 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcggacaagggtacgcgcacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 281 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcggagaccggtcgcaggacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 282 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattaacaatacgggttcgaccacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagc

SEQ ID NO: 283 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtccagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 284 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtccagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 285 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 286 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 287 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgatgactctgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 288 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacacacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 289 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgcagatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 290 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattttgaatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 291 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 292 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtagatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 293 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtagatactacgatcactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 294 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcggtatatactgggcgttgggtgtcttttgagtactggggtcagggaaccctggtcaccgtctcgagc

SEQ ID NO: 295 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgctatatactgggcgttgggtgtcttttgagtactggggtcagggaaccctggtcaccgtctcgagc

SEQ ID NO: 296 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggtttaccatctcccgcgacaattccaagaacacgctgtatctgcaaatgaacagcctgcgtgccgaggacaccgcggtatattactgtgcggtatatactgggcgttgggtgccttttgagtactggggtcagggaaccctggtcaccgtctcgagc

SEQ ID NO: 297 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgccgaggacaccgcggtatattactgtgcgctatatactgggcgttgggtgccttttgagtactggggtcagggaaccctggtcaccgtctcgagc

SEQ ID NO: 298 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatactgctgatcgtagatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 299 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 300 - DNA, 357 bp, Artificial Sequence
  Derived from a Human germline sequence
gaggtgcagctgttggagtctgggggaggcttggtacagcctggggggtccctgcgtctctcctgtgcggcctccggattcacctttgttaagtattcgatggggtgggtccgccaggctccagggaagggtctagagtgggtctcacagatttcgaatacgggcgatcgtagatactacgcacacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 301 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatactgctgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 302 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcacacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 303 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtagatactacgcacacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 304 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacggctgatcgtagatactacgcacacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 305 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgtgaatacgggtgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 306 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcgaatacgggtgatcgtagatactacgcagacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 307 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 308 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 309 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 310 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactactcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 311 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacacacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 312 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacacagacgcggtgaaggggcggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 313 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 314 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 315 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagattgcggatacgggtgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 316 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 317 - DNA, 357 bp, Artificial Sequence
  Derived from a Human germline sequence
gaggtgcagctgttggagtctgggggaggcttggtacagcctggggggtccctgcgcctctcctgtgcagcctccggattcacctttttcaagtattcgatggggtgggtccgccaggctccagggaagggtctagagtgggtctcacagatttcggatacgggtgatcgtagatactacgatcactctgtgaagggccggttcaccatctcccgcgacaattccaagaacacgctgtatctgcaaatgaacagcctgcgtgccgaggacaccgcggtatattactgtgcgatatatacgggtcgttgggaaccttttgtctactggggtcagggaaccctggtcaccgtctcgagc

SEQ ID NO: 318 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatacgggtgatcgtagatactacgatgacgcggtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 319 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagattgcggatactgctgatcgtagatactacgatgactctgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 320 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagattgcggatacgggtgatcgtagatactacgatcactctgtgaagggccggttcactatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 321 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagattgcggatacgggtgatcgtagatactacgatgacgcggtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 322 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggggccttttgtctactggggtcagggaaccctggtcaccgtctcgagc

SEQ ID NO: 323 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggtgccttttgcctactggggtcagggaaccctggtcaccgtctcgagc

SEQ ID NO: 324 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttggggaccttttcagtactggggtcagggaaccctggtcaccgtctcgagc

SEQ ID NO: 325 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggagccttttcagtactggggtcagggaactctggtcaccgtctcgagc

SEQ ID NO: 326 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggcgccttttgagtactggggtcagggaaccctggtcaccgtctcgagc

SEQ ID NO: 327 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggcgccttttcagtactggggtcagggaactctggtcaccgtctcgagc

SEQ ID NO: 328 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggtgccttttcagtactggggtcagggcaccctggtcaccgtctcgagc

SEQ ID NO: 329 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggataccggtgatcgtagatactacgatcactctgtgaagggccggttcactatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 330 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 331 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 332 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 333 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcgttggaggccttttgagtactggggtcagggaaccctggtcaccgtctcgagc

SEQ ID NO: 334 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 335 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatactgctgatcgtacatactacgatcactccgtgaagggccggttcaccatctcccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 336 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatactgctgatcgtacatactactcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 337 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactactcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 338 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacacacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 339 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatactgctgatcgtacatactacacacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 340 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggcgccttttgagtactggggtcagggaaccctggtcaccgtctcgagc

SEQ ID NO: 341 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcgttgggcgccttttgagtactggggtcagggaaccctggtcaccgtctcgagc

SEQ ID NO: 342 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttgaagtattcgatggggtgggtccgccaggctccagggaagggtctagagtgggtctcacagatttcggataccggtgatcgtagatactacgatcactctgtgaagggccggttcactatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 343 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggataccggtgatcgtagatactacgatcactctgtgaagggccggttcactatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 344 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcggatactgctgatcgtacatactacgcacactccgtgaagggccggttcaccatctcccgcgacaattccaagaacacgctgtatctgcaaatgaacagcctgcgtgctgaggacaccgcggtatattactgcgcgatatatactgggcgttgggtgccttttgagtactggggtcagggaaccctggtcaccgtctcgagc

SEQ ID NO: 345 - DNA, 357 bp, Artificial Sequence
  Derived from a Human germline sequence
gaggtgcagctgttggagtctgggggaggcttggtacagcctggggggtccctgcgtctctcctgtgcagcctccggattcaccttttttaagtattcgatggggtgggtccgccaggctccagggaagggtctagagtgggtctcacagatttcggatactgctgatcgtacatactacgcacacgcggtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 346 - DNA, 357 bp, Artificial Sequence
  Derived from a Human germline sequence
gaggtgcagctgttggagtctgggggaggcttggtgcagcctggggggtccctgcgtctctcctgtgcagcctccggattcacctttgttaagtattcgatggggtgggtccgccaggctccagggaagggtctagagtgggtctcacagattgcggatactgctgatcgtacatactacgatcactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 347 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcggatactgctgatcgtacatactacgatcacgcggtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 348 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcggatactgctgatcgtagatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 349 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtagatactacgcacacgcggtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 350 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcggatactgctgatcgtagatactacgcacacgcggtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 351 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtagatactacgatcacgcggtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 352 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcggatactgctgatcgtagatactacgatcacgcggtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcaggggaccctggtcaccgtctcgagc

SEQ ID NO: 353 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagattgcggatactgctgatcgtagatactacgatcactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagc

SEQ ID NO: 354 - DNA, 357 bp, Artificial Sequence
  Derived from a Human germline sequence
gaggtgcagctgctggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtagatactacgatgacgcggtgaagggccggttcaccatcacccgcgacaattccaagaacacgctgtatctgcaaatgaacagcctgcgtgccgaggacaccgcggtatattactgtgcgatatatacgggtcgttgggagccttttgtctactggggtcagggaaccctggtcaccgtctcgagc

SEQ ID NO: 355 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacacgcggtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagc

SEQ ID NO: 356 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 357 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 358 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 359 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgg

SEQ ID NO: 360 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 361 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 362 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 363 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgg

SEQ ID NO: 364 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcggtggaggcggttcaggcggaggtggcagcggcggtggcggatccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 365 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcggtggaggcggttcaggcggaggtggcagcggcggtggcggatccgacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 366 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcggtggaggcggttcaggcggaggtggcagcggcggtggcggatccgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 367 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcggtggaggcggttcaggcggaggtggcagcggcggtggcggatccgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgg

SEQ ID NO: 368 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 369 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgccgaggacaccgcggtatattactgtgcgatatatacgggtcggtgggcgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 370 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 371 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactactcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 372 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcggatactgctgatcgtacatactacgcacacgcggtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 373 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgtgt

SEQ ID NO: 374 - DNA, 744 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgggcggccgcagaacaaaaactcatctcagaagaggatctgaat

SEQ ID NO: 375 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 376 - DNA, 744 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgggcggccgcagaacaaaaactcatctcagaagaggatctgaat

SEQ ID NO: 377 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgg

SEQ ID NO: 378 - DNA, 744 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgggcggccgcagaacaaaaactcatctcagaagaggatctgaat

SEQ ID NO: 379 - DNA, 702 bp, Artificial Sequence; identical to SEQ ID NO: 361
  Derived from a Human germline sequence

SEQ ID NO: 380 - DNA, 681 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatctggtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 381 - DNA, 681 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 382 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatctggtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgggcggccgcagaacaaaaactcatctcagaagaggatctgaat

SEQ ID NO: 383 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatctggtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 384 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccagtggtccatcggacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatctggtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 385 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccgacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgggcggccgcagaacaaaaactcatctcagaagaggatctgaat

SEQ ID NO: 386 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccgacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 387 - DNA, 744 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgggcggccgcagaacaaaaactcatctcagaagaggatctgaat

SEQ ID NO: 388 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 389 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgggcggccgcagaacaaaaactcatctcagaagaggatctgaat

SEQ ID NO: 390 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccgacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 391 - DNA, 744 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccagtggtccatcggacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgggcggccgcagaacaaaaactcatctcagaagaggatctgaat

SEQ ID NO: 392 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcacagtctcgagcgctagcaccagtggtccatcggacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 393 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagcgctagcaccgacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacaggggactcggtggcctcagacgttcggccaagggaccaaggtggaaatcaaacgggcggccgcagaacaaaaactcatctcagaagaggatctgaat

SEQ ID NO: 394 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagcgctagcaccgacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacaggggactcggtggcctcagacgttcggccaagggaccaaggtggaaatcaaacgg

SEQ ID NO: 395 - DNA, 744 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagcgctagcaccagtggtccatcggacatccagatgacccagtctccatcctccctgtctgcatctgtaggtgaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgggcggccgcagaacaaaaactcatctcagaagaggatctgaat

SEQ ID NO: 396 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatgggatgggtccgccaggctccagggaaaggtccagagtgggtctcacagatttcgaatacgggtgatcgtacatactacgcagactccgtgaagggccggttcaccatctcccgcgacaattccaagaacacgctgtatctgcaaatgaacagcctgcgtgccgaggacaccgcggtatattactgtgcgatatatacgggtcgttgggagccttttgactactggggtcagggaaccctggtcaccgtctcgagcgctagcaccagtggtccatcggacatccagatgacccagtctccatcctccctgtctgcatctgtaggtgaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgg

SEQ ID NO: 397 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 398 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgaaatatacgggtcattgggagccttttgactactggggtcagggaaccctggtcaccgtctcgagcgctagcaccagtggtccatcggacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 399 - DNA, 690 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 400 - DNA, 732 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccgacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgggcggccgcagaacaaaaactcatctcagaagaggatctgaat

SEQ ID NO: 401 - DNA, 690 bp, Artificial Sequence; identical to SEQ ID NO: 399
  Derived from a Human germline sequence

SEQ ID NO: 402 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttgttaagtattcgatggggtgggtccgccaggctccagggaagggtctagagtgggtctcacagatttcgaatacgggtggtcatacatactacgcagactccgtgaagggccggttcaccatctcccgcgacaattccaagaacacgctgtatctgcaaatgaacagcctgcgtgccgaggacaccgcggtatattactgtgcgaaatatacgggtcattgggagccttttgactactggggtcagggaaccctggtcaccgtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 403 - DNA, 702 bp, Artificial Sequence
  Derived from a Human germline sequence
gaggtgcagctgttggagtctgggggaggcttggtacagcctggggggtccctgcgtctctcctgtgcagcctccggattcacctttttcaagtattcgatggggtgggtccgccaggctccagggaagggtctagagtgggtctcacagatttcggatactgctgatcgtacatactacgcacactccgtgaagggccggttcaccatctcccgcgacaattccaagaacacgctgtatctgcaaatgaacagcctgcgtgctgaggacaccgcggtatattactgtgcgatatatactgggcgttgggtgccttttgagtactggggtcagggaaccctggtcaccgtctcgagcgctagcaccagtggtccatcggacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 404 - DNA, 324 bp, Artificial Sequence
  Derived from a Human germline sequence
gacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatctggtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 405 - DNA, 324 bp, Artificial Sequence
  Derived from a Human germline sequence
gacatccagatgacccagtctccatcctccctgtctgcatctgtaggagaccgtgtcaccatcacttgccgggcaagtcgtccgattgggacgacgttaagttggtaccagcagaaaccagggaaagcccctaagctcctgatcctttggaattcccgtttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 406 - DNA, 324 bp, Artificial Sequence
  Derived from a Human germline sequence
gacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatcttgtttggttcccggttgcaaagtggggtcccatcacgtttcagtggcagtggatctgggacagatttcactctcaccatcagcagtctgcaacctgaagattttgctacgtactactgtgcgcaggctgggacgcatcctacgacgttcggccaagggaccaaggtggaaatcaaacgg

SEQ ID NO: 407 - DNA, 324 bp, Artificial Sequence
  Derived from a Human germline sequence
gacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 408 - DNA, 324 bp, Artificial Sequence
  Derived from a Human germline sequence
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 409 - DNA, 324 bp, Artificial Sequence
  Derived from a Human germline sequence
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 410 - DNA, 324 bp, Artificial Sequence
  Derived from a Human germline sequence
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgg

SEQ ID NO: 411 - DNA, 324 bp, Artificial Sequence
  Derived from a Human germline sequence
gacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgg

SEQ ID NO: 412 - DNA, 357 bp, Artificial Sequence
  Derived from a Human germline sequence
gaggtgcagctgttggagtctgggggaggcttggtacagcctggggggtccctgcgtctctcctgtgcagcctccggattcacctttgcgcatgagacgatggtgtgggtccgccaggctccagggaagggtctagagtgggtctcacatattcccccggttggtcaggatcccttctacgcagactccgtgaagggccggttcaccatctcccgcgacaattccaagaacacgctatatctgcaaatgaacagcctgcgtgccgaggacacagcggtatattactgtgcgctgcttcctaagagggggccttggtttgactactggggtcagggaaccctggtcaccgtctcgagc

SEQ ID NO: 413 - DNA, 360 bp, Artificial Sequence
  Derived from a Human germline sequence
gaggtgcagctgttggagtctgggggaggcttggtacagcctggggggtccctgcgtctctcctgtgcagcctccggattcacctttaataggtatagtatggggtggctccgccaggctccagggaagggtctagagtgggtctcacggattgattcttatggtcgtggtacatactacgaagaccccgtgaagggccggttcagcatctcccgcgacaattccaagaacacgctgtatctgcaaatgaacagcctgcgtgccgaggacaccgccgtatattactgtgcgaaaatttctcagtttgggtcaaatgcgtttgactactggggtcagggaacccaggtcaccgtctcgagc

SEQ ID NO: 414 - DNA, 324 bp, Artificial Sequence
  Derived from a Human germline sequence
gacatccagatgacccagtctccatcctccctgtctgcatctgtaggagaccgtgtcaccatcacttgccgggcaagtcagtatattcatacgagtgtacagtggtaccagcagaaaccagggaaagcccctaaactcctgatctatgggtcgtccaggttgcatagtggggtcccatcacgtttcagtggcagtggatctgggacagatttcactctcaccatcagcagtctgcaacctgaagattttgctacgtactactgtcaacagaatcattatagtccttttacgtacggccaagggaccaaggtggaaatcaaacgg

SEQ ID NO: 415 - protein, 21 aa, Artificial Sequence
  Primer
CAGGAAACAGCTATGACCATG

SEQ ID NO: 416 - protein, 20 aa, Artificial Sequence
  Primer
TTGTAAAACGACGGCCAGTG

SEQ ID NO: 417 - protein, 20 aa, Artificial Sequence
  Primer
TTCAGGCTGCGCAACTGTTG

SEQ ID NO: 418 - protein, 22 aa, Artificial Sequence
  Primer
CGCCAAGCTTGCATGCAAATTC

SEQ ID NO: 419 - protein, 57 aa, Artificial Sequence
  Primer
CCTGTGCAGCCTCCGGATTCACCTTTGTTAAGTATTCGATGGGGTGGGTCCGCCAGG

SEQ ID NO: 420 - protein, 95 aa, Artificial Sequence
  Primer
TCCAGGGAAGGGTCTAGAGTGGGTCTCACAGATTTCGAATACGGGTGATCGTACATACTACGCAGACTCCGTGAAGGGCCGGTTCACCATCTCCC

SEQ ID NO: 421 - protein, 81 aa, Artificial Sequence
  Primer
GAGGACACCGCGGTATATTACTGTGCGATATATACGGGTCGTTGGGAGCCTTTTGACTACTGGGGTCAGGGAACCCTGGTC

SEQ ID NO: 422 - protein, 26 aa, Artificial Sequence
  Primer
AAAGGTGAATCCGGAGGCTGCACAGG

SEQ ID NO: 423 - protein, 28 aa, Artificial Sequence
  Primer
TGAGACCCACTCTAGACCCTTCCCTGGA

SEQ ID NO: 424 - protein, 27 aa, Artificial Sequence
  Primer
CGCACAGTAATATACCGCGGTGTCCTC

SEQ ID NO: 425 - protein, 155 aa, Homo Sapiens
VRSSSRTPSDKPVAHVVANPQAEGQLQWLNRRANALLANGVELRDNQLVVPSEGLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 426 - protein, 189 aa, Homo Sapiens
MIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSG

SEQ ID NO: 427 - protein, 192 aa, Homo Sapiens
MLMGIHPSGVTGLVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDS

SEQ ID NO: 428 - protein, 169 aa, Cynomolgus
DSVCPQGKYIHPQNNSVCCTKCHKGTYLYNDCPGPGQDTDCRECESGSFTASENHLRHCLSCSKCRKEMGQVEISSCTVDRDTVCGCRKNQYRYYWSENLFQCFNCSLCLNGTVHLSCQEKQNTVCTCHAGFFLRENECVSCSNCKKTLECTKLCLPQTENVKGTEDSG

SEQ ID NO: 429 - protein, 155 aa, Murine
MLRSSSQNSSDKPVAHVVANHQVEEQLEWLSQRANALLANGMDLKDNQLVVPADGLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 430 - protein, 15 aa, Homo sapiens
NSICCTKCHKGTYLY

SEQ ID NO: 431 - protein, 14 aa, Homo sapiens
NSICCTKCHKGTYL

SEQ ID NO: 432 - protein, 15 aa, Homo sapiens
CRKNQYRHYWSENLF

SEQ ID NO: 433 - protein, 15 aa, Homo sapiens
NQYRHYWSENLFQCF

SEQ ID NO: 434 - protein, 19 aa, Homo sapiens
SGSGNDCPGPGQDTDCREC

SEQ ID NO: 435 - protein, 19 aa, Homo sapiens
SGSGNSICCTKCHKGTYLY

SEQ ID NO: 436 - protein, 19 aa, Homo sapiens
SGSGCRKNQYRHYWSENLF

SEQ ID NO: 437 - protein, 19 aa, Homo sapiens
SGSGNQYRHYWSENLFQCF

(19) United States
(12) Patent Application Publication
(10) Pub. No.: US 2012/0107330 A1
(43) Pub. Date: May 3, 2012

(54) ANTAGONISTS, USES & METHODS FOR PARTIALLY INHIBITING TNFR1

(76) Inventor: Adriaan Allart Stoop, Cambridgeshire (GB)

(21) Appl. No.: 13/383,856

(22) PCT Filed: Jul. 14, 2010
(86) PCT No.: PCT/EP10/60111

§ 371 Date: Jan. 13, 2012

Related U.S. Application Data
(60) Provisional application No. 61/226,094, filed on Jul. 16, 2009.

(51) Int. Cl. A61K 39/395; C12N 1/21; C12N 1/15; C12N 1/19; A61P 29/00; C07K 16/30; C07H 21/04; A61P 1/00; A61P 19/02; A61P 17/06; C12N 5/10; C12N 15/63

(52) U.S. Cl. 424/174.1; 435/325; 435/348; 435/358; 435/365; 435/367; 435/414; 435/419; 435/252.3; 435/252.31; 435/252.33; 435/254.11; 435/254.2; 435/254.21; 435/254.23; 435/320.1; 530/389.7; 536/23.53

(57) ABSTRACT

The present invention relates to anti-Tumor Necrosis Factor 1 (TNFR1, p55, CD120a, P60, TNF receptor superfamily member 1A, TNFRSF1A) antagonists for partially inhibiting TNFR1 useful for the treatment and/or prevention of TNFR1-mediated diseases or conditions such as arthritis, psoriasis, Crohn's disease, COPD, lung inflammatory conditions and asthma. The invention further relates to methods, uses, formulations, compositions and devices comprising or using such anti-TNFR1 antagonists.

The present invention relates to anti-Tumor Necrosis Factor 1 (TNFR1, p55, CD120a, P60, TNF receptor superfamily member 1A, TNFRSF1A) antagonists for partially inhibiting TNFR1 useful for the treatment and/or prevention of TNFR1-mediated diseases or conditions such as arthritis, psoriasis, Crohn's disease, COPD, lung inflammatory conditions and asthma. The invention further relates to methods, uses, formulations, compositions and devices comprising or using such anti-TNFR1 antagonists.

### BACKGROUND OF THE INVENTION

### TNFR1

TNFR1 is a transmembrane receptor containing an extracellular region that binds ligand and an intracellular domain that lacks intrinsic signal transduction activity but can associate with signal transduction molecules. The complex of TNFR1 with bound TNF contains three TNFR1 chains and three TNF chains. (Banner et al., Cell, 73(3) 431-445 (1993).) The TNF ligand is present as a trimer, which is bound by three TNFR1 chains. (Id.) The three TNFR1 chains are clustered closely together in the receptor-ligand complex, and this clustering is a prerequisite to TNFR1-mediated signal transduction. In fact, multivalent agents that bind TNFR1, such as anti-TNFR1 antibodies, can induce TNFR1 clustering and signal transduction in the absence of TNF and are commonly used as TNFR1 agonists. (See, e.g., Belka et al., EMBO, 14(6):1156-1165 (1995); Mandik-Nayak et al., J. Immunol, 167:1920-1928 (2001).) Accordingly, multivalent agents that bind TNFR1 are generally not effective antagonists of TNFR1 even if they block the binding of TNFα to TNFR1.

SEQ ID numbers in this paragraph refer to the numbering used in WO2006038027. The extracellular region of TNFR1 comprises a thirteen amino acid amino-terminal segment (amino acids 1-13 of SEQ ID NO:603 (human); amino acids 1-13 of SEQ ID NO:604 (mouse)), Domain 1 (amino acids 14-53 of SEQ ID NO:603 (human); amino acids 14-53 of SEQ ID NO:604 (mouse)), Domain 2 (amino acids 54-97 of SEQ ID NO: 603 (human); amino acids 54-97 of SEQ ID NO:604 (mouse)), Domain 3 (amino acids 98-138 of SEQ ID NO: 603 (human); amino acid 98-138 of SEQ ID NO:604 (mouse)), and Domain 4 (amino acids 139-167 of SEQ ID NO:603 (human); amino acids 139-167 of SEQ ID NO:604 (mouse)) which is followed by a membrane-proximal region (amino acids 168-182 of SEQ ID NO:603_(human); amino acids 168-183 SEQ ID NO: 604 (mouse)). (See, Banner et al., Cell 73(3) 431-445 (1993) and Loetscher et al., Cell 61(2) 351-359 (1990).) Domains 2 and 3 make contact with bound ligand (TNFβ, TNFα). (Banner et al., Cell, 73(3) 431-445 (1993).) The extracellular region of TNFR1 also contains a region referred to as the pre-ligand binding assembly domain or PLAD domain (amino acids 1-53 of SEQ ID NO:603_(human); amino acids 1-53 of SEQ ID NO:604 (mouse)) (The Government of the USA, WO 01/58953; Deng et al., Nature Medicine, doi: 10.1038/nm 1304 (2005)).

TNFR1 is shed from the surface of cells in vivo through a process that includes proteolysis of TNFR1 in Domain 4 or in the membrane-proximal region (amino acids 168-182 of SEQ ID NO:603; amino acids 168-183 of SEQ ID NO:604), to produce a soluble form of TNFR1. Soluble TNFR1 retains the capacity to bind TNFα, and thereby functions as an endogenous inhibitor of the activity of TNFα.

WO2006038027, WO2008149144 and WO2008149148 disclose anti-TNFR1 immunoglobulin single variable domains and antagonists comprising these. These documents also disclose the use of such domains and antagonists for the treatment and/or prevention of conditions mediated by TNFα.

### SUMMARY OF THE INVENTION

The present inventors have realized that the partial inhibition of TNFR1 would be desirable for treating and/or preventing TNFR1-mediated diseases and conditions.

The invention provides antagonists which do not completely inhibit all TNFα, but only the excess amount of TNFα found during chronic inflammation, eg, in arthritis.

Thus, in one aspect the invention provides an anti-TNFα receptor type 1 (TNFR1; p55) antagonist for administration to a patient suffering from a TNFR1-mediated disease or condition, wherein the antagonist is a non-competitive inhibitor of TNFR1,

wherein the antagonist at a concentration of 100 nM inhibits human TNFR1 signaling by

(i) >50% in a standard MRC5 cell assay in the presence of human TNFα at a TNFα concentration in the assay of 100 pg/ml as determined by inhibition of IL-8 secretion using an immuno-sandwich method, and

(ii) ≦50% in a standard MRC5 cell assay in the presence of human TNFα at a TNFα concentration in the assay of 2 ng/ml or more (eg, 5 ng/ml) as determined by said immuno-sandwich method,

wherein the antagonist inhibits binding of human TNFR1 to an immunoglobulin single variable domain selected from DOM1h-574-156, DOM1h-574-72, DOM1h-574-109, DOM1h-574-138, DOM1h-574-162 and DOM1h-574-180,

for one or more of the following purposes:—
(a) for treating and/or preventing said TNFR1-mediated disease or condition by partially inhibiting TNFR1-mediated signaling in the patient. For example, the purpose is for treating and/or preventing arthritis (eg, rheumatoid arthritis) in the patient by partially inhibiting TNFR1-mediated signaling in the patient;
(b) for partially inhibiting TNFR1-mediated signaling in the patient;
(c) for inhibiting TNFα in the patient without (or without substantially) inhibiting host immune defence in the patient. For example; for inhibiting TNFα in the patient without (or without substantially) inhibiting phagocytosis by macrophages and/or mycobacterial killing in the patient;
(d) for treating and/or preventing said TNFR1-mediated disease or condition without (or without substantially) inhibiting a host immune defence in the patient. For example for treating and/or preventing said TNFR1-mediated disease or condition without (or without substantially) inhibiting phagocytosis by macrophages and/or mycobacterial killing in the patient; and
(e) for treating and/or preventing said TNFR1-mediated disease or condition without (or without substantially) inhibiting TNFα-mediated anti-infective activity in the patient. For example, the purpose is for treating and/or preventing said TNFR1-mediated disease or condition without (or without substantially) inhibiting TNFα-mediated suppression of tuberculosis in the patient. For example, the purpose is for treating and/or preventing said TNFR1-mediated disease or condition without (or without substantially) reversing tuberculosis latency in the patient. For example, the purpose is for treating and/or preventing said TNFR1-mediated disease or condition without (or without substantially) inhibiting TNFα-mediated Mycobacterium, such as Mycobacterium bovis BCG, infection in the patient. For example, the purpose is for treating and/or preventing said TNFR1-mediated disease or condition without (or without substantially) inhibiting TNFα-mediated suppression of respiratory tract infection in the patient. For example, the purpose is for treating and/or preventing said TNFR1-mediated disease or condition while reducing the risk of tuberculosis in the patient. For example, the purpose is for treating and/or preventing said TNFR1-mediated disease or condition while reducing the risk of infection, such as respiratory tract infection, in the patient.

In another aspect, the invention provides the use of an anti-TNFα receptor type 1 (TNFR1; p55) antagonist in the manufacture of a medicament, for administration to a patient suffering from a TNFR1-mediated disease or condition for one or more of the purposes (a) to (e) above, wherein the antagonist is a non-competitive inhibitor of TNFR1,

wherein the antagonist at a concentration of 100 nM inhibits human TNFR1 signaling by

(i) >50% in a standard MRC5 cell assay in the presence of human TNFα at a TNFα concentration in the assay of 100 pg/ml as determined by inhibition of IL-8 secretion using an immuno-sandwich method, and

(ii) ≦50% in a standard MRC5 cell assay in the presence of human TNFα at a TNFα concentration in the assay of 2 ng/ml or more (eg, 5 ng/ml) as determined by said immuno-sandwich method,

wherein the antagonist inhibits binding of human TNFR1 to an immunoglobulin single variable domain selected from DOM1h-574-156, DOM1h-574-72, DOM1h-574-109, DOM1h-574-138, DOM1h-574-162 and DOM1h-574-180.

In another aspect, the invention provides a method for one or more of the purposes (a) to (e) above, the method comprising partially inhibiting TNFR1-mediated signaling in the patient by administering an effective amount of an anti-TNFα receptor type 1 (TNFR1; p55) antagonist to the patient, wherein the antagonist is a non-competitive inhibitor of TNFR1,

wherein the antagonist at a concentration of 100 nM inhibits human TNFR1 signaling by

(i) >50% in a standard MRC5 cell assay in the presence of human TNFα at a TNFα concentration in the assay of 100 pg/ml as determined by inhibition of IL-8 secretion using an immuno-sandwich method, and

(ii) ≦50% in a standard MRC5 cell assay in the presence of human TNFα at a TNFα concentration in the assay of 2 ng/ml or more (eg, 5 ng/ml) as determined by said immuno-sandwich method,

wherein the antagonist inhibits binding of human TNFR1 to an immunoglobulin single variable domain selected from DOM1h-574-156, DOM1h-574-72, DOM1h-574-109, DOM1h-574-138, DOM1h-574-162 and DOM1h-574-180.

Embodiments of the antagonist, use and method of the invention are as follows. MRC-5 cells are available from ATCC and have been deposited under ATCC accession number CCL-171. In one embodiment, the MRC5 cell assays in (i) and (ii) are carried out at 37 degrees centigrade, each assay optionally for 18 hours. In one embodiment, in each assay the antagonist is pre-incubated with MRC5 cells (for example, for 60 minutes) prior to adding the TNFα. This pre-incubation time is not counted in the 18 hours assay time mentioned above. The TNFα can be from any source. In one embodiment, the TNFα is from Peprotech. The sequence of human TNFα is as follows:

VRSSSRTPSD KPVAHVVANP QAEGQLQWLN RRANALLANG
VELRDNQLVV PSEGLYLIYS QVLFKGQGCP STHVLLTHTI
SRIAVSYQTK VNLLSAIKSP CQRETPEGAE AKPWYEPIYL
GGVFQLEKGD RLSAEINRPD YLDFAESGQV YFGIIAL

In one embodiment, the human TNFα has an ED₅₀ as determined by the cytolysis of murine L929 cells in the presence of Actinomycin D of ≦0.05 ng/ml, corresponding to a specific activity of ≧2×10⁷ units/mg.

In one embodiment, the antagonist inhibits the binding of said selected immunoglobulin single variable domain to human and murine TNFR1. In one embodiment, the antagonist inhibits the binding of said selected immunoglobulin single variable domain to human, Cynomolgus monkey and murine TNFR1. In one embodiment, the antagonist inhibits the binding of said selected immunoglobulin single variable domain to human and Cynomolgus monkey TNFR1.

In one embodiment, the human TNFR1 that is used has the following sequence

MIYPSGVIGLVPHLGDREKRDSVCPQGKYIHPQNNSICCTKCHKGTYLY
NDCPGPGQDTDCRECESGSFTASENHLRHCLSCSKCRKEMGQVEISS
CTVDRDTVCGCRKNQYRHYWSENLFQCFNCSLCLNGTVHLSCQEKQN
TVCTCHAGFFLRENECVSCSNCKKSLECTKLCLPQIENVKGTEDSG

In one embodiment, the murine TNFR1 that is used has the following sequence.

MLMGIHPSGVTGLVPSLGDREKRDSLCPQGKYVHSKNNSICCTKCHKGT
YLVSDCPSPGRDTVCRECEKGTFTASQNYLRQCLSCKTCRKEMSQVEIS
PCQADKDTVCGCKENQFQRYLSETHFQCVDCSPCFNGTVTIPCKETQNT
VCNCHAGFFLRESECVPCSHCKKNEECMKLCLPPPLANVTNPQDS

In one embodiment, the Cynomolgus monkey TNFR1 that is used has the following sequence.

DSVCPQGKYIHPQNNSVCCTKCHKGTYLYNDCPGPGQDTDCRECESGSF
TASENHLRHCLSCSKCRKEMGQVEISSCTVDRDTVCGCRKNQYRYYWSE
NLFQCFNCSLCLNGTVHLSCQEKQNTVCTCHAGFFLRENECVSCSNCKK
TLECTKLCLPQTENVKGTEDSG

In one embodiment, said selected immunoglobulin single variable domain is DOM1h-574-156.

In one embodiment, the antagonist comprises an immunoglobulin single variable domain having an amino acid sequence that is at least 80% identical to the amino acid sequence of DOM1h-574-156, DOM1h-574-72, DOM1h-574-109, DOM1h-574-138, DOM1h-574-162 or DOM1h-574-180. For example, the amino acid sequence is at least 85, 90, 95, 96, 97, 98 or 99% identical to the amino acid sequence of DOM1h-574-156, DOM1h-574-72, DOM1h-574-109, DOM1h-574-138, DOM1h-574-162 or DOM1h-574-180, or is 100% identical.

In another aspect of the invention, there is provided an anti-TNFα receptor type 1 (TNFR1; p55) antagonist for administration to a patient suffering from a TNFR1-mediated disease or condition, wherein the antagonist is a non-competitive inhibitor of TNFR1,

wherein the antagonist at a concentration of 100 nM inhibits murine TNFR1 signaling by

(i) >50% in a standard L929 cell assay in the presence of murine TNFα at a concentration in the assay of 20 pg/ml, and

(ii) ≦50% in a standard L929 cell assay in the presence of murine TNFα at a concentration in the assay of 100 pg/ml or more,

wherein the antagonist inhibits binding of murine TNFR1 to an immunoglobulin single variable domain selected from DOM1h-574-156; DOM1m-21-23, DOM1h-574-156, DOM1h-574-72, DOM1h-574-109, DOM1h-574-138, DOM1h-574-162 and DOM1h-574-180,

for one or more of the following purposes:—
(a) for treating and/or preventing said TNFR1-mediated disease or condition by partially inhibiting TNFR1-mediated signaling in the patient. For example, the purpose is for treating and/or preventing arthritis (eg, rheumatoid arthritis) in the patient by partially inhibiting TNFR1-mediated signaling in the patient;
(b) for partially inhibiting TNFR1-mediated signaling in the patient;
(c) for inhibiting TNFα in the patient without (or without substantially) inhibiting host immune defence in the patient;
(d) for treating and/or preventing said TNFR1-mediated disease or condition without (or without substantially) inhibiting a host immune defence in the patient; and
(e) for treating and/or preventing said TNFR1-mediated disease or condition without (or without substantially) inhibiting TNFα-mediated anti-infective activity in the patient.

In another aspect, the invention provides the use of an anti-TNFα receptor type 1 (TNFR1; p55) antagonist in the manufacture of a medicament, for administration to a patient suffering from a TNFR1-mediated disease or condition for one or more of the purposes (a) to (e) above, wherein the antagonist is a non-competitive inhibitor of TNFR1, and

wherein the antagonist at a concentration of 100 nM inhibits murine TNFR1 by

(i) >50% in a standard L929 cell assay in the presence of murine TNFα at a concentration in the assay of 20 pg/ml, and

(ii) ≦50% in a standard L929 cell assay in the presence of murine TNFα at a concentration in the assay of 100 pg/ml or more,

wherein the antagonist inhibits binding of murine TNFR1 to an immunoglobulin single variable domain selected from DOM1h-574-156; DOM1m-21-23, DOM1h-574-156, DOM1h-574-72, DOM1h-574-109, DOM1h-574-138, DOM1h-574-162 and DOM1h-574-180.

In another aspect, the invention provides a method for one or more of the purposes (a) to (e) above, the method comprising partially inhibiting TNFR1-mediated signaling in the patient by administering an effective amount of an anti-TNFα receptor type 1 (TNFR1; p55) antagonist to the patient, wherein the antagonist is a non-competitive inhibitor of TNFR1, and

wherein the antagonist at a concentration of 100 nM inhibits murine TNFR1 by

(i) >50% in a standard L929 cell assay in the presence of murine TNFα at a concentration in the assay of 20 pg/ml, and

(ii) ≦50% in a standard L929 cell assay in the presence of murine TNFα at a concentration in the assay of 100 pg/ml or more,

wherein the antagonist inhibits binding of murine TNFR1 to an immunoglobulin single variable domain selected from DOM1h-574-156; DOM1m-21-23, DOM1h-574-156, DOM1h-574-72, DOM1h-574-109, DOM1h-574-138, DOM1h-574-162 and DOM1h-574-180.

In one embodiment, the antagonist comprises an immunoglobulin single variable domain having an amino acid sequence that is at least 80% identical to the amino acid sequence of DOM1h-574-156, DOM1h-574-72, DOM1h-574-109, DOM1h-574-138, DOM1h-574-162 or DOM1h-574-180. For example, the amino acid sequence is at least 85, 90, 95, 96, 97, 98 or 99% identical to the amino acid sequence of DOM1h-574-156, DOM1h-574-72, DOM1h-574-109, DOM1h-574-138, DOM1h-574-162 or DOM1h-574-180, or is 100% identical. In one embodiment, the antagonist comprises an immunoglobulin single variable domain having an amino acid sequence that is at least 80% identical to the amino acid sequence of DOM1h-574-156 or DOM1m-21-23. For example, the amino acid sequence is at least 85, 90, 95, 96, 97, 98 or 99% identical to the amino acid sequence of DOM1h-574-156 or DOM1m-21-23, or is 100% identical.

L929 cells are available from ATCC and have been deposited under ATCC accession number ATCC CCL-1. In one embodiment, the L929 cell assays in (i) and (ii) are carried out at 37 degrees centigrade, each assay optionally for 18 hours. In one embodiment, in each assay the antagonist is pre-incubated with L929 cells (for example, 60 minutes pre-incubation) prior to adding the TNFα. This pre-incubation time is not counted in the 18 hours assay time mentioned above. The TNFα can be from any source. In one embodiment, the TNFα is from R&D Systems. The sequence of murine TNFα is as follows

MLRSSSQNSS DKPVAHVVAN HQVEEQLEWL SQRANALLAN
GMDLKDNQLV VPADGLYLVY SQVLFKGQGC PDYVLLTHTV
SRFAISYQEK VNLLSAVKSP CPKDTPEGAE LKPWYEPIYL
GGVFQLEKGD QLSAEVNLPK YLDFAESGQV YFGVIAL

In one embodiment, the murine TNFα has an ED₅₀, as determined by the cytolysis of murine L929 cells in the presence of actinomycin D, of 0.02-0.05 ng/ml, corresponding to a specific activity of >1×10⁷ units/mg.

Embodiments of the antagonist, use and method of any aspect of the invention are as follows.

In one embodiment, the antagonist inhibits the binding of said selected immunoglobulin single variable domain to human and murine TNFR1. In one embodiment, the antagonist inhibits the binding of said selected immunoglobulin single variable domain to human, Cynomolgus monkey and murine TNFR1.

In one embodiment, the human TNFR1 that is used has the following sequence

MIYPSGVIGLVPHLGDREKRDSVCPQGKYIHPQNNSICCTKCHKGTYLY
NDCPGPGQDTDCRECESGSFTASENHLRHCLSCSKCRKEMGQVEISS
CTVDRDTVCGCRKNQYRHYWSENLFQCFNCSLCLNGTVHLSCQEKQNT
VCTCHAGFFLRENECVSCSNCKKSLECTKLCLPQIENVKGTEDSG

In one embodiment, the murine TNFR1 that is used has the following sequence

MLMGIHPSGVTGLVPSLGDREKRDSLCPQGKYVHSKNNSICCTKCHKG
TYLVSDCPSPGRDTVCRECEKGTFTASQNYLRQCLSCKTCRKEMSQVE
ISPCQADKDTVCGCKENQFQRYLSETHFQCVDCSPCFNGTVTIPCKETQ
NTVCNCHAGFFLRESECVPCSHCKKNEECMKLCLPPPLANVTNPQDS

In one embodiment, the Cynomolgus monkey TNFR1 that is used has the following sequence

DSVCPQGKYIHPQNNSVCCTKCHKGTYLYNDCPGPGQDTDCRECESGSF
TASENHLRHCLSCSKCRKEMGQVEISSCTVDRDTVCGCRKNQYRYYWSE
NLFQCFNCSLCLNGTVHLSCQEKQNTVCTCHAGFFLRENECVSCSNCKK
TLECTKLCLPQTENVKGTEDSG

In one embodiment, said selected immunoglobulin single variable domain is DOM1h-574-156.

In one embodiment, the antagonist inhibits the binding of said selected immunoglobulin single variable domain to TNFR1 by at least 50%, for example, by at least 60, 65, 70, 75, 80, 85, 90, 95, 96, 97, 98 or 99%. For example, the antagonist totally (100%) inhibits the binding of said selected immunoglobulin single variable domain to TNFR1. See WO2006038027 for details of how to perform competition ELISA and competition BiaCore™ experiments to determine inhibition of binding of said selected immunoglobulin single variable domain to TNFR1 in the presence of the antagonist of the invention. Although the guidance in WO2006038027 is provided mainly in view of antagonists such as domains (eg, single variable domain antagonists such as dAbs), the skilled addressee would realize that that guidance can be readily adapted for use with any antagonist of the invention disclosed herein.

The MRC5 assays are standard assays for functional inhibition of TNFα mediated IL-8 release, eg, carried out in the manner specified below in the Examples section. A standard L929 assay determines TNFR1 inhibition as indicated by inhibition of TNF alpha-induced cytotoxicity. A standard Cynomolgus KI assay determines TNFR1 inhibition as indicated by inhibition of TNF alpha-induced IL-8 secretion. Details of standard assays for TNFR1 antagonists are known in the art, eg in WO2006038027, WO2008149144 and WO2008149148. Details are also provided in the experimental section below.

Known immuno-sandwich methods can be used, and these will be evident to the skilled addressee. For example, the immuno-sandwich method is selected from ELISA, using a calorimetric detection, the Applied Biosystems 8200 cellular detection system (FMAT), using fluorescence detection and Meso Scale Discovery (MSD), using electrochemiluminescence detection

The concentrations of TNFα used in assays herein can be determined by conventional techniques. For example, determination can be performed by testing functional activity in the L929 cytotoxicity assay.

In an example of any aspect of the invention, the patient is a mammal, eg, a human, mouse or Cynomolgus monkey.

In one embodiment or any aspect of the present invention, the antagonist is an antibody or antigen-binding fragment thereof, such as a monovalent antigen-binding fragment (e.g., scFv, Fab, Fab′, dAb) that has binding specificity for TNFR1. Other examples of antagonists are antagonists or ligands described in WO2006059110, WO2006038027 and WO2008149148 that bind TNFR1. For example, the antagonist can consist of or comprise a PLAD peptide. The ligands may comprise an immunoglobulin single variable domain or domain antibody (dAb) that has binding specificity for TNFR1, or the complementarity determining regions of such a dAb in a suitable format. In some embodiments, the ligand is a dAb monomer that consists essentially of, or consists of, an immunoglobulin single variable domain or dAb that has binding specificity for TNFR1. In other embodiments, the ligand is a polypeptide that comprises a dAb (or the CDRs of a dAb) in a suitable format, such as an antibody format.

In one embodiment of the antagonist, use or method of any aspect, said condition is an inflammatory condition, optionally a chronic inflammatory condition. For example, the condition is selected from the group consisting of arthritis (optionally rheumatoid arthritis or juvenile rheumatoid arthritis), ankylosing spondylitis, osteoarthritis, inflammatory bowel disease (optionally Crohn's disease or ulcerative colitis) and psoriasis. Other examples of diseases and conditions addressable in the context of the present invention are SLE, erythmatosus, atherosclerosis, alzheimers diseases, COPD, MS and other indications to description; wherein said chronic obstructive pulmonary disease is selected from the group consisting of chronic bronchitis, chronic obstructive bronchitis and emphysema; wherein said pneumonia is bacterial pneumonia; wherein said bacterial pneumonia is Staphylococcal pneumonia.

In one embodiment, the disease is a respiratory disease. For example, the respiratory disease is selected from the group consisting of lung inflammation, chronic obstructive pulmonary disease, acute lung injury (ALI), asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis. The invention provides a pulmonary delivery device containing the TNFR1 antagonist of any aspect of the invention. The device, in one example, is an inhaler or an intranasal administration device. The invention also provides an oral formulation comprising the TNFR1 antagonist of any aspect of the invention. The formulation, in one example, is a tablet, pill, capsule, liquid or syrup.

In another aspect, the invention provides an isolated or recombinant nucleic acid comprising a nucleotide sequence encoding for an antagonist according to the invention. The nucleotide sequence is, in one embodiment, any of the nucleotide sequences herein that encodes an anti-TNFR1 antagonist (eg, an immunoglobulin single variable domain) or any such nucleotide sequence disclosed in WO2006038027, WO2008149148 or WO2006059110.

In another aspect, the invention provides a vector comprising the nucleic acid of the invention.

In another aspect, the invention provides a host cell comprising the nucleic acid or the vector of the present invention.

In another aspect, the invention provides a pharmaceutical composition comprising an anti-TNFR1 antagonist of the invention and a pharmaceutically acceptable carrier, excipient or diluent.

In one embodiment of any aspect, the antagonist comprises or consists of an anti-TNFα receptor type 1 (TNFR1; p55) immunoglobulin single variable domain, wherein the single variable domain is a mutant of DOM1h-574-14 comprising one or more of the following mutations (numbering according to Kabat) position 30 is L or F,

position 52 is A or T,
position 52a is D or E,
position 54 is A or R,
position 57 is R, K or A,
position 60 is D, S, T or K,
position 61 is E, H or G,
position 62 is A or T,
position 100 is R, G, N, K, Q, V, A, D, S or V, and
position 101 is A, Q, N, E, V, H or K.

In one embodiment, the variable domain has one or more features of any of the other aspects or embodiments of the invention and the disclosure of the present text is to be interpreted to enable such features to be combined, eg for inclusion in claims herein.

Optionally, the single variable domain is a mutant of DOM1h-574-14 comprising one or more of the following mutations (numbering according to Kabat)

position 30 is L or F,
position 52 is A or T,
position 52a is D,
position 54 is A,
position 57 is R,
position 60 is D, S or T,
position 61 is H,
position 62 is A,
position 100 is V, A, R, G, N or K, and
position 101 is E, V, K, A Q or N.

In one embodiment of any aspect, the antagonist comprises or consists of an anti-TNFα receptor type 1 (TNFR1; p55) immunoglobulin heavy chain single variable domain comprising valine at position 101 (numbering according to Kabat). In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain comprising one or more of 30G, 44D, 45P, 55D, 56R, 94I and 98R, wherein numbering is according to Kabat, wherein the amino acid sequence of the single variable domain is otherwise identical to the amino acid sequence of DOM1h-574. In one embodiment, the variable domain is provided for binding human, murine or Cynomologus monkey TNFR1. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain which comprises an amino acid sequence that is at least 95% identical to the amino acid sequence of DOM1h-574-72, DOM1h-574-156, DOM1h-574-109, DOM1h-574-132, DOM1h-574-135, DOM1h-574-138, DOM1h-574-162 or DOM1h-574-180. This embodiment provides variable domains that are potent neutralizers of TNFR1 (eg, at least human TNFR1) in cell assay. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain which comprises an amino acid sequence that is at least 94% identical to the amino acid sequence of DOM1h-574-109, DOM1h-574-93, DOM1h-574-123, DOM1h-574-125, DOM1h-574-126, DOM1h-574-129, DOM1h-574-133, DOM1h-574-137, or DOM1h-574-160. This embodiment provides variable domains that are proteolytically stable. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain which comprises an amino acid sequence that is at least 95% identical to the amino acid sequence of DOM1h-574-72, DOM1h-574-109, DOM1h-574-125, DOM1h-574-126, DOM1h-574-133, DOM1h-574-135, DOM1h-574-138, DOM1h-574-139, DOM1h-574-155, DOM1h-574-156, DOM1h-574-162, or DOM1h-574-180. This embodiment provides variable domains that bind human TNFR1 with high affinity and optionally also display desirable affinity for murine TNFR1. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain for binding human, murine or Cynomologus monkey TNFR1, wherein the single variable domain is encoded by a nucleotide sequence that is at least 80, 85, 90, 95, 96, 97, 98 or 99% identical to the nucleotide sequence of any one of the DOM1h sequences shown in Table 12 below, with the exception of DOM1h-574. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain for binding human, murine or Cynomologus monkey TNFR1, wherein the single variable domain is encoded by a nucleotide sequence that is at least 80, 85, 90, 95, 96, 97, 98 or 99% identical to the nucleotide sequence of DOM1h-574-72, DOM1h-574-109, DOM1h-574-138, DOM1h-574-156, DOM1h-574-162 or DOM1h-574-180. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain comprising an amino acid sequence that is identical to the amino acid sequence selected from the amino acid sequence of DOM1h-574-72, DOM1h-574-109, DOM1h-574-138, DOM1h-574-156, DOM1h-574-162 and DOM1h-574-180 or differs from the selected amino acid sequence at no more than 25 amino acid positions and has a CDR1 sequence that is at least 50% identical to the CDR1 sequence of the selected amino acid sequence. In one embodiment, the immunoglobulin single variable domain comprises a CDR2 sequence that is at least 50% identical to the CDR2 sequence of the selected amino acid sequence. In one embodiment, the immunoglobulin single variable comprises a CDR3 sequence that is at least 50% identical to the CDR3 sequence of the selected amino acid sequence. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain comprising an amino acid sequence that is identical to the amino acid sequence selected from the amino acid sequence of DOM1h-574-72, DOM1h-574-109, DOM1h-574-138, DOM1h-574-156, DOM1h-574-162 and DOM1h-574-180 or differs from the selected amino acid sequence at no more than 25 amino acid positions and has a CDR2 sequence that is at least 50% identical to the CDR2 sequence of the selected amino acid sequence. In one embodiment, the immunoglobulin single variable domain comprises a CDR3 sequence that is at least 50% identical to the CDR3 sequence of the selected amino acid sequence. In one embodiment, the immunoglobulin single variable domain comprises a CDR1 sequence that is at least 50% identical to the CDR1 sequence of DOM1h-574-72. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of an anti-TNFα receptor type 1 (TNFR1; p55) immunoglobulin single variable domain which comprising an amino acid sequence that is identical to the amino acid sequence selected from the amino acid sequence of DOM1h-574-72, DOM1h-574-109, DOM1h-574-138, DOM1h-574-156, DOM1h-574-162 and DOM1h-574-180 or differs from the selected amino acid sequence at no more than 25 amino acid positions and has a CDR3 sequence that is at least 50% identical to the CDR3 sequence of the selected amino acid sequence. In one embodiment, the variable domain has one or more features of any of the other aspects or embodiments of the invention and the disclosure of the present text is to be interpreted to enable such features to be combined, eg for inclusion in claims herein.

In one embodiment of any aspect, the antagonist comprises or consists of a protease resistant anti-TNFα receptor type 1 (TNFR1; p55) immunoglobulin single variable domain, wherein the single variable domain is resistant to protease when incubated with

(i) a concentration (c) of at least 10 micrograms/ml protease at 37° C. for time (t) of at least one hour; or
(ii) a concentration (c′) of at least 40 micrograms/ml protease at 30° C. for time (t) of at least one hour.
wherein the variable domain comprises an amino acid sequence that is at least 94% identical to the amino acid sequence of DOM1h-574-126 or DOM1h-574-133, and optionally comprises a valine at position 101 (Kabat numbering). In one embodiment, the variable domain has one or more features of any of the other aspects or embodiments of the invention and the disclosure of the present text is to be interpreted to enable such features to be combined, eg for inclusion in claims herein.

In one embodiment, the antagonist comprises or consists of a polypeptide comprising an anti-TNFR1 immunoglobulin single variable domain as herein described and an antibody constant domain, optionally an antibody Fc region, optionally wherein the N-terminus of the Fc is linked (optionally directly linked) to the C-terminus of the variable domain.

In one embodiment, the antagonist comprises or consists of a multispecific ligand comprising an anti-TNFR1 immunoglobulin single variable domain as herein described and optionally at least one immunoglobulin single variable domain that specifically binds serum albumin (SA). In one embodiment, the multispecific ligand is, or comprises, an amino acid sequence selected from the amino acid sequence of any construct labeled “DMS” disclosed herein, for example, any one of DMS0111, 0112, 0113, 0114, 0115, 0116, 0117, 0118, 0121, 0122, 0123, 0124, 0132, 0133, 0134, 0135, 0136, 0162, 0163, 0168, 0169, 0176, 0177, 0182, 0184, 0186, 0188, 0189, 0190, 0191, 0192, 5519, 5520, 5521, 5522, 5525 and 5527. In one embodiment, the multispecific ligand is, or comprises, an amino acid sequence encoded by the nucleotide sequence of any DMS disclosed herein, for example, any one of the nucleotide sequences of DMS0111, 0112, 0113, 0114, 0115, 0116, 0117, 0118, 0121, 0122, 0123, 0124, 0132, 0133, 0134, 0135, 0136, 0162, 0163, 0168, 0169, 0176, 0177, 0182, 0184, 0186, 0188, 0189, 0190, 0191, 0192, 5519, 5520, 5521, 5522, 5525 and 5527. In one embodiment, the invention provides a nucleic acid encoding an antagonist of the invention which comprises a cmultispecific ligand comprising an anti-TNFR1 immunoglobulin single variable domain and an anti-SA single variable domain, wherein the nucleic acid comprises the nucleotide sequence of any DMS disclosed herein, for example, any one of the nucleotide sequences of DMS0111, 0112, 0113, 0114, 0115, 0116, 0117, 0118, 0121, 0122, 0123, 0124, 0132, 0133, 0134, 0135, 0136, 0162, 0163, 0168, 0169, 0176, 0177, 0182, 0184, 0186, 0188, 0189, 0190, 0191, 0192, 5519, 5520, 5521, 5522, 5525 and 5527. There is provided a vector comprising such a nucleic acid, as well as a host cell comprising such a vector.

In one embodiment, the invention provides an antagonist of the invention which comprises or consists of a multispecific ligand comprising (i) an anti-TNFα receptor type 1 (TNFR1; p55) immunoglobulin single variable domain which comprises an amino acid sequence that is at least 93% identical to the amino acid sequence of DOM1h-574-156, (ii) at least one anti-serum albumin (SA) immunoglobulin single variable domain that specifically binds SA, wherein the anti-SA single variable domain comprises an amino acid sequence that is at least 80% identical to the sequence of DOM7h-11-3, and (iii) optionally wherein a linker is provided between the anti-TNFR1 single variable domain and the anti-SA single variable

In one embodiment, the invention provides an antagonist of the invention which comprises or consists of a multispecific ligand comprising (i) an anti-TNFα receptor type 1 (TNFR1; p55) immunoglobulin single variable domain which comprises an amino acid sequence that is at least 93% identical to the amino acid sequence of DOM1h-574-156, (ii) at least one anti-serum albumin (SA) immunoglobulin single variable domain that specifically binds SA, wherein the anti-SA single variable domain comprises an amino acid sequence that is at least 80% identical to the sequence of DOM7h-14-10, and (iii) optionally wherein a linker is provided between the anti-TNFR1 single variable domain and the anti-SA single variable domain, the linker comprising the amino acid sequence AST, optionally ASTSGPS.

In one embodiment, the invention provides a TNFα receptor type 1 (TNFR1; p55) antagonist of the invention, for oral delivery, delivery to the GI tract of a patient, pulmonary delivery, delivery to the lung of a patient or systemic delivery.

In one embodiment, the invention provides a TNFα receptor type 1 (TNFR1; p55) antagonist of the invention for binding human, murine or Cynomologus monkey TNFR1, the antagonist having a CDR1 sequence that is at least 50% identical to the CDR1 sequence of DOM1h-574-72, DOM1h-574-109, DOM1h-574-138, DOM1h-574-156, DOM1h-574-162 and DOM1h-574-180.

In one embodiment, the invention provides a TNFα receptor type 1 (TNFR1; p55) antagonist of the invention for binding human, murine or Cynomologus monkey TNFR1, the antagonist having a CDR2 sequence that is at least 50% identical to the CDR2 sequence of DOM1h-574-72, DOM1h-574-109, DOM1h-574-138, DOM1h-574-156, DOM1h-574-162 and DOM1h-574-180.

In one embodiment, the invention provides a TNFα receptor type 1 (TNFR1; p55) antagonist of the invention for binding human, murine or Cynomologus monkey TNFR1, the antagonist having a CDR3 sequence that is at least 50% identical to the CDR3 sequence of DOM1h-574-72, DOM1h-574-109, DOM1h-574-138, DOM1h-574-156, DOM1h-574-162 and DOM1h-574-180.

In one embodiment, the invention provides a TNFα receptor type 1 (TNFR1; p55) antagonist of the invention for binding human, murine or Cynomologus monkey TNFR1, the antagonist comprising an immunoglobulin single variable domain comprising the sequence of CDR1, CDR2, and/or CDR3 of a single variable domain selected from DOM1h-574-72, DOM1h-574-109, DOM1h-574-138, DOM1h-574-156, DOM1h-574-162 and DOM1h-574-180.

In one embodiment, the invention provides a TNFR1 antagonist of the invention for treating and/or prophylaxis of an inflammatory condition.

In one embodiment, the invention provides the use of the TNFR1 antagonist of the invention in the manufacture of a medicament for treating and/or prophylaxis of an inflammatory condition.

In one aspect, an anti-TNFR1 antagonist, single variable domain, polypeptide or multispecific ligand of any one aspect or embodiment of the invention is provided for targeting one or more epitopic sequence of TNFR1 selected from the group consisting of NSICCTKCHKGTYLY, NSICCTKCHKGTYL, CRKNQYRHYWSENLF and NQYRHYWSENLFQCF.

In one aspect, an anti-TNFR1 antagonist, single variable domain, polypeptide or multispecific ligand of any one aspect or embodiment of the invention is provided for targeting one or more epitopic sequence of TNFR1 selected from the group consisting of NSICCTKCHKGTYLY, NSICCTKCHKGTYL, CRKNQYRHYWSENLF and NQYRHYWSENLFQCF, to treat and/or prevent any condition or disease specified above.

In one aspect, the invention provides a method of treating and/or preventing any condition or disease specified above in a patient, the method comprising administering to the patient an anti-TNFR1 antagonist, single variable domain, polypeptide or multispecific ligand of any aspect or embodiment of the invention for targeting one or more epitopic sequence of TNFR1 selected from the group consisting of NSICCTKCHKGTYLY, NSICCTKCHKGTYL, CRKNQYRHYWSENLF and NQYRHYWSENLFQCF in the patient.

### BRIEF DESCRIPTION OF THE FIGURES

FIG. 1. BIAcore binding of dAbs from naïive selections to human TNFR1. Biotinylated human TNFR1 was coated on a SA BIAcore chip. Four purified dAbs (DOM1h-509, DOM1h-510, DOM1h-549 and DOM1h-574), from naïve selections, were injected over human TNFR1 and binding was determined. The curves corresponding to each dAb are indicated by arrows.

FIG. 2. MRC5 cell assay for dAbs from naïive selections to human TNFR1. Four purified dAbs (DOM1h-509, DOM1h-510, DOM1h-549 and DOM1h-574) from the naïve selections and a control dAb (DOM1h-131-511) were analysed in the MRC5 cell assay for functional inhibition of TNFα mediated IL-8 release. The assay was performed as described and the curve corresponding to each dAb is indicated with an arrow. In the graph dAb concentration is plotted against percentage neutralisation observed.

FIG. 3. Receptor Binding Assay for dAbs from naïive selections to human TNFR1. Four purified dAbs (DOM1h-509, DOM1h-510, DOM1h-549 and DOM1h-574) from the naïive selections and a control dAb (DOM1h-131-511) were assayed in the receptor binding assay to determine competition with TNFα. The positive control dAb is known to be competitive with TNFα and shows a fully inhibition curve. The selected anti-TNFR1 dAbs do not inhibit TNFα binding to the receptor. The assay was performed as described and the curve corresponding to each dAb is indicated with an arrow.

FIG. 4. MRC5 cell assay for dAbs from error-prone test maturations to human TNFR1. Three purified dAbs (DOM1h-574-7, DOM1h-574-8 and DOM1h-574-10) from the naïve selections and a control dAb (DOM1h-131-511) were analysed in the MRC5 cell assay for functional inhibition of TNFα mediated IL-8 release. The assay was performed as described and the curve corresponding to each dAb is indicated with an arrow. In the graph dAb concentration is plotted against percentage neutralisation observed. Compared to the parental DOM1h-574 shown in FIG. 2, these dAbs demonstrate increased potency in the MRC5 cell assay.

FIG. 5. Amino-acid sequence alignment for dAbs identified from error-prone libraries of DOM1h-574 and their subsequent recombinations. The error-prone, test maturation selections for improved DOM1h-574 dAbs identified positions responsible for affinity improvements in DOM1h-574-7, DOM1h-574-8, DOM1h-574-10, DOM1h-574-11, DOM1h-574-12 and DOM1h-574-13. Recombinations of these mutations (V30G, G44D, L45P, G55D, H56R and K94I) yielded DOM1h-574-14 to DOM1h-574-19. A “.” at a particular position indicates the same amino as found in DOM1h-574 at that position. The CDRs are indicated by underlining and bold text (the first underlined sequence is CDR1, the second underlined sequence is CDR2 and the third underlined sequence is CDR3).

FIG. 6. Amino-acid sequence alignment of the extracellular domain of TNFR1 from human, Cynomologous monkey, dog and mouse. The alignment highlights the limited conservation of sequence between human and mouse TNFR1. A “.” at a particular position indicates the same amino as found in human ECD TNFR1 at that position.

FIG. 7. Monitoring of binding of DOM1h-574-16 and DOM1h-131-206 to dog TNFR1 as determined by BIAcore. A BIAcore SA chip was coated with biotinylated dog TNFR1. Subsequently, the purified dAbs DOM1h-574-16 and DOM1h-131-206, each at 100 nM, were injected over dog TNFR1. From the traces it is clear that whereas DOM1h-574-16 shows significant binding, only limited binding is observed for DOM1h-131-206.

FIG. 8. Monitoring of binding of purified DOM1h-574-16 to mouse TNFR1 as determined by BIAcore. A BIAcore SA chip was coated with biotinylated mouse TNFR1. Subsequently, the purified dAb DOM1h-574-16, at 1 μM, was injected over mouse TNFR1. The trace clearly demonstrates binding of DOM1h-574-16 for mouse TNFR1.

FIG. 9. Functional activity of DOM1h-574-16 in a mouse L929 cell assay. Purified DOM1h-574-16 (black line, triangles) was assayed for functional cross-reactivity with mouse TNFR1 by testing its ability to protect mouse L929 cells from the cytotoxic effect of TNFα in the presence of actinomycine. As a positive control, the mouse TNFR1 binding dAb, DOM1m-21-23 (grey line, squares) was included and shown to be active. In the graph, dAb concentration is plotted against percentage neutralisation of TNFα activity. The assay was performed as described in the examples.

FIG. 10. Functional activity of DOM1h-574-16 in a Cynomologous monkey CYNOM-K1 cell assay. Purified DOM1h-574-16 (grey dashed line, triangles) was assayed for functional cross-reactivity with Cynomologous monkey TNFR1 by testing its ability to inhibit IL-8 release from CYNOM-K1 cells in response to TNFα. The assay was performed as described in the examples. As a positive control, DOM1h-131-511 (black solid line, squares) was included. Both dAbs showed full neutralisation. In the graph, dAb concentration is plotted against percentage neutralisation of TNFα activity.

FIG. 11A-C. Amino-acid sequence alignment for the most potent dAbs from the DOM1h-574 lineage identified during affinity maturation. The amino-acid sequences of the dAbs with the highest potency in the MRC5 cell assay are listed along-side the parental DOM1h-574, the template used for starting affinity maturation (DOM1h-574-14) and an earlier dAb identified with increased potency (DOM1h-574-72). A “.” at a particular position indicates the same amino as found in DOM1h-574 at that position. The CDRs are indicated by underlining and bold text (the first underlined sequence is CDR1, the second underlined sequence is CDR2 and the third underlined sequence is CDR3).

FIG. 12 A-C. Amino-acid sequence alignment for the most protease stable dAbs from the DOM1h-574 lineage identified during affinity maturation. The amino-acid sequences of those dAbs identified after affinity maturation which were shown to be the most resistant to trypsin digestion. For alignment purposes, the parental dAb DOM1h-574 is also included. A “.” at a particular position indicates the same amino as found in DOM1h-574 at that position. The CDRs are indicated by underlining and bold text (the first underlined sequence is CDR1, the second underlined sequence is CDR2 and the third underlined sequence is CDR3).

FIG. 13 A-C. Amino-acid sequence alignment for the dAbs chosen for detailed characterisation. The alignment contains the twelve dAbs chosen for detailed characterisation as well as DOM1h-574 (the parental dAb) and DOM1h-574-16, which was used early on for characterisation of the lineage. A “.” at a particular position indicates the same amino as found in DOM1h-574 at that position. The CDRs are indicated by underlining and bold text (the first underlined sequence is CDR1, the second underlined sequence is CDR2 and the third underlined sequence is CDR3).

FIG. 14. Epitope mapping by BIAcore for DOM1h-574-16 and DOM1h-131-511. A BIAcore SA chip was coated with biotinylated human TNFR1. Across this surface injections were performed of DOM1h-131-511 and DOM1h-574-16 (each at 200 nM and followed by a regeneration injection (not shown)). The number of RUs bound for each of the dAbs was determined. Subsequently, the same concentration of DOM1h-131-511 was injected, directly followed by an injection of DOM1h-574-16. As can clearly been seen, the number of binding units for the second injections of DOM1h-574-16 equals the first injection, indicating the dAbs bind non-competing epitopes.

FIG. 15. Epitope mapping by BIAcore for DOM1h-574-16 and MAB225 (R&D Systems). A BIAcore SA chip was coated with biotinylated human TNFR1. Across the surface DOM1h-574-16 was injected and the binding quantified. After regeneration (not shown), MAB225 was injected followed again by injection of DOM1h-574-16. The level of binding for DOM1h-574-16 is very comparable to that seen in the absence of MAB225, indicating a binding epitope non-competitive with MAB225.

FIG. 16. Epitope mapping by BIAcore for DOM1h-574-16 and the mAb Clone 4.12. A BIAcore SA chip was coated with biotinylated human TNFR1. Across the surface, Clone 4.12 (Invitrogen, Zymed) was injected and the binding quantified. After regeneration (not shown), DOM1h-574-16 was injected followed again by injection of Clone 4.12. The level of binding observed for the second injection of Clone 4.12 is about 20% less than that observed in the absence of DOM1h-574-16. This result indicates a limited competition for the binding epitope on human TNFR1. DOM1h-574-16 and Clone 4.12 might have slightly overlapping epitopes. The jumps in RU signal immediately before and after injections are buffer jumps, which have not been subtracted.

FIG. 17. Epitope mapping by BIAcore for DOM1h-574-16 and DOM1h-510. A BIAcore SA chip was coated with biotinylated human TNFR1. Across the surface, DOM1h-510 was injected and the binding quantified. Subsequently, DOM1h-574-16 was injected followed again by injection of DOM1h-510. Clearly, the second injection of DOM1h-510 showed far less binding, indicating a competing epitope is being bound by DOM1h-510.

FIG. 18. Epitope mapping by BIAcore for DOM1h-574-16 and DOM1m-21-23. A BIAcore SA chip was coated with biotinylated mouse TNFR1. Across the surface, DOM1h-574-16 was injected and the binding quantified. Subsequently, DOM1m-21-23 was injected followed again by injection of DOM1h-574-16. The number of bound RUs of DOM1h-574-16 after the second injection is very similar to that observed in the absence of DOM1m-12-23. This would indicate that DOM1m-21-23 and DOM1h-574-16 have different binding epitopes on mouse TNFR1.

FIG. 19. Epitope mapping of DOM1h-574-16 to linear peptide fragments of TNFR1 by BIAcore. The four channels of a BIAcore SA chip were each coated with one of four biotinylated peptides. The peptides were: 1) a peptide fragment of human TNFR1 which did not show binding on the ForteBio and serves as a negative control, A3 (SGSGNDCPGPGQDTDCREC), 2) a domain-1 peptide D2 (SGSGNSICCTKCHKGTYLY), 3) a domain-3 peptide D5 (SGSGCRKNQYRHYWSENLF) and 4) the overlapping domain-3 peptide E5 (SGSGNQYRHYWSENLFQCF). DOM1h-574-16 (2.5 μM) was flown over all four peptides and the amount of binding determined. No binding of DOM1h-574-16 was observed on the control peptide A3, while the dAb did bind the three other peptides. In the figure, the traces corresponding to the different peptides are indicated by the peptide identifier.

FIG. 20. Evaluation of binding of DOM1m-21-23 to four linear peptide fragments of TNFR1 by BIAcore. The four channels of a BIAcore SA chip were each coated with one of four biotinylated peptides. The peptides were: 1) a peptide fragment of human TNFR1 which did not show binding to DOM1h-574-16 on the ForteBio and serves as a negative control, A3 (SGSGNDCPGPGQDTDCREC), 2) a domain-1 peptide D2 (SGSGNSICCTKCHKGTYLY), 3) a domain-3 peptide D5 (SGSGCRKNQYRHYWSENLF) and 4) the overlapping domain-3 peptide E5 (SGSGNQYRHYWSENLFQCF). To establish if DOM1m-21-23 also binds these peptides, DOM1m-21-23 (2.5 μM) was injected over all four peptides. As can be seen from the figure, DOM1m-21-23 did not show binding to any of the four peptides. The curves overlay each other.

FIG. 21. Epitope mapping of DOM1h-131-511 to linear peptide fragments of TNFR1 by BIAcore. The four channels of a BIAcore SA chip were each coated with one of four biotinylated peptides. The peptides were: 1) a peptide fragment of human TNFR1 which did not show binding to DOM1h-574-16 on the ForteBio and serves as a negative control, A3 (SGSGNDCPGPGQDTDCREC), 2) a domain-1 peptide D2 (SGSGNSICCTKCHKGTYLY), 3) a domain-3 peptide D5 (SGSGCRKNQYRHYWSENLF) and 4) the overlapping domain-3 peptide E5 (SGSGNQYRHYWSENLFQCF). DOM1h-131-511 (2.5 μM) was flown over all four peptides and the amount of binding determined. As can be seen from the figure, DOM1h-131-511 did not show binding to any of the four peptides. The curves are close to overlaying and are indicated by arrows and the corresponding peptide number.

FIG. 22. BIAcore analysis for binding of DOM0100-AlbudAb in-line fusions to mouse serum albumin (MSA). MSA (Sigma-Aldrich) was coated on a BIAcore CM5 chip using EDC/NHS chemistry according to manufacturer's instructions. Subsequently, the DMS constructs, each consisting N-terminally to C-terminally of an anti-TNFR1 dAb-Linker-AlbudAb and identified in Table 6, were injected at 1 μM over the MSA surface and binding was monitored. As can be seen from the BIAcore traces, DMS0192 and DMS0188 had the best overall kinetics, while DMS0182 and DMS0184 were the weakest binders to MSA. The corresponding BIAcore trace for each DMS clone is indicated with an arrow.

FIG. 23. BIAcore analysis for binding of DOM0100-AlbudAb in-line fusions to human serum albumin (HSA). HSA (Sigma-Aldrich) was coated on a BIAcore CM5 chip using EDC/NHS chemistry according to manufacturer's instructions. Subsequently, the DMS constructs, each consisting N-terminally to C-terminally of an anti-TNFR1 dAb-Linker-AlbudAb and identified in Table 6, were injected at 1 μM over the HSA surface and binding was monitored. As can be seen from the BIAcore traces, DMS0189 and DMS0190 had the best overall kinetics, while the other DMS clones shown in the figure (DMS0182, DMS0184, DMS0186 and DMS0188) were very similar and significantly weaker in their affinity for HSA. The corresponding BIAcore trace for each DMS clone is indicated with an arrow.

FIG. 24. PK of DOM0100-AlbudAb fusions in mice. Mice were dosed with DMS0168 (2.5 mg/kg, intravenous), DMS0169 (2.5 mg/kg, intravenous) or DMS0182 (10 mg/kg, intraperitoneal). At each time point (0.17, 1, 4, 12, 24, 48 and 96 h) three mice were sacrificed and their serum analysed for levels of the respective DOM0100-AlbudAb fusion. The average amount of each DOM0100-AlbudAb fusion was determined for each time point and plotted against time, DMS0168 (grey dashed line), DMS0182 (black dotted line) and DMS0169 (black solid line) (corresponding lines are also indicated by arrows). Using non-compartmental analysis (NCA) in the WinNonLin analysis package (eg version 5.1 (available from Pharsight Corp., Mountain View, Calif. 94040, USA), the terminal half-life for each of the molecules was determined DMS0182 had a terminal half-life of 5.9 h, DMS0168 was 15.4 h and DMS0169 was 17.8 h. Due to the intraperitoneal dosing, the curve for DMS0182 has a different shape from that observed for DMS0168 and DMS0169.

FIG. 25. Arthritic score for Tg197/hp55 KI mice during saline and DMS0169 treatment. The transgenic mouse strain used in this study is a cross-bred of Tg197 (over-expressing human TNFα) and hp55 (knock-in of human TNFR1, also known as p55), which spontaneously develops arthritis. From week 6 till week 15, twelve mice in each group were treated twice a week with either 10 mg/kg of DMS0169 or saline. Each week the arthritic score was determined for the two hind joints per mouse and the average arthritic score, and standard error of the mean, over 12 mice was plotted in time. Clearly, the DMS0169 treated animals develop less arthritis.

FIG. 26. Body weight Tg197/hp55 KI mice during saline and DMS0169 treatment. The transgenic mouse strain used in this study is a cross-bred of Tg197 (over-expressing human TNFα) and hp55 (knock-in of human TNFR1, also known as p55), which spontaneously develops arthritis. From week 6 till week 15, twelve mice in each group were treated twice a week with either 10 mg/kg of DMS0169 or saline. Each week the mice were weighted and the average data plotted, with error bars indicating the standard error of the mean. From the figure, the trend for DMS0169 to be heavier, compared to saline treated animals is apparent, though not statistically significant.

FIG. 27. Histology and arthritic scores for Tg197/hp55 KI mice at week 15 after saline and DMS0169 treatment. The transgenic mouse strain used in this study is a cross-bred of Tg197 (over-expressing human TNFα) and hp55 (knock-in of human TNFR1, also known as p55), which spontaneously develops arthritis. From week 6 till week 15, twelve mice in each group were treated twice a week with either 10 mg/kg of DMS0169 or saline. At week 15 the mice were sacrificed and both arthritic score (black bars) and histology (open bars) in the joint were scored (Keffer et al. EMBO. J. 10, p 4025 (1991)). Each group consisted of twelve animals and the standard error was calculated. The difference between the treatment groups is shown to be statistically significant (p<0.001).

FIG. 28. Receptor Binding Assay (RBA) for a competitive and non-competitive anti-TNFR1 dAb. A dose range of either the competitive dAb DOM1h-131-511 (1.3 pM-100 nM, solid black line) or the non-competitive dAb DOM1h-574-10 (0.2 nM-16.3 μM, gray diamonds) were incubated with TNFR1-containing beads followed by incubation with TNFα. The dAb dose is plotted against the percentage inhibition in TNFα binding to TNFR1 observed.

FIG. 29. MRC-5 cell assay for a competitive and non-competitive anti-TNFR1 dAb. A dose range of either the competitive dAb DOM1h-131-511 (0.1 nM-100 nM, solid black line and squares) or the non-competitive dAb DOM1h-574-10 (0.14 nM-14 μM, dark-gray dashed line, open diamonds) were incubated with MRC5 cells in the presence of TNFα. After overnight incubation, the media was aspirated and the IL-8 levels in it were determined. The amounts were back calculated to the IL-8 levels determined in the absence of dAb. The dAb concentration was plotted against the percentage neutralization of IL-8 release.

FIG. 30. Inhibition of four different TNFα concentrations by a competitive anti-TNFR1 dAb in a MRC-5 cell assay. A standard MRC5 cell assay was done using four different concentrations of TNFα to stimulate the cells and a dose range of DOM1h-131-511. The concentrations of TNFα used were 10 pg/ml (dotted black line, open diamonds), 50 pg/ml (solid gray line, gray filled triangles), 200 pg/ml (dark gray dashed line, open triangles) and 2000 pg/ml (solid black line, black squares). Results are plotted as dAb concentration against percentage of inhibition of IL-8 secretion with individual lines for each concentration of TNFα used.

FIG. 31. Inhibition of four different TNFα concentrations by a non-competitive anti-TNFR1 dAb in a MRC-5 cell assay. A standard MRC5 cell assay was done using four different concentrations of TNFα to stimulate the cells and a dose range of DOM1h-574-138. The concentrations of TNFα used were 10 pg/ml (dotted black line, open diamonds), 50 pg/ml (solid gray line, gray filled triangles), 200 pg/ml (dark gray dashed line, open triangles) and 2000 pg/ml (solid black line, black squares). Results are plotted as dAb concentration against percentage of inhibition of IL-8 secretion with individual lines for each concentration of TNFα used.

FIG. 32. Inhibition of mouse TNFα-induced cytotoxicity in mouse L929 cells by a competitive and non-competitive anti-mouse TNFR1 dAb. A standard L929 mouse assay was done using two different mouse TNFα concentrations, 20 pg/ml (solid lines) or 100 pg/ml (dashed lines) and two different dAbs, DOM1m-15-12 (competitive dAb, gray lines) and DOM1m-21-23 (non-competitive dAb, black lines). The dAb concentration used to incubate the cells is plotted against the percentage neutralization of the cytotoxic effect of mouse TNFα on the L929 cells.

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

The immunoglobulin single variable domains (dAbs) described herein contain complementarity determining regions (CDR1, CDR2 and CDR3). The locations of CDRs and frame work (FR) regions and a numbering system have been defined by Kabat et al. (Kabat, E. A. et al., Sequences of Proteins of Immunological Interest, Fifth Edition, U.S. Department of Health and Human Services, U.S. Government Printing Office (1991)). The amino acid sequences of the CDRs (CDR1, CDR2, CDR3) of the V_{H} and V_{L} (V_{κ}) dAbs disclosed herein will be readily apparent to the person of skill in the art based on the well known Kabat amino acid numbering system and definition of the CDRs. According to the Kabat numbering system heavy chain CDR-H3 have varying lengths, insertions are numbered between residue H100 and H101 with letters up to K (i.e. H100, H100A H100K, H101). CDRs can alternatively be determined using the system of Chothia (Chothia et al., (1989) Conformations of immunoglobulin hypervariable regions; Nature 342, p 877-883), according to AbM or according to the Contact method as follows. See http://www.bioinf.org.uk/abs/ for suitable methods for determining CDRs.

Once each residue has been numbered, one can then apply the following CDR definitions (“-” means same residue numbers as shown for Kabat):

Kabat—most commonly used method based on sequence variability

(using Kabat numbering):

CDR H1: 31-35/35A/35B

CDR H2: 50-65

CDR H3: 95-102

CDR L1: 24-34

CDR L2: 50-56

CDR L3: 89-97

Chothia—based on location of the structural loop regions

(using Chothia numbering):

CDR H1: 26-32

CDR H2: 52-56

CDR H3: 95-102

CDR L1: 24-34

CDR L2: 50-56

CDR L3: 89-97

AbM—compromise between Kabat and Chothia

| (using Kabat numbering): | (using Chothia numbering): |
|---|---|
| CDR H1: 26-35/35A/35B | 26-35 |
| CDR H2: 50-58 | — |
| CDR H3: 95-102 | — |
| CDR L1: 24-34 | — |
| CDR L2: 50-56 | — |
| CDR L3: 89-97 | — |

Contact—based on crystal structures and prediction of contact residues with antigen

| (using Kabat numbering): | (using Chothia numbering): |
|---|---|
| CDR H1: 30-35/35A/35B | 30-35 |
| CDR H2: 47-58 | — |
| CDR H3: 93-101 | — |
| CDR L1: 30-36 | — |
| CDR L2: 46-55 | — |
| CDR L3: 89-96 | — |

As used herein, the term “antagonist of Tumor Necrosis Factor Receptor 1 (TNFR1)” or “anti-TNFR1 antagonist” or the like refers to an agent (e.g., a molecule, a compound) which binds TNFR1 and can inhibit a (i.e., one or more) function of TNFR1. For example, an antagonist of TNFR1 can inhibit the binding of TNFα to TNFR1 and/or inhibit signal transduction mediated through TNFR1. Accordingly, TNFR1-mediated processes and cellular responses (e.g., TNFα-induced cell death in a standard L929 cytotoxicity assay) can be inhibited with an antagonist of TNFR1.

As used herein, “peptide” refers to about two to about 50 amino acids that are joined together via peptide bonds.

As used herein, “polypeptide” refers to at least about 50 amino acids that are joined together by peptide bonds. Polypeptides generally comprise tertiary structure and fold into functional domains.

As used herein, a peptide or polypeptide (e.g. a domain antibody (dAb)) that is “resistant to protease degradation” is not substantially degraded by a protease when incubated with the protease under conditions suitable for protease activity. A polypeptide (e.g., a dAb) is not substantially degraded when no more than about 25%, no more than about 20%, no more than about 15%, no more than about 14%, no more than about 13%, no more than about 12%, no more than about 11%, no more than about 10%, no more than about 9%, no more than about 8%, no more than about 7%, no more than about 6%, no more than about 5%, no more than about 4%, no more than about 3%, no more that about 2%, no more than about 1%, or substantially none of the protein is degraded by protease after incubation with the protease for about one hour at a temperature suitable for protease activity, for example at 37 or 50 degrees C. Protein degradation can be assessed using any suitable method, for example, by SDS-PAGE or by functional assay (e.g., ligand binding) as described herein.

As used herein, “display system” refers to a system in which a collection of polypeptides or peptides are accessible for selection based upon a desired characteristic, such as a physical, chemical or functional characteristic. The display system can be a suitable repertoire of polypeptides or peptides (e.g., in a solution, immobilized on a suitable support). The display system can also be a system that employs a cellular expression system (e.g., expression of a library of nucleic acids in, e.g., transformed, infected, transfected or transduced cells and display of the encoded polypeptides on the surface of the cells) or an acellular expression system (e.g., emulsion compartmentalization and display). Exemplary display systems link the coding function of a nucleic acid and physical, chemical and/or functional characteristics of a polypeptide or peptide encoded by the nucleic acid. When such a display system is employed, polypeptides or peptides that have a desired physical, chemical and/or functional characteristic can be selected and a nucleic acid encoding the selected polypeptide or peptide can be readily isolated or recovered. A number of display systems that link the coding function of a nucleic acid and physical, chemical and/or functional characteristics of a polypeptide or peptide are known in the art, for example, bacteriophage display (phage display, for example phagemid display), ribosome display, emulsion compartmentalization and display, yeast display, puromycin display, bacterial display, display on plasmid, covalent display and the like. (See, e.g., EP 0436597 (Dyax), U.S. Pat. No. 6,172,197 (McCafferty et al.), U.S. Pat. No. 6,489,103 (Griffiths et al.).)

As used herein, “repertoire” refers to a collection of polypeptides or peptides that are characterized by amino acid sequence diversity. The individual members of a repertoire can have common features, such as common structural features (e.g., a common core structure) and/or common functional features (e.g., capacity to bind a common ligand (e.g., a generic ligand or a target ligand, TNFR1)).

As used herein, “functional” describes a polypeptide or peptide that has biological activity, such as specific binding activity. For example, the term “functional polypeptide” includes an antibody or antigen-binding fragment thereof that binds a target antigen through its antigen-binding site.

As used herein, “generic ligand” refers to a ligand that binds a substantial portion (e.g., substantially all) of the functional members of a given repertoire. A generic ligand (e.g., a common generic ligand) can bind many members of a given repertoire even though the members may not have binding specificity for a common target ligand. In general, the presence of a functional generic ligand-binding site on a polypeptide (as indicated by the ability to bind a generic ligand) indicates that the polypeptide is correctly folded and functional. Suitable examples of generic ligands include superantigens, antibodies that bind an epitope expressed on a substantial portion of functional members of a repertoire, and the like.

“Superantigen” is a term of art that refers to generic ligands that interact with members of the immunoglobulin superfamily at a site that is distinct from the target ligand-binding sites of these proteins. Staphylococcal enterotoxins are examples of superantigens which interact with T-cell receptors. Superantigens that bind antibodies include Protein G, which binds the IgG constant region (Bjorck and Kronvall, J. Immunol., 133:969 (1984)); Protein A which binds the IgG constant region and V_{H} domains (Forsgren and Sjoquist, J. Immunol., 97:822 (1966)); and Protein L which binds V_{L} domains (Bjorck, J. Immunol., 140:1194 (1988)).

As used herein, “target ligand” refers to a ligand which is specifically or selectively bound by a polypeptide or peptide. For example, when a polypeptide is an antibody or antigen-binding fragment thereof, the target ligand can be any desired antigen or epitope. Binding to the target antigen is dependent upon the polypeptide or peptide being functional.

As used herein an antibody refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format”, “formatted” or similar refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single antibody variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

The phrase “immunoglobulin single variable domain” refers to an antibody variable domain (V_{H}, V_{HH}, V_{L}) that specifically binds an antigen or epitope independently of other V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. A “single immunoglobulin variable domain” is the same as an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one embodiment, each individual organism or cell contains only one or a limited number of library members. In one embodiment, the nucleic acids are incorporated into expression vectors, in order to allow expression of the polypeptides encoded by the nucleic acids. In an aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of diverse polypeptides.

A “universal framework” is a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. Libraries and repertoires can use a single framework, or a set of such frameworks, which has been found to permit the derivation of virtually any binding specificity though variation in the hypervariable regions alone.

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds target antigen) administered to a subject over the course of one day (24 hours) (daily dose), two days, one week, two weeks, three weeks or one or more months (e.g., by a single administration, or by two or more administrations). The interval between doses can be any desired amount of time.

As used herein, “hydrodynamic size” refers to the apparent size of a molecule (e.g., a protein molecule, ligand) based on the diffusion of the molecule through an aqueous solution. The diffusion, or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation of the protein.

As referred to herein, the term “competes” means that the binding of a first target to its cognate target binding domain is inhibited in the presence of a second binding domain that is specific for the cognate target. For example, binding may be inhibited sterically, for example by physical blocking of a binding domain or by alteration of the structure or environment of a binding domain such that its affinity or avidity for a target is reduced. See WO2006038027 for details of how to perform competition ELISA and competition BiaCore experiments to determine competition between first and second binding domains.

Calculations of “homology” or “identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In an embodiment, the length of a reference sequence aligned for comparison purposes is at least 30%, or at least 40%, or at least 50%, or at least 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences. Amino acid and nucleotide sequence alignments and homology, similarity or identity, as defined herein may be prepared and determined using the algorithm BLAST 2 Sequences, using default parameters (Tatusova, T. A. et al., FEMS Microbiol Lett, 174:187-188 (1999)).

### Advantages of Partial Inhibition of TNFα According to the Invention

TNFα is a well documented pleiotropic cytokine involved in inflammatory, immunological and pathophysiological reactions. Excess TNFα production is one of the causes of the pathogenesis of inflammatory disease such as rheumatoid arthritis and inhibition of TNFα using anti-TNFα antibodies has been highly effective in the treatment of patients. However, TNFα also plays an important role in host immune defence by increasing phagocytosis by macrophages and enhancing mycobacterial killing in concert with IFNγ. The importance of this additional activity of TNFα is highlighted by the epidemiological evidence that individuals treated with TNFα inhibitors have an increased risk for the development of infections in the respiratory tract, in particular the reactivation of tuberculosis. Because of this dual role for TNFα, the inventors investigated partial inhibition of TNFR1, since incomplete inhibition of TNFα might be beneficial for reducing the susceptibility to infections. Most extensive modeling of the effects of residual free soluble TNFα on bacterial load was published by Marino et al (2007). The models disclosed in this publication suggest that only a very small amount of soluble TNFα is required for control of the infection. In the discussion Marino et al reiterate their major finding: ‘ . . . that anti-TNF therapy will likely lead to numerous incidents of primary TB if used in areas where exposure is likely, and that sTNF—even at very low levels—is essential for control of infection.’Very similar conclusions were reached by Guler et al, in a study comparing the effects of total and partial neutralisation of TNFα on cell-mediated immunity to Mycobacterium bovis BCG infection in mice. In this experimental study, regulation of TNFα levels was accomplished using transgenic mice expressing TNFR1 at varying levels. They conclude: ‘ . . . total neutralisation of TNF led to increased susceptibility [to BCG infection], whereas partial TNF inhibition resulted in enhanced granuloma formation and macrophage activities.’ These results were mimicked by Plessner et al in a chronic murine tuberculosis model comparing a monoclonal antibody against mouse TNFα and a TNFα-neutralizing TNFα receptor (TNFR) fusion molecule. From their studies Plessner et al conclude: ‘ . . . incomplete neutralization of TNF allows the host to maintain control of the infection.’

### REFERENCES

1: Marino S, Sud D, Plessner H, Lin P L, Chan J, Flynn J L, Kirschner D E.
Differences in reactivation of tuberculosis induced from anti-TNF treatments are based on bioavailability in granulomatous tissue.
PLoS Comput Biol. 2007 October; 3(10):1909-24. Epub 2007 August 22.
2: Plessner H L, Lin P L, Kohno T, Louie J S, Kirschner D, Chan J, Flynn J L.
Neutralization of tumor necrosis factor (TNF) by antibody but not TNF receptor fusion molecule exacerbates chronic murine tuberculosis.
J Infect Dis. 2007 Jun. 1; 195(11):1643-50. Epub 2007 April 23.
3: Guler R, Olleros M L, Vesin D, Parapanov R, Garcia I.
Differential effects of total and partial neutralization of tumor necrosis factor on cell-mediated immunity to Mycobacterium bovis BCG infection.
Infect Immun. 2005 June; 73(6):3668-76.

Neutralisation of TNFR1 (eg, human TNFR1) is determined in a cell assay, eg in a standard MRC5 assay as determined by inhibition of TNF alpha-induced IL-8 secretion; or in a standard L929 assay as determined by inhibition of TNF alpha-induced cytotoxicity; in a standard Cynomologus KI assay as determined by inhibition of TNF alpha-induced IL-8 secretion. Details of standard assays for TNFR1 antagonists are known in the art, eg in WO2006038027, WO2008149144 and WO2008149148. Details are also provided in the experimental section below. In one embodiment, the antagonist of the invention comprises or consists of an anti-TNFα receptor type 1 (TNFR1; p55) immunoglobulin single variable domain which comprises an amino acid sequence that is at least 95, 96, 97, 98 or 99% identical to the amino acid sequence of any one of the DOM1h variable domains shown in Table 11 below, optionally with the exception of DOM1h-574. In one embodiment, the antagonist of the invention comprises or consists of an anti-TNFα receptor type 1 (TNFR1; p55) immunoglobulin single variable domain which comprises an amino acid sequence that is at least 95, 96, 97, 98 or 99% identical to the amino acid sequence of any one of DOM1h-574-89 to DOM1h-574-179.

In one embodiment, the antagonist of the invention comprises or consists of an anti-TNFα receptor type 1 (TNFR1; p55) immunoglobulin single variable domain which comprises an amino acid sequence that is identical to, or at least 94, 95, 96, 97, 98 or 99% identical to, the amino acid sequence of DOM1h-574-109, DOM1h-574-93, DOM1h-574-123, DOM1h-574-125, DOM1h-574-126 or DOM1h-574-129, DOM1h-574-133, DOM1h-574-137 or DOM1h-574-160. This aspect provides variable domains that that are proteolytically stable. Reference is made to the discussion above on protease stability.

In one embodiment, the antagonist of the invention comprises or consists of an anti-TNFα receptor type 1 (TNFR1; p55) immunoglobulin single variable domain which comprises an amino acid sequence that is identical to, or at least 95, 96, 97, 98 or 99% identical to, to the amino acid sequence of DOM1h-574-72, DOM1h-574-109, DOM1h-574-125, DOM1h-574-126, DOM1h-574-133, DOM1h-574-135 or DOM1h-574-138, DOM1h-574-139, DOM1h-574-155, DOM1h-574-156, DOM1h-574-162 or DOM1h-574-180. This aspect provides variable domains that bind human TNFR1 with high affinity and optionally also display desirable affinity for murine TNFR1.

The antagonist, eg, single variable domain, is a non-competitive inhibitor of TNFR1. In one embodiment, the TNFR1 antagonist binds TNFR1 (eg, human TNFR1) but does not (or does not substantially) compete with or inhibit TNF alpha for binding to TNFR1 (eg, in a standard receptor binding assay). In this embodiment, in one example the antagonist (eg, an anti-TNFR1 variable domain or PLAD peptide) specifically binds to domain 1 of TNFR1, eg, human TNFR1. In this embodiment, in one example the antagonist specifically binds to the PLAD of TNFR1, eg, human TNFR1.

In one embodiment, the antagonist of any aspect of the invention comprises or consists of an anti-TNFR1 single variable comprising a binding site that specifically binds

(i) human TNFR1 with a dissociation constant (KD) of (or of about) 500 pM or less, 400 pM or less, 350 pM or less, 300 pM or less, 250 pM or less, 200 pM or less, or 150 pM or less as determined by surface plasmon resonance; or
(ii) non-human primate TNFR1 (eg, Cynomolgus monkey, rhesus or baboon TNFR1) with a dissociation constant (KD) of (or of about) 500 pM or less, 400 pM or less, 350 pM or less, 300 pM or less, 250 pM or less, 200 pM or less, or 150 pM or less as determined by surface plasmon resonance; or
(iii) murine TNFR1 with a dissociation constant (KD) of (or of about) 7 nM or less, 6 nM or less, 5 nM or less, 4 nM or less, 3 nM or less, 2 nM or less, or 1 nM or less as determined by surface plasmon resonance. In one example, the variable domain specifically binds according to (i) and (ii); (i) and (iii); (i), (ii) and (iii), or (ii) and (iii). In one embodiment, the antagonist of any aspect of the invention comprises or consists of an anti-TNFR1 single variable comprising a binding site that specifically binds
(a) human TNFR1 with an off-rate constant (Koff) of (or of about) 2×10⁻⁴ S⁻¹ or less, or 1×10⁻⁴ S⁻¹ or less, or 1×10⁻⁵ S⁻¹ or less as determined by surface plasmon resonance;
(b) non-human primate TNFR1 (eg, Cynomolgus monkey, rhesus or baboon TNFR1) with an off-rate constant (Koff) of (or of about) 2×10⁻⁴ S⁻¹ or less, 1×10⁻⁴ S⁻¹ or less, or 1×10⁻⁵ S⁻¹ or less as determined by surface plasmon resonance; or
(c) murine TNFR1 with an off-rate constant (Koff) of (or of about) 1×10⁻³ S⁻¹ or less, or 1×10⁻⁴ S⁻¹ or less as determined by surface plasmon resonance. In one example, the variable domain specifically binds according to (a) and (b); (a) and (c); (a), (b) and (c), or (b) and (c).
In one embodiment, the antagonist of any aspect of the invention comprises or consists of an anti-TNFR1 single variable comprising a binding site that specifically binds
(a′) human TNFR1 with an on-rate constant (Kon) of (or of about) 5×10⁴ M⁻¹s⁻¹ or more, 1×10⁵ M⁻¹s⁻¹ or more, 2×10⁵ M⁻¹s⁻¹ or more, 3×10⁵ M⁻¹s⁻¹ or more, 4×10⁵ M⁻¹s⁻¹ or more, or 5×10⁵ M⁻¹s⁻¹ or more as determined by surface plasmon resonance;
(b′) non-human primate TNFR1 (eg, Cynomolgus monkey, rhesus or baboon TNFR1) with an on-rate constant (Kon) of (or of about) 5×10⁴ M⁻¹s⁻¹ or more, 1×10⁵ M⁻¹s⁻¹ or more, 2×10⁵ M⁻¹s⁻¹ or more, 3×10⁵ M⁻¹s⁻¹ or more, 4×10⁵ M⁻¹s⁻¹ or more, or 5×10⁵ M⁻¹s⁻¹ or more as determined by surface plasmon resonance; or
(c′) murine TNFR1 with an on-rate constant (Kon) of (or of about) 0.5×10⁵ M⁻¹s⁻¹ or more, 1×10⁵ M⁻¹s⁻¹ or more, or 2×10⁵ M⁻¹s⁻¹ or more as determined by surface plasmon resonance. In one example, the antagonist specifically binds according to (a′) and (b′); (a′) and (c′); (a′), (b′) and (c′), or (b′) and (c′).

In one embodiment, the antagonist of the invention comprises or consists of a single variable domain that specifically binds human, Cynomologus monkey and optionally canine TNFR1. Specific binding is indicated by a dissociation constant KD of 10 micromolar or less, optionally 1 micromolar or less. Specific binding of an antigen-binding protein to an antigen or epitope can be determined by a suitable assay, including, for example, Scatchard analysis and/or competitive binding assays, such as radioimmunoassays (RIA), enzyme immunoassays such as ELISA and sandwich competition assays, and the different variants thereof. In one example, antagonist also specifically binds murine TNFR1.

In one embodiment of any aspect of the invention, the antagonist of the invention comprises or consists of a single variable domain that inhibits the binding of human, Cynomologus monkey and optionally canine TNFR1 to DOM1h-574-72, DOM1h-574-109, DOM1h-574-138, DOM1h-574-156, DOM1h-574-162 or DOM1h-574-180, for example in a standard cell assay (eg, as described herein or in WO2006038027, WO2008149144 or WO2008149148. In an embodiment the single variable domain inhibits the binding of human, murine, Cynomologus monkey and optionally canine TNFR1 to DOM1h-574-72, DOM1h-574-109, DOM1h-574-138, DOM1h-574-156, DOM1h-574-162 or DOM1h-574-180, for example in a standard receptor binding assay (eg, as described herein or in WO2006038027, WO2008149144 or WO2008149148. In an example, “inhibits” in these embodiments is inhibition can be total (100% inhibition) or substantial (at least 90%, 95%, 98%, or 99%).

In one embodiment of any aspect of the invention, the antagonist neutralizes TNFR1 (eg, human TNFR1) with an ND50 of (or about of) 5, 4, 3, 2 or 1 nM or less in a standard MRC5 assay as determined by inhibition of TNF alpha-induced IL-8 secretion.

In one embodiment of any aspect of the invention, the antagonist neutralizes TNFR1 (eg, murine TNFR1) with an ND50 of 150, 100, 50, 40, 30 or 20 nM or less; or from (about) 150 to 10 nM; or from (about) 150 to 20 nM; or from (about) 110 to 10 nM; or from (about) 110 to 20 nM in a standard L929 assay as determined by inhibition of TNF alpha-induced cytotoxicity.

In one embodiment of any aspect of the invention, the antagonist neutralizes TNFR1 (eg, Cynomologus monkey TNFR1) with an ND50 of 5, 4, 3, 2 or 1 nM or less; or (about) 5 to (about) 1 nM in a standard Cynomologus KI assay as determined by inhibition of TNF alpha-induced IL-8 secretion.

In one embodiment of any aspect of the invention, the antagonist comprises or consists of a single variable domain which comprises a terminal, optionally C-terminal, cysteine residue. For example, the cysteine residue can be used to attach PEG to the variable domain, eg, using a maleimide linkage (see, eg, WO04081026). In an embodiment, the single variable domain is linked to a polyalkylene glycol moiety, optionally a polyethylene glycol moiety. See, eg, WO04081026, for suitable PEG moieties and conjugation methods and tests. These disclosures are incorporated herein in order to provide disclosure, for example of specific PEGs to be included in claims below.

the antagonist comprises or consists of an anti-TNFα receptor type 1 (TNFR1; p55) immunoglobulin single variable domain comprising an amino acid sequence that is identical to the amino acid sequence selected from the amino acid sequence of DOM1h-574-72, DOM1h-574-109, DOM1h-574-138, DOM1h-574-156, DOM1h-574-162 and DOM1h-574-180 or differs from the selected amino acid sequence at no more than 25, 20, 15, 10 or 5 amino acid positions and has a CDR1 sequence that is identical to, or at least 50, 60, 70, 80, 90, 95 or 98% identical to, the CDR1 sequence of the selected amino acid sequence. In one embodiment, the immunoglobulin single variable domain comprises a CDR3 sequence that is identical to, or at least 50, 60, 70, 80, 90, 95 or 98% identical to, the CDR3 sequence of the selected amino acid sequence.

the antagonist comprises or consists of an anti-TNFα receptor type 1 (TNFR1; p55) immunoglobulin single variable domain which comprising an amino acid sequence that is identical to the amino acid sequence selected from the amino acid sequence of DOM1h-574-72, DOM1h-574-109, DOM1h-574-138, DOM1h-574-156, DOM1h-574-162 and DOM1h-574-180 or differs from the selected amino acid sequence at no more than 25, 20, 15, 10 or 5 amino acid positions and has a CDR2 sequence that is identical to, or at least 50, 60, 70, 80, 90, 95 or 98% identical to, the CDR2 sequence of the selected amino acid sequence. In one embodiment, the immunoglobulin single variable domain comprises a CDR2 sequence that is identical to, or at least 50, 60, 70, 80, 90, 95 or 98% identical to, the CDR2 sequence of the selected amino acid sequence. Additionally, or alternatively, in one embodiment, the immunoglobulin single variable domain comprises a CDR3 sequence that is identical to, or at least 50, 60, 70, 80, 90, 95 or 98% identical to, the CDR3 sequence of the selected amino acid sequence. Additionally, or alternatively, in one embodiment, the immunoglobulin single variable domain comprises a CDR1 sequence that is identical to, or at least 50, 60, 70, 80, 90, 95 or 98% identical to, the CDR1 sequence of the selected amino acid sequence.

the antagonist comprises or consists of an anti-TNFα receptor type 1 (TNFR1; p55) immunoglobulin single variable domain which comprising an amino acid sequence that is identical to the amino acid sequence selected from the amino acid sequence of DOM1h-574-72, DOM1h-574-109, DOM1h-574-138, DOM1h-574-156, DOM1h-574-162 and DOM1h-574-180 or differs from the selected amino acid sequence at no more than 25, 20, 15, 10 or 5 amino acid positions and has a CDR3 sequence that is identical to, or at least 50, 60, 70, 80, 90, 95 or 98% identical to, the CDR3 sequence of the selected amino acid sequence.

the antagonist comprises or consists of a protease resistant anti-TNFα receptor type 1 (TNFR1; p55) immunoglobulin single variable domain, wherein the single variable domain is resistant to protease when incubated with

(i) a concentration (c) of at least 10 micrograms/ml protease at 37° C. for time (t) of at least one hour; or
(ii) a concentration (c′) of at least 40 micrograms/ml protease at 30° C. for time (t) of at least one hour.
wherein the variable domain comprises an amino acid sequence that is at least 94, 95, 96, 97, 98 or 99% identical to the amino acid sequence of DOM1h-574-126 or DOM1h-574-133, and optionally comprises a valine at position 101 (Kabat numbering). In another aspect, the invention provides a protease resistant anti-TNFα receptor type 1 (TNFR1; p55) immunoglobulin single variable domain, wherein the single variable domain is resistant to protease when incubated with
(i) a concentration (c) of at least 10 micrograms/ml protease at 37° C. for time (t) of at least one hour; or
(ii) a concentration (c′) of at least 40 micrograms/ml protease at 30° C. for time (t) of at least one hour.
wherein the variable domain comprises an amino acid sequence that is at least 70, 75, 80, 85, 90, 91, 92, 93, 94, 95, 96, 97, 98 or 99% identical to the amino acid sequence of DOM1h-574, DOM1h-574-93, DOM1h-574-123, DOM1h-574-125, DOM1h-574-126, DOM1h-574-129, DOM1h-574-133, DOM1h-574-137 or DOM1h-574-160, and optionally comprises a valine at position 101 (Kabat numbering).

The protease resistant anti-TNFR1 variable domain is a non-competitive variable domain (ie, it does not (substantially) inhibit the binding of TNF alpha to TNFR1). See the discussion above on non-competitive variable domains, which applies to these embodiments too.

In one example of these embodiments the concentration (c or c′) is at least 100 or 1000 micrograms/ml protease. In one embodiment, time (t) is one, three or 24 hours or overnight. In one example, the variable domain is resistant under conditions (i) and the concentration (c) is 10 or 100 micrograms/ml protease and time (t) is 1 hour. In one example, the variable domain is resistant under conditions (ii) and the concentration (c′) is 40 micrograms/ml protease and time (t) is 3 hours. In one embodiment, the protease is selected from trypsin, elastase, leucozyme and pancreatin. In one embodiment, the protease is trypsin. In one embodiment, the variable domain is resistant to trypsin and at least one other protease selected from elastase, leucozyme and pancreatin. In one embodiment, the variable domain specifically binds TNFR1 following incubation under condition (i) or (ii). In one embodiment, the variable domain has an OD₄₅₀ reading in ELISA of at least 0.404 following incubation under condition (i) or (ii). In one embodiment, the variable domain specifically binds protein A or protein L following incubation under condition (i) or (ii). In one embodiment, the variable domain displays substantially a single band in gel electrophoresis following incubation under condition (i) or (ii). In one embodiment, the single variable domain that has a Tm of at least 50° C. More details relating to protease resistance can be found in WO2008149144 and WO2008149148.

In one aspect, the antagonist of the invention comprises or consists of a polypeptide comprising an anti-TNFR1 immunoglobulin single variable domain as herein described and an effector group or an antibody constant domain, optionally an antibody Fc region, optionally wherein the N-terminus of the Fc is linked (optionally directly linked) to the C-terminus of the variable domain. Any “effector group” as described in WO04058820 can be used in this embodiment, and the description of the effector groups in WO04058820 and methods of linking them to variable domains disclosed in that publication are explicitly incorporated herein by reference to provide description herein that can be used, for example, in claims herein. In one embodiment, the polypeptide comprises an Fc fusion of DOM1h-574-16 or DOM1h-574-72.

In one aspect, the antagonist of the invention comprises or consists of a multispecific ligand comprising an immunoglobulin single variable domain as herein described and optionally at least one immunoglobulin single variable domain that specifically binds serum albumin (SA). Surprisingly, the inventors found that fusion of such an anti-TNFR1 single variable to an anti-SA single variable domain provides the advantage of improved half-life (over an anti-TNFR1 dAb monomer alone), but also with the added benefit of an improvement in the affinity (KD) for TNFR1 binding. This observation has not been disclosed before in the state of the art. In this respect, there is provided the antagonist of the invention comprising a multispecific ligand which comprises such an anti-TNFR1 immunoglobulin single variable domain and an anti-SA (eg, anti-human SA) immunoglobulin single variable domain for providing a ligand that has a longer half-life and a lower KD for TNFR1 binding (eg, human TNFR1 binding) than the anti-TNFR1 immunoglobulin single variable domain when provided as a variable domain monomer (ie, when the anti-TNFR1 variable domain is unformatted, eg, not PEGylated or fused to an antibody constant region such as an Fc region, and is not fused to any other domain). In one embodiment, the multispecific ligand binds TNFR1 (eg, human TNFR1) with a KD that is at least two-fold lower than the KD of the TNFR1 monomer. Additionally or alternatively, in one embodiment, the multispecific ligand has a half-life that is at least 5, 10, 20, 30, 40, 50 or 100 times that of the monomer. Additionally or alternatively, in one embodiment, the multispecific ligand has a terminal half-life of at least 15, 16, 17, 18, 19, 20, 21, 22, 23, 24 or 25 days in man (for example as determined empirically in human volunteers or as calculated using conventional techniques familiar to the skilled person by extrapolating from the half-life of the ligand in an animal system such as mouse, dog and/or non-human primate (eg, Cynomolgus monkey, baboon, rhesus monkey)), for example where the anti-SA domain is cross-reactive between human SA and SA from the animal.

In one embodiment, the antagonist according to the invention has a tβ half-life in the range of (or of about) 2.5 hours or more. In one embodiment, the lower end of the range is (or is about) 3 hours, 4 hours, 5 hours, 6 hours, 7 hours, 10 hours, 11 hours, or 12 hours. In addition, or alternatively, the tβ half-life is (or is about) up to and including 21 or 25 days. In one embodiment, the upper end of the range is (or is about) 12 hours, 24 hours, 2 days, 3 days, 5 days, 10 days, 15 days, 19 days 20 days, 21 days or 22 days. For example, the antagonist according to the invention will have a β half life in the range 12 to 60 hours (or about 12 to 60 hours). In a further embodiment, it will be in the range 12 to 48 hours (or about 12 to 48 hours). In a further embodiment still, it will be in the range 12 to 26 hours (or about 12 to 26 hours).

As an alternative to using two-compartment modeling, the skilled person will be familiar with the use of non-compartmental modeling, which can be used to determine terminal half-lives (in this respect, the term “terminal half-life” as used herein means a terminal half-life determined using non-compartmental modeling). The WinNonlin analysis package, eg version 5.1 (available from Pharsight Corp., Mountain View, Calif. 94040, USA) can be used, for example, to model the curve in this way. In this instance, in one embodiment antagonist has a terminal half life of at least (or at least about) 8 hours, 10 hours, 12 hours, 15 hours, 28 hours, 20 hours, 1 day, 2 days, 3 days, 7 days, 14 days, 15 days, 16 days, 17 days, 18 days, 19 days, 20 days, 21 days, 22 days, 23 days, 24 days or 25 days. In one embodiment, the upper end of this range is (or is about) 24 hours, 48 hours, 60 hours or 72 hours or 120 hours. For example, the terminal half-life is (or is about) from 8 hours to 60 hours, or 8 hours to 48 hours or 12 to 120 hours, eg, in man.

In addition, or alternatively to the above criteria, the antagonist according to the invention has an AUC value (area under the curve) in the range of (or of about) 1 mg·min/ml or more. In one embodiment, the lower end of the range is (or is about) 5, 10, 15, 20, 30, 100, 200 or 300 mg·min/ml. In addition, or alternatively, the antagonist according to the invention has an AUC in the range of (or of about) up to 600 mg·min/ml. In one embodiment, the upper end of the range is (or is about) 500, 400, 300, 200, 150, 100, 75 or 50 mg·min/ml. Advantageously the variable domain or antagonist will have a AUC in (or about in) the range selected from the group consisting of the following: 15 to 150 mg·min/ml, 15 to 100 mg·min/ml, 15 to 75 mg·min/ml, and 15 to 50 mg·min/ml.

One or more of the t alpha, t beta and terminal half-lives as well as the AUCs quoted herein can be obtained in a human and/or animal (eg, mouse or non-human primate, eg, baboon, rhesus, Cynomolgus monkey) by providing one or more anti-TNFR1 single variable domains (or other binding moieties defined herein) linked to either a PEG or a single variable domain (or binding moiety) that specifically binds to serum albumin, eg mouse and/or human serum albumin (SA). The PEG size can be (or be about) at least 20 kDa, for example, 30, 40, 50, 60, 70 or 80 kDa. In one embodiment, the PEG is 40 kDa, eg 2×20 kDa PEG. In one embodiment, to obtain at alpha, t beta and terminal half-lives or an AUC quoted herein, there is provide an antagonist comprising an anti-TNFR1 immunoglobulin single variable domain linked to an anti-SA immunoglobulin single variable domain. In one embodiment, the PEG is 40 kDa, eg 2×20 kDa PEG. For example, the antagonist comprises only one such anti-TNFR1 variable domains, for example one such domain linked to only one anti-SA variable domains. In one embodiment, to obtain at alpha, t beta and terminal half-lives or a AUC quoted herein, there is provide an antagonist comprising an anti-TNFR1 immunoglobulin single variable domain linked to PEG, eg, 40-80 kDa PEG, eg, 40 kDa PEG. For example, the antagonist comprises only one such anti-TNFR1 variable domains, for example one such domain linked to 40 kDa PEG.

In one embodiment of the multispecific ligand, the ligand comprises an anti-SA (eg, HSA) single variable domain that comprises an amino acid sequence that is identical to, or at least 80, 85, 90, 91, 92, 93, 94, 95, 96, 97, 98 or 99% identical to, the sequence of DOM7h-11, DOM7h-11-3, DOM7h-11-12, DOM7h-11-15, DOM7h-14, DOM7h-14-10, DOM7h-14-18 or DOM7m-16. Alternatively or additionally, in an embodiment, the multispecific ligand comprises a linker provided between the anti-TNFR1 single variable domain and the anti-SA single variable domain, the linker comprising the amino acid sequence AST, optionally ASTSGPS. For example, the ligand comprises (N- to C-terminally) DOM1h-574-16-AST-DOM7h-11; or DOM1h-574-72-ASTSGPS-DOM7m-16; or DOM1h-574-72-ASTSGPS-DOM7h-11-12.

In one aspect, the antagonists of the invention comprises or consists of a multispecific ligand comprising (i) an anti-TNFα receptor type 1 (TNFR1; p55) immunoglobulin single variable domain which comprises an amino acid sequence that is identical to, or at least 93, 94, 95, 96, 97, 98 or 99% identical to, the amino acid sequence of DOM1h-574-156, (ii) at least one anti-serum albumin (SA) immunoglobulin single variable domain that specifically binds SA, wherein the anti-SA single variable domain comprises an amino acid sequence that is identical to, or at least 80, 85, 90, 91, 92, 93, 94, 95, 96, 97, 98 or 99% identical to, the sequence of DOM7h-11-3, and (iii) optionally wherein a linker is provided between the anti-TNFR1 single variable domain and the anti-SA single variable domain, the linker comprising the amino acid sequence AST, optionally ASTSGPS. For example, the ligand comprises DOM1h-574-156 and DOM7h-11-3 optionally linked by AST or ASTSGPS. In this example, the ligand is optionally adapted for administration to a patient by intravascularly, sub-cutaneously, intramuscularly, peritoneally or by inhalation. In one example, the ligand is provided as a dry-powder or lyophilized composition (which optionally is mixed with a diluent prior to administration).

In one aspect, the antagonists of the invention comprises or consists of a multispecific ligand comprising (i) an anti-TNFα receptor type 1 (TNFR1; p55) immunoglobulin single variable domain which comprises an amino acid sequence that is identical to, or at least 93, 94, 95, 96, 97, 98 or 99% identical to, the amino acid sequence of DOM1h-574-156, (ii) at least one anti-serum albumin (SA) immunoglobulin single variable domain that specifically binds SA, wherein the anti-SA single variable domain comprises an amino acid sequence that is identical to, or at least 80, 85, 90, 91, 92, 93, 94, 95, 96, 97, 98 or 99% identical to, the sequence of DOM7h-14-10, and (iii) optionally wherein a linker is provided between the anti-TNFR1 single variable domain and the anti-SA single variable domain, the linker comprising the amino acid sequence AST, optionally ASTSGPS. For example, the ligand comprises DOM1h-574-156 and DOM7h-14-10 optionally linked by AST or ASTSGPS. In this example, the ligand is optionally adapted for administration to a patient by intravascularly, sub-cutaneously, intramuscularly, peritoneally or by inhalation. In one example, the ligand is provided as a dry-powder or lyophilized composition (which optionally is mixed with a diluent prior to administration).

For example, the antagonist of the invention is monovalent for TNFR1 binding. For example, the antagonist of the invention is monovalent or substantially monovalent as determined by standard SEC-MALLS. Substantial monovalency is indicated by no more than 5, 4, 3, 2 or 1% of the antagonist being present in a non-monovalent form as determined by standard SEC-MALLS.

In one embodiment, the antagonist of the invention comprises first and second anti-TNFR1 immunoglobulin single variable domains, wherein each variable domain is as herein described. The first and second immunoglobulin single variable domains are in one example identical. In another example they are different.

In one example, the amino acid sequence of the or each anti-TNFR1 single variable domain in an antagonist of the invention is identical to the amino acid sequence of DOM1h-574-16 or DOM1h-574-72.

In one aspect, the invention provides a TNFα receptor type 1 (TNFR1; p55) antagonist comprising an anti-TNFR1 variable domain according any aspect of the invention, for oral delivery, delivery to the GI tract of a patient, pulmonary delivery, delivery to the lung of a patient or systemic delivery. In another aspect, the invention provides the use of the TNFR1 antagonist of any aspect of the invention in the manufacture of a medicament for oral delivery. In another aspect, the invention provides the use of the TNFR1 antagonist of any aspect of the invention in the manufacture of a medicament for delivery to the GI tract of a patient. In one example of the antagonist or the variable domain is resistant to trypsin, elastase and/or pancreatin.

In one aspect, the invention provides the use of a TNFR1 antagonist of any aspect of the invention in the manufacture of a medicament for pulmonary delivery. In another aspect, the invention provides the use of a TNFR1 antagonist of any aspect of the invention in the manufacture of a medicament for delivery to the lung of a patient. In one example of the antagonist or the variable domain is resistant to leucozyme.

In one aspect, the invention provides a method of oral delivery or delivery of a medicament to the GI tract of a patient or to the lung or pulmonary tissue of a patient, wherein the method comprises administering to the patient a pharmaceutically effective amount of a TNFR1 antagonist of the invention.

In one aspect, the invention provides a TNFα receptor type 1 (TNFR1; p55) antagonist of the invention for binding human, murine or Cynomologus monkey TNFR1, the antagonist having a CDR1 sequence that is identical to, or at least 50, 60, 70, 80, 90, 95 or 98% identical to, the CDR1 sequence of DOM1h-574-72, DOM1h-574-109, DOM1h-574-138, DOM1h-574-156, DOM1h-574-162 and DOM1h-574-180. Optionally, the antagonist also has a CDR2 sequence that is identical to, or at least 50, 60, 70, 80, 90, 95 or 98% identical to, the CDR2 sequence of the selected sequence. Optionally, additionally or alternatively, the antagonist also has a CDR3 sequence that is identical to, or at least 50, 60, 70, 80, 90, 95 or 98% identical to, the CDR3 sequence of the selected sequence.

In one aspect, the invention provides a TNFα receptor type 1 (TNFR1; p55) antagonist of the invention for binding human, murine or Cynomologus monkey TNFR1, the antagonist having a CDR2 sequence that is identical to, or at least 50, 60, 70, 80, 90, 95 or 98% identical to, the CDR2 sequence of DOM1h-574-72, DOM1h-574-109, DOM1h-574-138, DOM1h-574-156, DOM1h-574-162 and DOM1h-574-180. Optionally, the antagonist also has a CDR3 sequence that is identical to, or at least 50, 60, 70, 80, 90, 95 or 98% identical to, the CDR3 sequence of the selected sequence.

In one aspect, the invention provides a TNFα receptor type 1 (TNFR1; p55) antagonist of the invention for binding human, murine or Cynomologus monkey TNFR1, the antagonist having a CDR3 sequence that is identical to, or at least 50, 60, 70, 80, 90, 95 or 98% identical to, the CDR3 sequence of DOM1h-574-72, DOM1h-574-109, DOM1h-574-138, DOM1h-574-156, DOM1h-574-162 and DOM1h-574-180.

In one aspect, the invention provides a TNFα receptor type 1 (TNFR1; p55) antagonist of the invention for binding human, murine or Cynomologus monkey TNFR1, the antagonist comprising an immunoglobulin single variable domain comprising the sequence of CDR1, CDR2, and/or CDR3 of a single variable domain selected from DOM1h-574-72, DOM1h-574-109, DOM1h-574-138, DOM1h-574-156, DOM1h-574-162 and DOM1h-574-180.

The invention provides the TNFR1 antagonist of any aspect for treating and/or prophylaxis of an inflammatory condition. The invention provides the use of the TNFR1 antagonist of any aspect in the manufacture of a medicament for treating and/or prophylaxis of an inflammatory condition. In one embodiment of the antagonist or use, the condition is selected from the group consisting of arthritis, multiple sclerosis, inflammatory bowel disease and chronic obstructive pulmonary disease. In one example, the arthritis is rheumatoid arthritis or juvenile rheumatoid arthritis. In one example, the inflammatory bowel disease is selected from the group consisting of Crohn's disease and ulcerative colitis. In one example, the chronic obstructive pulmonary disease is selected from the group consisting of chronic bronchitis, chronic obstructive bronchitis and emphysema. In one example, the pneumonia is bacterial pneumonia. In one example, the bacterial pneumonia is Staphylococcal pneumonia.

The invention provides a TNFR1 antagonist of any aspect for treating and/or prophylaxis of a respiratory disease. The invention provides the use of the TNFR1 antagonist of any aspect in the manufacture of a medicament for treating and/or prophylaxis of a respiratory disease. In one example the respiratory disease is selected from the group consisting of lung inflammation, chronic obstructive pulmonary disease, acute lung injury (ALI), asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

In one aspect, the anti-TNFR1 of any one aspect of the invention is provided for targeting one or more epitopic sequence of TNFR1 selected from the group consisting of NSICCTKCHKGTYLY, NSICCTKCHKGTYL, CRKNQYRHYWSENLF and NQYRHYWSENLFQCF. In one example, the anti-TNFR1 antagonist is provided for targeting NSICCTKCHKGTYLY. In one example, the anti-TNFR1 antagonist is provided for targeting NSICCTKCHKGTYL. In one example, the anti-TNFR1 antagonist is provided for targeting CRKNQYRHYWSENLF. In one example, the anti-TNFR1 antagonist is provided for targeting NQYRHYWSENLFQCF. In one example, the anti-TNFR1 antagonist is provided for targeting CRKNQYRHYWSENLF and NQYRHYWSENLFQCF. In one example, the anti-TNFR1 antagonist is provided for targeting NSICCTKCHKGTYLY, CRKNQYRHYWSENLF and NQYRHYWSENLFQCF. In one example, the anti-TNFR1 antagonist is provided for targeting NSICCTKCHKGTYL, CRKNQYRHYWSENLF and NQYRHYWSENLFQCF. In one example, such targeting is to treat and/or prevent any condition or disease specified above. In one aspect, the invention provides a method of treating and/or preventing any condition or disease specified above in a patient, the method comprising administering to the patient an anti-TNFR1 antagonist of the invention for targeting one or more epitopic sequence of TNFR1 as described in any of the preceding embodiments.

Polypeptides, dAbs & Antagonists

The polypeptide, ligand, dAb, ligand or antagonist can be expressed in E. coli or in Pichia species (e.g., P. pastoris). In one embodiment, the ligand or dAb monomer is secreted in a quantity of at least about 0.5 mg/L when expressed in E. coli or in Pichia species (e.g., P. pastoris). Although, the ligands and dAb monomers described herein can be secretable when expressed in E. coli or in Pichia species (e.g., P. pastoris), they can be produced using any suitable method, such as synthetic chemical methods or biological production methods that do not employ E. coli or Pichia species.

In some embodiments, the polypeptide, ligand, dAb, ligand or antagonist does not comprise a Camelid immunoglobulin variable domain, or one or more framework amino acids that are unique to immunoglobulin variable domains encoded by Camelid germline antibody gene segments, eg at position 108, 37, 44, 45 and/or 47. In one embodiment, the anti-TNFR1 variable domain comprises a G residue at position 44 according to Kabat and optionally comprises one or more Camelid-specific amino acids at other positions, eg at position 37 or 103.

Antagonists of TNFR1 according to the invention can be monovalent or multivalent. In some embodiments, the antagonist is monovalent and contains one binding site that interacts with TNFR1, the binding site provided by a polypeptide or dAb as herein described. Monovalent antagonists bind one TNFR1 and may not induce cross-linking or clustering of TNFR1 on the surface of cells which can lead to activation of the receptor and signal transduction.

In other embodiments, the antagonist of TNFR1 is multivalent. Multivalent antagonists of TNFR1 can contain two or more copies of a particular binding site for TNFR1 or contain two or more different binding sites that bind TNFR1, at least one of the binding sites being provided by a polypeptide or dAb as herein described. For example, as described herein the antagonist of TNFR1 can be a dimer, trimer or multimer comprising two or more copies of a particular polypeptide or dAb as herein described that binds TNFR1, or two or more different polypeptides or dAbs as herein described that bind TNFR1. In one embodiment, a multivalent antagonist of TNFR1 does not substantially agonize TNFR1 (act as an agonist of TNFR1) in a standard cell assay (i.e., when present at a concentration of 1 nM, 10 nM, 100 nM, 1 μM, 10 μM, 100 μM, 1000 μM or 5,000 μM, results in no more than about 5% of the TNFR1-mediated activity induced by TNFα (100 pg/ml) in the assay).

In certain embodiments, the multivalent antagonist of TNFR1 contains two or more binding sites for a desired epitope or domain of TNFR1. For example, the multivalent antagonist of TNFR1 can comprise two or more binding sites that bind the same epitope in Domain 1 of TNFR1.

In other embodiments, the multivalent antagonist of TNFR1 contains two or more binding sites provided by polypeptides or dAbs as herein described that bind to different epitopes or domains of TNFR1. In one embodiment, such multivalent antagonists do not agonize TNFR1 when present at a concentration of about 1 nM, or about 10 nM, or about 100 nM, or about 1 μM, or about 10 μM, in a standard L929 cytotoxicity assay or a standard MRC5 or HeLa IL-8 assay as described in WO2006038027.

Antagonists of TNFR1 that do no inhibit binding of TNFα to TNFR1 have utility as diagnostic agents, because they can be used to bind and detect, quantify or measure TNFR1 in a sample and will not compete with TNF in the sample for binding to TNFR1. Accordingly, an accurate determination of whether or how much TNFR1 is in the sample can be made.

In other embodiments, the antagonist binds TNFR1 and antagonizes the activity of the TNFR1 in a standard cell assay with an ND₅₀ of ≦100 nM, and at a concentration of ≦10 μM the dAb agonizes the activity of the TNFR1 by ≦5% in the assay.

In particular embodiments, antagonist does not substantially agonize TNFR1 (act as an agonist of TNFR1) in a standard cell assay (i.e., when present at a concentration of 1 nM, 10 nM, 100 nM, 1 μM, 10 μM, 100 μM, 1000 μM or 5,000 μM, results in no more than about 5% of the TNFR1-mediated activity induced by TNFα (100 pg/ml) in the assay).

In certain embodiments, the antagonists of the invention are efficacious in models of chronic inflammatory diseases when an effective amount is administered. Generally an effective amount is about 1 mg/kg to about 10 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). The models of chronic inflammatory disease (see those described in WO2006038027) are recognized by those skilled in the art as being predictive of therapeutic efficacy in humans.

In particular embodiments, the antagonist is efficacious in the standard mouse collagen-induced arthritis model (see WO2006038027 for details of the model). For example, administering an effective amount of antagonist can reduce the average arthritic score of the summation of the four limbs in the standard mouse collagen-induced arthritis model, for example, by about 1 to about 16, about 3 to about 16, about 6 to about 16, about 9 to about 16, or about 12 to about 16, as compared to a suitable control. In another example, administering an effective amount of the antagonist can delay the onset of symptoms of arthritis in the standard mouse collagen-induced arthritis model, for example, by about 1 day, about 2 days, about 3 days, about 4 days, about 5 days, about 6 days, about 7 days, about 10 days, about 14 days, about 21 days or about 28 days, as compared to a suitable control. In another example, administering an effective amount of the antagonist can result in an average arthritic score of the summation of the four limbs in the standard mouse collagen-induced arthritis model of 0 to about 3, about 3 to about 5, about 5 to about 7, about 7 to about 15, about 9 to about 15, about 10 to about 15, about 12 to about 15, or about 14 to about 15.

In other embodiments, the antagonist is efficacious in the mouse ΔARE model of arthritis (see WO2006038027 for details of the model). For example, administering an effective amount of the antagonist can reduce the average arthritic score in the mouse ΔARE model of arthritis, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the antagonist can delay the onset of symptoms of arthritis in the mouse ΔARE model of arthritis by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the antagonist can result in an average arthritic score in the mouse ΔARE model of arthritis of 0 to about 0.5, about 0.5 to about 1, about 1 to about 1.5, about 1.5 to about 2, or about 2 to about 2.5.

In other embodiments, the antagonist is efficacious in the mouse ΔARE model of inflammatory bowel disease (IBD) (see WO2006038027 for details of the model). For example, administering an effective amount of the antagonist can reduce the average acute and/or chronic inflammation score in the mouse ΔARE model of IBD, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the polypeptide, ligand, dAb or antagonist can delay the onset of symptoms of IBD in the mouse ΔARE model of IBD by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the antagonist can result in an average acute and/or chronic inflammation score in the mouse ΔARE model of IBD of 0 to about 0.5, about 0.5 to about 1, about 1 to about 1.5, about 1.5 to about 2, or about 2 to about 2.5.

In other embodiments, the antagonist is efficacious in the mouse dextran sulfate sodium (DSS) induced model of IBD (see WO2006038027 for details of the model). For example, administering an effective amount of the antagonist can reduce the average severity score in the mouse DSS model of IBD, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the antagonist can delay the onset of symptoms of IBD in the mouse DSS model of IBD by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the antagonist can result in an average severity score in the mouse DSS model of IBD of 0 to about 0.5, about 0.5 to about 1, about 1 to about 1.5, about 1.5 to about 2, or about 2 to about 2.5.

In particular embodiments, the antagonist is efficacious in the mouse tobacco smoke model of chronic obstructive pulmonary disease (COPD) (see WO2006038027 and WO2007049017 for details of the model). For example, administering an effective amount of the ligand can reduce or delay onset of the symptoms of COPD, as compared to a suitable control.

Animal model systems which can be used to screen the effectiveness of the antagonists of TNFR1 (e.g, ligands, antibodies or binding proteins thereof) in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, N.Y., pp. 179-213; McFarlin et al. (1973) Science, 179: 478: and Satoh et al. (1987) J. Immunol., 138: 179).

Generally, the present antagonists will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The antagonists of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with the antagonists of the present invention, or even combinations of antagonists according to the present invention having different specificities, such as antagonists selected using different target antigens or epitopes, whether or not they are pooled prior to administration.

The route of administration of pharmaceutical compositions according to the invention may be any of those commonly known to those of ordinary skill in the art. For therapy, including without limitation immunotherapy, the selected ligands thereof of the invention can be administered to any patient in accordance with standard techniques.

The administration can be by any appropriate mode, including parenterally, intravenously, intramuscularly, intraperitoneally, subcutaneously, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician. Administration can be local (e.g., local delivery to the lung by pulmonary administration, e.g., intranasal administration) or systemic as indicated.

The antagonists of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present antagonists or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 10.0 mg of ligand, e.g. dAb or antagonist per kilogram of body weight, with doses of 0.05 to 2.0 mg/kg/dose being more commonly used. For prophylactic applications, compositions containing the present antagonists or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When an antagonists of TNFR1 is administered to treat, suppress or prevent a chronic inflammatory disease, it can be administered up to four times per day, twice weekly, once weekly, once every two weeks, once a month, or once every two months, at a dose off, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, the antagonist of TNFR1 is administered to treat, suppress or prevent a chronic inflammatory disease once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.)

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, the Expanded Disability Status Scale (for multiple sclerosis), the Irvine Inflammatory Bowel Disease Questionnaire (32 point assessment evaluates quality of life with respect to bowel function, systemic symptoms, social function and emotional status-score ranges from 32 to 224, with higher scores indicating a better quality of life), the Quality of Life Rheumatoid Arthritis Scale, or other accepted clinical assessment scale as known in the field. A sustained (e.g., one day or more, or longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing an antagonist or cocktail thereof according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands whereby the undesired cells are killed or otherwise removed from the blood for return to the mammal in accordance with standard techniques.

A composition containing an antagonist according to the present invention may be utilised in prophylactic and therapeutic settings to aid in the alteration, inactivation, killing or removal of a select target cell population in a mammal.

The anti-TNFR1 antagonists (eg, dAb monomers) can be administered and or formulated together with one or more additional therapeutic or active agents. When an antagonist (eg, a dAb) is administered with an additional therapeutic agent, the antagonist can be administered before, simultaneously with or subsequent to administration of the additional agent. Generally, the antagonist and additional agent are administered in a manner that provides an overlap of therapeutic effect.

In one embodiment of the method or use of the invention, the method or use is provided for treating, suppressing or preventing a chronic inflammatory disease, comprising administering to a mammal in need thereof a therapeutically-effective dose or amount of an antagonist of TNFR1 according to the invention.

In one embodiment of the method or use of the invention, the method or use is provided for treating, suppressing or preventing arthritis (e.g., rheumatoid arthritis, juvenile rheumatoid arthritis, ankylosing spondylitis, psoriatic arthritis) comprising administering to a mammal in need thereof a therapeutically-effective dose or amount of an antagonist of TNFR1 according to the invention.

In one embodiment of the method or use of the invention, the method or use is provided for treating, suppressing or preventing psoriasis comprising administering to a mammal in need thereof a therapeutically-effective dose or amount of an antagonist of TNFR1 according to the invention.

In one embodiment of the method or use of the invention, the method or use is provided for treating, suppressing or preventing inflammatory bowel disease (e.g., Crohn's disease, ulcerative colitis) comprising administering to a mammal in need thereof a therapeutically-effective dose or amount of an antagonist of TNFR1 according to the invention.

In one embodiment of the method or use of the invention, the method or use is provided for treating, suppressing or preventing chronic obstructive pulmonary disease (e.g., chronic bronchitis, chronic obstructive bronchitis, emphysema), comprising administering to a mammal in need thereof a therapeutically-effective dose or amount of an antagonist of TNFR1 according to the invention.

In one embodiment of the method or use of the invention, the method or use is provided for treating, suppressing or preventing pneumonia (e.g., bacterial pneumonia, such as Staphylococcal pneumonia) comprising administering to a mammal in need thereof a therapeutically-effective dose or amount of an antagonist of TNFR1 according to the invention.

In one embodiment of the method or use of the invention, the method or use is provided for treating, suppressing or preventing other pulmonary diseases in addition to chronic obstructive pulmonary disease, and pneumonia. Other pulmonary diseases that can be treated, suppressed or prevented in accordance with the invention include, for example, cystic fibrosis and asthma (e.g., steroid resistant asthma). Thus, in another embodiment, the method or use is provided for treating, suppressing or preventing a pulmonary disease (e.g., cystic fibrosis, asthma) comprising administering to a mammal in need thereof a therapeutically-effective dose or amount of an antagonist of TNFR1 according to the invention.

In particular embodiments, the antagonist of TNFR1 is administered via pulmonary delivery, such as by inhalation (e.g., intrabronchial, intranasal or oral inhalation, intranasal drops) or by systemic delivery (e.g., parenteral, intravenous, intramuscular, intraperitoneal, subcutaneous).

In one embodiment of the method or use of the invention, the method or use is provided for treating, suppressing or preventing septic shock comprising administering to a mammal in need thereof a therapeutically-effective dose or amount of an antagonist of TNFR1 according to the invention.

In a further aspect of the invention, there is provided a composition comprising an antagonist of TNFR1 according to the invention and a pharmaceutically acceptable carrier, diluent or excipient.

Moreover, the present invention provides a method for the treatment of disease using an antagonist of TNFR1 or a composition according to the present invention. In an embodiment the disease is cancer or an inflammatory disease, eg rheumatoid arthritis, asthma or Crohn's disease.

In a further aspect of the invention, there is provided a composition comprising an antagonist according to the invention and a pharmaceutically acceptable carrier, diluent or excipient.

In particular embodiments, the antagonist or composition is administered via pulmonary delivery, such as by inhalation (e.g, intrabronchial, intranasal or oral inhalation, intranasal drops) or by systemic delivery (e.g, parenteral, intravenous, intramuscular, intraperitoneal, subcutaneous).

An aspect of the invention provides a pulmonary delivery device containing an antagonist according to the invention. The device can be an inhaler or an intranasal administration device.

In other embodiments, the antagonist further comprises a half-life extending moiety, such as a polyalkylene glycol moiety, serum albumin or a fragment thereof, transferrin receptor or a transferrin-binding portion thereof, or a moiety comprising a binding site for a polypeptide that enhance half-life in vivo. In some embodiments, the half-life extending moiety is a moiety comprising a binding site for a polypeptide that enhances half-life in vivo selected from the group consisting of an affibody, a SpA domain, an LDL receptor class A domain, an EGF domain, and an avimer.

In other embodiments, the half-life extending moiety is a polyethylene glycol moiety. In one embodiment, the antagonist comprises (optionally consists of) a single variable domain of the invention linked to a polyethylene glycol moiety (optionally, wherein the moiety has a size of about 20 to about 50 kDa, optionally about 40 kDa linear or branched PEG). Reference is made to WO04081026 for more detail on PEGylation of dAbs and binding moieties. In one embodiment, the antagonist consists of a dAb monomer linked to a PEG, wherein the dAb monomer is a single variable domain according to the invention. This antagonist can be provided for treatment of inflammatory disease, a lung condition (e.g., asthma, influenza or COPD) or cancer or optionally is for intravenous administration.

In other embodiments, the half-life extending moiety is an antibody or antibody fragment (e.g, an immunoglobulin single variable domain) comprising a binding site for serum albumin or neonatal Fc receptor.

The invention also relates to a composition (e.g, pharmaceutical composition) comprising an antagonistand a physiologically acceptable carrier. In some embodiments, the composition comprises a vehicle for intravenous, intramuscular, intraperitoneal, intraarterial, intrathecal, intraarticular, subcutaneous administration, pulmonary, intranasal, vaginal, or rectal administration.

The invention also relates to a drug delivery device comprising the composition (e.g, pharmaceutical composition) of the invention. In some embodiments, the drug delivery device comprises a plurality of therapeutically effective doses of antagonist. In other embodiments, the drug delivery device is selected from the group consisting of parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, rectal delivery device, syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

The antagonist of the invention can be formatted as described herein. For example, it can be formatted to tailor in vivo serum half-life. If desired, the ligand can further comprise a toxin or a toxin moiety as described herein. In some embodiments, the antagonist comprises a surface active toxin, such as a free radical generator (e.g, selenium containing toxin) or a radionuclide. In other embodiments, the toxin or toxin moiety is a polypeptide domain (e.g, a dAb) having a binding site with binding specificity for an intracellular target. In particular embodiments, the antagonist is an IgG-like format that has binding specificity for TNFR1 (e.g, human TNFR1).

In an aspect, the antagonist comprises or consists of a fusion protein comprising an anti-TNFR1 single variable domain as herein described. The variable domain can be fused, for example, to a peptide or polypeptide or protein. In one embodiment, the variable domain is fused to an antibody or antibody fragment, eg a monoclonal antibody. Generally, fusion can be achieved by expressing the fusion product from a single nucleic acid sequence or by expressing a polypeptide comprising the single variable domain and then assembling this polypeptide into a larger protein or antibody format using techniques that are conventional.

In one embodiment, antagonist or the fusion protein comprises an antibody constant domain, for example, an antibody Fc, optionally wherein the N-terminus of the Fc is linked (optionally directly linked) to the C-terminus of an anti-TNFR1 single variable domain as herein described. In one embodiment, the antagonist or the fusion protein comprises a half-life extending moiety, for example, a polyethylene glycol moiety, serum albumin or a fragment thereof, transferrin receptor or a transferrin-binidng portion thereof, or an antibody or antibody fragment comprising a binding site for a polypeptide that enhances half-life in vivo. The half-life extending moiety can be an antibody or antibody fragment comprising a binding site for serum albumin or neonatal Fc receptor. The half-life extending moiety can be a dAb, antibody or antibody fragment. In one embodiment, the antagonist or the fusion protein is provided such that the variable domain comprised by the antagonist or fusion protein further comprises a polyalkylene glycol moiety. The polyalkylene glycol moiety can be a polyethylene glycol moiety. Further discussion is provided below.

Reference is made to WO2006038027, which discloses anti-TNFR1 immunoglobulin single variable domains which can be used in antagonists of the invention and methods and uses of the invention employing such antagonists. The disclosure of this document is incorporated herein in its entirety, in addition to provide for uses, formats, methods of selection, methods of production, methods of formulation and assays for anti-TNFR1 single variable domains, ligands, antagonists and the like, so that these disclosures can be applied specifically and explicitly in the context of the present invention, including to provide explicit description for importation into claims of the present disclosure.

The anti-TNFR1 antagonist of the invention optionally comprise an immunoglobulin single variable domain that is a human variable domain or a variable domain that comprises or are derived from human framework regions (e.g., DP47 or DPK9 framework regions). In certain embodiments, the variable domain is based on a universal framework, as described herein.

In certain embodiments, a polypeptide domain (e.g., immunoglobulin single variable domain) that has a binding site with binding specificity for TNFR1 resists aggregation, unfolds reversibly (see WO04101790, the teachings of which are incorporated herein by reference).

### Nucleic Acid Molecules, Vectors and Host Cells

The invention also provides isolated and/or recombinant nucleic acid molecules encoding antagonists as described herein.

In one aspect, the invention provides an isolated or recombinant nucleic acid encoding an antagonist according to the invention comprising an immunoglobulin single variable domain. In one embodiment, the nucleic acid comprises the nucleotide sequence of DOM1h-574-156, DOM1h-574-72, DOM1h-574-109, DOM1h-574-138, DOM1h-574-162 or DOM1h-574-180. In one embodiment, the nucleic acid comprises the nucleotide sequence of DOM1h-574-156, DOM1h-574-72, DOM1h-574-109, DOM1h-574-132, DOM1h-574-135, DOM1h-574-138, DOM1h-574-162 or DOM1h-574-180. In one embodiment, the nucleic acid comprises the nucleotide sequence of DOM1h-574-109, DOM1h-574-93, DOM1h-574-123, DOM1h-574-125, DOM1h-574-126 or DOM1h-574-129, DOM1h-574-133, DOM1h-574-137 or DOM1h-574-160. In one embodiment, the nucleic acid comprises the nucleotide sequence of DOM1h-574-156, DOM1h-574-72, DOM1h-574-109, DOM1h-574-125, DOM1h-574-126, DOM1h-574-133, DOM1h-574-135 or DOM1h-574-138, DOM1h-574-139, DOM1h-574-155, DOM1h-574-162 or DOM1h-574-180. In one embodiment, the nucleic acid comprises the nucleotide sequence of DOM1h-574-126 or DOM1h-574-133.

In one aspect, the invention provides an isolated or recombinant nucleic acid encoding an antagonist of the invention, wherein the nucleic acid comprises a nucleotide sequence that is at least 80, 85, 90, 95, 98 or 99% identical to the nucleotide sequence of DOM1h-574-156, DOM1h-574-72, DOM1h-574-109, DOM1h-574-138, DOM1h-574-162 or DOM1h-574-180 and wherein the nucleic acid encodes a polypeptide comprising an immunoglobulin single variable domain that specifically binds to TNFR1. In one aspect, the invention provides an isolated or recombinant nucleic acid encoding an antagonist of the invention, wherein the nucleic acid comprises a nucleotide sequence that is at least 80, 85, 90, 95, 98 or 99% identical to the nucleotide sequence of DOM1h-574-156, DOM1h-574-72, DOM1h-574-109, DOM1h-574-132, DOM1h-574-135, DOM1h-574-138, DOM1h-574-162 or DOM1h-574-180 and wherein the nucleic acid encodes a polypeptide comprising an immunoglobulin single variable domain that specifically binds to TNFR1. In one aspect, the invention provides an isolated or recombinant nucleic acid encoding an antagonist of the invention, wherein the nucleic acid comprises a nucleotide sequence that is at least 80, 85, 90, 95, 98 or 99% identical to the nucleotide sequence of DOM1h-574-109, DOM1h-574-93, DOM1h-574-123, DOM1h-574-125, DOM1h-574-126 or DOM1h-574-129, DOM1h-574-133, DOM1h-574-137 or DOM1h-574-160 and wherein the nucleic acid encodes a polypeptide comprising an immunoglobulin single variable domain that specifically binds to TNFR1. In one aspect, the invention provides an isolated or recombinant nucleic acid encoding an antagonist of the invention, wherein the nucleic acid comprises a nucleotide sequence that is at least 80, 85, 90, 95, 98 or 99% identical to the nucleotide sequence of DOM1h-574-156, DOM1h-574-72, DOM1h-574-109, DOM1h-574-125, DOM1h-574-126, DOM1h-574-133, DOM1h-574-135 or DOM1h-574-138, DOM1h-574-139, DOM1h-574-155, DOM1h-574-162 or DOM1h-574-180 and wherein the nucleic acid encodes a polypeptide comprising an immunoglobulin single variable domain that specifically binds to TNFR1. In one aspect, the invention provides an isolated or recombinant nucleic acid encoding an antagonist of the invention, wherein the nucleic acid comprises a nucleotide sequence that is at least 80, 85, 90, 95, 98 or 99% identical to the nucleotide sequence of DOM1h-574-126 or DOM1h-574-133 and wherein the nucleic acid encodes a polypeptide comprising an immunoglobulin single variable domain that specifically binds to TNFR1.

In one aspect, the invention provides a vector comprising a nucleic acid of the invention. In one aspect, the invention provides a host cell comprising a nucleic acid of the invention or the vector. There is provided a method of producing polypeptide comprising an antagonist of the invention, the method comprising maintaining the host cell under conditions suitable for expression of the nucleic acid or vector, whereby an antagonist polypeptide comprising an immunoglobulin single variable domain is produced. Optionally, the method further comprises the step of isolating the polypeptide and optionally producing a variant, eg a mutated variant, having an improved affinity (KD); ND₅₀ for TNFR1 neutralization in a standard MRC5, L929 or Cynomologus KI assay than the isolated polypeptide.

Nucleic acids referred to herein as “isolated” are nucleic acids which have been separated away from the nucleic acids of the genomic DNA or cellular RNA of their source of origin (e.g., as it exists in cells or in a mixture of nucleic acids such as a library), and include nucleic acids obtained by methods described herein or other suitable methods, including essentially pure nucleic acids, nucleic acids produced by chemical synthesis, by combinations of biological and chemical methods, and recombinant nucleic acids which are isolated (see e.g., Daugherty, B. L. et al., Nucleic Acids Res., 19(9): 2471-2476 (1991); Lewis, A. P. and J. S. Crowe, Gene, 101: 297-302 (1991)).

Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes.

In certain embodiments, the isolated and/or recombinant nucleic acid comprises a nucleotide sequence encoding an antagonist, as described herein, wherein the antagonist comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb that binds TNFR1 disclosed herein, eg, DOM1h-574-156, DOM1h-574-72, DOM1h-574-109, DOM1h-574-138, DOM1h-574-162 or DOM1h-574-180. Nucleotide sequence identity can be determined over the whole length of the nucleotide sequence that encodes the selected anti-TNFR1 dAb.

The invention also provides a vector comprising a recombinant nucleic acid molecule of the invention. In certain embodiments, the vector is an expression vector comprising one or more expression control elements or sequences that are operably linked to the recombinant nucleic acid of the invention. The invention also provides a recombinant host cell comprising a recombinant nucleic acid molecule or vector of the invention. Suitable vectors (e.g, plasmids, phagmids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g, promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for prokaryotic (e.g, lac, tac, T3, T7 promoters for E. coli) and eukaryotic (e.g, Simian Virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in prokaryotic (e.g, lactamase gene (ampicillin resistance), Tet gene for tetracycline resistance) and eukaryotic cells (e.g, neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g, LEU2, URA3, HIS3) are often used as selectable markers in yeast. Use of viral (e.g, baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acac. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In certain embodiments, the host cell is a non-human host cell.

The invention also provides a method for producing an antagonist of the invention, comprising maintaining a recombinant host cell comprising a recombinant nucleic acid of the invention under conditions suitable for expression of the recombinant nucleic acid, whereby the recombinant nucleic acid is expressed and a antagonist is produced. In some embodiments, the method further comprises isolating the antagonist.

Reference is made to WO2006038027, for details of disclosure that is applicable to embodiments of the present invention. For example, relevant disclosure relates to the preparation of immunoglobulin single variable domain-based ligands, library vector systems, library construction, combining single variable domains, characterisation of ligands, structure of ligands, skeletons, protein scaffolds, diversification of the canonical sequence, assays and therapeutic and diagnostic compositions and uses, as well as definitions of “operably linked”, “naive”, “prevention”, “suppression”, “treatment”, “effective” and “therapeutically-effective dose”.

### Formats

Increased half-life is useful in in vivo applications of immunoglobulins, especially antibodies and most especially antibody fragments of small size. Such fragments (Fvs, disulphide bonded Fvs, Fabs, scFvs, dAbs) suffer from rapid clearance from the body; thus, whilst they are able to reach most parts of the body rapidly, and are quick to produce and easier to handle, their in vivo applications have been limited by their only brief persistence in vivo. One embodiment of the invention solves this problem by providing increased half-life of the ligands in vivo and consequently longer persistence times in the body of the functional activity of the antagonist. Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC). Half-life and AUC definitions are provided above.

In one embodiment, the present invention provides an antagonist or a composition comprising a antagonist according to the invention having a tα half-life in the range of 15 minutes or more. In one embodiment, the lower end of the range is 30 minutes, 45 minutes, 1 hour, 2 hours, 3 hours, 4 hours, 5 hours, 6 hours, 7 hours, 10 hours, 11 hours or 12 hours. In addition, or alternatively, a antagonist or composition according to the invention will have a tα half life in the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours.

In one embodiment, the present invention provides an antagonist or a composition comprising an antagonist according to the invention having a tβ half-life in the range of about 2.5 hours or more. In one embodiment, the lower end of the range is about 3 hours, about 4 hours, about 5 hours, about 6 hours, about 7 hours, about 10 hours, about 11 hours, or about 12 hours. In addition, or alternatively, an antagonist or composition according to the invention has a tβ half-life in the range of up to and including 21 days. In one embodiment, the upper end of the range is about 12 hours, about 24 hours, about 2 days, about 3 days, about 5 days, about 10 days, about 15 days or about 20 days. In one embodiment an antagonist or composition according to the invention will have a tβ half life in the range about 12 to about 60 hours. In a further embodiment, it will be in the range about 12 to about 48 hours. In a further embodiment still, it will be in the range about 12 to about 26 hours.

In addition, or alternatively to the above criteria, the present invention provides an antagonist or a composition comprising an antagonist according to the invention having an AUC value (area under the curve) in the range of about 1 mg·min/ml or more. In one embodiment, the lower end of the range is about 5, about 10, about 15, about 20, about 30, about 100, about 200 or about 300 mg·min/ml. In addition, or alternatively, an antagonist or composition according to the invention has an AUC in the range of up to about 600 mg·min/ml. In one embodiment, the upper end of the range is about 500, about 400, about 300, about 200, about 150, about 100, about 75 or about 50 mg·min/ml. In one embodiment an antagonist according to the invention will have a AUC in the range selected from the group consisting of the following: about 15 to about 150 mg·min/ml, about 15 to about 100 mg·min/ml, about 15 to about 75 mg·min/ml, and about 15 to about 50 mg·min/ml.

Polypeptides and dAbs and antagonists comprising these can be formatted to have a larger hydrodynamic size, for example, by attachment of a PEG group, serum albumin, transferrin, transferrin receptor or at least the transferrin-binding portion thereof, an antibody Fc region, or by conjugation to an antibody domain. For example, polypeptides dAbs and antagonists formatted as a larger antigen-binding fragment of an antibody or as an antibody (e.g, formatted as a Fab, Fab′, F(ab)₂, F(ab′)₂, IgG, scFv).

Hydrodynamic size of the antagonists (e.g, dAb monomers and multimers) of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a antagonist ligand format (e.g, the size of a PEG moiety attached to a dAb monomer), can be varied depending on the desired application. For example, where ligand is intended to leave the circulation and enter into peripheral tissues, it is desirable to keep the hydrodynamic size of the ligand low to facilitate extravazation from the blood stream. Alternatively, where it is desired to have the ligand remain in the systemic circulation for a longer period of time the size of the ligand can be increased, for example by formatting as an Ig like protein.

Half-Life Extension by Targeting an Antigen or Epitope that Increases Half-Live In Vivo

The hydrodynaminc size of an antagonist ligand and its serum half-life can also be increased by conjugating or associating an TNFR1 binding antagonist of the invention to a binding domain (e.g, antibody or antibody fragment) that binds an antigen or epitope that increases half-live in vivo, as described herein. For example, the TNFR1 binding agent (e.g, polypeptide) can be conjugated or linked to an anti-serum albumin or anti-neonatal Fc receptor antibody or antibody fragment, eg an anti-SA or anti-neonatal Fc receptor dAb, Fab, Fab′ or scFv, or to an anti-SA affibody or anti-neonatal Fc receptor Affibody or an anti-SA avimer, or an anti-SA binding domain which comprises a scaffold selected from, but not limited to, the group consisting of CTLA-4, lipocallin, SpA, an affibody, an avimer, GroE1 and fibronectin (see WO2008096158 for disclosure of these binding domains, which domains and their sequences are incorporated herein by reference and form part of the disclosure of the present text). Conjugating refers to a composition comprising polypeptide, dAb or antagonist of the invention that is bonded (covalently or noncovalently) to a binding domain that binds serum albumin.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g, soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g, type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g, fibrin, α-2 macroglobulin, serum albumin, fibrinogen (e.g, fibrinogen A, fibrinogen B), serum amyloid protein A, haptoglobin, profilin, ubiquitin, uteroglobulin and β-2-microglobulin), enzymes and enzyme inhibitors (e.g, plasminogen, lysozyme, cystatin C, alpha-1-antitrypsin and pancreatic trypsin inhibitor), proteins of the immune system, such as immunoglobulin proteins (e.g, IgA, IgD, IgE, IgG, IgM, immunoglobulin light chains (kappa/lambda)), transport proteins (e.g, retinol binding protein, α-1 microglobulin), defensins (e.g, beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhance serum half-life in vivo also include proteins localized to the kidney (e.g, polycystin, type IV collagen, organic anion transporter K1, Heymann's antigen), proteins localized to the liver (e.g, alcohol dehydrogenase, G250), proteins localized to the lung (e.g, secretory component, which binds IgA), proteins localized to the heart (e.g, HSP 27, which is associated with dilated cardiomyopathy), proteins localized to the skin (e.g, keratin), bone specific proteins such as morphogenic proteins (BMPs), which are a subset of the transforming growth factor β superfamily of proteins that demonstrate osteogenic activity (e.g, BMP-2, BMP-4, BMP-5, BMP-6, BMP-7, BMP-8), tumor specific proteins (e.g, trophoblast antigen, herceptin receptor, oestrogen receptor, cathepsins (e.g, cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

dAbs that Bind Serum Albumin

The invention in one embodiment provides an antagonist (e.g., dual specific ligand comprising an anti-TNFR1 dAb (a first dAb)) that binds to TNFR1 and a second dAb that binds serum albumin (SA), the second dAb binding SA with a KD as determined by surface plasmon resonance of about 1 nM to about 1, about 2, about 3, about 4, about 5, about 10, about 20, about 30, about 40, about 50, about 60, about 70, about 100, about 200, about 300, about 400 or about 500 μM (i.e., ×10⁻⁹ to 5×10⁻⁴M), or about 100 nM to about 10 μM, or about 1 to about 5 μM or about 3 to about 70 nM or about 10 nM to about 1, about 2, about 3, about 4 or about 5 μM. For example about 30 to about 70 nM as determined by surface plasmon resonance. In one embodiment, the first dAb (or a dAb monomer) binds SA (e.g., HSA) with a KD as determined by surface plasmon resonance of approximately about 1, about 50, about 70, about 100, about 150, about 200, about 300 nM or about 1, about 2 or about 3 μM. In one embodiment, for a dual specific ligand comprising a first anti-SA dAb and a second dAb to TNFR1, the affinity (e.g., KD and/or K_{off} as measured by surface plasmon resonance, e.g., using BiaCore) of the second dAb for its target is from about 1 to about 100000 times (e.g., about 100 to about 100000, or about 1000 to about 100000, or about 10000 to about 100000 times) the affinity of the first dAb for SA. In one embodiment, the serum albumin is human serum albumin (HSA). For example, the first dAb binds SA with an affinity of approximately about 10 μM, while the second dAb binds its target with an affinity of about 100 pM. In one embodiment, the serum albumin is human serum albumin (HSA). In one embodiment, the first dAb binds SA (e.g., HSA) with a KD of approximately about 50, for example about 70, about 100, about 150 or about 200 nM. Details of dual specific ligands are found in WO03002609, WO04003019, WO2008096158 and WO04058821.

The antagonist ligands of the invention can in one embodiment comprise a dAb that binds serum albumin (SA) with a KD as determined by surface plasmon resonance of about 1 nM to about 1, about 2, about 3, about 4, about 5, about 10, about 20, about 30, about 40, about 50, about 60, about 70, about 100, about 200, about 300, about 400 or about 500 μM (i.e., ×about 10⁻⁹ to about 5×10⁻⁴M), or about 100 nM to about 10 μM, or about 1 to about 5 μM or about 3 to about 70 nM or about 10 nM to about 1, about 2, about 3, about 4 or about 5 μM. For example about 30 to about 70 nM as determined by surface plasmon resonance. In one embodiment, the first dAb (or a dAb monomer) binds SA (e.g., HSA) with a KD as determined by surface plasmon resonance of approximately about 1, about 50, about 70, about 100, about 150, about 200, about 300 nM or about 1, about 2 or about 3 μM. In one embodiment, the first and second dAbs are linked by a linker, for example a linker of from 1 to 4 amino acids or from 1 to 3 amino acids, or greater than 3 amino acids or greater than 4, 5, 6, 7, 8, 9, 10, 15 or 20 amino acids. In one embodiment, a longer linker (greater than 3 amino acids) is used to enhance potency (KD of one or both dAbs in the antagonist).

In particular embodiments of the antagonists, the dAb binds human serum albumin and competes for binding to albumin with a dAb selected from the group consisting of DOM7h-11, DOM7h-11-3, DOM7h-11-12, DOM7h-11-15, DOM7h-14, DOM7h-14-10, DOM7h-14-18 and DOM7m-16.

In particular embodiments of the antagonists, the dAb binds human serum albumin and competes for binding to albumin with a dAb selected from the group consisting of

MSA-16, MSA-26 (See WO04003019 for disclosure of these sequences, which sequences and their nucleic acid counterpart are incorporated herein by reference and form part of the disclosure of the present text),

DOM7m-16 (SEQ ID NO: 473), DOM7m-12 (SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), DOM7r-33 (SEQ ID NO: 517) (See WO2007080392 for disclosure of these sequences, which sequences and their nucleic acid counterpart are incorporated herein by reference and form part of the disclosure of the present text; the SEQ ID No's in this paragraph are those that appear in WO2007080392),

dAb8 (dAb10), dAb 10, dAb36, dAb7r20 (DOM7r20), dAb7r21 (DOM7r21), dAb7r22 (DOM7r22), dAb7r23 (DOM7r23), dAb7r24 (DOM7r24), dAb7r25 (DOM7r25), dAb7r26 (DOM7r26), dAb7r27 (DOM7r27), dAb7r28 (DOM7r28), dAb7r29 (DOM7r29), dAb7r29 (DOM7r29), dAb7r31 (DOM7r31), dAb7r32 (DOM7r32), dAb7r33 (DOM7r33), dAb7r33 (DOM7r33), dAb7h22 (DOM7h22), dAb7h23 (DOM7h23), dAb7h24 (DOM7h24), dAb7h25 (DOM7h25), dAb7h26 (DOM7h26), dAb7h27 (DOM7h27), dAb7h30 (DOM7h30), dAb7h31 (DOM7h31), dAb2 (dAbs 4,7,41), dAb4, dAb7, dAb11, dAb12 (dAb7 m12), dAb13 (dAb 15), dAb15, dAb16 (dAb21, dAb7 m16), dAb17, dAb18, dAb19, dAb21, dAb22, dAb23, dAb24, dAb25 (dAb26, dAb7 m26), dAb27, dAb30 (dAb35), dAb31, dAb33, dAb34, dAb35, dAb38 (dAb54), dAb41, dAb46 (dAbs 47, 52 and 56), dAb47, dAb52, dAb53, dAb54, dAb55, dAb56, dAb7 m12, dAb7 m16, dAb7 m26, dAb7r1 (DOM 7r1), dAb7r3 (DOM7r3), dAb7r4 (DOM7r4), dAb7r5 (DOM7r5), dAb7r7 (DOM7r7), dAb7r8 (DOM7r8), dAb7r13 (DOM7r13), dAb7r14 (DOM7r14), dAb7r15 (DOM7r15), dAb7r16 (DOM7r16), dAb7r17 (DOM7r17), dAb7r18 (DOM7r18), dAb7r19 (DOM7r19), dAb7h1 (DOM7h1), dAb7h2 (DOM7h2), dAb7h6 (DOM7h6), dAb7h7 (DOM7h7), dAb7h8 (DOM7h8), dAb7h9 (DOM7h9), dAb7h10 (DOM7h10), dAb7h11 (DOM7h11), dAb7h12 (DOM7h12), dAb7h13 (DOM7h13), dAb7h14 (DOM7h14), dAb7p1 (DOM7p1), and dAb7p2 (DOM7p2) (see WO2008096158 for disclosure of these sequences, which sequences and their nucleic acid counterpart are incorporated herein by reference and form part of the disclosure of the present text). Alternative names are shown in brackets after the dAb, e.g, dAb8 has an alternative name which is dAb10 i.e. dAb8 (dAb10).

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of DOM7h-11, DOM7h-11-3, DOM7h-11-12, DOM7h-11-15, DOM7h-14, DOM7h-14-10, DOM7h-14-18 and DOM7m-16.

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of

MSA-16, MSA-26,

DOM7m-16 (SEQ ID NO: 473), DOM7m-12 (SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), DOM7r-33 (SEQ ID NO: 517) (the SEQ ID No's in this paragraph are those that appear in WO2007080392),

dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7 m12, dAb7 m16, dAb7 m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2.

For example, the dAb that binds human serum albumin can comprise an amino acid sequence that has at least about 90%, or at least about 95%, or at least about 96%, or at least about 97%, or at least about 98%, or at least about 99% amino acid sequence identity with DOM7h-11-3 or DOM7h-14-10.

For example, the dAb that binds human serum albumin can comprise an amino acid sequence that has at least about 90%, or at least about 95%, or at least about 96%, or at least about 97%, or at least about 98%, or at least about 99% amino acid sequence identity with

DOM7h-2 (SEQ ID NO:482), DOM7h-3 (SEQ ID NO:483), DOM7h-4 (SEQ ID NO:484), DOM7h-6 (SEQ ID NO:485), DOM7h-1 (SEQ ID NO:486), DOM7h-7 (SEQ ID NO:487), DOM7h-8 (SEQ ID NO:496), DOM7r-13 (SEQ ID NO:497), DOM7r-14 (SEQ ID NO:498), DOM7h-22 (SEQ ID NO:489), DOM7h-23 (SEQ ID NO:490), DOM7h-24 (SEQ ID NO:491), DOM7h-25 (SEQ ID NO:492), DOM7h-26 (SEQ ID NO:493), DOM7h-21 (SEQ ID NO:494) or DOM7h-27 (SEQ ID NO:495) (the SEQ ID No's in this paragraph are those that appear in WO2007080392), or

dAb8, dAb 10, dAb36, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7h1, dAb7h2, dAb7h6, dAb7h7, dAb7h8, dAb7h9, dAb7h10, dAb7h11, dAb7h12, dAb7h13 or dAb7h14.

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of

DOM7h-2 (SEQ ID NO:482), DOM7h-6 (SEQ ID NO:485), DOM7h-1 (SEQ ID NO:486), DOM7h-7 (SEQ ID NO:487), DOM7h-8 (SEQ ID NO:496), DOM7h-22 (SEQ ID NO:489), DOM7h-23 (SEQ ID NO:490), DOM7h-24 (SEQ ID NO:491), DOM7h-25 (SEQ ID NO:492), DOM7h-26 (SEQ ID NO:493), DOM7h-21 (SEQ ID NO:494), DOM7h-27 (SEQ ID NO:495) (the SEQ ID No's in this paragraph are those that appear in WO2007080392),

dAb7h21, dAb7h22, dAb7h23, Ab7h24, Ab7h25, Ab7h26, dAb7h27, dAb7h30, dAb7h31, dAb2, dAb4, dAb7, dAb38, dAb41, dAb7h1, dAb7h2, dAb7h6, dAb7h7, dAb7h8, dAb7h9, dAb7h10, dAb7h11, dAb7h12, dAb7h13 and dAb7h14.

In more particular embodiments, the dAb is a V_{κ} dAb that binds human serum albumin and has an amino acid sequence selected from the group consisting of

DOM7h-2 (SEQ ID NO:482), DOM7h-6 (SEQ ID NO:485), DOM7h-1 (SEQ ID NO:486), DOM7h-7 (SEQ ID NO:487), DOM7h-8 (SEQ ID NO:496) (the SEQ ID No's in this paragraph are those that appear in WO2007080392),

dAb2, dAb4, dAb7, dAb38, dAb41, dAb54, dAb7h1, dAb7h2, dAb7h6, dAb7h7, dAb7h8, dAb7h9, dAb7h10, dAb7h11, dAb7h12, dAb7h13 and dAb7h14.

In more particular embodiments, the dAb is a V_{H} dAb that binds human serum albumin and has an amino acid sequence selected from dAb7h30 and dAb7h31.

In more particular embodiments, the dAb is dAb7h11 or dAb7h14. In an example, the dAb is DOM7h-11-3. In another example, the dAb is DOM7h-14-10.

In other embodiments, the dAb, ligand or antagonist binds human serum albumin and comprises one, two or three of the CDRs of any of the foregoing amino acid sequences, eg one, two or three of the CDRs of DOM7h-11-3, DOM7h-14-10, dAb7h11 or dAb7h14.

Suitable Camelid V_{HH} that bind serum albumin include those disclosed in WO 2004/041862 (Ablynx N.V.) and in WO2007080392 (which V_{HH} sequences and their nucleic acid counterpart are incorporated herein by reference and form part of the disclosure of the present text), such as Sequence A (SEQ ID NO:518), Sequence B (SEQ ID NO:519), Sequence C (SEQ ID NO:520), Sequence D (SEQ ID NO:521), Sequence E (SEQ ID NO:522), Sequence F (SEQ ID NO:523), Sequence G (SEQ ID NO:524), Sequence H (SEQ ID NO:525), Sequence I (SEQ ID NO:526), Sequence J (SEQ ID NO:527), Sequence K (SEQ ID NO:528), Sequence L (SEQ ID NO:529), Sequence M (SEQ ID NO:530), Sequence N (SEQ ID NO:531), Sequence 0 (SEQ ID NO:532), Sequence P (SEQ ID NO:533), Sequence Q (SEQ ID NO:534), these sequence numbers corresponding to those cited in WO2007080392 or WO 2004/041862 (Ablynx N.V.). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with ALB1 disclosed in WO2007080392 or any one of SEQ ID NOS:518-534, these sequence numbers corresponding to those cited in WO2007080392 or WO 2004/041862.

In some embodiments, the antagonist comprises an anti-serum albumin dAb that competes with any anti-serum albumin dAb disclosed herein for binding to serum albumin (e.g, human serum albumin).

In an alternative embodiment, the antagonist comprises a binding moiety specific for SA (e.g., human SA), wherein the moiety comprises non-immunoglobulin sequences as described in WO2008096158, the disclosure of these binding moieties, their methods of production and selection (e.g., from diverse libraries) and their sequences are incorporated herein by reference as part of the disclosure of the present text)

### Conjugation to a Half-Life Extending Moiety (e.g., Albumin)

In one embodiment, a (one or more) half-life extending moiety (e.g., albumin, transferrin and fragments and analogues thereof) is conjugated or associated with the TNFR1-binding antagonist of the invention. Examples of suitable albumin, albumin fragments or albumin variants for use in a TNFR1-binding format are described in WO 2005077042, which disclosure is incorporated herein by reference and forms part of the disclosure of the present text. In particular, the following albumin, albumin fragments or albumin variants can be used in the present invention:
SEQ ID NO:1 (as disclosed in WO 2005077042, this sequence being explicitly incorporated into the present disclosure by reference);
Albumin fragment or variant comprising or consisting of amino acids 1-387 of SEQ ID NO:1 in WO 2005077042;
Albumin, or fragment or variant thereof, comprising an amino acid sequence selected from the group consisting of: (a) amino acids 54 to 61 of SEQ ID NO:1 in WO 2005077042; (b) amino acids 76 to 89 of SEQ ID NO:1 in WO 2005077042; (c) amino acids 92 to 100 of SEQ ID NO:1 in WO 2005077042; (d) amino acids 170 to 176 of SEQ ID NO:1 in WO 2005077042; (e) amino acids 247 to 252 of SEQ ID NO:1 in WO 2005077042; (f) amino acids 266 to 277 of SEQ ID NO:1 in WO 2005077042; (g) amino acids 280 to 288 of SEQ ID NO:1 in WO 2005077042; (h) amino acids 362 to 368 of SEQ ID NO:1 in WO 2005077042; (i) amino acids 439 to 447 of SEQ ID NO:1 in WO 2005077042 (j) amino acids 462 to 475 of SEQ ID NO:1 in WO 2005077042; (k) amino acids 478 to 486 of SEQ ID NO:1 in WO 2005077042; and (1) amino acids 560 to 566 of SEQ ID NO:1 in WO 2005077042.

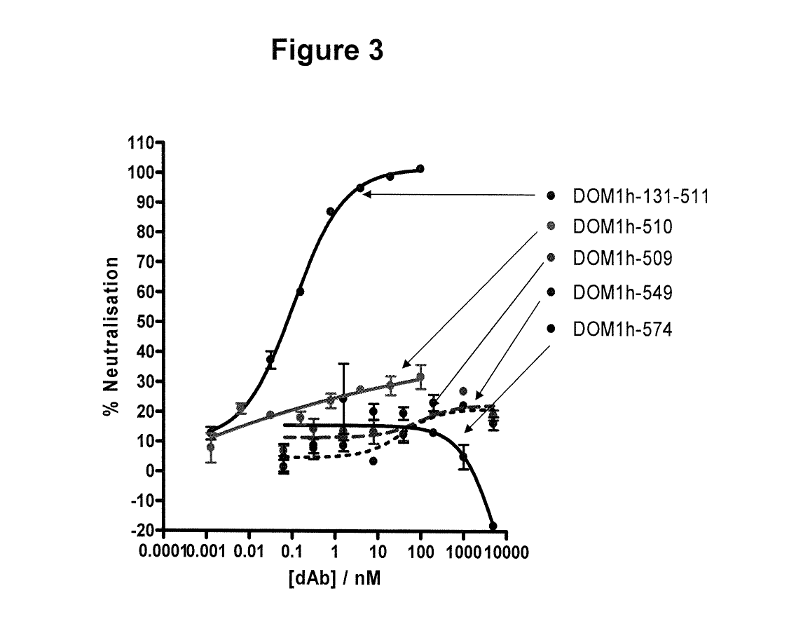

Further examples of suitable albumin, fragments and analogs for use in a TNFR1-binding format are described in WO 03076567, which disclosure is incorporated herein by reference and which forms part of the disclosure of the present text. In particular, the following albumin, fragments or variants can be used in the present invention:
Human serum albumin as described in WO 03076567, e.g., in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, e.g., HA(1-373., HA(1-388), HA(1-389), HA(1-369), and HA(1-419) and fragments between 1-369 and 1-419;
An albumin fragment or variant as described in EP 399666, e.g., HA(1-177) and HA(1-200) and fragments between HA(1-X), where X is any number from 178 to 199.

Where a (one or more) half-life extending moiety (e.g., albumin, transferrin and fragments and analogues thereof) is used to format the TNFR1-binding antagonists of the invention, it can be conjugated using any suitable method, such as, by direct fusion to the TNFR1-binding moiety (e.g., anti-TNFR1dAb), for example by using a single nucleotide construct that encodes a fusion protein, wherein the fusion protein is encoded as a single polypeptide chain with the half-life extending moiety located N- or C-terminally to the TNFR1 binding moiety. Alternatively, conjugation can be achieved by using a peptide linker between moieties, e.g., a peptide linker as described in WO 03076567 or WO 2004003019 (these linker disclosures being incorporated by reference in the present disclosure to provide examples for use in the present invention). Typically, a polypeptide that enhances serum half-life in vivo is a polypeptide which occurs naturally in vivo and which resists degradation or removal by endogenous mechanisms which remove unwanted material from the organism (e.g, human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

In embodiments of the invention described throughout this disclosure, instead of the use of an anti-TNFR1 single variable domain (“dAb”) in an antagonist of the invention, it is contemplated that the skilled addressee can use a polypeptide or domain that comprises one or more or all 3 of the CDRs of a dAb of the invention that binds TNFR1 (e.g, CDRs grafted onto a suitable protein scaffold or skeleton, eg an affibody, an SpA scaffold, an LDL receptor class A domain or an EGF domain) The disclosure as a whole is to be construed accordingly to provide disclosure of antagonists using such domains in place of a dAb. In this respect, see WO2008096158 for details of how to produce diverse libraries of based on protein scaffolds and selection and characterization of domains from such libraries, the disclosure of which is incorporated by reference).

In one embodiment, therefore, an antagonist of the invention comprises an immunoglobulin single variable domain or domain antibody (dAb) that has binding specificity for TNFR1 or the complementarity determining regions of such a dAb in a suitable format. The antagonist can be a polypeptide that consists of such a dAb, or consists essentially of such a dAb. The antagonist can be a polypeptide that comprises a dAb (or the CDRs of a dAb) in a suitable format, such as an antibody format (e.g, IgG-like format, scFv, Fab, Fab′, F(ab′)₂), or a dual specific ligand that comprises a dAb that binds TNFR1 and a second dAb that binds another target protein, antigen or epitope (e.g, serum albumin).

Polypeptides, dAbs and antagonists can be formatted as a variety of suitable antibody formats that are known in the art, such as, IgG-like formats, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g, a Fv fragment (e.g, single chain Fv (scFv), a disulfide bonded Fv), a Fab fragment, a Fab′ fragment, a F(ab′)₂ fragment), a single variable domain (e.g, V_{H}, V_{L}), a dAb, and modified versions of any of the foregoing (e.g, modified by the covalent attachment of polyalkylene glycol (e.g, polyethylene glycol, polypropylene glycol, polybutylene glycol) or other suitable polymer).

In some embodiments, the invention provides an antagonist that is an IgG-like format. Such formats have the conventional four chain structure of an IgG molecule (2 heavy chains and two light chains), in which one or more of the variable regions (V_{H} and or V_{L}) have been replaced with a dAb of the invention. In one embodiment, each of the variable regions (2 V_{H} regions and 2 V_{L} regions) is replaced with a dAb or single variable domain, at least one of which is an anti-TNFR1 dAb as herein described. The dAb(s) or single variable domain(s) that are included in an IgG-like format can have the same specificity or different specificities. In some embodiments, the IgG-like format is tetravalent and can have one (anti-TNFR1 only), two (e.g., anti-TNFR1 and anti-SA), three or four specificities. For example, the IgG-like format can be monospecific and comprises 4 dAbs that have the same specificity; bispecific and comprises 3 dAbs that have the same specificity and another dAb that has a different specificity; bispecific and comprise two dAbs that have the same specificity and two dAbs that have a common but different specificity; trispecific and comprises first and second dAbs that have the same specificity, a third dAb with a different specificity and a fourth dAb with a different specificity from the first, second and third dAbs; or tetraspecific and comprise four dAbs that each have a different specificity. Antigen-binding fragments of IgG-like formats (e.g, Fab, F(ab′)₂, Fab′, Fv, scF_{V}) can be prepared. In one embodiment, the IgG-like formats or antigen-binding fragments may be monovalent for TNFR1. If complement activation and/or antibody dependent cellular cytotoxicity (ADCC) function is desired, the ligand can be an IgG1-like format. If desired, the IgG-like format can comprise a mutated constant region (variant IgG heavy chain constant region) to minimize binding to Fc receptors and/or ability to fix complement. (see e.g, Winter et al., GB U.S. Pat. No. 2,209,757 B; Morrison et al., WO 89/07142; Morgan et al., WO 94/29351, Dec. 22, 1994).

The ligands of the invention (e.g., polypeptides, dAbs and antagonists) can be formatted as a fusion protein that contains a first immunoglobulin single variable domain that is fused directly to a second immunoglobulin single variable domain. If desired such a format can further comprise a half-life extending moiety. For example, the ligand can comprise a first immunoglobulin single variable domain that is fused directly to a second immunoglobulin single variable domain that is fused directly to an immunoglobulin single variable domain that binds serum albumin.

Generally the orientation of the polypeptide domains that have a binding site with binding specificity for a target, and whether the ligand comprises a linker, is a matter of design choice. However, some orientations, with or without linkers, may provide better binding characteristics than other orientations. All orientations (e.g, dAb1-linker-dAb2; dAb2-linker-dAb1) are encompassed by the invention are ligands that contain an orientation that provides desired binding characteristics can be easily identified by screening.

Polypeptides and dAbs, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides.

The invention moreover provides antagonists comprising or consisting of dimers, trimers and polymers of the aforementioned dAb monomers.

### Exemplification

Naïve Selection of Anti-TNFR1 dAb

Two different mechanisms to inhibit signaling of the TNF receptor 1 (p55) have been described (WO2006038027). The first consists of inhibition of signaling by binding a domain antibody to TNFR1 at an epitope where it competes directly with the binding of TNFα for its receptor. This competition can be determined in e.g. an in vitro receptor binding assay in which receptor is coated to a solid support and competition of the domain antibody with biotinylated TNFα for binding to the receptor is determined through measurement of residual biotinylated-TNFα binding using e.g. streptavidin-HRP. A competitive TNFR1 inhibitor will block TNFα binding to its receptor, leaving no TNFα signal. Conversely, a non-competitive TNFR1 inhibitor will have little influence on the binding of TNFα to the receptor, resulting in a continued read-out for biotinylated TNFα even in the presence of μM concentrations of inhibitory dAb. In a functional cell assay, e.g. the human MRC5 fibroblast cell line which upon stimulation with low levels of TNFα (10-200 pg/ml, for 18 h) releases IL-8, however, both competitive and non-competitive inhibitors reduce the IL-8 secretion in a dose dependent fashion. The latter demonstrates functional activity for both types of inhibitors in a cell-based system. Therefore the specific aim was to isolate domain antibodies which bind TNFR1 and inhibit its functional activity in cell assays, however these domain antibodies should not (substantially) compete with TNFα for binding to TNFR 1.

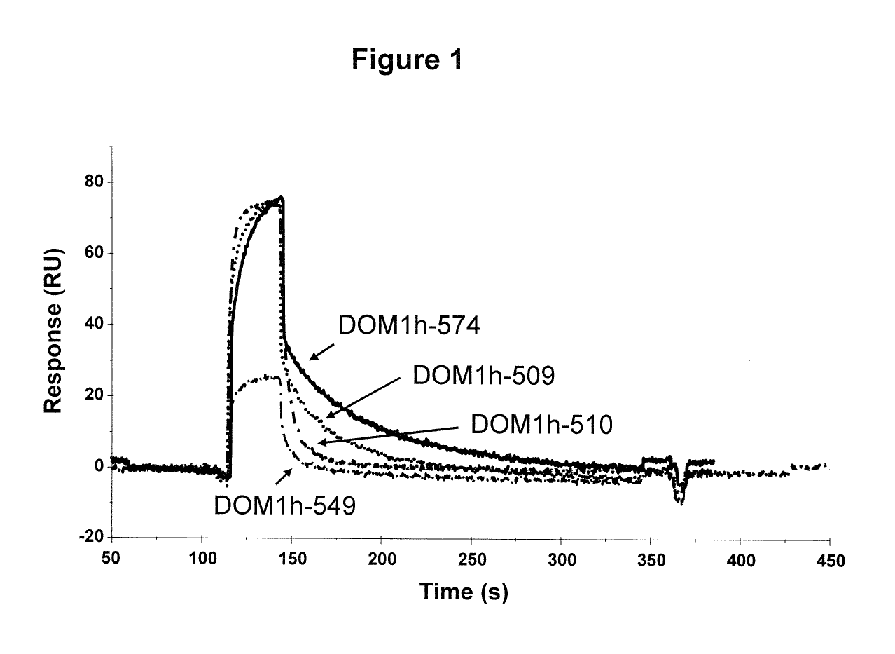

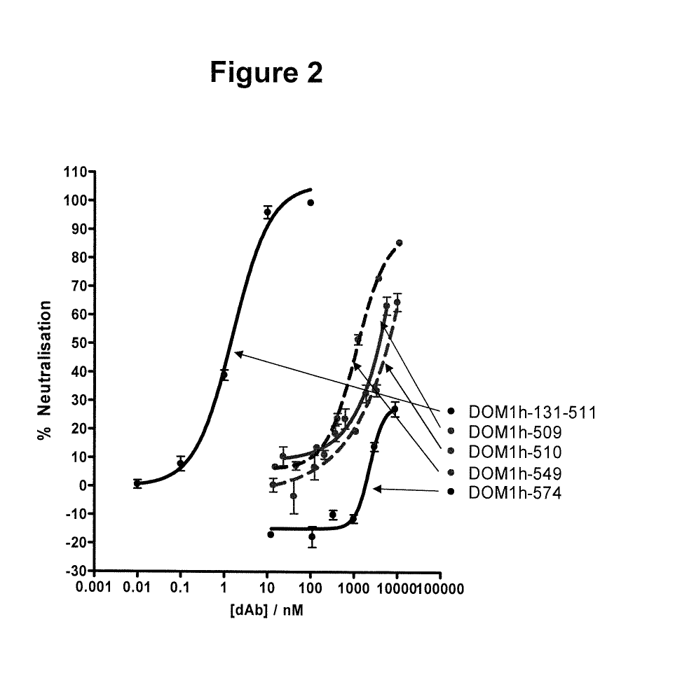

To isolate non-competitive, TNFR1-binding dAbs, a selection strategy was designed to enrich for this sub-class of dAbs. The approach consisted of using the Domantis' 4G and 6G naïive phage libraries, phage libraries displaying antibody single variable domains expressed from the GAS1 leader sequence (see WO2005093074) for 4G and additionally with heat/cool preselection for 6G (see WO04101790). These phage libraries were incubated in round 1 with 200 nM of biotinylated human TNFR1 (R&D systems, cat no. 636-R1/CF, biotinylated using EZ-Link NHS-LC-LC-biotin (Pierce cat no. 21343), according to the manufacturer's instructions), followed by pull-down on streptavidin-coated magnetic beads. In rounds 2 and 3, the phage were pre-incubated with TNFR1 (200 nM—round 2, 75 nM—round 3), and then with biotinylated TNFα (Peprotech cat no. 300-01A) (200 nM—round 2, 75 nM—round 3 nM) and pull-down on streptavidin-coated magnetic beads followed. In all rounds, beads were washed to remove weakly binding phage and bound phage were eluted by trypsin digestion prior to amplification. The rationale is that those dAbs which are able to bind TNFR1 in the presence of TNFα would be specifically enriched whereas those competing with TNFα would not be pulled down, as this epitope is required for the TNFα binding to the magnetic beads. Using this experimental design, 3 rounds of phage selection were done and both rounds 2 and 3 were cloned into the pDOM5 E. coli expression vector (see PCT/EP2008/067789), followed by dAbs expression and screening for TNFR1 binding on BIAcore™. The pDOM5 vector is a pUC119-based vector. Expression of proteins is driven by the LacZ promoter. A GAS1 leader sequence (see WO 2005/093074) ensures secretion of isolated, soluble dAbs into the periplasm and culture supernatant of E. coli. dAbs are cloned SalI/NotI in this vector, which appends a myc tag at the C-terminus of the dAb. Binding dAbs were expressed at 50 ml scale and affinity purified for functional characterisation. This consisted of determination of inhibition of TNFα-mediated signaling in a standard MRC5 cell assay (as described below) as well as inhibition of TNFα binding to TNFR1 in a receptor binding assay (as described below). Screening of 6000 supernatants yielded many TNFR1 binders. However, the vast majority either bound an irrelevant epitope, consequently having no activity in either the cell assay or the receptor binding assay, or were competitive as demonstrated in the receptor binding assay. Notwithstanding this majority, sequence analysis of those dAbs which 1) bound TNFR1 on BIAcore (FIG. 1), 2) inhibited TNFα in the MRC5 cell assay (FIG. 2) whilst, 3) demonstrating no TNFα competition in the Receptor Binding Assay (FIG. 3), identified five unique dAbs. These five dAbs were: DOM1h-509, DOM1h-510, DOM1h-543, DOM1h-549 and DOM1h-574.
Test Maturation of Selected dAbs by Error-Prone Mutagenesis

In order to determine the maturability of DOM1h-509, DOM1h-510, DOM1h-543, DOM1h-549 and DOM1h-574, error-prone PCR libraries of dAb mutants were generated using the Genemorph II kit (Stratagene (San Diego, USA) cat. no. 200550) according to the manufacturer's instructions. Sequence analysis revealed these libraries to have an average mutation rate of 2.4% on the amino-acid level. These libraries were cloned in the phage vector pDOM4 and expressed on phage. pDOM4 is a filamentous phage (fd) display vector, which is based on fd vector with a myc tag and wherein a protein sequence can be cloned in between restriction sites to provide a protein-gene III fusion. The genes encoding dAbs were cloned as SalI/NotI fragments.

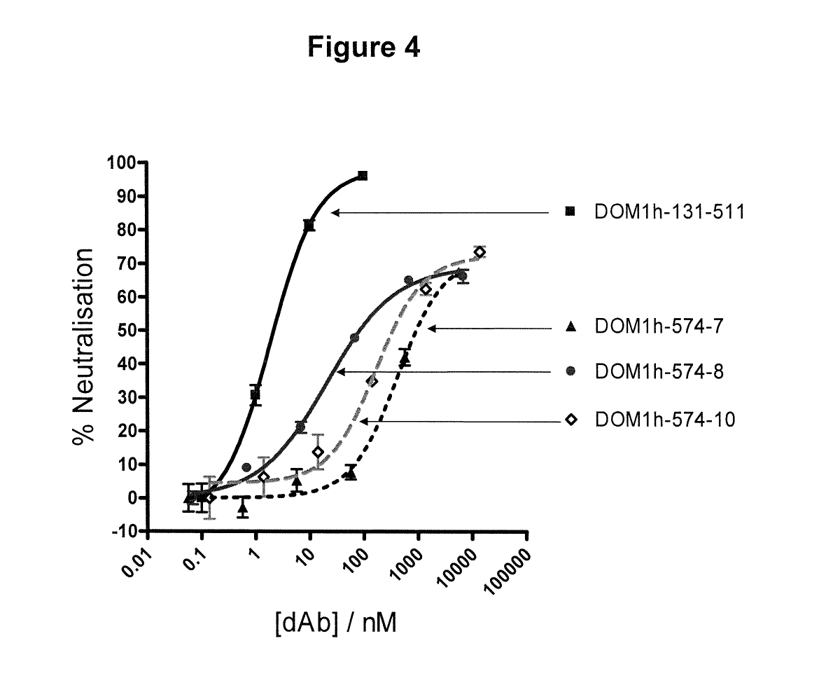

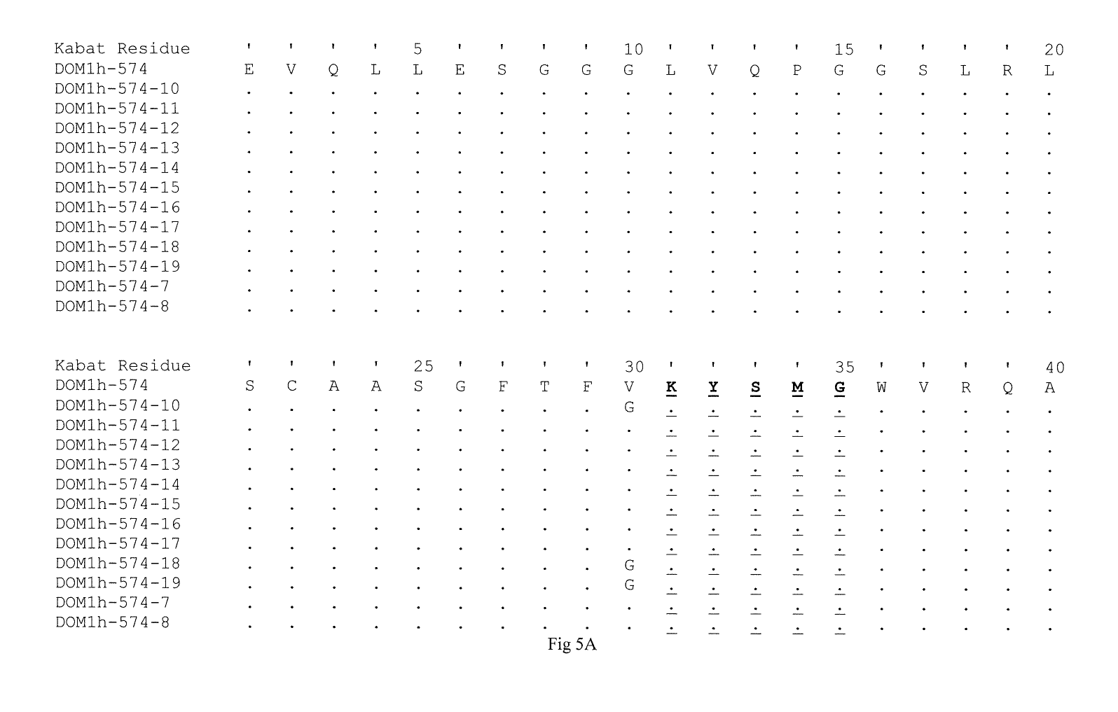

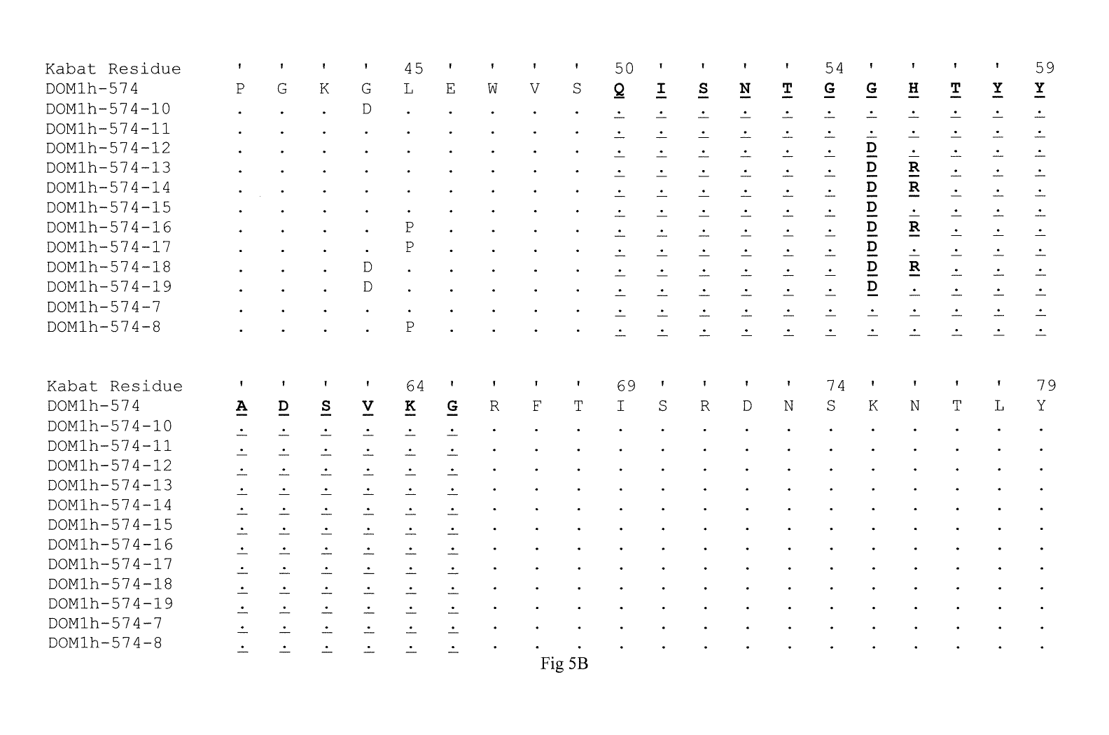

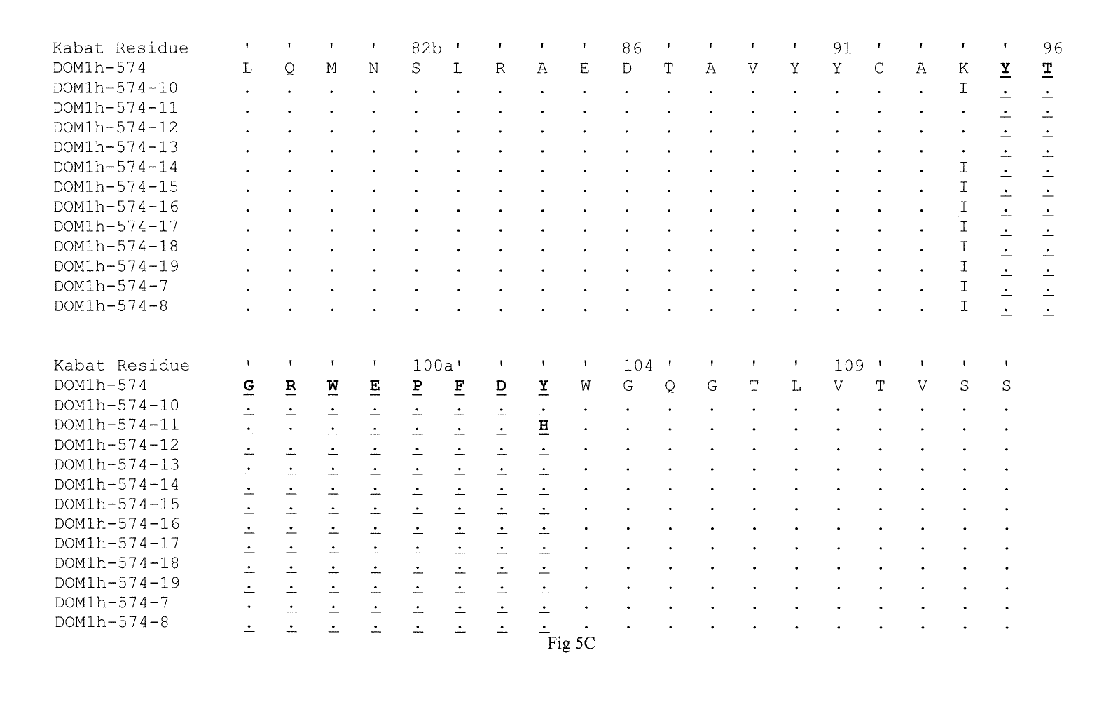

Selections for improved binders were done over three sequential rounds of incubation with decreasing amounts of biotinylated human TNFR1 (R&D Systems) (50 nM (round 1), 5 nM (round 2) and 0.5 nM (round 3)). After three rounds of selections, the dAb genes were cloned into the E. coli expression vector pDOM5, expressed and the supernatants screened by BIAcore for improvements in binding kinetics. Although variants derived from all five parental lineages were screened, only dAbs from the DOM1h-574 lineage showed significant improvements in the dissociation rate when screened on the BIAcore. Those dAbs with the most pronounced improvements in dissociation rate were purified and characterised in the MRC5 cell assay (Table 1 and FIG. 4), the best dAbs being: DOM1h-574-7, DOM1h-574-8, DOM1h-574-10, DOM1h-574-11, DOM1h-574-12 and DOM1h-574-13. From the examination of these dAbs, we exercised our judgement and identified positions and mutations which might be responsible for the affinity improvements, specifically: V30G, G44D, L45P, G55D, H56R and K94I (Kabat numbering). In search of an additive effect, we generated novel dAb variants which combine these specific mutations into a single dAb. The novel variants engineered using DOM1h-574 template were: DOM1h-574-14 (G55D, H56R and K94I), DOM1h-574-15 (G55D and K94I), DOM1h-574-16 (L45P, G55D, H56R and K94I), DOM1h-574-17 (L45P, G55D and K94I), DOM1h-574-18 (V30G, G44D, G55D, H56R and K94I) and DOM1h-574-19 (V30G, G44D, G55D and K94I) (FIG. 5). Characterisation of these variants for potency in the MRC5 cell assay and affinity for TNFR1 on BIAcore identified further improvements (Table 1). The most potent dAb was DOM1h-574-16.

TABLE 1
Summary of BIAcore affinities and potencies in the MRC5
cell assay for DOM1h-574 parent and the dAbs identified
during test maturation and constructed through recombination
of beneficial mutations. DOM1h-574-16 combines the highest
affinity on BIAcore with the highest potency in the MRC5 cell assay.
Where values were not determined, this is indicated (ND).

| | BIAcore K_{D} (nM) | MRC-5 ND₅₀ (nM) |
|---|---|---|
| DOM1h-574 | 210 | 2200 |
| DOM1h-574-7 | 48 | 580 |
| DOM1h-574-8 | 5.7 | 10 |
| DOM1h-574-10 | 19 | 100 |
| DOM1h-574-11 | 200 | 800 |
| DOM1h-574-12 | 23 | 130 |
| DOM1h-574-13 | 44 | 300 |
| DOM1h-574-14 | ND | ND |
| DOM1h-574-15 | 20 | 300 |
| DOM1h-574-16 | 1.0 | 8 |
| DOM1h-574-17 | 8.4 | 20 |
| DOM1h-574-18 | 4.1 | 17 |
| DOM1h-574-19 | ND | 140 |

Species Cross-Reactivity of DOM1h-574-16

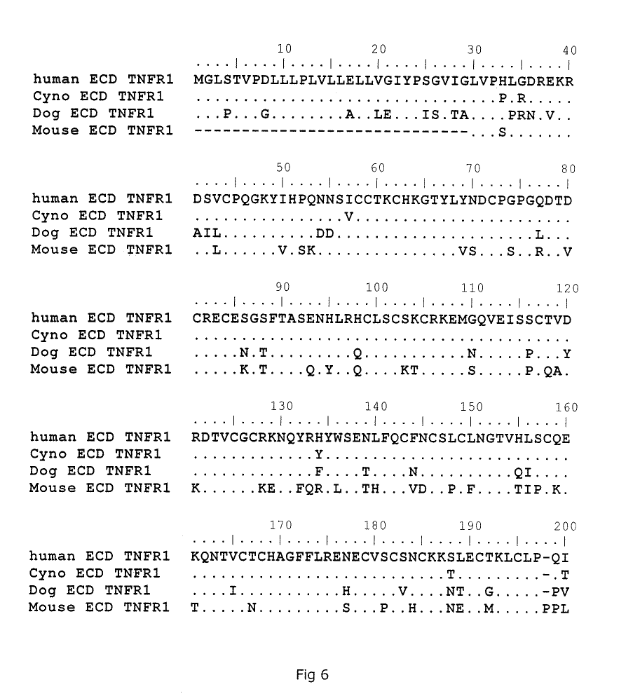

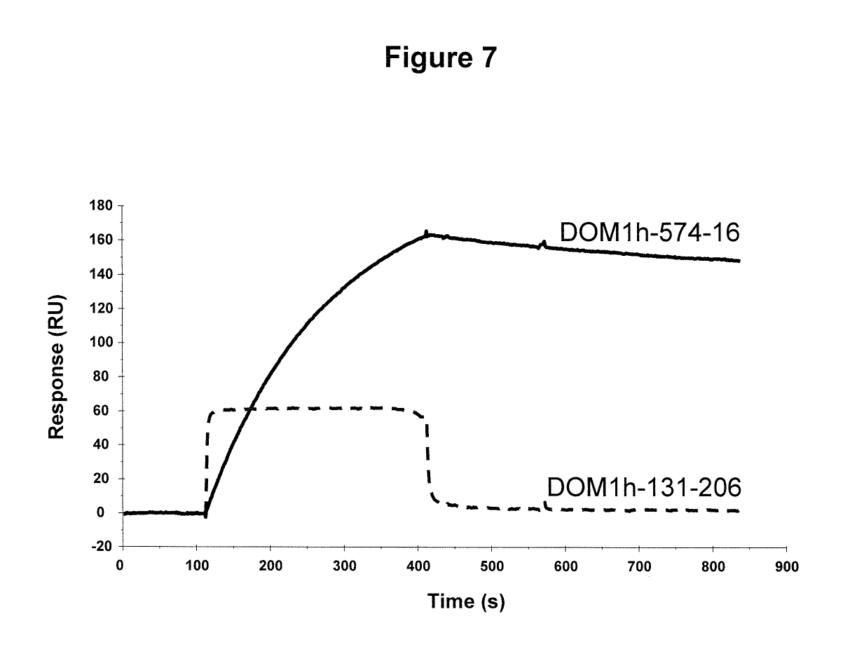

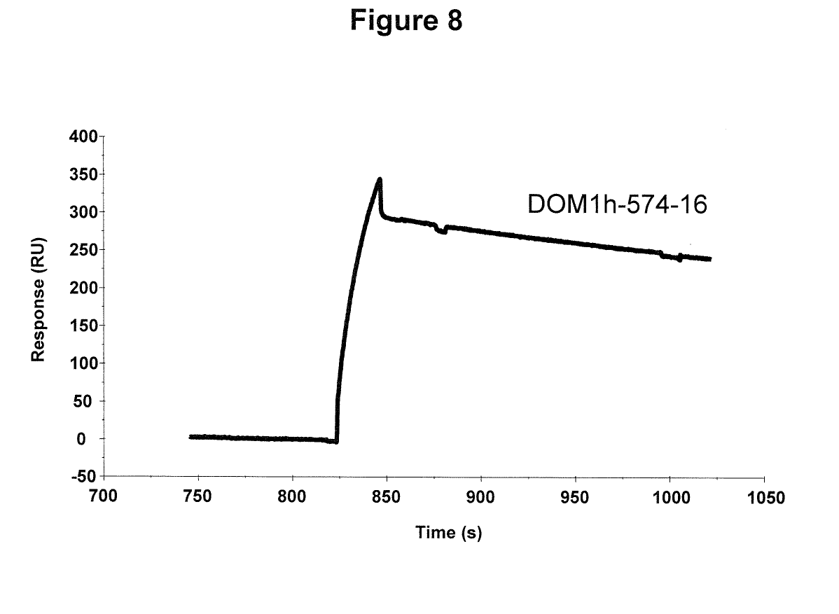

A significant advantage for an anti-TNFR1 dAb would be cross-reactivity between different species. Given the limited conservation of the sequence of the extracellular domain of TNFR1 between mouse, dog, Cynomologus monkey and human (FIG. 6), it would be exceptional for any antibody or single variable domain to recognize TNFR1 of these different species at similar affinities. Therefore, we tested the ability of DOM1h-574-16 to bind on BIAcore to mouse TNFR1 (R&D systems cat no. 425-R1-050/CF), dog TNFR1 (R&D Systems cat no. 4017-TR-025/CF) and human TNFR1 (R&D Systems). For each of these experiments the TNFR1 was biotinylated using EZ-Link NHS-LC-LC-biotin (Pierce cat no. 21343), according to the manufacturer's instructions, followed by binding of the biotinylated TNFR1 to a Streptavidin-coated BIAcore chip. Subsequently, DOM1h-574-16 was injected over human, mouse and dog TNFR1 and binding was monitored on the BIAcore. Examples for binding to the different species are shown in FIGS. 7 and 8, with a summary of the results in Table 2. Clearly, DOM1h-57-16 demonstrates high-affinity binding to the different TNFR1 species in contrast to our previously described (WO2008149148) competitive anti-TNFR1 dAb DOM1h-131-206, which showed virtually no binding to mouse TNFR1 and only very weak binding to dog TNFR1.

TABLE 2
Binding affinity of DOM1h-131-206 and DOM1h-574-16 for mouse,
dog and human TNFR1 as determined by BIAcore.

| | | Dog TNFR1 | Human |
|---|---|---|---|
| | Mouse TNFR1 (K_{D}) | (K_{D}) | TNFR1 (K_{D}) |
| DOM1h-131-206 | ND* | >500 nM | 0.47 nM |
| DOM1h-574-16 | 20 nM | 20 nM | 1 nM |

*= affinity too poor to be determined by BIAcore (>μM)

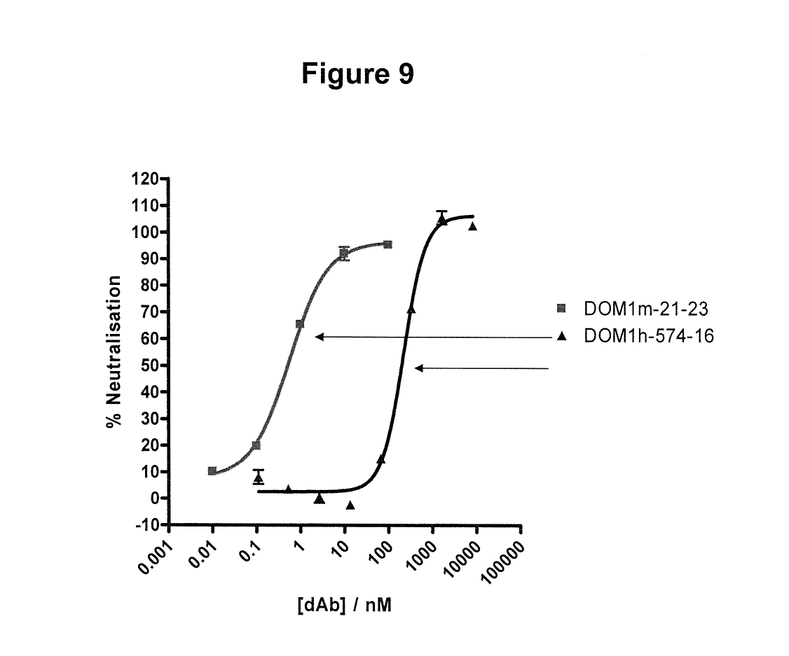

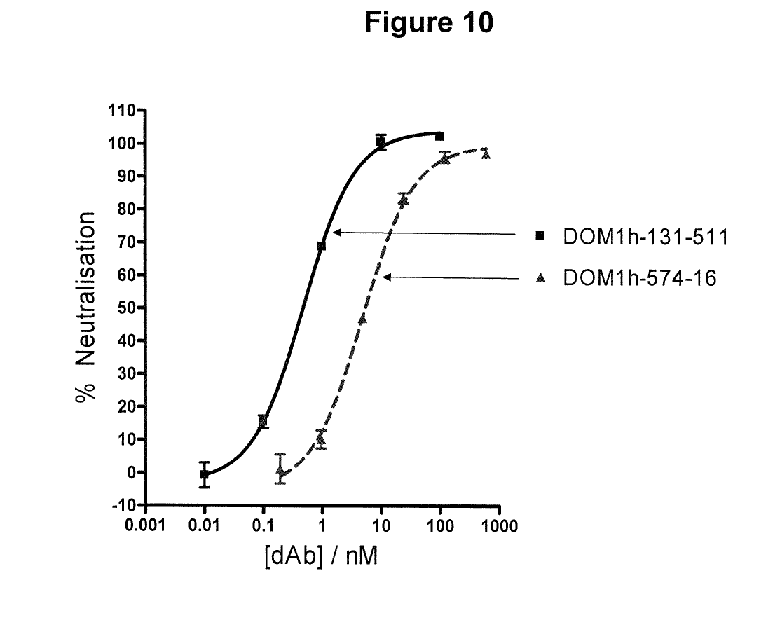

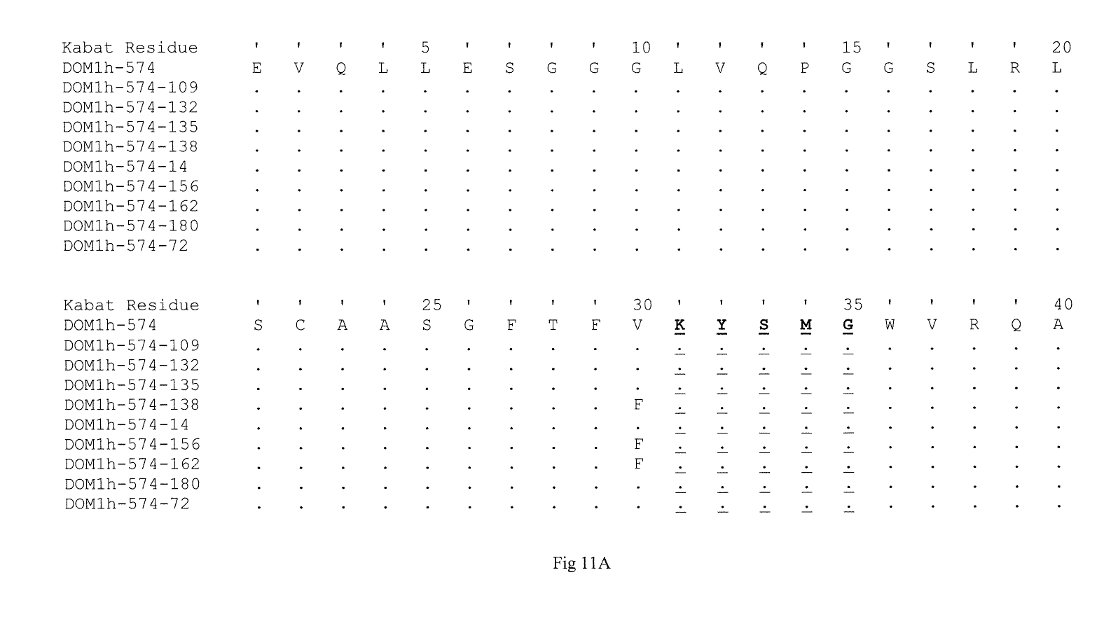

Next, the potency of DOM1h-574-16 to inhibit TNFα-mediated cytotoxicity of mouse cells (L929) and inhibition of TNFα-mediated, IL-8 release of Cynomologus monkey cells (CYNOM-K1) was evaluated. Both the standard mouse L929 and CYNOM-K1 cell assays were performed as described previously (WO2006038027) and above. Briefly, mouse L929 cells were incubated overnight with 20 pg/ml of mouse TNFα in the presence of actinomycine and a dose range of DOM1h-574-16. After 18 h, cell viability was checked and plotted against the DOM1h-574-16 concentration. In the Cynomologus monkey CYNOM-K1 cell assay, cells were stimulated with TNFα (200 pg/ml) for 18 h in the presence of a dose range of DOM1h-574-16. After the incubation, media was removed and the level of IL-8 was determined. The percentage of neutralization was plotted against the concentration of DOM1h-574-16. For both cell types, DOM1h-574-16 was able to efficiently inhibit the TNFα-mediated effects. Its potency was ˜250 nM in the mouse standard L929 cell-based assay and ˜10 nM in the Cynomologus monkey CYNOM-K1 assay (FIGS. 9 and 10). These results demonstrate functional, species cross-reactivity of DOM1h-574-16 in cell-based assays.

Affinity Maturation of DOM1h-574

Based on this test maturation and the results of the combination mutants, it was decided to use DOM1h-574-14 as the template for further affinity maturation. Whilst this particular dAb was not the most potent, it does not have any framework mutations compared to germline DP47 frameworks and was therefore chosen. For affinity maturation, the CDRs of DOM1h-574-14 were randomised by amplifying the CDRs using the following oligonucleotides: AS1029 and AS339 (CDR1), AS1030 and AS339 (CDR2) and AS1031 and AS339 (CDR3). The second PCR fragment for each library was made using the following oligonucleotide combinations: AS1031′ and AS9 (CDR1), AS1032 and AS9 (CDR2), AS1033 and AS9 (CDR3). Using SOE PCR (Horton et al. Gene, 77, p 61 (1989)) the two CDR1PCR products were combined to create the CDR1 library, the CDR2 products for the CDR2 library and the CDR3 products for the CDR3 library. For all reactions the SOE product was then amplified with the nested primers AS639 and AS65 and ligated SalI/NotI in the pIE2aA² vector, described in WOWO2006018650. The randomisation oligonucleotides (AS1029, AS1030 and AS1031) consisted of fixed positions (indicated by a capital letter and in which case 100% of oligonucleotides have the indicated nucleotide at that position) and mixed nucleotide composition, indicated by lower case in which case 85% of oligonucleotides will have the dominant nucleotide at this position and 15% will have an equal split between the remaining three nucleotides. Three different libraries were prepared using DNA-display construct pIE2aA². An aliquot of the library was used to transform E. Coli and sequenced. Relative to the parent clones, the affinity maturation libraries contained many mutations across the CDRs. Selections were performed using in vitro compartmentalisation in emulsions and DNA display through the scArc DNA binding protein (see WO2006018650). Thirteen rounds of selection were carried out in total, whilst keeping the libraries separate. Five rounds of equilibrium selections with 20, 15, 10, 8 and 5 nM biotinylated human TNFR1 (R&D Systems), were followed by seven rounds of off-rate selection for up to 150 min, by using unlabelled 10 μM hTNFR1 as a competitor. Library fitness during the selection process was assayed by real-time PCR. The principle of the method used is the following: In vitro titration of polyclonal population fitness by qPCR provides a semiquantitative measure of the average affinity of a polyclonal dAb population by measuring the amount of encoding DNA in complex with dAb-scArc protein that is captured by surface-bound antigen after in vitro expression reaction in solution conditions (no genotype-phenotype linkage). The higher is the fraction of input DNA which is recovered, the more potent is the polyclonal dAb population. Suitable reference points are the binding levels of parent clone to a non-specific surface coated with irrelevant antigen and specific binding to the surface coated with target antigen.

DNA templates recovered during the different stages of selection were diluted to 1.7 nM concentration in 0.1 mg/ml RNA solution. In vitro expression reactions were carried out in 10 μl volume of EcoPro T7 E. coli extract supplemented with 0.3 μl of 100 mM oxidized glutathione, 0.05 μl of 340 nM anti-HA mAb 3F10 from Roche and 0.5 μl of 1.7 nM DNA template.

The wells of Strep ThermoFast plates were coated with biotinylated hTNFR1 target antigen (0.1 μl of 30 μM stock/well) or BSA negative control (0.1 μl of 2 mg/ml stock/well) for 1 hour at room temperature, followed by three washes with buffer C (10 mM Tris, 100 mM KCl, 0.05% Tween 20, 5 mM MgCl₂ and 0.1 mM EDTA).

In vitro expression reactions were incubated at 25° C. for three hours, then diluted to 100 μl using buffer C, applied in two 50 μl aliquots to the wells of Strep ThermoFast plate (ABgene, UK) previously coated with biotinylated hTNFR1 or BSA, incubated for further one hour at room temperature and washed three times with buffer C to remove any unbound DNA.

Bound DNA molecules were amplified using oligonucleotides AS79 and AS80 and iQ SYBR Green Supermix (Bio-Rad Laboratories, cat no. 170-8880), which was used according to manufacture's instructions, and amplification cycles were: 2 min 94° C., followed by 40 cycles of 15 sec 94° C., 30 sec 60° C. and 30 sec 72° C. The amount of DNA was quantified on a BioRad MiniOpticon Real-Time PCR Machine (Bio-Rad Laboratories, Hercules Calif.) and analysed using Opticon Monitor version 3.1.32 (2005) software provided by Bio-Rad Laboratories. Standard curve from a sample of known DNA concentration covered the range from 500 to 5×10⁸ molecules per reaction. Up to tenth round of selection, the fitness of the library increased as each round recovered more DNA than the previous rounds, indicating that the average number of binding dAbs was increasing. From this point onwards, no increases were seen in the level of recovered DNA, as determined by real-time PCR, suggesting that additional rounds of selection were not yielding significant further improvements in dAb affinities. The selected population of rounds 10 and 13 were cloned, sequenced, expressed and BIAcore-assayed for dissociation rate constants in unpurified form.

It was found that the library diversity was greatly reduced, with a number of clones displaying improved (2-3 fold) dissociation rate constants as determined by BIAcore dAb supernatant screening. DNA sequencing of these improved dAbs identified DOM1h-574-25 to DOM1h-574-40.

The beneficial mutations identified based on these dAbs are listed below for each CDR separately:
CDR1: V30 is beneficially mutated to L or F.
CDR2: S52 is beneficially mutated to A or T,
   N52a is beneficially mutated to D or E,
   G54 is beneficially mutated to A or R,
   T57 is beneficially mutated to R, K or A,
   A60 is beneficially mutated to D, S, T or K,
   D61 is beneficially mutated to E, H or G,
   S62 is beneficially mutated to A or T,
CDR3: E100 is beneficially mutated to Q, V, A, D or S,
   D101 is beneficially mutated to E, V, H or K.

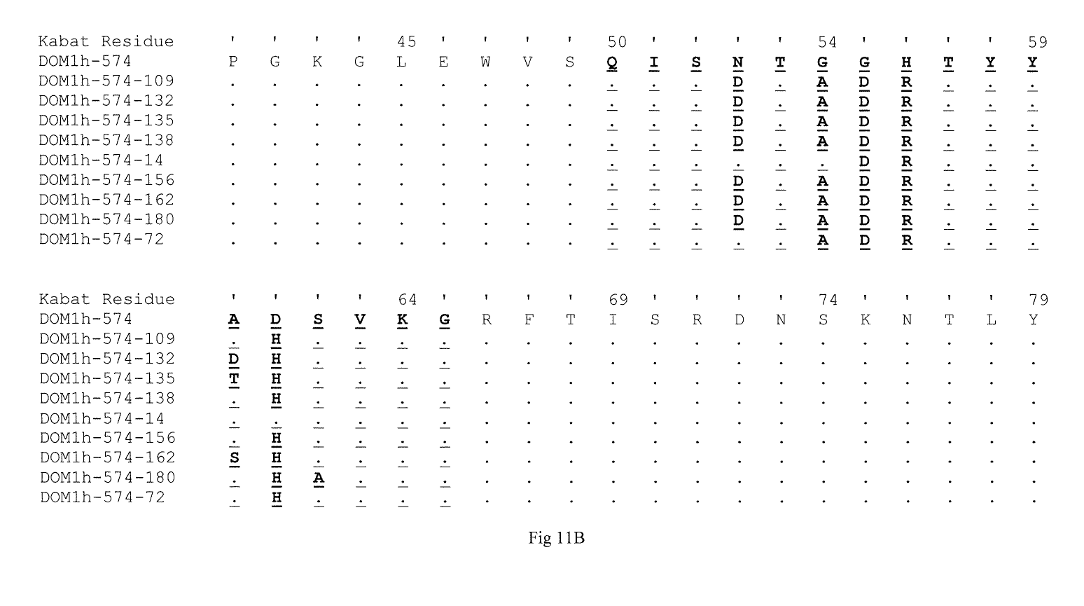

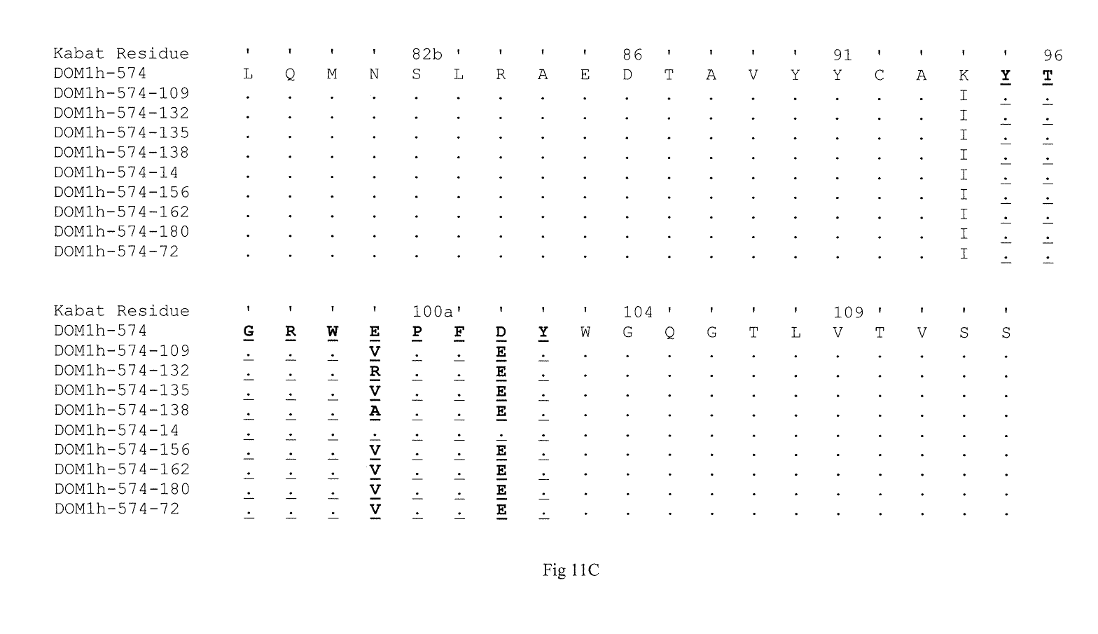

At first, the CDR1+2 of clones DOM1h-574-30, -31, -38 and -39 was recombined in a mini-library with the CDR3s of clones DOM1h-574-25, -26, -27 and -28. These dAbs were chosen as they represented the dAbs with the largest improvements in BIAcore affinity and therefore combinations of these dAbs would have the best chance at identifying novel sequences with enhanced affinity. The resulting recombined dAbs were DOM1h-574-65 to DOM1h-574-79 and DOM1h-574-84 to DOM1h-574-88: SEQ ID NOs: . . . to . . . ], of which DOM1h-574-72 was the most potent. This dAb was subsequently used to evaluate the usefulness of the rest of the mutations, in clones DOM1h-574-89 to DOM1h-574-93, DOM1h-574-109 to DOM1h-574-149, and DOM1h-574-151 to DOM1h-574-180. Most of these clones were expressed, purified and assayed for binding on BIAcore, potency in the MRC5 cell assay and protease stability as determined by resistance to trypsin digestion. The protease stability was determined by incubation of dAb at 1 mg/ml in PBS with decreasing amounts of trypsin (Promega, V511A trypsin). Incubation was performed at 5 different concentrations of trypsin (34, 17, 8.5, 4.25 and 2.13 μg/ml) as well as a control lacking trypsin. After incubation at 37° C. for three hours, the proteolytic reaction was stopped by adding loading dye and the amounts of residual, uncleaved dAb was determined on a LabChip 90 system (Caliper Life Sciences). The most improved clones have about 30-fold potency improvement over DOM1h-574-14, the starting dAb used for affinity maturation. The most potent in the MRC5 cell assay are: DOM1h-574-109, DOM1h-574-132, DOM1h-574-135, DOM1h-574-138, DOM1h-574-156, DOM1h-574-162 and DOM1h-574-180 (FIG. 11).

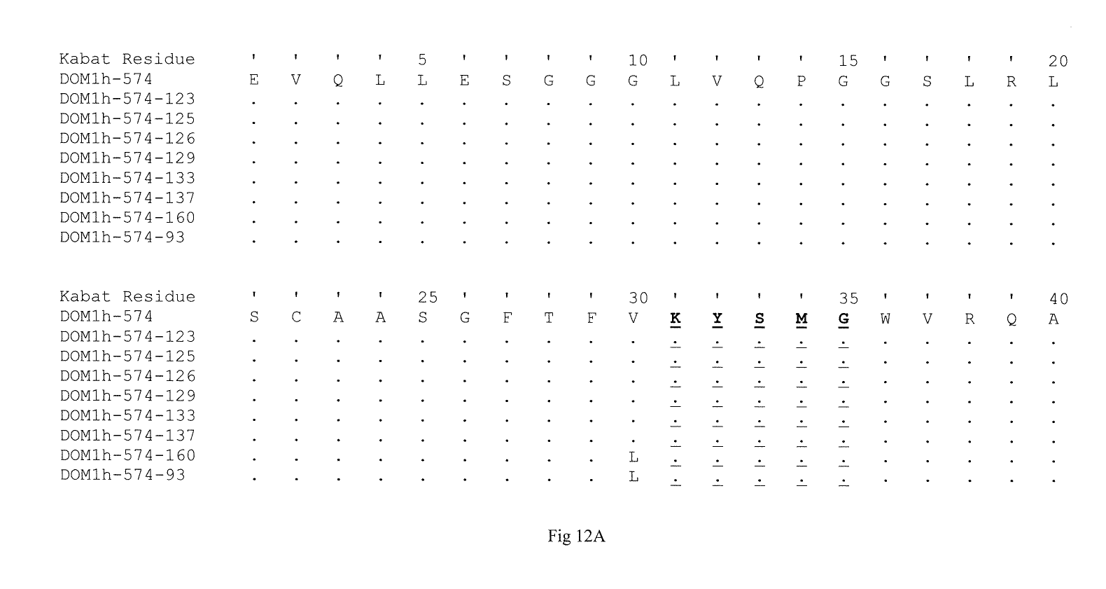

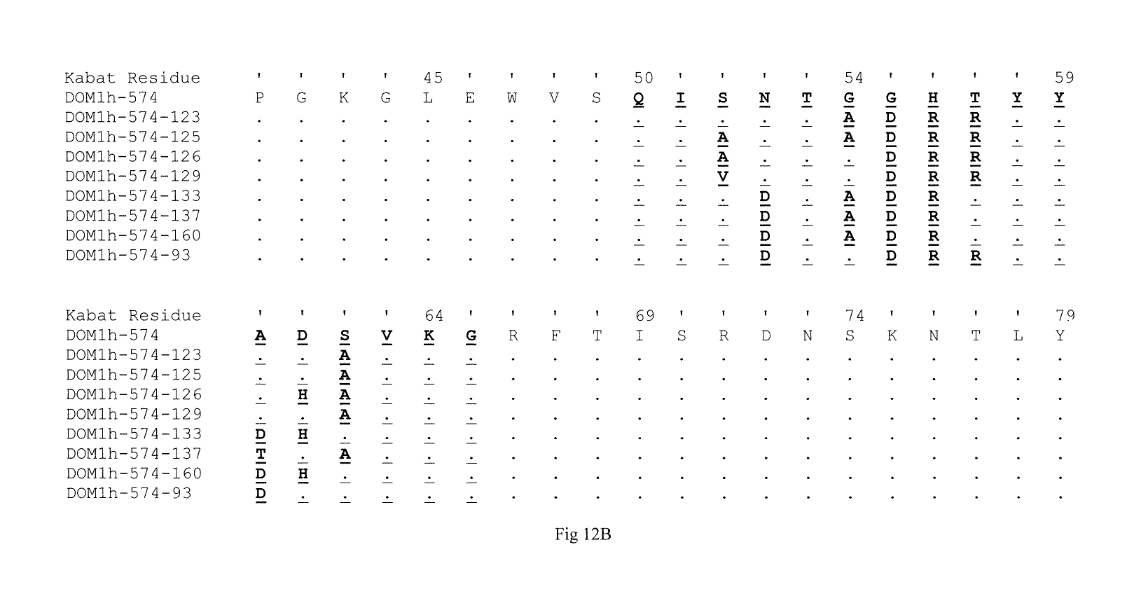

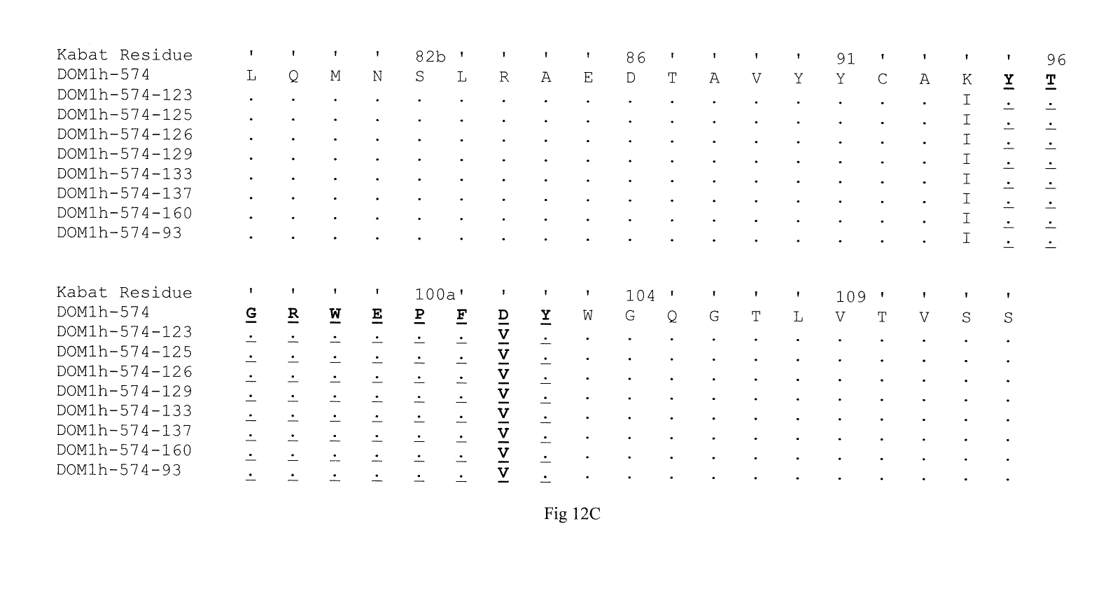

Surprisingly, it was found that the structural determinants for affinity/potency on one hand and the protease stability on the other hand are different. Whilst most of the listed mutations improved affinity to sub-nM range as determined by BIAcore, they also led to decreased trypsin resistance (see WO2008149143 and WO2008149148) for more description on suitable assays for determining protease stability of dAbs). On the other hand, mutation D101V (Kabat numbering) was very frequently associated with the best protease stability, albeit at the expense of about a two-fold reduction of dAb affinity, compared with any other tested sequence. The most protease stable dAbs are: DOM1h-574-93, DOM1h-574-123, DOM1h-574-125, DOM1h-574-126, DOM1h-574-129, DOM1h-574-133, DOM1h-574-137 and DOM1h-574-160 (FIG. 12).

Characterisation of Most Promising DOM0100 dAbs

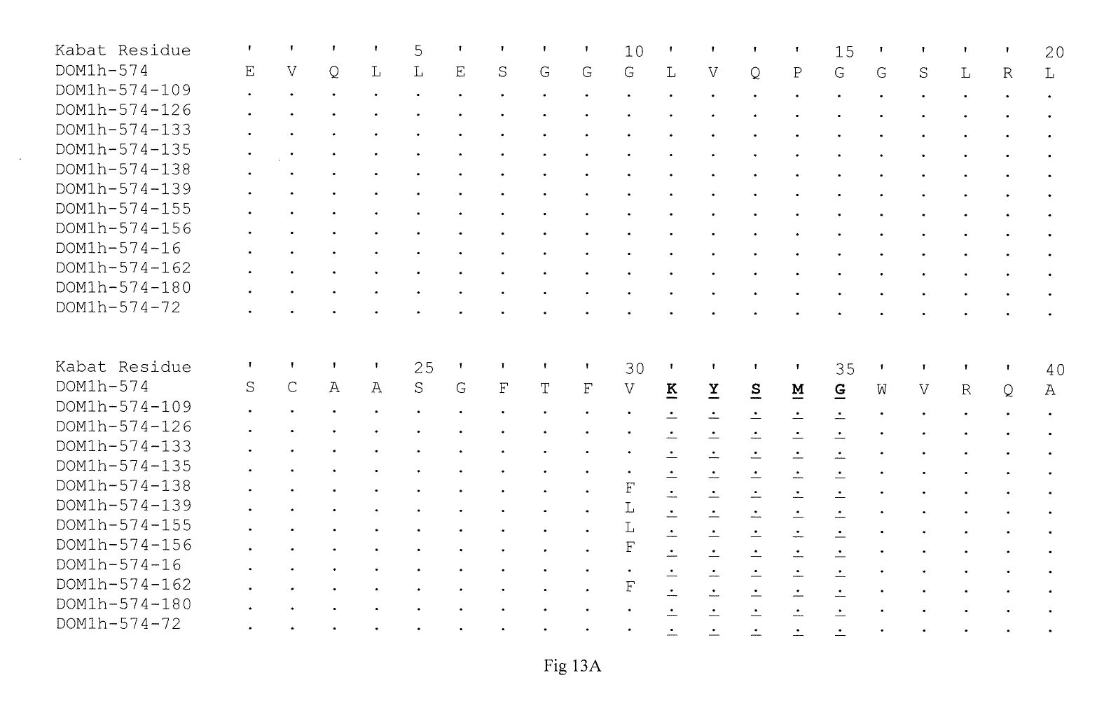

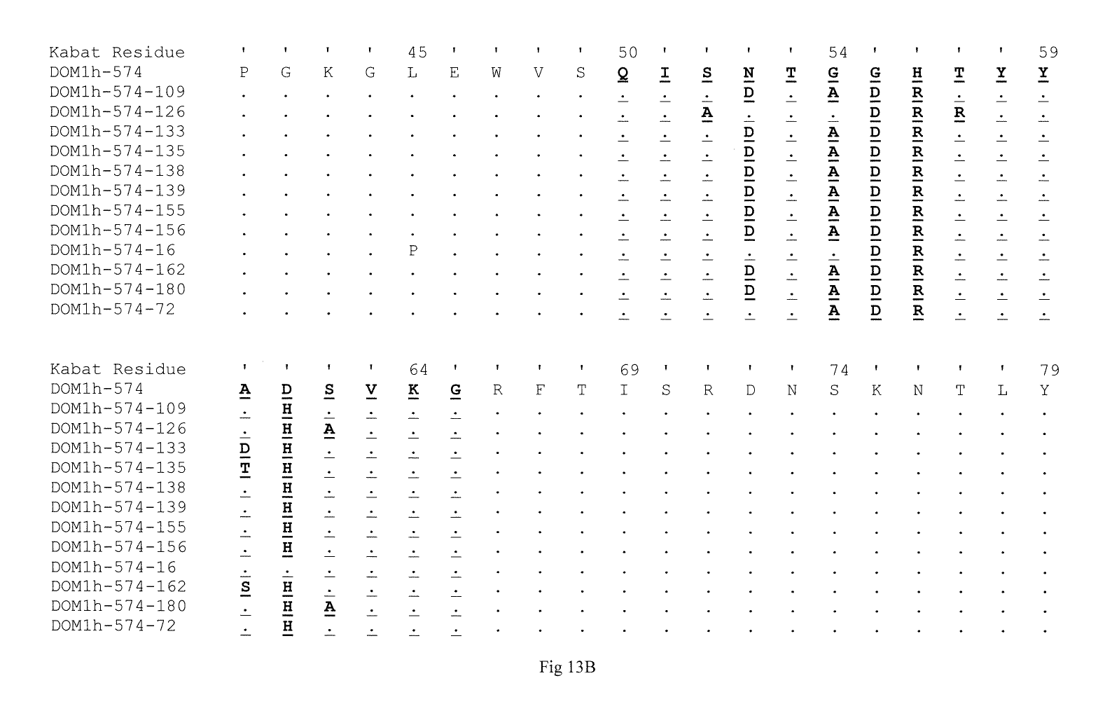

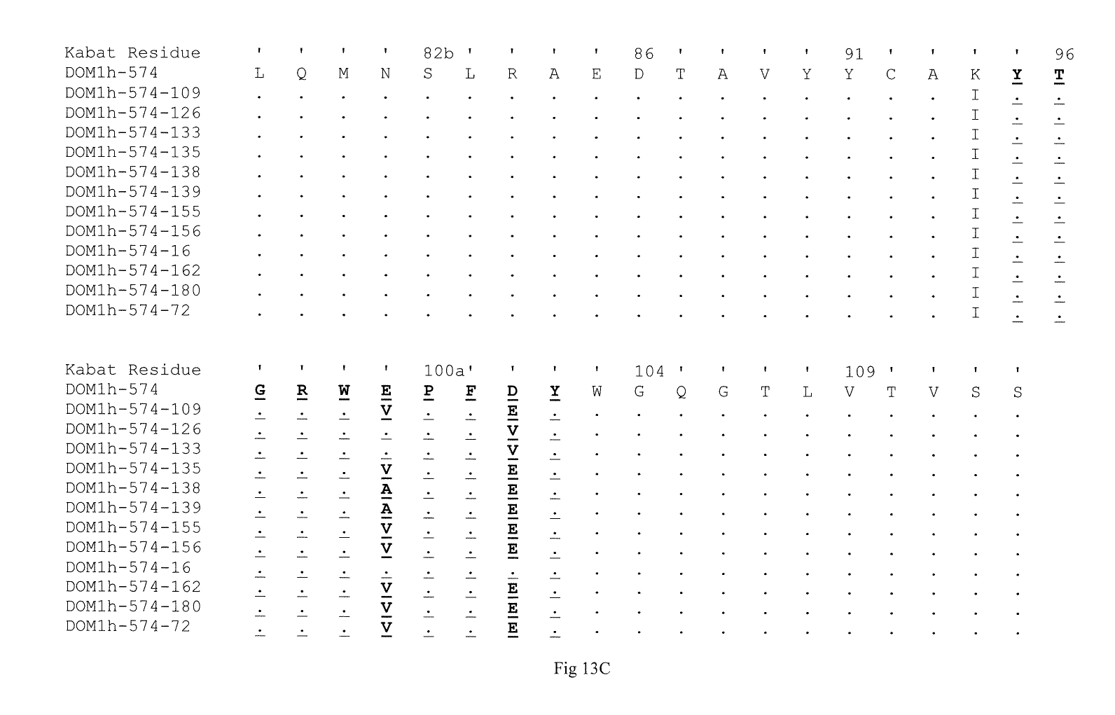

Based on the data for BIAcore binding and MRC5 cell assay potency, a subset of 12 DOM0100 dAbs were chosen for further characterisation of binding kinetics to TNFR1, potency in cell assays and biophysical properties. For all these experiments the dAbs were expressed in E. coli and purified using Protein A streamline followed by dialysis in PBS. The 12 dAbs used for this characterisation were: DOM1h-574-72, DOM1h-574-109, DOM1h-574-126, DOM1h-574-133, DOM1h-574-135, DOM1h-574-138, DOM1h-574-139, DOM1h-574-155, DOM1h-574-156, DOM1h-574-162 and DOM1h-574-180. For certain experiments DOM1h-574-16 is included as a reference (FIG. 13).

Binding Properties DOM0100 dAbs (Anti-TNFR1 dAbs)

BIAcore was done to determine the association and dissociation rates of the different dAbs and in that way establish their binding affinity for both human and mouse TNFR1. Experiments were done using biotinylated TNFR1 (R&D Systems), of the respective species, coupled to streptavidin-coated BIAcore chips followed by injection of a concentration range of the dAbs. The results are summarised in Table 3. All dAbs show high affinity binding to human TNFR1 (KD<350 pM) as well as good affinity for mouse TNFR1 (KD<7 nM). This difference in dAb affinity of about 20-fold between human and mouse TNFR1 is quite surprising given the limited sequence homology between mouse and human TNFR1 and might indicate the targeting of a highly conserved motif in the receptor.

TABLE 3
BIAcore analysis of association and dissociation
of DOM0100 dAbs for human and mouse TNFR1.

| | Human | | | Mouse | | |
|---|---|---|---|---|---|---|
| | Kon | Koff | KD | Kon | Koff | KD |
| DOM0100 dAb | (×10⁵ M⁻¹s⁻¹) | (×10⁻⁵ s⁻¹) | (pM) | (×10⁵ M⁻¹s⁻¹) | (×10⁻⁴ s⁻¹) | (nM) |
| DOM1h-574-72 | 2.5 | 8.4 | 350 | 1.0 | 6.8 | 6.9 |
| DOM1h-574-109 | 2.4 | 5.5 | 230 | 1.2 | 3.3 | 2.8 |
| DOM1h-574-126 | 3.8 | 7.9 | 210 | 1.6 | 6.8 | 4.4 |
| DOM1h-574-133 | 2.6 | 8.8 | 340 | 1.4 | 7.5 | 5.2 |
| DOM1h-574-135 | 2.5 | 5.2 | 210 | 1.1 | 4.5 | 3.8 |
| DOM1h-574-138 | 2.5 | 3.8 | 150 | 1.3 | 3.0 | 2.4 |
| DOM1h-574-139 | 1.4 | 3.7 | 270 | 0.7 | 3.0 | 4.4 |
| DOM1h-574-155 | 2.4 | 4.3 | 180 | 1.1 | 3.3 | 3.7 |
| DOM1h-574-156 | 3.0 | 4.3 | 150 | 1.4 | 3.0 | 2.1 |
| DOM1h-574-162 | 2.9 | 4.4 | 150 | 1.4 | 3.4 | 2.5 |
| DOM1h-574-180 | 2.7 | 4.1 | 150 | 1.2 | 3.2 | 2.7 |

The most potent anti-human TNFR1 dAbs tend to also be the most potant anti-mouse TNFR1 dAbs, e.g. DOM1h-574-138 and DOM1h-574-156.

Biophysical Properties DOM0100 dAbs

The DOM0100 dAbs were further characterized for their biophysical properties, which included their protease stability, thermal stability and in-solution state. The protease stability was determined by incubation of dAb at 1 mg/ml in PBS with decreasing amounts of trypsin (Promega, V511A trypsin). Incubation was performed at 5 different concentrations of trypsin (34, 17, 8.5, 4.25 and 2.13 μg/ml) as well as a control lacking trypsin. After incubation at 37° C. for three hours, the proteolytic reaction was stopped by adding loading dye and the amounts of residual, uncleaved dAb was determined on a LabChip 90 system (Caliper Life Sciences). Amounts were quantified as a percentage of the amount present in the control reaction and are summarized in Table 4. Thermal stability of the DOM0100 dAbs was determined using a differential scanning calorimetry (DSC) instrument (MicroCal, MA). dAbs, at 1 mg/ml in PBS, were incubated in the instrument and the melting temperature determined. The results are summarized in table 4. Finally, the in-solution state of the dAbs was determined using size-exclusion chromatography and multi-angle laser light scattering (SEC-MALLS). The dAbs were injected on the SEC-MALLS at 1 mg/ml in PBS and the mass of the main peak determined. The DOM0100 dAbs could be divided in two groups, either monomeric or dimeric, based on their in-solution state. For a summary see Table 4.

TABLE 4
Summary of biophysical properties of DOM0100 dAbs. The
combination of properties in a dAb to be aimed for is high trypsin
stability, high thermal stability and monomeric in-solution state to
avoid receptor cross-linking and subsequent agonism or lack of
activity. The table lists the residual activity after 3 h incubation at
37° C. with 34 μg/ml trypsin as a percentage of the activity
at t0. The melting temperature (Tm) was determined by DSC and
the in-solution state by SEC-MALLS. The table indicates that the
most trypsin-stable dAb (DOM1h-574-133) is dimeric and therefore
unfavorable. The dAbs with the best combination of properties
are: DOM1h-574-109, DOM1h-574-156 and DOM1h-574-162.
Where indicated values were not determined (ND).

| | trypsin | | |
|---|---|---|---|
| | stability | | |
| | (% residual | Tm | |
| DOM0100 dAb | activity) | ° C. | in-solution state |
| DOM1h-574-72 | 15 | 56 | Monomer (70%) |
| DOM1h-574-109 | 23 | 55.2 | Monomer (70%) |
| DOM1h-574-125 | ND | 53.5/57.2 | poor data |
| DOM1h-574-126 | 50 | 55.4/59.6 | poor data |
| DOM1h-574-133 | 60 | 57.6/59.6 | Dimer (90%) |
| DOM1h-574-135 | 5 | 51.5 | Monomer (90%) |
| DOM1h-574-138 | 17 | 54/56.9 | monomer/dimer |
| | | | equilibrium |
| DOM1h-574-139 | 2 | 52.1/55.1 | poor data |
| DOM1h-574-155 | 7 | 53 | Monomer (75%) |
| DOM1h-574-156 | 12 | 55 | Monomer (90%) |
| DOM1h-574-162 | 10 | 54.2 | Monomer (90%) |
| DOM1h-574-180 | 5 | 53.2 | Monomer (75%) |

Functional Characterization of DOM0100 dAbs

The DOM0100 dAbs were characterized for functional activity and cross-species reactivity using the human MRC-5 cell assay, the mouse L929 cell line and the Cynomologous monkey CYNOM-K1 cell line described below. For functional inhibition of human TNFR1 signaling, the human fibroblast cell line MRC-5 was incubated with a dose-range of dAb and then stimulated with 200 pg/ml of TNFα (Peprotech) for 18 h. After this stimulation, the media was removed and the levels of IL-8 in the media, produced by the cells in response to TNFα, was determined using the ABI8200 (Applied Biosystems). The ability of the dAbs to block the secretion of IL-8 is a functional read-out of how well they inhibit TNFR1-mediated signaling. The results of testing the 12 DOM0100 dAbs in the MRC5 cell assay are shown in Table 5. Functional mouse cross-reactivity was determined using the mouse L929 cell line, in which the level of protection provided by the 12 DOM0100 dAbs against TNFα-induced cytotoxicity was evaluated. In this assay, cells are again incubated with a dose-range of dAb followed by stimulation with TNFα in the presence of actinomycine. After overnight incubation, the viability of the cells is measured and plotted against dAb concentration. The DOM0100 dAbs protected against TNFα cytotoxicity and resulted in ND50 values in the 20-40 nM range. The potency differences of the DOM0100 dAbs observed between the human MRC5 cells and the mouse L929 cells is of a similar order of magnitude as the differences in affinity determined by BIAcore.

Finally, the Cynomologous monkey cross-reactivity of the dAbs was tested using the CYNOM-K1 cell line. Briefly, the dAb was incubated with CYNOM-K1 cells (ECACC 90071809) (5×10³ cells/well) for one hour at 37° C. in a flat bottom cell culture plate. Recombinant human TNF alpha (Peprotech) was added (final concentration of 200 pg/ml) and the plates were incubated for 18-20 hours. The level of secreted IL-8 was then measured in the culture supernatant using the DuoSet ELISA development system (R&D Systems, cat#DY208), according to the manufacturer's instructions (document number 750364.16 version 11/08). The ND50 was determined by plotting dAb concentration against the percentage of inhibition of IL-8 secretion. The results for the DOM0100 dAbs is shown in Table 5.

TABLE 5
Summary of functional activity of DOM0100 dAbs in cell-based
assays for different species. All values presented are ND50 values
(in nM) determined in the respective cell assay, whilst ND stands for,
not determined. Although the difference between the DOM0100 dAbs
in the MRC5 assay is limited, it follows the same trend as observed in
the mouse and cyno cell assays. Across species, DOM1h-574-156,
DOM1h-574-109 and DOM1h-574-138 are the most potent dAbs.

| | Human | Mouse | Cynomologus |
|---|---|---|---|
| | MRC5 | L929 | CYNOM-K1 |
| DOM0100 dAb | nM | nM | nM |
| DOM1h-574-72 | 2.7 | 46 | 2.3 |
| DOM1h-574-109 | 1.8 | 20 | 2.3 |
| DOM1h-574-126 | 1.9 | 35 | 1.2 |
| DOM1h-574-133 | 2.1 | 110 | 1.7 |
| DOM1h-574-135 | 1.8 | 47 | 1.5 |
| DOM1h-574-138 | 1.4 | 23 | 1.2 |
| DOM1h-574-139 | 1.1 | 28 | 1.8 |
| DOM1h-574-155 | 2.1 | 67 | 1.6 |
| DOM1h-574-156 | 0.9 | 22 | ND |
| DOM1h-574-162 | 1.2 | 27 | ND |
| DOM1h-574-180 | 1.9 | 34 | ND |

Epitope Mapping for DOM0100 dAbs

As the binding epitope on TNFR1 of the DOM0100 dAbs can be correlated to the mechanism of action, multiple efforts were under taken to establish which residues in TNFR1 are recognized by the DOM0100 dAbs. Two experimental approaches were chosen to establish the epitope: 1) BIAcore epitope competition and 2) peptide scanning using partially overlapping peptides.

1) BIAcore Epitope Competition:

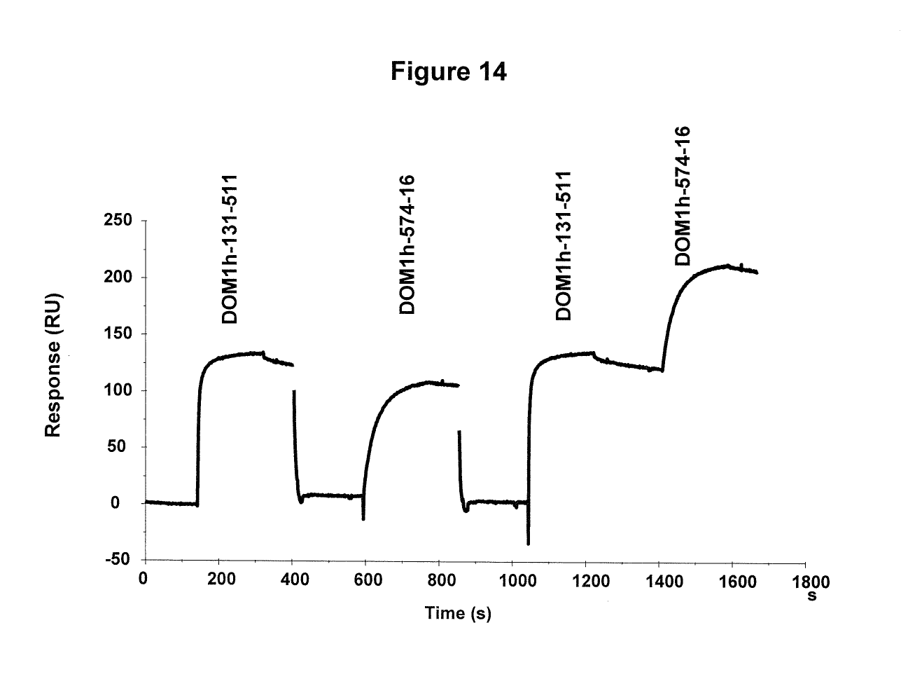

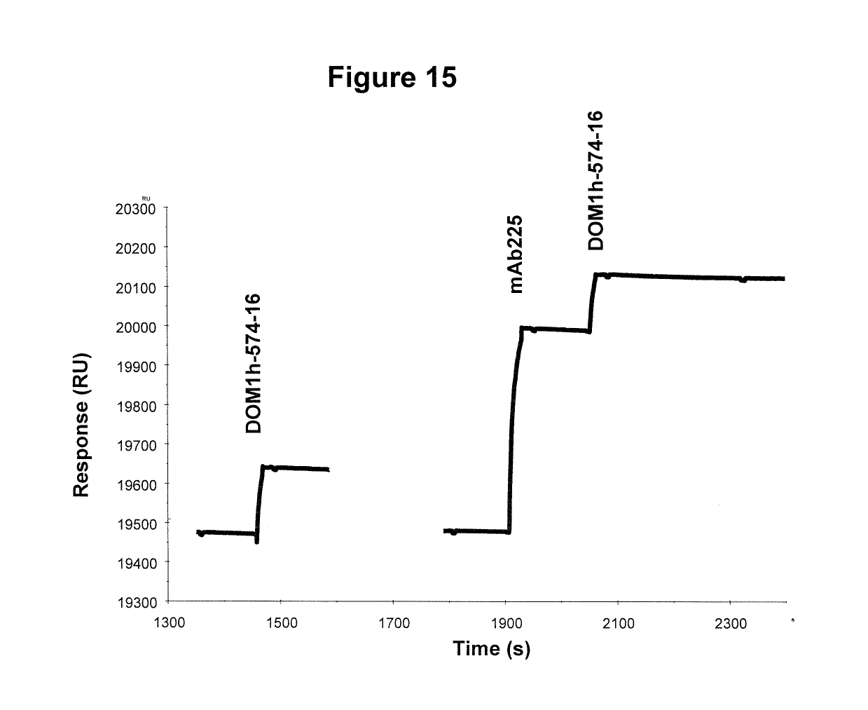

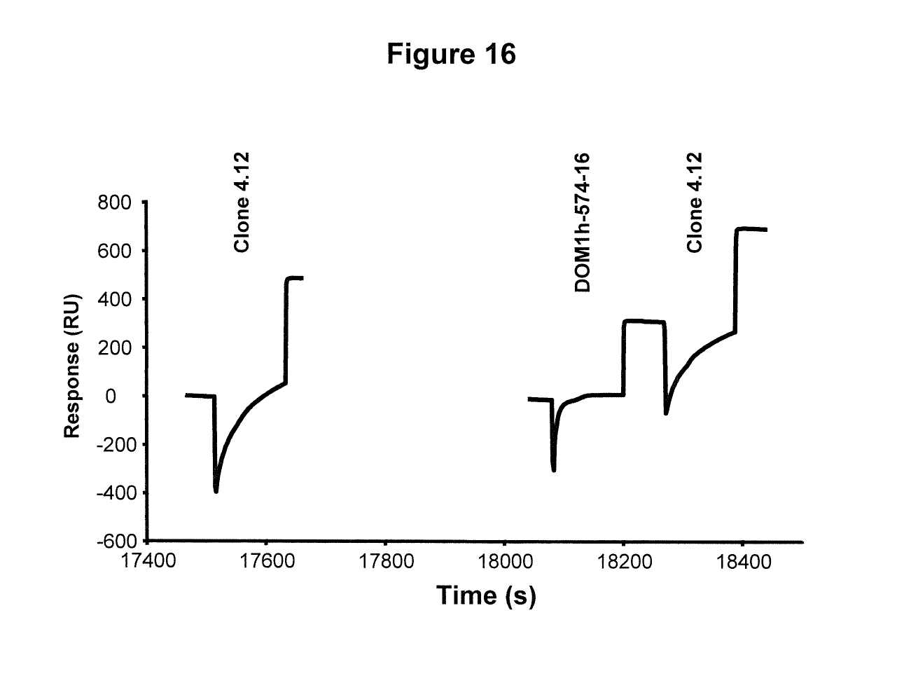

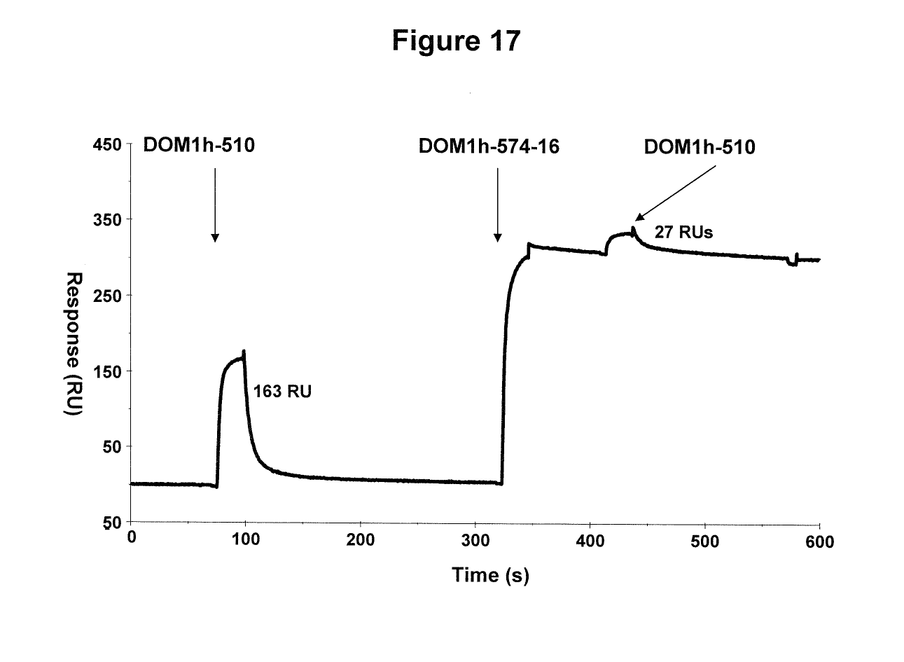

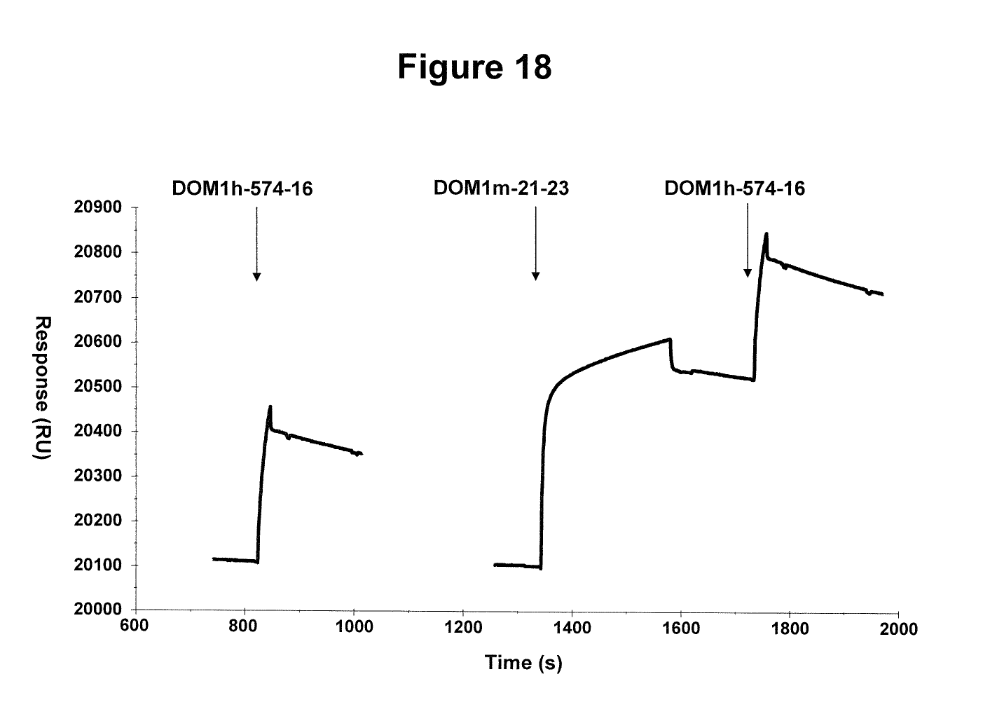

A qualitative approach to determining if competition between two different antibodies or antibody fragments exists for a single epitope on TNFR1 can be done by BIAcore (Malmborg, J. Immunol. Methods 183, p 7 (1995)). For this purpose, biotinylated-TNFR1 is coated on a BIAcore SA-chip followed by the sequential injections of different dAbs or antibodies to establish binding levels for each antibody in the absence of any competing antibody (fragment). Subsequently, the injections are repeated using the same concentration of antibody (fragment), but now immediately after injection of the antibody with which competition is to be determined. Bound antibody (fragment) is quantified in Resonance Units (RUs) and compared in the presence and absence of a second antibody. If no competition exists between the two antibodies (fragments), the number of RUs bound will be identical in the presence and absence of the other antibody. Conversely, if competition does exist there will be little or no RUs bound during the injection of the second antibody (fragment). For DOM1h-574-16 it was shown that the number of resonance units bound in the presence or absence of a TNFα-competitive dAb (DOM1h-131-511 (see WO2008149144)) and mAb (mAb225 (R&D systems; cat no. MAB225) was unchanged, indicating an epitope novel to the mentioned dAb and mAb (FIGS. 14 and 15). TNFR1 is a multi-domain receptor, consisting of four cysteine-rich domains. Domains two and three are responsible for TNFα binding (Banner et al., Cell, 73, p 431 (1993)), while the first domain, also known as the preligand assembly domain (PLAD), facilitates the pre-assembly of the receptor prior to TNFα binding (Chan et al. Science, vol 288, p 2351 (2000)). Competition with a known PLAD-binding mAb Clone 4.12, (Supplied by Invitrogen, cat. no. Zymed 33-0100) on the BIAcore was very limited, showing at best a decrease of 20% in the number of RUs of Clone 4.12 bound in the presence of the DOM0100 dAb (DOM1h-574-16) compared to its absence (FIG. 16). This indicates that the vast majority of the epitope recognized by DOM1h-574-16 is not recognized by Clone 4.12. The only dAb to show full competition with DOM1h-574-16 was another DOM0100 dAb isolated during the selections: DOM1h-510 (FIG. 17). As the DOM0100 dAb shows cross-reactive binding to mouse TNFR1, the same experiments could be performed on mouse TNFR1 coated to BIAcore chips to establish if competition exists with anti-murine TNFR1, non-competitive dAb DOM1m-21-23 (see WO2006038027). Strikingly, no competition was seen between DOM1m-21-23 and the DOM0100 dAb DOM1h-574-16 (FIG. 18). The unique property of the DOM1h-574 dAbs to be cross-reactive with mouse also highlights that a novel epitope must be recognized as none of the above mentioned dAbs or antibodies (DOM1h-131-511, mAB225, Clone 4.12 and DOM1m-21-23) show any significant mouse/human cross-reactivity.

2) Peptide Scanning of TNFR1.

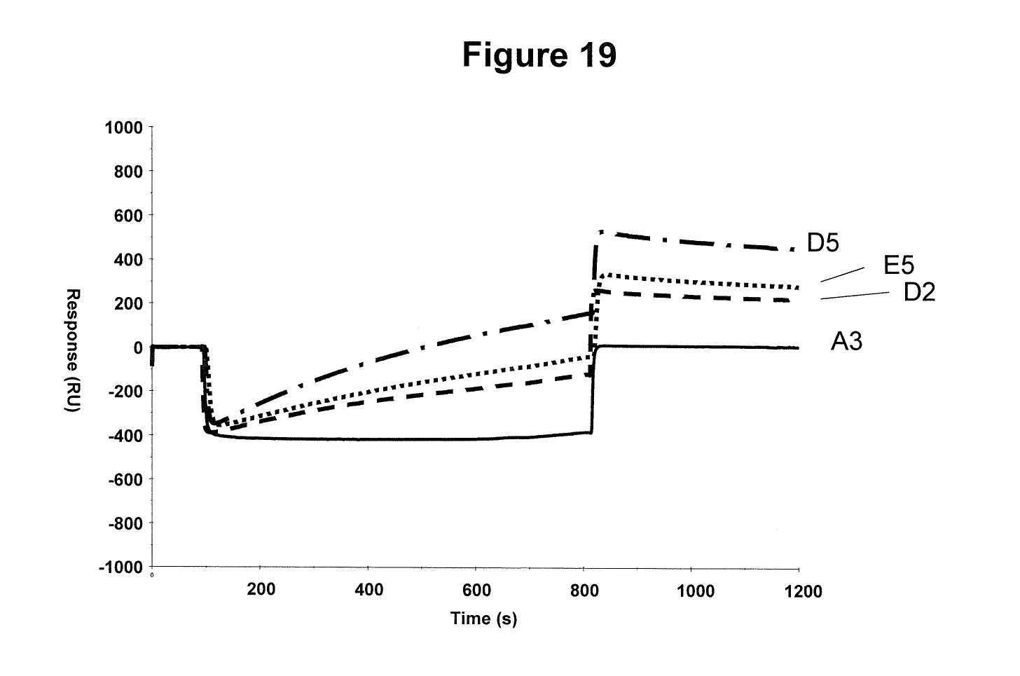

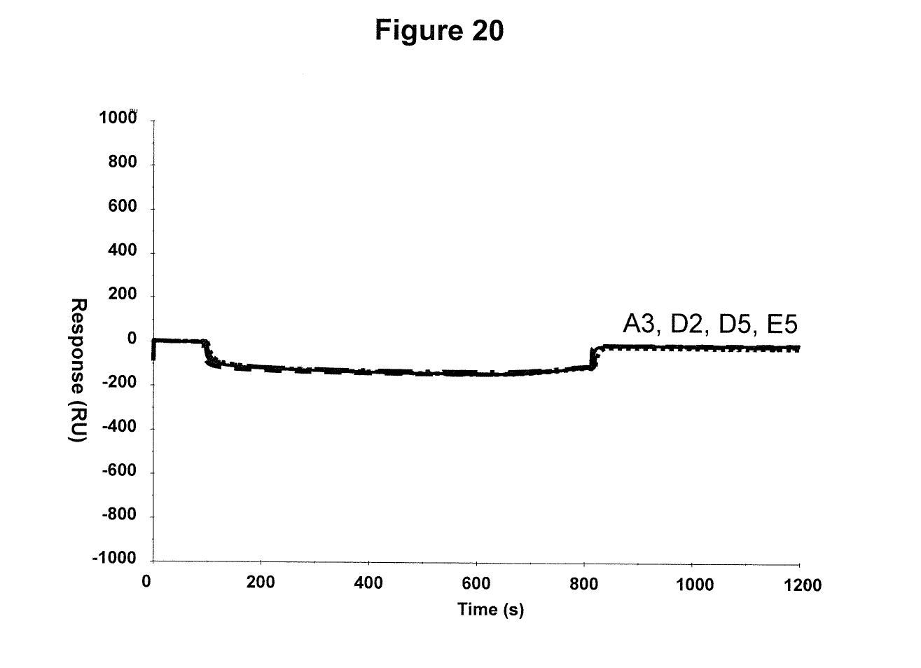

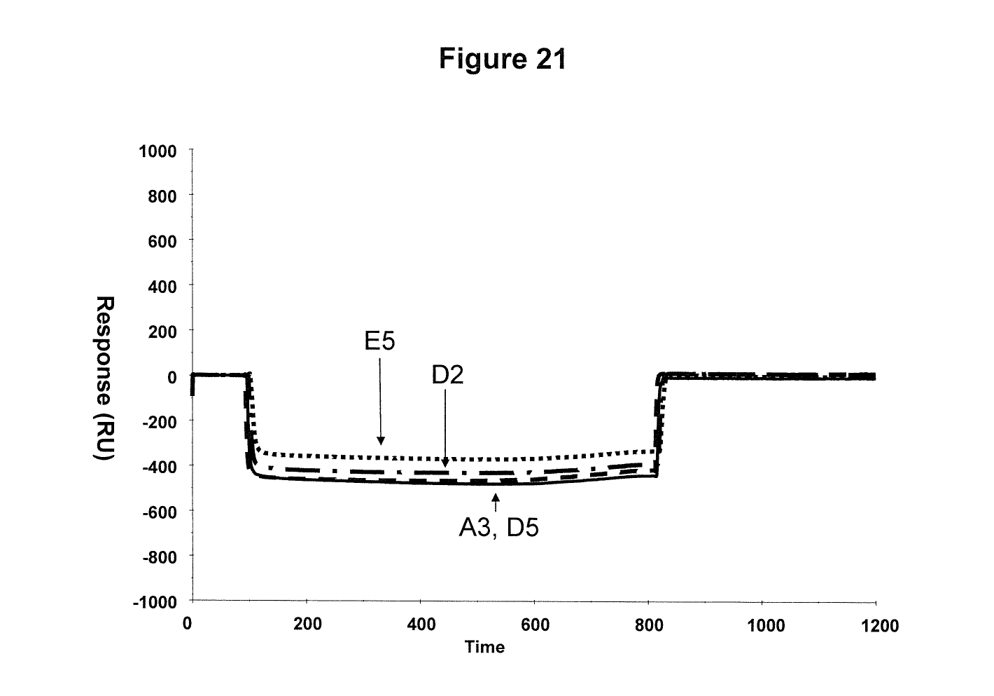

To establish if any linear epitope on the TNFR1 is recognized by our DOM1h-574 dAb lineage, scanning 15-mer peptides, each offset by three residues and resulting in a total of 57 different peptides, were synthesized to cover the complete extracellular domain of TNFR1. These peptides each contained a biotin group, which was used for coupling to different sensor tips of a ForteBio Octet instrument (Menlo Park, Calif., USA). The ForteBio Octet instrument uses Bio-Layer Interferometry (BLI), a label-free, biosensor technology that enables the real-time measurement of molecular interactions. The Octet instrument shines white light down the biosensor and collects the light reflected back. Any change in the number of molecules bound to the biosensor tip causes a shift in this interference pattern of the reflected light and is determined in real-time. In our experiment, all 57 tips, each coated with a different peptide, were incubated with DOM1h-574-16 dAb and binding of dAb to each tip was monitored. The vast majority of tips showed no binding, with the exception of three. These three peptides, together with a negative control peptide that had not shown any binding on the BioForte Octet, were coupled to a streptavidin-coated, BIAcore chip and binding of DOM1h-574-16, DOM1h-131-511 and DOM1m-21-23 to these peptides were determined (FIGS. 19, 20 and 21). Only the DOM0100 dAb (DOM1h-574-16) showed any binding to the three specific peptides, while none of the other dAbs showed any binding. No binding for any dAbs was observed on the negative peptide control. The three TNFR1 peptides could be divided into two groups: 1) peptide 1 (NSICCTKCHKGTYLY) located in domain 1 and 2) peptides 2 (CRKNQYRHYWSENLF) and 3 (NQYRHYWSENLFQCF), which overlap and are in domain 3 of TNFR1. Especially peptide 1 is noteworthy as, with the exception of the very last residue, this sequence corresponds to the only stretch of 15 sequential amino-acid residues in TNFR1 which are fully conserved between mouse and human TNFR1 (this conserved stretch has the sequence: NSICCTKCHKGTYL). Binding to this epitope would explain the mouse cross-reactivity observed for the DOM1h-574 lineage.

Formatting of DOM0100 dAbs for Extended In Vivo Half-Life

For the DOM0100 dAbs to be useful in treating a chronic inflammatory disorder, such as e.g. RA and psoriasis, it would be desirable that the dAb will be delivered systemically and be active for prolonged periods of time. Many different approaches are available to accomplish this, which include e.g. addition of a PEG moiety to the dAb, expression of the dAb as a genetic fusion with a serum albumin-binding dAb (AlbudAb™) or genetic fusion to the Fc portion of an IgG. For the DOM0100 dAb DOM1h-574-16 both the PEG and AlbudAb fusion were tested.

1) Half-Life Extension by Conjugation with 40K (40 KDa) Linear PEG.

For this purpose a variant of DOM1h-574-16 was made which had a free cysteine at the C-terminus of the dAb. The variant was expressed in E. coli and purified using Protein-A streamline. Using maleimide chemistry (see WO04081026), 40K linear PEG DOWpharma) was conjugated to the C-terminus of this DOM1h-574-16 variant and the reaction cleaned by running on a FPLC column. The molecule was named DMS0162. The effect of the PEG conjugation on extending the half-life of DMS0162 was evaluated in a rat PK study. Three female Sprague-Dawley rats were administered i.v. with a target dose of 2.5 mg/kg of protein. Blood samples were taken from the rats at 0.17, 1, 4, 8, 24, 48, 72, 96, 120 and 168 hours post administration and assayed to determine amounts of DMS0162 in blood. DMS0162 samples were tested in a TNFR1-capture and goat anti-hfAb detection ELISA. Raw data from the assays were converted into concentrations of drug in each serum sample. The mean μg/mL values at each timepoint were then analysed in the WinNonLin analysis package, eg version 5.1 (available from Pharsight Corp., Mountain View, Calif. 94040, USA), using non-compartmental analysis (NCA). These data gave an average terminal half-life of DMS0162 in rat of 20.4 h.

2) Half-Life Extension Through Genetic Fusion with an AlbudAb

a) Functional Characterisation of Anti-TNFR1 dAb Fusions with AlbudAbs

Previously we have described the use of genetic fusions with an albumin-binding dAb (AlbudAb) to extend the PK half-life of dAbs in vivo (see, eg, WO04003019, WO2006038027, WO2008149148). Desirable aspects of these fusions are:

1) fusion of the AlbudAb should not substantially affect the binding affinity of the TNFR1-binding dAb,
2) the affinity of the AlbudAb for albumin, from different species, should be such that an increase in PK half-life can be expected.

To evaluate the pairing of DOM1h-574-16 with different AlbudAbs the pairings listed in Table 6 were made (constructs were, N- to C-terminally, anti-TNFR1 dAb (ie, DOM0100 dAb-linker-AlbudAb-myc). With the exception of DMS0184, all contained a myc-tag at the C-terminus which could possibly be used for detection purposes.

TABLE 6
BIAcore off-rate parameters of anti-TNFR1 dAb/AlbudAb fusions
and potency of anti-TNFR1 dAb in the MRC5 cell assay.

| | | | | Koff | Koff | ND50 |
|---|---|---|---|---|---|---|
| | DOM0100 dAb | | AlbudAb | MSA | HSA | (MRC5) |
| DMS | N-terminal dAb | Linker | C-terminal dAb | s⁻¹ | s⁻¹ | nM |
| DMS0182 | DOM1h-574- | AST | DOM7h-11 | 0.75 | 0.17 | 6 |
| | 16 | | | | | |
| DMS0184 | DOM1h-574- | ASTSGPS | DOM7h-11 | 0.72 | 0.16 | 19 |
| | 16 | | | | | |
| DMS0186 | DOM1h-574- | AST | DOM7h-11-12 | 0.08 | 0.12 | 20 |
| | 16 | | | | | |
| DMS0188 | DOM1h-574- | ASTSGPS | DOM7h-11-12 | 0.08 | 0.12 | 17 |
| | 16 | | | | | |
| DMS0189 | DOM1h-574- | AST | DOM7h-11-3 | 0.13 | 0.017 | ND* |
| | 16 | | | | | |
| DMS0190 | DOM1h-574- | ASTSGPS | DOM7h-11-3 | 0.16 | 0.019 | ND* |
| | 16 | | | | | |
| DMS0191 | DOM1h-574- | AST | DOM7m-16 | 0.11 | NB | ND* |
| | 16 | | | | | |
| DMS0192 | DOM1h-574- | ASTSGPS | DOM7m-16 | 0.09 | NB | ND* |
| | 16 | | | | | |
| DMS0163 | DOM1h-574- | ASTSGPS | DOM7h-11-15 | 0.0062 | 0.0024 | 12 |
| | 16 | | | | | |
| DMS0168 | DOM1h-574- | ASTSGPS | DOM7m-16 | ND | ND | 16 |
| | 72 | | | | | |
| DMS0169 | DOM1h-574- | ASTSGPS | DOM7h-11-12 | ND | ND | 2.7 |
| | 72 | | | | | |

All dAb/AlbudAb fusions listed contained a -myc tag at the C-terminus of the AlbudAb, with the exception of DMS0184. In some cases no binding (NB) to the serum albumin was observed by BIAcore, whereas for other it was not determined (ND). For the MRC5 assay, some data were not determined sufficiently often to justify quoting a value (ND*).

The sequences of all AlbudAbs is given below. The nucleotide and amino acid sequences of DOM7h-11 and DOM7m-16 are disclosed herein.

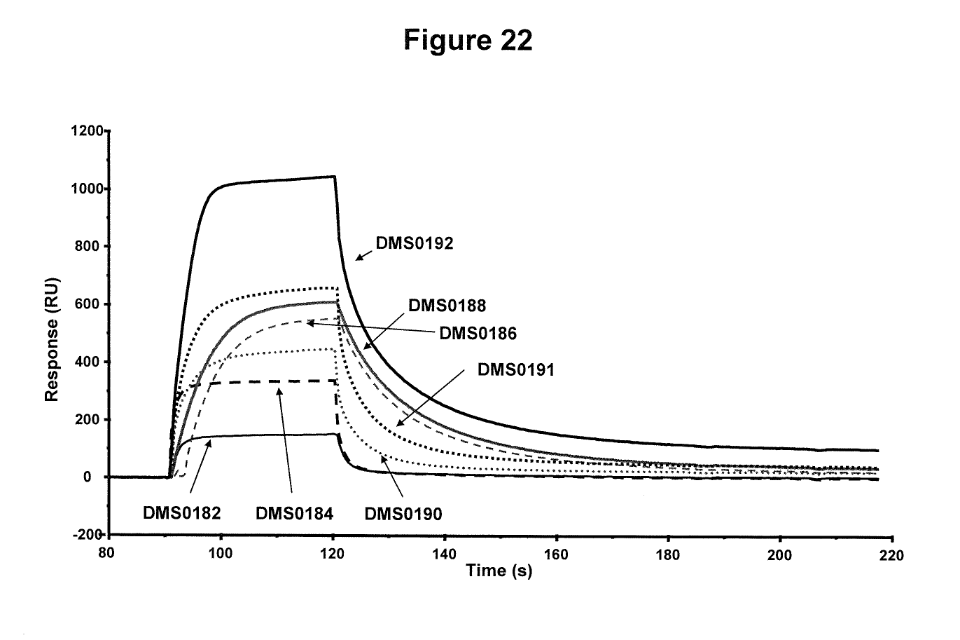

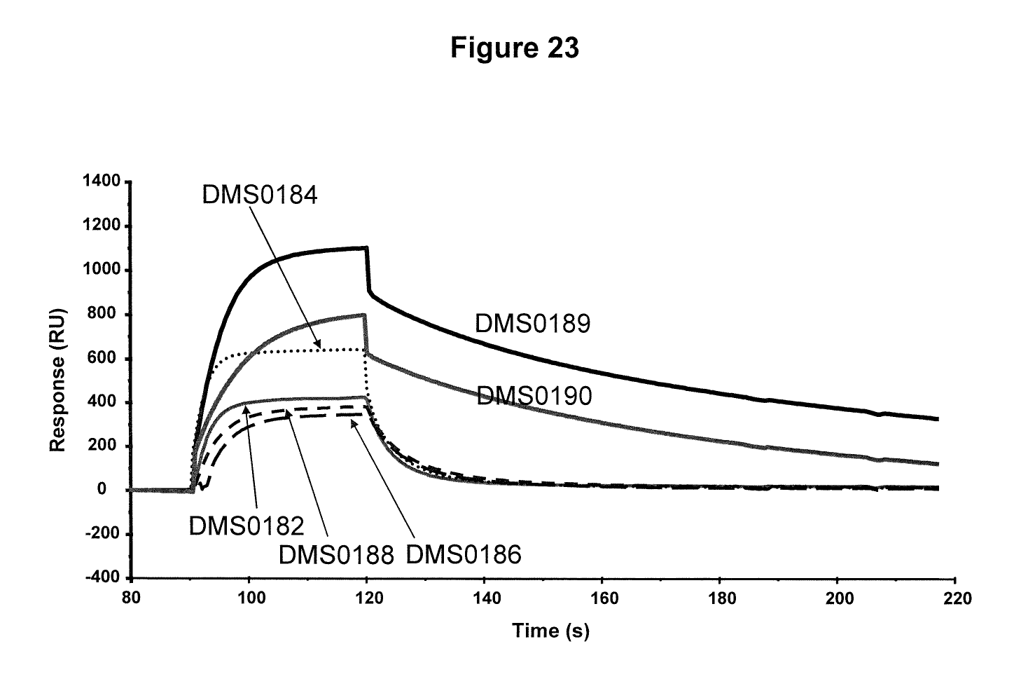

After expression and purification, all constructs were tested on the BIAcore for binding to both mouse and human serum albumin. The off-rates were determined and used to discriminate between the AlbudAbs for their suitability in prolonging the half-life of the fusion molecule. Whereas the linker had little influence on the affinity of the AlbudAb for albumin, a significant difference existed between the dAbs and their albumin affinity. The best AlbudAb for mouse binding was DOM7h-11-15 followed by DOM7m-16 and DOM7h-11-12 (FIG. 22). However, DOM7m-16 showed no binding on human albumin, while DOM7h-11-15 and DOM7h-11-3 were the best pairings for human albumin binding (FIG. 23). Although assay variability was seen, there generally was only a limited drop in affinity in the human MRC-5 cell assay ND50 values obtained for the monomer DOM1h-574-16 and the same dAb when fused to any AlbudAbs of the DOM7h-11 lineage. An impact of the AlbudAb DOM7m-16 was however seen when paired with DOM1h-574-72 and when compared to DOM7h-11-12. The DOM7m-16 pairing resulted in a significant drop in potency for the anti-TNFR1 part of the fusion in the MRC-5 cell assay, which was not seen when the same anti-TNFR1 dAb was paired with DOM7h-11-12. These results highlight the advantages of pairings with AlbudAbs from the DOM7h-11 lineage (eg, anti-serum albumin dAbs having an amino acid sequence that is at least 80, 90 or 95% identical to the amino acid sequence of DOM7h-11).

b) Mouse PK for Different DOM0100-AlbudAb Fusions

An alternative to PEG would be expressing the DOM0100 dAb as a genetic fusion with a domain antibody recognising serum albumin (AlbudAb). To evaluate this approach, a genetic construct was made consisting of DOM1h-574-16, an Alanine Serine Threonine (AST) linker and DOM7h-11 followed by a myc tag (DMS0182). This construct was ligated into the E. coli expression vector pDOM5, transformed to the E. coli strain HB2151 and expressed. The DMS0182 was purified from the supernatant using ProteinL coupled to a solid support followed by ProteinA-streamline to remove any free monomer. DMS0182 was administered to three female Sprague-Dawley rats i.v. at a dose of 5 mg/kg. Blood samples were taken 0.17, 1, 4, 8, 24, 48, 72, 96, 120 and 168 hours post administration. Serum samples were prepared and these were then tested in 3 separate ELISAs: 1) goat anti-myc capture with rabbit anti-human kappa chain detection, 2) goat anti-myc capture with TNFR1-Fc detection and 3) TNFR1 capture with goat anti-fAb detection. Raw data from the assays were converted into concentrations of drug in each serum sample. The mean μg/mL values at each timepoint were then analysed in WinNonLin using non-compartmental analysis (NCA). DMS0182 was tested in the three mentioned assays, with a mean terminal half-life of 5.2-6.4 hours.

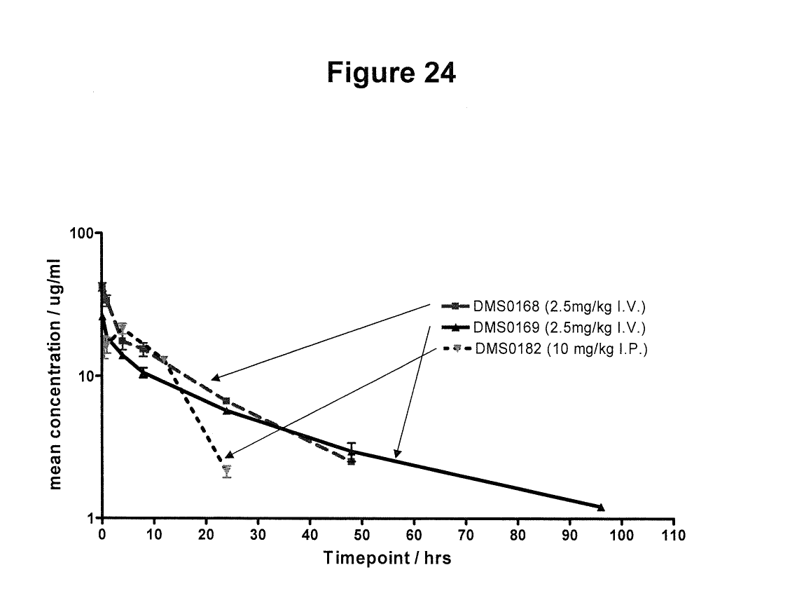

Using the same DMS0182, an additional PK study was done, this time in mice dosed intraperitoneal at 10 mg/kg. Three mice were bled at each of the following time points: 0.17, 1, 4, 12, 24, 48 and 96 h. Analysis of serum using the assays mentioned previously identified a serum half-life of DMS0182 in mice of about 5.9 h (FIG. 24). Clearly the addition of the AlbudAb DOM7h-11 has extended the half-life of the dAb over that seen in the past when free dAb was injected in mice and rat (T1/2 of about 20 minutes, see, eg, WO04003019 WO04003019). However, further improvements in half-life would be beneficial. Examination of the binding affinity of DOM7h-11, when fused to DOM1h-574-16, for rat and mouse albumin identified affinities in excess of 1 μM, as determined by BIAcore. Therefore, changes were made to both the AlbudAb as well as the linker used for these in-line fusions. Two new genetic constructs were made consisting of a different DOM0100 dAb (DOM1h-574-72), a different linker (ASTSGPS), two different AlbudAbs (DOM7m-16 and DOM7h-11-12) and both followed by a -myc tag, creating DMS0168 and DMS0169, respectively (constructs were, N- to C-terminally, anti-TNFR1 dAb (ie, DOM0100 dAb)-linker-AlbudAb-myc). These constructs were cloned in pDOM5, expressed in E. coli and purified using Protein-L and Protein-A. Both were analysed on BIAcore for their binding to MSA and significant improvements were observed resulting in mouse albumin-binding affinities of about 200 nM for both constructs. To determine the effects of improved albumin binding on half-life extension, DMS0168 and DMS0169 were dosed i.v. at 2.5 mg/kg in mice, followed by bleeding three mice at each of the following time points: 0.17, 1, 4, 8, 24, 48, 96 and 168 h. Serum half-life for both these molecules were determined by quantification of the fusion protein in serum in an ELISA based method using goat ant-myc for capture followed by detection with TNFR1-Fc and readout through anti-human-Fc/HRP. In addition to this method, BIAcore quantification of DMS0169 through binding to a chip coated with a high-density of human TNFR1 was used and the data were plotted to calculate the terminal half-life in mice. DMS0168 had a terminal half-life of 15.4 h (ELISA) and DMS0169 had either a terminal half-life of 17.8 h (ELISA) or 22.0 h (BIAcore) (FIG. 24). Both of these half-lives are a significant extension compared to the half-lives when the DOM0100 dAb was fused to DOM7h-11, and highlight the impact of increased affinity for albumin on the terminal half-life of the AlbudAb fusion.

### Functional Characterisation and Biophysical Properties of DOM0100-AlbudAb Fusions

To determine the optimal format of an anti-TNFR1 dAb fused with an anti-albumin dAb, a single anti-TNFR1 dAb was taken (DOM1h-574-72) and paired with four different AlbudAbs (DOM7h-11-3, DOM7h-11-12, DOM7h-14-10 and DOM7h-14-18) using three different linkers (AST, ASTSGPS and (GGGGS)₃). None of these constructs contained α-myc tag. All 12 constructs were expressed in E. coli and purified using a two-step process of Protein L followed by Protein A purification and quantification of expression levels. In addition, the in-solution state of the molecules was determined using SEC-MALLS. The results are summarised in Table 7. The analysis of the results lead to a few striking observations: 1) Pairings of DOM1h-574-72 with the DOM7h-11 lineage dAbs resulted in significantly higher levels of expression when compared to the DOM7h-14 lineage pairings, 2) a monomeric in-solution state was observed for the DOM7h-11 pairings, whilst pairing with DOM7h-14 resulted in monomer/dimer equilibrium. A monomeric in-solution state is preferable as these molecules would be less likely to induce receptor cross-linking and consequently lead to receptor activation (agonism) or to neutralisation of inhibitor activity. Furthermore, monomeric in-solution state is desirable from a development point of view as these molecules tend to aggregate less and be cleaner when analysed by size exclusion chromatography (SEC). The observation that pairing with DOM7h-11 AlbudAbs lead to both higher expression levels and a higher percentage of monomeric in-solution state compared to DOM7h-14 AlbudAbs pairings, favour the DOM7h-11 pairings.

TABLE 7
Overview of combination of fusion molecules produced to
evaluate optimal combination of linker and AlbudAb for
expression and in-solution state. Three different linkers were
used, indicated by their aminoacid composition, AST,
ASTSGPS and a Glycine-Serine linker consisting of three
repeats of four Glycines and one Serine ((G₄S)₃).
The in-solution state was determined using SEC-MALLS and
denoted as either monomer or monomer/dimer equilibrium.
For some AlbudAb fusions the expression was so low that
insufficient material was available for determination of the
in-solution state and these are indicated by (ND).

| | | | | Expres- | |
|---|---|---|---|---|---|
| | DOM0100 | | | sion | |
| DMS | dAb | Linker | AlbudAb | (mg/l) | SEC-MALLS |
| DMS0111 | DOM1h- | AST | DOM7h- | 12 | Monomer |
| | 574-72 | | 11-3 | | (95%) |
| DMS0112 | DOM1h- | AST | DOM7h- | 11 | Monomer |
| | 574-72 | | 11-12 | | (95%) |
| DMS0113 | DOM1h- | AST | DOM7h- | 0 | ND |
| | 574-72 | | 14-10 | | |
| DMS0114 | DOM1h- | AST | DOM7h- | 1 | ND |
| | 574-72 | | 14-18 | | |
| DMS0115 | DOM1h- | ASTSGPS | DOM7h- | 26 | Monomer |
| | 574-72 | | 11-3 | | (98%) |
| DMS0116 | DOM1h- | ASTSGPS | DOM7h- | 15 | Monomer |
| | 574-72 | | 11-12 | | |
| DMS0117 | DOM1h- | ASTSGPS | DOM7h- | 9 | Monomer/ |
| | 574-72 | | 14-10 | | dimer |
| | | | | | equilibrium |
| DMS0118 | DOM1h- | ASTSGPS | DOM7h- | 3 | Monomer/ |
| | 574-72 | | 14-18 | | dimer |
| | | | | | equilibrium |
| DMS0121 | DOM1h- | (G₄S)₃ | DOM7h- | 14 | Monomer |
| | 574-72 | | 11-3 | | (98%) |
| DMS0122 | DOM1h- | (G₄S)₃ | DOM7h- | 12 | Monomer |
| | 574-72 | | 11-12 | | (98%) |
| DMS0123 | DOM1h- | (G₄S)₃ | DOM7h- | 5 | Monomer/ |
| | 574-72 | | 14-10 | | dimer |
| | | | | | equilibrium |
| DMS0124 | DOM1h- | (G₄S)₃ | DOM7h- | 7 | Monomer/ |
| | 574-72 | | 14-18 | | dimer |
| | | | | | equilibrium |

Furthermore, the affinity and potency of the purified fusion molecules were determined using a BIAcore T100 and the MRC5 cell assay, respectively. The BIAcore T100 is a highly sensitive BIAcore version ideally suited for determination of high affinity binders (Papalia et al., Anal Biochem. 359, p 112 (2006)). Biotinylated, human TNFR1 was coated on the chip and each of the twelve AlbudAb fusions were passed over this surface at four different concentrations (2, 10, 50 and 250 nM). The aim was to establish if the pairings had any significant effect on the binding affinity of the anti-TNFR1 dAb (DOM1h-574-72) to its target. As can be seen from Table 8 below, there was no significant difference between the pairings and their effect on affinity by BIAcore. All combinations resulted in a similar affinity, with the exception of the DOM7h-14-18 pairings (DMS0118 and DMS0124) which showed a 3-fold higher affinity than the other pairings. What is surprising though is the at least 2-3 fold improvement in affinity (KD) observed for DOM1h-574-72 in all AlbudAb fusion molecules when compared to the un-fused DOM1h-574-72 dAb. This improvement is observed regardless of the AlbudAb used for pairing and largest for the pairings with DOM7h-14-18. A second experiment used to establish if the different pairings affected the functional activity of the anti-TNFR1 dAb was the MRC5 cell assay (Table 8). A more marked difference between the pairings is observed in the MRC5 assay, in which the best potencies are observed in pairings with DOM7h-11-3 and DOM7h-11-12 while pairings with DOM7h-14-10 (DMS0117) lead to significant decreases in potency.

TABLE 8
BIAcore T100 and MRC5 analysis of the pairings of DOM1h-574-72
with four different AlbudAbs using three different linkers. For the
composition of the DMS clones please see Table 7. The affinity
constants were not determined (ND) for all constructs due to
insufficient material. Overall no hits in affinity were observed on
BIAcore after AlbudAb pairing. The most consistent data were
obtained for DOM7h-11-3 and DOM7h-11-12 pairings in the MRC5
assay.

| | BIAcore | BIAcore | | |
|---|---|---|---|---|
| | Kon | koff | BIAcore KD | MRC5 |
| DMS | (M⁻¹ s⁻¹) | (s⁻¹) | (nM) | (ND50 in nM) |
| DMS0111 | 3.7E+5 | 6.2E−5 | 0.17 | 1.6 |
| DMS0112 | 4.0E+5 | 5.5E−5 | 0.14 | 1.3 |
| DMS0114 | ND | ND | ND | 3.7 |
| DMS0115 | 3.6E+5 | 5.8E−5 | 0.16 | 1.7 |
| DMS0116 | 3.7E+5 | 5.4E−5 | 0.14 | 1.7 |
| DMS0117 | ND | ND | ND | 25.9 |
| DMS0118 | 6.4E+5 | 4.9E−5 | 0.076 | 1.4 |
| DMS0121 | 3.0E+5 | 6.0E−5 | 0.2 | 1.8 |
| DMS0122 | ND | ND | ND | 1.5 |
| DMS0123 | ND | ND | ND | 5.0 |
| DMS0124 | 4.5E+5 | 3.5E−5 | 0.077 | 1.9 |
| DOM1h-574-72 | 2.0E+5 | 1.1E−4 | 0.53 | 2.7 |

Using the results of the biophysical and functional characterisation of both the monomer DOM1h-574 anti-TNFR1 dAbs and the pairings with the AlbudAbs, a subset of five fusion molecules were constructed, expressed, purified and characterised. These five each contained one of the following anti-TNFR1 dAbs: DOM1h-574-109, DOM1h-574-138, DOM1h-574-156, DOM1h-574-162 and DOM1h-574-180 each paired with DOM7h-11-3 using the AST linker. Constructs were, N- to C-terminally, anti-TNFR1 dAb (ie, DOM0100 dAb)-linker-AlbudAb, none of these constructs contained a tag.). The expressed molecules were characterised on SEC-MALLS for in-solution state, on DSC for thermal stability, on BIAcore for affinity to human and mouse TNFR1 and in the MRC5 cell assay for functional activity.

Biophysical characterisation of these five in-line fusion molecules demonstrated all to have melting temperatures >55° C. and to be in-solution monomers (Table 9). A high melting temperature is indicative of an increased stability of the molecule which is beneficial during both downstream processing and storage of the molecule. Furthermore, it might be beneficial to the stability of the molecule when functioning as a pharmaceutical drug in vivo in patients by making it less susceptible to degradation and thereby extending its terminal half-life.

TABLE 9
Overview of preferred combinations of anti-TNFR1 dAbs with
DOM7h-11-3 AlbudAb for half-life extension. After purification,
these fusion molecules were tested for thermal stability (DSC)
and in-solution state (SEC-MALLS). All are monomeric while
DMS0133 and DMS0134 have the highest melting temperatures.

| | Composition | | |
|---|---|---|---|
| DMS | Denoted N- to C-terminally | DSC (° C.) | SEC-MALLS |
| DMS0132 | DOM1h-574-109/AST/ | 58.2/58.9 | 98% monomer |
| | DOM7h-11-3 | | |
| DMS0133 | DOM1h-574-138/AST/ | 59.0/59.4 | 98% monomer |
| | DOM7h-11-3 | | |
| DMS0134 | DOM1h-574-156/AST/ | 58.9/59.3 | 98% monomer |
| | DOM7h-11-3 | | |
| DMS0135 | DOM1h-574-162/AST/ | 58.0/58.7 | 98% monomer |
| | DOM7h-11-3 | | |
| DMS0136 | DOM1h-574-180/AST/ | 57.8/58.0 | 98% monomer |
| | DOM7h-11-3 | | |

Characterisation of the anti-TNFR1 affinity by BIAcore and the functional activity in the human MRC5 and standard mouse L929 cell assays (Table 10) indicated the differences between the dAbs to be limited. However, when all data are taken together from melting temperature, in-solution state, expression, BIAcore, human MRC5 cell assay and standard mouse L929 cell assay, DMS0133 and DMS0134 emerge as the preferred combinations. The melting temperature is the highest for these two, while they belong to the most potent combinations in the functional human and mouse cell assays. The functional activity in the cell assays is a key driver for determining the preferred molecule.

TABLE 10
Functional characterisation and expression of
five best anti-TNFR1/AlbudAb fusion molecules.

| | | BIAcore | BIAcore | BIAcore | MRC5 | L929 |
|---|---|---|---|---|---|---|
| | Expression | Kon | Koff | KD | ND50 | ND50 |
| DMS | (mg/l) | (M⁻¹s⁻¹) | (s⁻¹) | (nM) | (nM) | (nM) |
| DMS0132 | 12 | 1.9E+05 | 4.6E−05 | 0.25 | 1.04 | 6.8 |
| DMS0133 | 6 | 3.6E−05 | 3.6E−05 | 0.20 | 0.99 | 4.2 |
| DMS0134 | 9 | 1.9E+05 | 4.9E−05 | 0.26 | 0.96 | 5.8 |
| DMS0135 | 11 | 1.8E+05 | 5.7E−05 | 0.32 | 1.17 | 5.9 |
| DMS0136 | 3 | 1.9E+05 | 5.5E−05 | 0.30 | 1.97 | 5.4 |

Expression levels were determined after purification. Affinities were determined by BIAcore and the functional activity was determined in both a human MRC5 and standard mouse L929 cell assay. Expression was best for DMS0132, DMS0135 and DMS0134, while the most potent combinations in the cell assays were DMS0133, DMS0134 and DMS0135.

### Demonstration of In Vivo Efficacy of DOM0100 in a Murine Model for Rheumatoid Arthritis

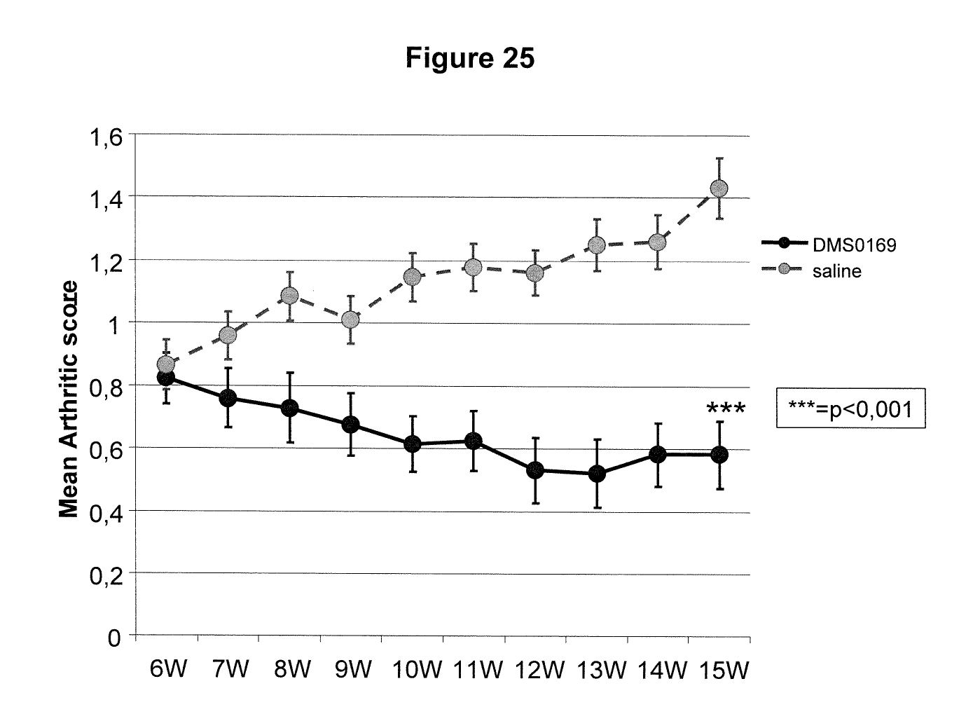

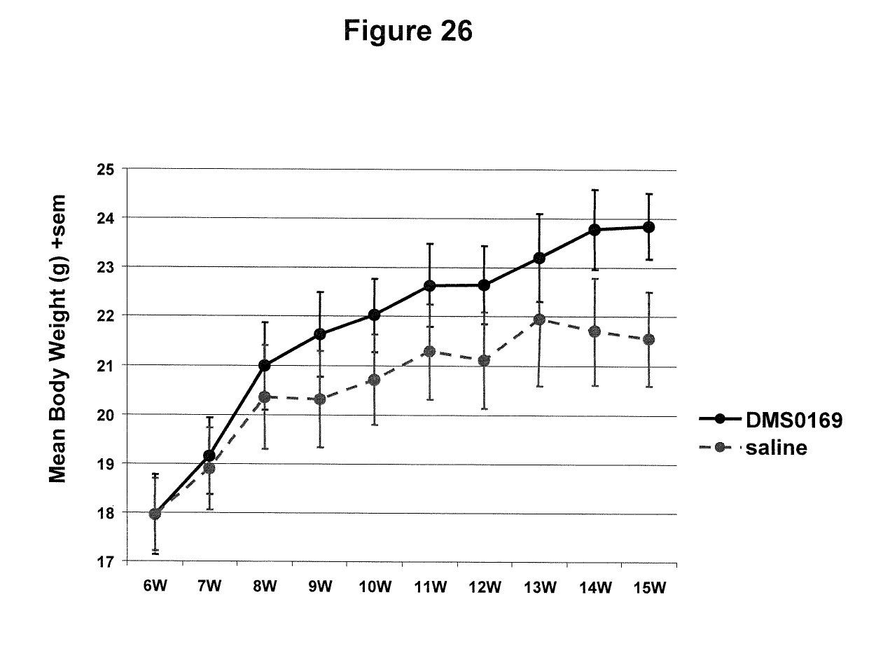

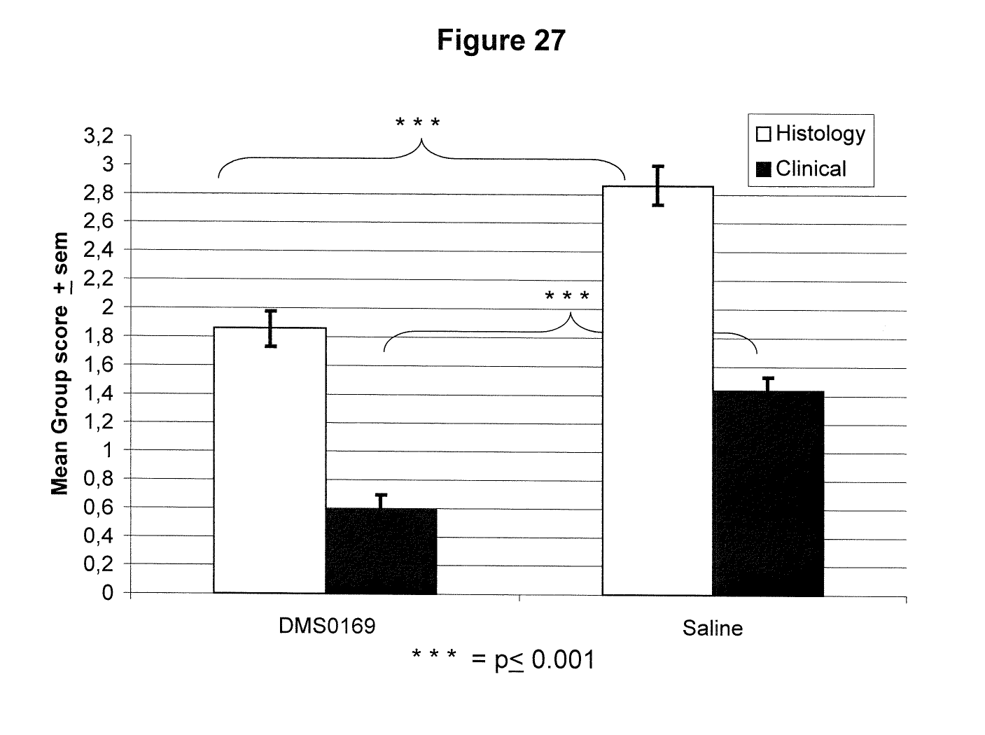

To demonstrate that the activity of the described anti-TNFR1 dAb is useful and could be disease modifying, a murine model of rheumatoid arthritis was treated with DMS0169, a fusion, N- to C-terminally, of DOM1h-574-72-ASTSGPS-DOM7h-11-12-myc tag. This murine model is a transgenic mouse model in which human TNFα is overexpressed (Tg197) and the gene encoding the mouse TNFR1 has been replaced with the human TNFR1 (hp55) gene. Over time these mice develop spontaneous arthritis which is scored by measuring joint sizes during treatment (clinical score) and by performing histological analysis of the joints after 15 weeks (Keffer et al., EMBO. J., 10, p 4025 (1991)). In addition, the overall health of the mice can be inferred from their body weight, which is measured weekly. From week 6 onwards, 12 mice were treated twice a week with either 10 mg/kg of DMS0169 or with saline injections (control group). From week 6 till week 15, each mouse was scored weekly for both clinical score and body weight (FIGS. 25 and 26). After 15 weeks the mice were sacrificed and histological analysis was done of joint inflammation (FIG. 27). The effects of DMS0169 on both clinical score and histology at 15 weeks were highly significant (p<0.001) while body weight for the DMS0169 treated mice was favorable compared to saline treated control animals, indicating the potential for therapeutic benefit of DMS0169 in rheumatoid arthritis.

### PARTIAL INHIBITION OF TNFR1

### EXAMPLES

### Example 1

### Discrimination Between Competitive and Non-Competitive Anti-TNFR1 dAbs

Signaling through TNF receptor 1 (TNFR1, p55) can be inhibited either directly through competitive inhibition of TNFα binding to its receptor or indirectly by a non-competitive mechanism in which the binding of TNFα to its receptor is not affected by the presence of the inhibitor. The mechanism of action of the non-competitive inhibitors can be by blocking pre-ligand assembly of the receptor by binding to domain-1 of TNFR1. To discriminate between these two classes of TNFR1-signaling inhibitors, the combined information from a receptor-binding assay and a cell-based, TNFα-induced, cytokine release assay can be used.

Briefly, in the standard receptor binding assay TNFR1-Fc fusion (R&D Systems (Cat #372-RI), sequence is human TNFR1 (Leu30-Thr211 & Asp41-Thr211)-IEGRMD-Human IgG1 (Pro100-Lys330)-6 His-tag) is coated on anti-IgG beads and incubated with a concentration range (e.g. 0.01 nM-10 μM) of a domain antibody directed against TNFR1. Subsequently, TNFα is added followed by addition of a biotinylated anti-TNFα antibody and fluorescently-labeled streptavidin. The level of fluorescence for each measurement is determined in an ABI 8200 cellular detection assay (FMAT) and plotted against the corresponding dAb concentration used. A similar method can be used for antagonists and inhibitors of TNFR1 other than dAbs. If the anti-TNFR1 dAb is competitive with TNFα binding to its receptor, the fluorescence will decrease with increasing concentrations of dAb and consequently inhibition will be observed. Conversely, if the anti-TNFR1 dAb is non-competitive with TNFα binding to its receptor, the fluorescence will not change with increasing concentrations of dAb and no inhibition will be observed. Hence, anti-TNFR1 dAbs can be classified based on their ability to inhibit TNFα binding to its receptor 1 in a standard RBA.

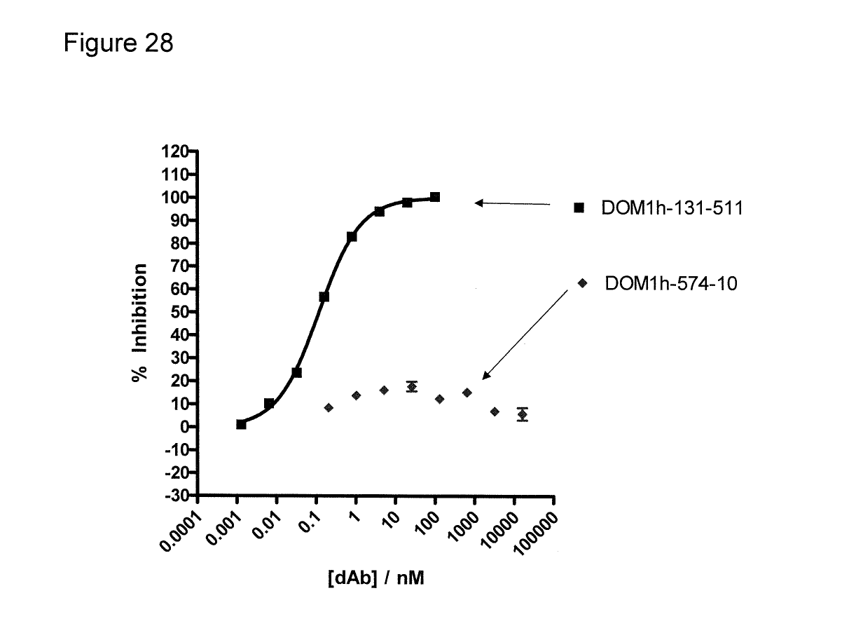

An example of a competitive anti-human TNFR1 dAb is the heavy chain (Vh) dAb DOM1h-131-511 and an example of a non-competitive anti-TNFR1 dAb is the Vh dAb DOM1h-574-10. Both dAbs were cloned in the standard E. coli expression vector used for dAbs and expressed in E. coli culture media after autoinduction with OnEx (Novagen) The expression vector used for DOM1h-574-10 resulted in the dAb containing a myc-tag, which does not influence its activity. Both were purified in a single step using Protein-A streamline and buffer exchanged to PBS for cell assay experiments. As can be seen from FIG. 28, the competitive dAb DOM1h-131-511 inhibited TNFα binding to TNFR1 in the RBA while DOM1h-574-10 had no effect on TNFα binding to TNFR1.

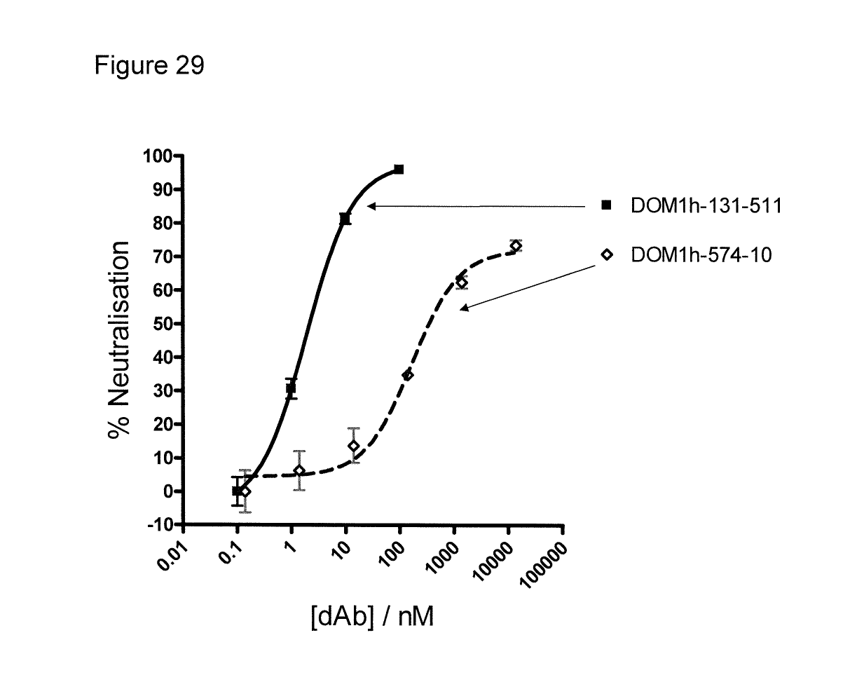

However, a dAb which lacks the ability to inhibit the binding of TNFα to its receptor might also lack functional activity in inhibiting TNFα-mediated signaling through TNFR1. Therefore, the RBA should be combined with a TNFα-induced cell assay in which dAb-mediated inhibition of cytokine release is determined. The specific cell assay that was used is the standard MRC-5 cell assay. Briefly, in this assay the human fibroblast cell line MRC-5 was plated and pre-incubated with a dose range of anti-TNFR1 dAbs followed by addition of a low dose of TNFα (200 pg/ml). After an 18 h incubation at 37° C. with TNFα, the culture supernatant was aspirated and IL-8 release was determined using an IL-8 ABI 8200 cellular detection assay (FMAT). For the dAb to be functionally active it will have to inhibit TNFα-mediated signaling and consequently reduce the level of IL-8 secretion by the MRC-5 cells in response to the TNFα stimulation. As can be seen in FIG. 29, both competitive dAb (DOM1h-131-511) and the non-competitive anti-TNFR1 dAb (DOM1h-574-10) are able to inhibit TNFα-mediated signaling and are therefore functionally active as TNFα inhibitors.

### Example 2

### Non-Competitive TNFR1 Inhibitors Demonstrate Partial Inhibition at Higher Concentrations of TNFα Stimulation

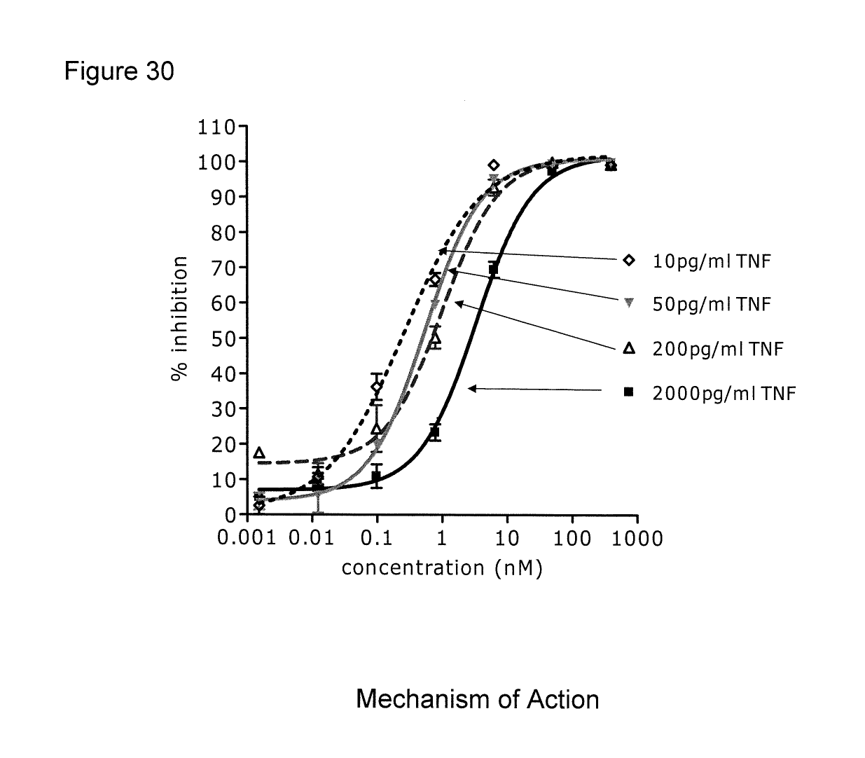

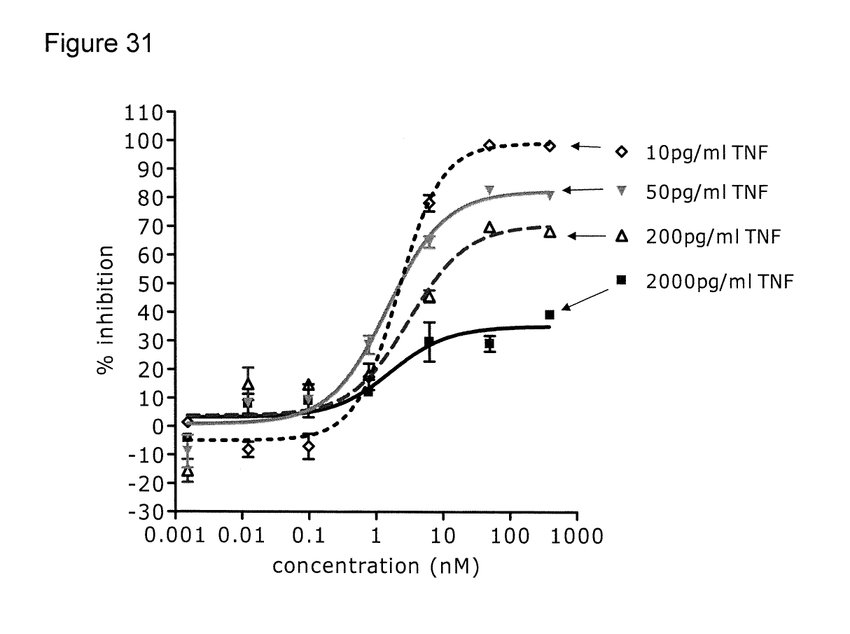

As can be seen from FIG. 29, inhibition of TNFα-mediated signaling did not reach 100% with the non-competitive, anti-TNFR1 dAbs. To investigate this partial inhibition in more detail, both a higher affinity, non-competitive dAb (DOM1h-574-138), which in potency is closer to the competitive dAb (DOM1h-131-511) was chosen and a modification was made to the standard MRC-5 cell assay. The modification to the assay consisted of performing repeats of the assay using four different concentrations of TNFα to stimulate the MRC-5 cells. Human TNFα was sourced from Peprotech, (Cat#300-01A), and used to stimulate the cells at concentrations of 10 pg/ml, 50 pg/ml, 200 pg/ml and 2000 pg/ml. The concentrations were selected because they produced approximately 10%, 50%, 95% and 100% of the maximal response of the cells to TNF-α. To calculate inhibition of TNFα-mediated signalling TNF-α induced IL-8 release by the cells was quantified and expressed as a percentage of maximum IL-8 release obtained at each TNFα concentration (for example; % inhibition of TNF-α signaling=((IL-8 release at a particular dAb dilution/maximum IL-8 release)*100). All other aspects of the assay were kept as described in the standard MRC-5 assay. Surprisingly, a very clear difference existed between the competitive and non-competitive inhibitors of TNFR1 when compared using this experimental design. While the competitive dAb DOM1h-131-511 reached 100% inhibition at all TNFα concentrations used to stimulate (FIG. 30), the non-competitive dAb DOM1h-574-138 demonstrated a decreasing level of inhibition with increasing TNFα concentrations (FIG. 31). For DOM1h-574-138, inhibition of TNFα-mediated signaling when stimulated with 10 pg/ml was nearly complete (FIG. 31). However, when the TNFα concentration was increased to 2 ng/ml, only 30% inhibition of TNFα-mediated cytokine release was detected. These contrasting results for competitive and non-competitive anti-TNFR1 dAbs suggest that the non-competitive dAbs would be partial inhibitors of TNFα when high levels of TNFα are present. Consequently at high TNFα concentrations this class of inhibitors would leave residual TNFα signaling uninhibited which leaves the possibility for positive (beneficial) effects of residual TNFα in a patient e.g. TNFα-mediated inhibition of the reactivation of latent TB. We believe, therefore, that the use of non-competitive TNFR1 antagonists to treat TNFR1-mediated diseases or conditions could be beneficial in that such positive effects of TNFα could be retained.

### Example 3

### Partial Inhibition of TNFα Cytotoxicity in Mouse L929 Cells with Non-Competitive Anti-Mouse TNFR1

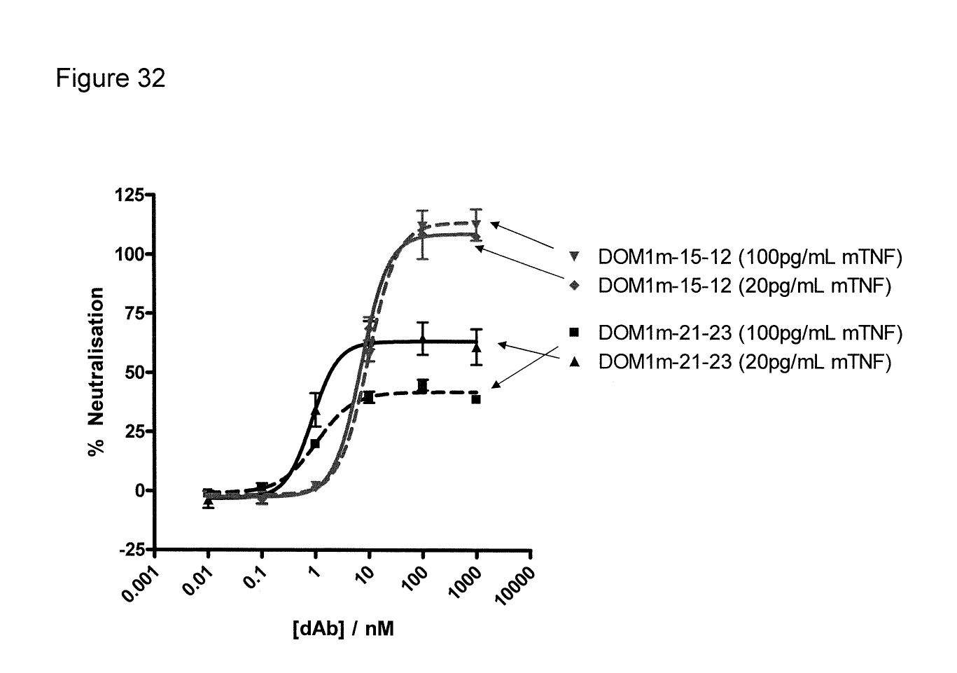

An effect similar to that observed with anti-human TNFR1 dAbs was also observed with competitive and non-competitive anti-mouse TNFR1 dAbs. For these experiments two different anti-mouse TNFR1 dAbs were used. The competitive dAb was the dAb DOM1m-15-12 and the non-competitive dAb was the dAb DOM1m-21-23. Both dAbs were cloned in pUC-based vectors (pDOM5) and expressed through secretion into culture medium by Escherichia coli followed by single step purification using either Protein-L (DOM1m-15-12) or Protein-A (DOM1m-21-23). Methods for dAb cloning, expression and purification have been described WO2006038027 and WO2008149148. Both dAbs were tested in the standard mouse L929 cell assay. To this assay a single modification was made by varying the mouse TNFα concentration used for stimulation. TNF [symbol] (R & D Systems Cat#410-MT) was used at either 20 pg/ml or 100 pg/ml which was equivalent to the concentration which produced cytotoxicity in 60% and 75% of the L929 cells, respectively. Similarly to the results observed with the anti-human TNFR1 dAbs, the competitive anti-mouse TNFR1 dAb (DOM1m-15-12) gave full inhibition at both concentrations of TNFα stimulation while the non-competitive dAb (DOM1m-21-23) demonstrated partial inhibition at both concentrations used with a reduced percentage of inhibition at the higher mouse TNFαt concentration (FIG. 32).

### Standard MRC-5 IL-8 Release Assay

The activities of certain dAbs that bind human TNFR1 were assessed in the following MRC-5 cell assay. The assay is based on the induction of IL-8 secretion by TNFα, in MRC-5 cells and is adapted from the method described in Akeson, A. et al. Journal of Biological Chemistry 271:30517-30523 (1996), describing the induction of IL-8 by IL-1 in HUVEC. The activity of the dAbs was assayed by assessing IL-8 induction by human TNFα, using MRC-5 cells instead of the HUVEC cell line. Briefly, MRC-5 cells (ATCC number: CCL-171) were plated in microtitre plates (5×10³ cells/well) and the cells were pre-incubated for 1 hour with a dose-range of dAb followed by addition of a fixed amount of human TNFα (200 pg/ml). Following overnight incubation (18 h at 37° C.), the culture supernatant was aspirated and IL-8 release was determined using an IL-8 ABI 8200 cellular detection assay (FMAT). The IL-8 FMAT assay used detection and capture reagents from R&D Systems. Beads, goat anti-mouse IgG (H&L) coated polystyrene particles 0.5% w/v 6-8 μm (Spherotech Inc, Cat#MP-60-5), were coated with the capture antibody mouse monoclonal anti-human IL-8 antibody (R&D systems, Cat#MAB208). For detection, biotinylated goat anti-human IL-8 antibody (R&D systems, Cat#BAF208) and Streptavidin Alexafluor 647 (Molecular Probes, Cat#532357) were used. Recombinant human IL-8 (R&D systems, Cat#208-IL) was used as the standard. Anti-TNFR1 dAb activity resulted in a decrease in IL-8 secretion into the supernatant compared with control wells that were incubated with TNFαt only.

### Standard L929 Cytotoxicity Assay

Anti-TNFR1 dAbs were also tested for the ability to neutralise the cytotoxic activity of TNFα on mouse L929 fibroblasts (ATCC CCL-1) (Evans, T. (2000) Molecular Biotechnology 15, 243-248). Briefly, L929 cells plated in microtitre plates (1×10⁴ cells/well) were incubated overnight with anti-TNFR1 dAb, 20 pg/ml TNFα and 1 mg/ml actinomycin D (Sigma, Poole, UK Cat#A9415). Cell viability was measured by reading absorbance at 490 nm following an incubation with [3-(4,5-dimethylthiazol-2-yl)-5-(3-carbboxymethoxyphenyl)-2-(4-sulfophenyl)-2H-tetrazolium (Promega, Madison, USA Cat#G3581). Anti-TNFR1 dAb activity lead to a decrease in TNFα cytotoxicity and therefore an increase in absorbance compared with the TNFα only control.

### Standard Receptor Binding Assay

The potency of the dAbs was determined against human TNFR1 in a receptor binding assay. This assay measures the binding of TNF-alpha to TNFR1 and the ability of soluble dAb to block this interaction. The TNFR1-Fc fusion (R&D Systems, Cat#372-RI.) is captured on a bead pre-coated with goat anti-human IgG (Spherotech, Cat#HUP-60-S). The receptor coated beads are incubated with TNF-alpha (10 ng/ml, Peprotech Cat#300-01A), dAb, biotin conjugated anti-TNF-alpha (Hycult Biotechnology Cat#HM2027.) and streptavidin Alexa Fluor 647 (Molecular Probes, (Invitrogen) Cat#32357) in a black sided clear bottomed 384 well plate. After 6 hours the plate is read on the ABI 8200 Cellular Detection system and bead associated fluorescence determined. If the dAb blocks TNF-alpha binding to TNFR1 the fluorescent intensity will be reduced.

Data was analysed using the ABI 8200 analysis software. Concentration effect curves and potency (EC₅₀) values were determined using GraphPad Prism and a sigmoidal dose response curve with variable slope.

### Standard Cynomologus Monkey CYNOM-K1 Assay

The anti-TNFR1 dAbs were tested for potency in the CYNOM-K1 cell assay. Briefly, the dAb was incubated with CYNOM-K1 cells (ECACC 90071809) (5×10³ cells/well) for one hour at 37° C. in a flat bottom cell culture plate. Recombinant human TNF alpha (Peprotech) was added (final concentration of 200 pg/ml) and the plates were incubated for 18-20 hours. The level of secreted IL-8 was then measured in the culture supernatant using the DuoSet ELISA development system (R&D Systems, cat#DY208), according to the manufacturer's instructions, (document number 750364.16 version 11/08). The ND50 was determined by plotting dAb concentration against the percentage of inhibition of IL-8 secretion.

TABLE 11
Amino Acid Sequences

>DOM1h-509
EVQLLESGGGLVQPGGSLRLSCAASGFTFSQYRMHWVRQAPGKSLEWVS
SIDTRGSSTYYADPVKGRFTISRDNSKNTLYLQMNSLRAEDTAVYYCAK
AVTMFSPFFDYWGQGTLVTVSS
>DOM1h-510
EVQLLESGGGLVQPGGSLRLSCAASGFTFADYGMRWVRQAPGKGLEWVS
SITRTGRVTYYADSVKGRFTISRDNSKNTLYLQMNSLRAEDTAVYYCAK
WRNRHGEYLADFDYWGQGTLVTVSS
>DOM1h-543
EVQLLESGGGLVQPGGSLRLSCAASGFTFMRYRMHWVRQAPGKGLEWVS
SIDSNGSSTYYADSVKGRFTISRDNSKNTLYLQMNSLRAEDTAVYYCAK
DRTERSPVFDYWGQGTLVTVSS
>DOM1h-549
EVQLLESGGGLVQPGGSLRLSCAASGFTFVDYEMHWVRQAPGKGLEWVS
SISESGTTTYYADSVKGRFTISRDNSKNTLYLQMNSLRAEDTAVYYCAK
RRFSASTFDYWGQGTLVTVSS
>DOM1h-574
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAK
YTGHWEPFDYWGQGTLVTVSS
>DOM1h-574-1
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAK
YTGRWEPYDYWGQGTLVTVSS
>DOM1h-574-2
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAK
YTGRWEPFDYWGQGTLVTVSS
>DOM1h-574-4
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAK
YTGRWEPFEYWGQGTLVTVSS
>DOM1h-574-7
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAI
YTGRWEPFDYWGQGTLVTVSS
>DOM1h-574-8
EVQLLESGGGLVQPGGSLRLSCAASGFTFVKYSMGWVRQAPGKGPEWVS
QISNTGGHTYYADSVKGRFTISRDNSKNTLYLQMNSLRAEDTAVYYCAI
YTGRWEPFDYWGQGTLVTVSS
>DOM1h-574-9
EVQLLESGGGLVQPGGSLRLSCAASGFTFVKYSMGWVRQAPGKGLEWVS
QISNTGGHTYYADSVKGRFTISRDNSKNTLYMQMNSLRAEDTAVYYCAI
YTGRWEPFDYWGQGTLVTVSS
>DOM1h-574-10
EVQLLESGGGLVQPGGSLRLSCAASGFTFGKYSMGWVRQAPGKDLEWVS
QISNTGGHTYYADSVKGRFTISRDNSKNTLYLQMNSLRAEDTAVYYCAI
YTGRWEPFDYWGQGTLVTVSS
>DOM1h-574-11
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAK
YTGRWEPFDHWGQGTLVTVSS
>DOM1h-574-12
EVQLLESGGGLVQPGGSLRLSCAASGFTFVKYSMGWVRQAPGKGLEWVS
QISNTGDHTYYADSVKGRFTISRDNSKNTLYLQMNSLRAEDTAVYYCAK
YTGRWEPFDYWGQGTLVTVSS
>DOM1h-574-13
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAK
YTGRWEPFDYWGQGTLVTVSS
>DOM1h-574-14
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWEPFDYWGQGTLVTVSS
>DOM1h-574-15
EVQLLESGGGLVQPGGSLRLSCAASGFTFVKYSMGWVRQAPGKGLEWVS
QISNTGDHTYYADSVKGRFTISRDNSKNTLYLQMNSLRAEDTAVYYCAI
YTGRWEPFDYWGQGTLVTVSS
>DOM1h-574-16
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSS
>DOM1h-574-17
EVQLLESGGGLVQPGGSLRLSCAASGFTFVKYSMGWVRQAPGKGPEWVS
QISNTGDHTYYADSVKGRFTISRDNSKNTLYLQMNSLRAEDTAVYYCAI
YTGRWEPFDYWGQGTLVTVSS
>DOM1h-574-18
EVQLLESGGGLVQPGGSLRLSCAASGFTFGKYSMGWVRQAPGKDLEWVS
QISNTGDRTYYADSVKGRFTISRDNSKNTLYLQMNSLRAEDTAVYYCAI
YTGRWEPFDYWGQGTLVTVSS
>DOM1h-574-19
EVQLLESGGGLVQPGGSLRLSCAASGFTFGKYSMGWVRQAPGKDLEWVS
QISNTGDHTYYADSVKGRFTISRDNSKNTLYLQMNSLRAEDTAVYYCAI
YTGRWEPFDYWGQGTLVTVSS
>DOM1h-574-25
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWEPFVYWGQGTLVTVSS
>DOM1h-574-26
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWEPFEYWGQGTLVTVSS
>DOM1h-574-27
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWKPFEYWGQGTLVTVSS
>DOM1h-574-28
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWVPFEYWGQGTLVTVSS
>DOM1h-574-29
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWRPFEYWGQGTLVTVSS
>DOM1h-574-30
EVQLLESGGGLVQPGGSLRLSCAASGFTFVKYSMGWVRQAPGKGLEWVS
QIANTGDRRYYADSVKGRFTISRDNSKNTLYLQMNSLRAEDTAAYYCAI
YTGRWEPFDYWGQGTLVTVSS
>DOM1h-574-31
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWEPFNYWGQGTLVTVSS
>DOM1h-574-32
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWAPFEYWGQGTLVTVSS
>DOM1h-574-33
EVQLLESGGGLVQPGGSLRLSCAASGFTFVKYSMGWVRQAPGKGLEWVS
QISNTGDRTYYADSVKGRFTISRDNSKNSLYLQMNSLRAEDTAVYYCAI
YTGRWVPFDNWGQGTLVTVSS
>DOM1h-574-35
EVQLLESGGGLVQPGGSLRLSCAASGFTFITYSMGWVRQAPGKGLEWVS
QISNTGDRTYYADSVKGRFTISRDNSKNTLYLQMNSLRAEDTAVYYCAI
YTGRWEPFQYWGQGTLVTVSS
>DOM1h-574-36
EVQLLESGGGLVQPGGSLRLSCAASGFTFGKYSMGWVRQAPGKGLEWVS
QISNTGDRTYYADSVKGRFTISRDNSKNTLYLQMNSLRAEDTAVYYCAI
YTGRWEPFDYWGQGTLVTVSS
>DOM1h-574-37
EVQLLESGGGLVQPGGSLRLSCAASGFTFFKYSMGWVRQAPGKGLEWVS
QISNTGDRTYYADSVKGRFTISRDNSKNTLYLQMNSLRAEDTAVYYCAI
YTGRWEPFDYWGQGTLVTVSS
>DOM1h-574-38
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWEPFDYWGQGTLVTVSS
>DOM1h-574-39
EVQLLESGGGLVQPGGSLRLSCAASGFTFVKYSMGWVRQAPGKGLEWVS
QISNTGDRRYYADAVKGRFTISRDNSKNTLYLQMNSLRAEDTAVYYCAI
YTGRWEPFDYWGQGTLVTVSS
>DOM1h-574-40
EVQLLESGGGLVQPGGSLRLSCAASGFTFVKYSMGWVRQAPGKGLEWVS
QISNTGDRTYYADSVKGRFTISRDNSKNTLYLQMNSLRAEDTAVYYCAI
YTGRWEPFKYWGQGTLVTVSS
>DOM1h-574-53
EVQLLESGGGLVQPGGSLRLSCAASGFTFSKYSMGWVRQAPGKGLEWVS
QISNTGERRYYADSVKGRFTISRDNPKNTLYLQMNSLRAEDTAVYYCAI
YTGRWEPFEYWGQGTLVTVSS
>DOM1h-574-54
EVQLLESGGGLVQPGGSLRLSCAASGFTFVNYSMGWVRQAPGKGLEWVS
QISNTGDRTYYADSVKGRFTISRDNSKNTLYLQMNSLRAEDTAVYYCAI
YTGRWEPYEYWGQGTLVTVTS
>DOM1h-574-65
EVQLLESGGGLVQPGGSLRLSCAASGFTFVKYSMGWVRQAPGKGLEWVS
QIANTGDRRYYADSVKGRFTISRDNSKNTLYLQMNSLRAEDTAVYYCAI
YTGRWEPFVYWGQGTLVTVSS
>DOM1h-574-66
EVQLLESGGGLVQPGGSLRLSCAASGFTFVKYSMGWVRQAPGKGLEWVS
QIANTGDRRYYADSVKGRFTISRDNSKNTLYLQMNSLRAEDTAVYYCAI
YTGRWKPFEYWGQGTLVTVSS
>DOM1h-574-67
EVQLLESGGGLVQPGGSLRLSCAASGFTFVKYSMGWVRQAPGKGLEWVS
QIANTGDRRYYADSVKGRFTISRDNSKNTLYLQMNSLRAEDTAVYYCAI
YTGRWVPFEYWGQGTLVTVSS
>DOM1h-574-68
EVQLLESGGGLVQPGGSLRLSCAASGFTFVKYSMGWVRQAPGKGLEWVS
QIANTGDRRYYADSVKGRFTISRDNSKNTLYLQMNSLRAEDTAVYYCAI
YTGRWRPFEYWGQGTLVTVSS
>DOM1h-574-69
EVQLLESGGGLVQPGGSLRLSCAASGFTFVKYSMGWVRQAPGKGLEWVS
QIANTGDRRYYADSVKGRFTISRDNSKNTLYLQMNSLRAEDTAVYYCAI
YTGRWAPFEYWGQGTLVTVSS
>DOM1h-574-70
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAV
YTGRWEPFVYWGQGTLVTVSS
>DOM1h-574-71
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWKPFEYWGQGTLVTVSS
>DOM1h-574-72
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSS
>DOM1h-574-73
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWRPFEYWGQGTLVTVSS
>DOM1h-574-74
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWAPFEYWGQGTLVTVSS
>DOM1h-574-75
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWEPFVYWGQGTLVTVSS
>DOM1h-574-76
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWKPFEYWGQGTLVTVSS
>DOM1h-574-77
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWVPFEYWGQGTLVTVSS
>DOM1h-574-78
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWRPFEYWGQGTLVTVSS
>DOM1h-574-79
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWAPFEYWGQGTLVTVSS
>DOM1h-574-84
EVQLLESGGGLVQPGGSLRLSCAASGFTFVKYSMGWVRQAPGKGLEWVS
QISNTGDRRYYADAVKGRFTISRDNSKNTLYLQMNSLRAEDTAVYYCAI
YTGRWEPFVYWGQGTLVTVSS
>DOM1h-574-85
EVQLLESGGGLVQPGGSLRLSCAASGFTFVKYSMGWVRQAPGKGLEWVS
QISNTGDRRYYADAVKGRFTISRDNSKNTLYLQMNSLRAEDTAVYYCAI
YTGRWKPFEYWGQGTLVTVSS
>DOM1h-574-86
EVQLLESGGGLVQPGGSLRLSCAASGFTFVKYSMGWVRQAPGKGLEWVS
QISNTGDRRYYADAVKGRFTISRDNSKNTLYLQMNSLRAEDTAVYYCAI
YTGRWVPFEYWGQGTLVTVSS
>DOM1h-574-87
EVQLLESGGGLVQPGGSLRLSCAASGFTFVKYSMGWVRQAPGKGLEWVS
QISNTGDRRYYADAVKGRFTISRDNSKNTLYLQMNSLRAEDTAVYYCAI
YTGRWRPFEYWGQGTLVTVSS
>DOM1h-574-88
EVQLLESGGGLVQPGGSLRLSCAASGFTFVKYSMGWVRQAPGKGLEWVS
QISNTGDRRYYADAVKGRFTISRDNSKNTLYLQMNSLRAEDTAVYYCAI
YTGRWAPFEYWGQGTLVTVSS
>DOM1h-574-90
EVQLLESGGGLVQPGGSLRLSCAASGFTFLKFSMGWVRQAPGKGLEWVS
QIANTGDRRYYADSVKGRFTISRDNSKNTLYLQMNSLRAEDTAVYYCAI
YTGRWAPFEYWGQGTLVTVSS
>DOM1h-574-91
EVQLLESGGGLVQPGGSLRLSCAASGFTFLKYSMGWVRQAPGKGLEWVS
QISNTADRTYYAHSVKGRFTISRDNSKNTLYLQMNSLRAEDTAVYYCAI
YTGRWAPFEYWGQGTLVTVSS
>DOM1h-574-92
EVQLLESGGGLVQPGGSLRLSCAASGFTFFKYSMGWVRQAPGKGLEWVS
QISDTGDRRYYDDSVKGRFTISRDNSKNTLYLQMNSLRAEDTAVYYCAI
YTGRWEPFVYWGQGTLVTVSS
>DOM1h-574-93
EVQLLESGGGLVQPGGSLRLSCAASGFTFLKYSMGWVRQAPGKGLEWVS
QISDTGDRRYYDDSVKGRFTISRDNSKNTLYLQMNSLRAEDTAVYYCAI
YTGRWEPFVYWGQGTLVTVSS
>DOM1h-574-94
EVQLLESGGGLVQPGGSLRLSCAASGFTFVKYSMGWVRQAPGKGLEWVS
QIANTGDRRYYADSVKGRFTISRDNSKNTLYLQMNSLRAEDTAAYYCAI
YTGRWPDFDYWGQGTLVTVSS
>DOM1h-574-95
EVQLLESGGGLVQPGGSLRLSCAASGFTFVKYSMGWVRQAPGKGLEWVS
QIANTGDRRYYADSVKGRFTISRDNSKNTLYLQMNSLRAEDTAAYYCAI
YTGRWPDFEYWGQGTLVTVSS
>DOM1h-574-96
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWPDFDYWGQGTLVTVSS
>DOM1h-574-97
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWPDFEYWGQGTLVTVSS
>DOM1h-574-98
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWPDFDYWGQGTLVTVSS
>DOM1h-574-99
EVQLLESGGGLVQPGGSLRLSCAASGFTFVKYSMGWVRQAPGKGLEWVS
QISDTGDRRYYDDSVKGRFTISRDNSKNTLYLQMNSLRAEDTAVYYCAI
YTGRWPDFEYWGQGTLVTVSS
>DOM1h-574-100
EVQLLESGGGLVQPGGSLRLSCAASGFTFVKYSMGWVRQAPGKGPEWVS
QISAWGDRTYYADSVKGRFTISRDNSKNTLYLQMNSLRAEDTAVYYCAI
YTGRWEPFDYWGQGTLVTVSS
>DOM1h-574-101
EVQLLESGGGLVQPGGSLRLSCAASGFTFVKYSMGWVRQAPGKGPEWVS
QISDGGQRTYYADSVKGRFTISRDNSKNTLYLQMNSLRAEDTAVYYCAI
YTGRWEPFDYWGQGTLVTVSS
>DOM1h-574-102
EVQLLESGGGLVQPGGSLRLSCAASGFTFVKYSMGWVRQAPGKGPEWVS
QISDSGYRTYYADSVKGRFTISRDNSKNTLYLQMNSLRAEDTAVYYCAI
YTGRWEPFDYWGQGTLVTVSS
>DOM1h-574-103
EVQLLESGGGLVQPGGSLRLSCAASGFTFVKYSMGWVRQAPGKGPEWVS
QISDGGTRTYYADSVKGRFTISRDNSKNTLYLQMNSLRAEDTAVYYCAI
YTGRWEPFDYWGQGTLVTVSS
>DOM1h-574-104
EVQLLESGGGLVQPGGSLRLSCAASGFTFVKYSMGWVRQAPGKGPEWVS
QISDKGTRTYYADSVKGRFTISRDNSKNTLYLQMNSLRAEDTAVYYCAI
YTGRWEPFDYWGQGTLVTVSS
>DOM1h-574-105
EVQLLESGGGLVQPGGSLRLSCAASGFTFVKYSMGWVRQAPGKGPEWVS
QISETGRRTYYADSVKGRFTISRDNSKNTLYLQMNSLRAEDTAVYYCAI
YTGRWEPFDYWGQGTLVTVSS
>DOM1h-574-106
EVQLLESGGGLVQPGGSLRLSCAASGFTFVKYSMGWVRQAPGKGLEWVS
QINNTGSTTYYADSVKGRFTISRDNSKNTLYLQMNSLRAEDTAVYYCAI
YTGRWEPFDYWGQGTLVTVSS
>DOM1h-574-107
EVQLLESGGGLVQPGGSLRLSCAASGFTFVKYSMGWVRQAPGKGPEWVS
QISNTADRTYYAHSVKGRFTISRDNSKNTLYLQMNSLRAEDTAVYYCAI
YTGRWVPFEYWGQGTLVTVSS
>DOM1h-574-108
EVQLLESGGGLVQPGGSLRLSCAASGFTFVKYSMGWVRQAPGKGPEWVS
QISNTADRTYYAHSVKGRFTISRDNSKNTLYLQMNSLRAEDTAVYYCAI
YTGRWAPFEYWGQGTLVTVSS
>DOM1h-574-109
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWVPFEYWGQGTLVTVSS
>DOM1h-574-110
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWAPFEYWGQGTLVTVSS
>DOM1h-574-111
EVQLLESGGGLVQPGGSLRLSCAASGFTFVKYSMGWVRQAPGKGLEWVS
QISDTADRTYYDDSVKGRFTISRDNSKNTLYLQMNSLRAEDTAVYYCAI
YTGRWRPFEYWGQGTLVTVSS
>DOM1h-574-112
EVQLLESGGGLVQPGGSLRLSCAASGFTFVKYSMGWVRQAPGKGLEWVS
QISDTADRTYYTHSVKGRFTISRDNSKNTLYLQMNSLRAEDTAVYYCAI
YTGRWAPFEYWGQGTLVTVSS
>DOM1h-574-113
EVQLLESGGGLVQPGGSLRLSCAASGFTFVKYSMGWVRQAPGKGLEWVS
QISNTADRRYYAHSVKGRFTISRDNSKNTLYLQMNSLRAEDTAVYYCAI
YTGRWAPFEYWGQGTLVTVSS
>DOM1h-574-114
EVQLLESGGGLVQPGGSLRLSCAASGFTFVKYSMGWVRQAPGKGLEWVS
QILNTADRTYYDHSVKGRFTISRDNSKNTLYLQMNSLRAEDTAVYYCAI
YTGRWAPFEYWGQGTLVTVSS
>DOM1h-574-115
EVQLLESGGGLVQPGGSLRLSCAASGFTFVKYSMGWVRQAPGKGLEWVS
QISNTADRTYYDHSVKGRFTISRDNSKNTLYLQMNSLRAEDTAVYYCAI
YTGRWAPFEYWGQGTLVTVSS
>DOM1h-574-116
EVQLLESGGGLVQPGGSLRLSCAASGFTFVKYSMGWVRQAPGKGLEWVS
QISDTADRRYYAHSVKGRFTISRDNSKNTLYLQMNSLRAEDTAVYYCAI
YTGRWAPFEYWGQGTLVTVSS
>DOM1h-574-117
EVQLLESGGGLVQPGGSLRLSCAASGFTFVKYSMGWVRQAPGKGLEWVS
QISDTADRRYYDHSVKGRFTISRDNSKNTLYLQMNSLRAEDTAVYYCAI
YTGRWAPFEYWGQGTLVTVSS
>DOM1h-574-118
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAV
YTGRWVSFEYWGQGTLVTVSS
>DOM1h-574-119
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAL
YTGRWVSFEYWGQGTLVTVSS
>DOM1h-574-120
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAV
YTGRWVPFEYWGQGTLVTVSS
>DOM1h-574-121
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAL
YTGRWVPFEYWGQGTLVTVSS
>DOM1h-574-122
EVQLLESGGGLVQPGGSLRLSCAASGFTFVKYSMGWVRQAPGKGLEWVS
QIANTADRRYYAHSVKGRFTISRDNSKNTLYLQMNSLRAEDTAVYYCAI
YTGRWAPFEYWGQGTLVTVSS
>DOM1h-574-123
EVQLLESGGGLVQPGGSLRLSCAASGFTFVKYSMGWVRQAPGKGLEWVS
QISNTADRRYYADAVKGRFTISRDNSKNTLYLQMNSLRAEDTAVYYCAI
YTGRWEPFVYWGQGTLVTVSS
>DOM1h-574-124
EVQLLESGGGLVQPGGSLRLSCAASGFTFVKYSMGWVRQAPGKGLEWVS
QISNTGDRRYYAHAVKGRFTISRDNSKNTLYLQMNSLRAEDTAVYYCAI
YTGRWEPFVYWGQGTLVTVSS
>DOM1h-574-125
EVQLLESGGGLVQPGGSLRLSCAASGFTFVKYSMGWVRQAPGKGLEWVS
QIANTADRRYYADAVKGRFTISRDNSKNTLYLQMNSLRAEDTAVYYCAI
YTGRWEPFVYWGQGTLVTVSS
>DOM1h-574-126
EVQLLESGGGLVQPGGSLRLSCAASGFTFVKYSMGWVRQAPGKGLEWVS
QIANTGDRRYYAHAVKGRFTISRDNSKNTLYLQMNSLRAEDTAVYYCAI
YTGRWEPFVYWGQGTLVTVSS
>DOM1h-574-127
EVQLLESGGGLVQPGGSLRLSCAASGFTFVKYSMGWVRQAPGKGLEWVS
QISNTADRRYYAHAVKGRFTISRDNSKNTLYLQMNSLRAEDTAVYYCAI
YTGRWEPFVYWGQGTLVTVSS
>DOM1h-574-128
EVQLLESGGGLVQPGGSLRLSCAASGFTFVKYSMGWVRQAPGKGLEWVS
QIANTADRRYYAHAVKGRFTISRDNSKNTLYLQMNSLRAEDTAVYYCAI
YTGRWEPFVYWGQGTLVTVSS
>DOM1h-574-129
EVQLLESGGGLVQPGGSLRLSCAASGFTFVKYSMGWVRQAPGKGLEWVS
QIVNTGDRRYYADAVKGRFTISRDNSKNTLYLQMNSLRAEDTAVYYCAI
YTGRWEPFVYWGQGTLVTVSS
>DOM1h-574-130
EVQLLESGGGLVQPGGSLRLSCAASGFTFVKYSMGWVRQAPGKGLEWVS
QIANTGDRRYYADAVKGRFTISRDNSKNTLYLQMNSLRAEDTAVYYCAI
YTGRWEPFVYWGQGTLVTVSS
>DOM1h-574-131
EVQLLESGGGLVQPGGSLRLSCAASGFTFVKYSMGWVRQAPGKGLEWVS
QISDTADRTYYDHSVKGRFTISRDNSKNTLYLQMNSLRAEDTAVYYCAI
YTGRWAPFEYWGQGTLVTVSS
>DOM1h-574-132
EVQLLESGGGLVQPGGSLRLSCAASGFTFVKYSMGWVRQAPGKGLEWVS
QISDTADRTYYDHSVKGRFTISRDNSKNTLYLQMNSLRAEDTAVYYCAI
YTGRWRPFEYWGQGTLVTVSS
>DOM1h-574-133
EVQLLESGGGLVQPGGSLRLSCAASGFTFVKYSMGWVRQAPGKGLEWVS
QISDTADRTYYDHSVKGRFTISRDNSKNTLYLQMNSLRAEDTAVYYCAI
YTGRWEPFVYWGQGTLVTVSS
>DOM1h-574-134
EVQLLESGGGLVQPGGSLRLSCAASGFTFVKYSMGWVRQAPGKGLEWVS
QISDTADRTYYSHSVKGRFTISRDNSKNTLYLQMNSLRAEDTAVYYCAI
YTGRWVPFEYWGQGTLVTVSS
>DOM1h-574-135
EVQLLESGGGLVQPGGSLRLSCAASGFTFVKYSMGWVRQAPGKGLEWVS
QISDTADRTYYTHSVKGRFTISRDNSKNTLYLQMNSLRAEDTAVYYCAI
YTGRWVPFEYWGQGTLVTVSS
>DOM1h-574-137
EVQLLESGGGLVQPGGSLRLSCAASGFTFVKYSMGWVRQAPGKGLEWVS
QISDTADRTYYTDAVKGRFTISRDNSKNTLYLQMNSLRAEDTAVYYCAI
YTGRWEPFVYWGQGTLVTVSS
>DOM1h-574-138
EVQLLESGGGLVQPGGSLRLSCAASGFTFFKYSMGWVRQAPGKGLEWVS
QISDTADRTYYAHSVKGRFTISRDNSKNTLYLQMNSLRAEDTAVYYCAI
YTGRWAPFEYWGQGTLVTVSS
>DOM1h-574-139
EVQLLESGGGLVQPGGSLRLSCAASGFTFLKYSMGWVRQAPGKGLEWVS
QISDTADRTYYAHSVKGRFTISRDNSKNTLYLQMNSLRAEDTAVYYCAI
YTGRWAPFEYWGQGTLVTVSS
>DOM1h-574-140
EVQLLESGGGLVQPGGSLRLSCAASGFTFFKYSMGWVRQAPGKGLEWVS
QIADTGDRRYYDDSVKGRFTISRDNSKNTLYLQMNSLRAEDTAVYYCAI
YTGRWEPFVYWGQGTLVTVSS
>DOM1h-574-141
EVQLLESGGGLVQPGGSLRLSCAASGFTFFKYSMGWVRQAPGKGLEWVS
QISDTADRRYYDDSVKGRFTISRDNSKNTLYLQMNSLRAEDTAVYYCAI
YTGRWEPFVYWGQGTLVTVSS
>DOM1h-574-142
EVQLLESGGGLVQPGGSLRLSCAASGFTFFKYSMGWVRQAPGKGLEWVS
QISDTGDRRYYDHSVKGRFTISRDNSKNTLYLQMNSLRAEDTAVYYCAI
YTGRWEPFVYWGQGTLVTVSS
>DOM1h-574-143
EVQLLESGGGLVQPGGSLRLSCAASGFTFFKYSMGWVRQAPGKGLEWVS
QISDTGDRRYYDDAVKGRFTISRDNSKNTLYLQMNSLRAEDTAVYYCAI
YTGRWEPFVYWGQGTLVTVSS
>DOM1h-574-144
EVQLLESGGGLVQPGGSLRLSCAASGFTFFKYSMGWVRQAPGKGLEWVS
QIADTADRRYYDDSVKGRFTISRDNSKNTLYLQMNSLRAEDTAVYYCAI
YTGRWEPFVYWGQGTLVTVSS
>DOM1h-574-145
EVQLLESGGGLVQPGGSLRLSCAASGFTFFKYSMGWVRQAPGKGLEWVS
QIADTGDRRYYDHSVKGRFTISRDNSKNTLYLQMNSLRAEDTAVYYCAI
YTGRWEPFVYWGQGTLVTVSS
>DOM1h-574-146
EVQLLESGGGLVQPGGSLRLSCAASGFTFFKYSMGWVRQAPGKGLEWVS
QIADTGDRRYYDDAVKGRFTISRDNSKNTLYLQMNSLRAEDTAVYYCAI
YTGRWEPFVYWGQGTLVTVSS
>DOM1h-574-147
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWGPFVYWGQGTLVTVSS
>DOM1h-574-148
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWVPFAYWGQGTLVTVSS
>DOM1h-574-149
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWGPFQYWGQGTLVTVSS
>DOM1h-574-150
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWEPFQYWGQGTLVTVSS
>DOM1h-574-151
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWAPFEYWGQGTLVTVSS
>DOM1h-574-152
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWAPFQYWGQGTLVTVSS
>DOM1h-574-153
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWVPFQYWGQGTLVTVSS
>DOM1h-574-154
EVQLLESGGGLVQPGGSLRLSCAASGFTFVKYSMGWVRQAPGKGLEWVS
QISDTGDRRYYDHSVKGRFTISRDNSKNTLYLQMNSLRAEDTAVYYCAI
YTGRWAPFEYWGQGTLVTVSS
>DOM1h-574-155
EVQLLESGGGLVQPGGSLRLSCAASGFTFLKYSMGWVRQAPGKGLEWVS
QISDTADRTYYAHSVKGRFTISRDNSKNTLYLQMNSLRAEDTAVYYCAI
YTGRWVPFEYWGQGTLVTVSS
>DOM1h-574-156
EVQLLESGGGLVQPGGSLRLSCAASGFTFFKYSMGWVRQAPGKGLEWVS
QISDTADRTYYAHSVKGRFTISRDNSKNTLYLQMNSLRAEDTAVYYCAI
YTGRWVPFEYWGQGTLVTVSS
>DOM1h-574-157
EVQLLESGGGLVQPGGSLRLSCAASGFTFLKYSMGWVRQAPGKGLEWVS
QISDTADRTYYDHSVKGRFTISRDNSKNTLYLQMNSLRAEDTAVYYCAI
YTGRWRPFEYWGQGTLVTVSS
>DOM1h-574-158
EVQLLESGGGLVQPGGSLRLSCAASGFTFFKYSMGWVRQAPGKGLEWVS
QISDTADRTYYDHSVKGRFTISRDNSKNTLYLQMNSLRAEDTAVYYCAI
YTGRWRPFEYWGQGTLVTVSS
>DOM1h-574-159
EVQLLESGGGLVQPGGSLRLSCAASGFTFFKYSMGWVRQAPGKGLEWVS
QISDTADRTYYDHSVKGRFTISRDNSKNTLYLQMNSLRAEDTAVYYCAI
YTGRWEPFVYWGQGTLVTVSS
>DOM1h-574-160
EVQLLESGGGLVQPGGSLRLSCAASGFTFLKYSMGWVRQAPGKGLEWVS
QISDTADRTYYDHSVKGRFTISRDNSKNTLYLQMNSLRAEDTAVYYCAI
YTGRWEPFVYWGQGTLVTVSS
>DOM1h-574-161
EVQLLESGGGLVQPGGSLRLSCAASGFTFLKYSMGWVRQAPGKGLEWVS
QISDTADRTYYSHSVKGRFTISRDNSKNTLYLQMNSLRAEDTAVYYCAI
YTGRWVPFEYWGQGTLVTVSS
>DOM1h-574-162
EVQLLESGGGLVQPGGSLRLSCAASGFTFFKYSMGWVRQAPGKGLEWVS
QISDTADRTYYSHSVKGRFTISRDNSKNTLYLQMNSLRAEDTAVYYCAI
YTGRWVPFEYWGQGTLVTVSS
>DOM1h-574-163
EVQLLESGGGLVQPGGSLRLSCAASGFTFFKYSMGWVRQAPGKGLEWVS
QISDTADRTYYTHSVKGRFTISRDNSKNTLYLQMNSLRAEDTAVYYCAI
YTGRWVPFEYWGQGTLVTVSS
>DOM1h-574-164
EVQLLESGGGLVQPGGSLRLSCAASGFTFLKYSMGWVRQAPGKGLEWVS
QISDTADRTYYTHSVKGRFTISRDNSKNTLYLQMNSLRAEDTAVYYCAI
YTGRWVPFEYWGQGTLVTVSS
>DOM1h-574-165
EVQLLESGGGLVQPGGSLRLSCAASGFTFFKYSMGWVRQAPGKGLEWVS
QISDTADRTYYAHSVKGRFTISRDNSKNTLYLQMNSLRAEDTAVYYCAI
YTGRWAPFEYWGQGTLVTVSS
>DOM1h-574-166
EVQLLESGGGLVQPGGSLRLSCAASGFTFLKYSMGWVRQAPGKGLEWVS
QISDTADRTYYAHSVKGRFTISRDNSKNTLYLQMNSLRAEDTAVYYCAI
YTGRWAPFEYWGQGTLVTVSS
>DOM1h-574-167
EVQLLESGGGLVQPGGSLRLSCAASGFTFLKYSMGWVRQAPGKGLEWVS
QISDTGDRRYYDHSVKGRFTISRDNSKNTLYLQMNSLRAEDTAVYYCAI
YTGRWAPFEYWGQGTLVTVSS
>DOM1h-574-168
EVQLLESGGGLVQPGGSLRLSCAASGFTFFKYSMGWVRQAPGKGLEWVS
QISDTGDRRYYDHSVKGRFTISRDNSKNTLYLQMNSLRAEDTAVYYCAI
YTGRWAPFEYWGQGTLVTVSS
>DOM1h-574-169
EVQLLESGGGLVQPGGSLRLSCAASGFTFVKYSMGWVRQAPGKGLEWVS
QIADTADRTYYAHSVKGRFTISRDNSKNTLYLQMNSLRAEDTAVYYCAI
YTGRWVPFEYWGQGTLVTVSS
>DOM1h-574-170
EVQLLESGGGLVQPGGSLRLSCAASGFTFFKYSMGWVRQAPGKGLEWVS
QISDTADRTYYAHAVKGRFTISRDNSKNTLYLQMNSLRAEDTAVYYCAI
YTGRWVPFEYWGQGTLVTVSS
>DOM1h-574-171
EVQLLESGGGLVQPGGSLRLSCAASGFTFVKYSMGWVRQAPGKGLEWVS
QIADTADRTYYDHSVKGRFTISRDNSKNTLYLQMNSLRAEDTAVYYCAI
YTGRWVPFEYWGQGTLVTVSS
>DOM1h-574-172
EVQLLESGGGLVQPGGSLRLSCAASGFTFVKYSMGWVRQAPGKGLEWVS
QIADTADRTYYDHAVKGRFTISRDNSKNTLYLQMNSLRAEDTAVYYCAI
YTGRWVPFEYWGQGTLVTVSS
>DOM1h-574-173
EVQLLESGGGLVQPGGSLRLSCAASGFTFVKYSMGWVRQAPGKGLEWVS
QIADTADRRYYAHSVKGRFTISRDNSKNTLYLQMNSLRAEDTAVYYCAI
YTGRWAPFEYWGQGTLVTVSS
>DOM1h-574-174
EVQLLESGGGLVQPGGSLRLSCAASGFTFVKYSMGWVRQAPGKGLEWVS
QISDTADRRYYAHAVKGRFTISRDNSKNTLYLQMNSLRAEDTAVYYCAI
YTGRWAPFEYWGQGTLVTVSS
>DOM1h-574-175
EVQLLESGGGLVQPGGSLRLSCAASGFTFVKYSMGWVRQAPGKGLEWVS
QIADTADRRYYAHAVKGRFTISRDNSKNTLYLQMNSLRAEDTAVYYCAI
YTGRWAPFEYWGQGTLVTVSS
>DOM1h-574-176
EVQLLESGGGLVQPGGSLRLSCAASGFTFVKYSMGWVRQAPGKGLEWVS
QISDTADRRYYDHAVKGRFTISRDNSKNTLYLQMNSLRAEDTAVYYCAI
YTGRWAPFEYWGQGTLVTVSS
>DOM1h-574-177
EVQLLESGGGLVQPGGSLRLSCAASGFTFVKYSMGWVRQAPGKGLEWVS
QIADTADRRYYDHAVKGRFTISRDNSKNTLYLQMNSLRAEDTAVYYCAI
YTGRWAPFEYWGQGTLVTVSS
>DOM1h-574-178
EVQLLESGGGLVQPGGSLRLSCAASGFTFVKYSMGWVRQAPGKGLEWVS
QIADTADRRYYDHSVKGRFTISRDNSKNTLYLQMNSLRAEDTAVYYCAI
YTGRWAPFEYWGQGTLVTVSS
>DOM1h-574-179
EVQLLESGGGLVQPGGSLRLSCAASGFTFFKYSMGWVRQAPGKGLEWVS
QISDTADRRYYDDAVKGRFTITRDNSKNTLYLQMNSLRAEDTAVYYCAI
YTGRWEPFVYWGQGTLVTVSS
>DOM1h-574-180
EVQLLESGGGLVQPGGSLRLSCAASGFTFVKYSMGWVRQAPGKGLEWVS
QISDTADRTYYAHAVKGRFTISRDNSKNTLYLQMNSLRAEDTAVYYCAI
YTGRWVPFEYWGQGTLVTVSS
>DMS0111
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKR
>DMS0112
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTMLSWYQQKPGKAPKLLILFGSRLQSGVPSRFSGSGSGTDFTLT
ISSLQPEDFATYYCAQAGTHPTTFGQGTKVEIKR
>DMS0113
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SQWIGSQLSWYQQKPGKAPKLLIMWRSSLQSGVPSRFSGSGSGTDFTLT
ISSLQPEDFATYYCAQGLRHPKTFGQGTKVEIKR
>DMS0114
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SQWIGSQLSWYQQKPGKAPKLLIMWRSSLQSGVPSRFSGSGSGTDFTLT
ISSLQPEDFATYYCAQGLMKPMTFGQGTKVEIKR
>DMS0115
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASRPIGTTLSWYQQKPGKAPKLLILWNSRLQSGVPSRFSGSGSGTD
FTLTISSLQPEDFATYYCAQAGTHPTTFGQGTKVEIKR
>DMS0116
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASRPIGTMLSWYQQKPGKAPKLLILFGSRLQSGVPSRFSGSGSGTD
FTLTISSLQPEDFATYYCAQAGTHPTTFGQGTKVEIKR
>DMS0117
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASQWIGSQLSWYQQKPGKAPKLLIMWRSSLQSGVPSRFSGSGSGTD
FTLTISSLQPEDFATYYCAQGLRHPKTFGQGTKVEIKR
>DMS0118
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASQWIGSQLSWYQQKPGKAPKLLIMWRSSLQSGVPSRFSGSGSGTD
FTLTISSLQPEDFATYYCAQGLMKPMTFGQGTKVEIKR
>DMS0121
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASGGGGSGGGGSGGGGSDIQMTQSPSSL
SASVGDRVTITCRASRPIGTTLSWYQQKPGKAPKLLILWNSRLQSGVPS
RFSGSGSGTDFTLTISSLQPEDFATYYCAQAGTHPTTFGQGTKVEIKR
>DMS0122
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASGGGGSGGGGSGGGGSDIQMTQSPSSL
SASVGDRVTITCRASRPIGTMLSWYQQKPGKAPKLLILFGSRLQSGVPS
RFSGSGSGTDFTLTISSLQPEDFATYYCAQAGTHPTTFGQGTKVEIKR
>DMS0123
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASGGGGSGGGGSGGGGSDIQMTQSPSSL
SASVGDRVTITCRASQWIGSQLSWYQQKPGKAPKLLIMWRSSLQSGVPS
RFSGSGSGTDFTLTISSLQPEDFATYYCAQGLRHPKTFGQGTKVEIKR
>DMS0124
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASGGGGSGGGGSGGGGSDIQMTQSPSSL
SASVGDRVTITCRASQWIGSQLSWYQQKPGKAPKLLIMWRSSLQSGVPS
RFSGSGSGTDFTLTISSLQPEDFATYYCAQGLMKPMTFGQGTKVEIKR
>DMS0132
EVQLLESGGGLVQPGGSLRLSCAASGFTFVKYSMGWVRQAPGKGLEWVS
QISDTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKR
>DMS0133
EVQLLESGGGLVQPGGSLRLSCAASGFTFFKYSMGWVRQAPGKGLEWVS
QISDTADRTYYAHSVKGRFTISRDNSKNTLYLQMNSLRAEDTAVYYCAI
YTGRWAPFEYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKR
>DMS0134
EVQLLESGGGLVQPGGSLRLSCAASGFTFFKYSMGWVRQAPGKGLEWVS
QISDTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKR
>DMS0135
EVQLLESGGGLVQPGGSLRLSCAASGFTFFKYSMGWVRQAPGKGLEWVS
QISDTADRTYYSHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKR
>DMS0136
EVQLLESGGGLVQPGGSLRLSCAASGFTFVKYSMGWVRQAPGKGLEWVS
QISDTADRTYYAHAVKGRFTISRDNSKNTLYLQMNSLRAEDTAVYYCAI
YTGRWVPFEYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKR
>DMS0162
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSC-40K linear PEG
>DMS0163
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASRPIGTMLSWYQQKPGKAPKLLILAFSRLQSGVPSRFSGSGSGTD
FTLTISSLQPEDFATYYCAQAGTHPTTFGQGTKVEIKRAAAEQKLISEE
DLN
>DMS0163-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASRPIGTMLSWYQQKPGKAPKLLILAFSRLQSGVPSRFSGSGSGTD
FTLTISSLQPEDFATYYCAQAGTHPTTFGQGTKVEIKR
>DMS0168
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASQSIIKHLKWYQQKPGKAPKLLIYGASRLQSGVPSRFSGSGSGTD
FTLTISSLQPEDFATYYCQQGARWPQTFGQGTKVEIKRAAAEQKLISEE
DLN
>DMS0168-no tag
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASQSIIKHLKWYQQKPGKAPKLLIYGASRLQSGVPSRFSGSGSGTD
FTLTISSLQPEDFATYYCQQGARWPQTFGQGTKVEIKR
>DMS0169
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASRPIGTMLSWYQQKPGKAPKLLILFGSRLQSGVPSRFSGSGSGTD
FTLTISSLQPEDFATYYCAQAGTHPTTFGQGTKVEIKRAAAEQKLISEE
DLN
>DMS0169-no tag
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASRPIGTMLSWYQQKPGKAPKLLILFGSRLQSGVPSRFSGSGSGTD
FTLTISSLQPEDFATYYCAQAGTHPTTFGQGTKVEIKR
>DMS0176
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSDIQMTQSPSSLSASVGDRVTITCRASRP
IGTTLSWYQQKPGKAPKLLIWFGSRLQSGVPSRFSGSGSGTDFTLTISS
LQPEDFATYYCAQAGTHPTTFGQGTKVEIKR
>DMS0177
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSDIQMTQSPSSLSASVGDRVTITCRASQW
IGSQLSWYQQKPGKAPKLLIMWRSSLQSGVPSRFSGSGSGTDFTLTISS
LQPEDFATYYCAQGAALPRTFGQGTKVEIKR
>DMS0182
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SRPIGTTLSWYQQKPGKAPKLLIWFGSRLQSGVPSRFSGSGSGTDFTLT
ISSLQPEDFATYYCAQAGTHPTTFGQGTKVEIKRAAAEQKLISEEDLN
>DMS0182-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SRPIGTTLSWYQQKPGKAPKLLIWFGSRLQSGVPSRFSGSGSGTDFTLT
ISSLQPEDFATYYCAQAGTHPTTFGQGTKVEIKR
>DMS0184
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASRPIGTTLSWYQQKPGKAPKLLIWFGSRLQSGVPSRFSGSGSGTD
FTLTISSLQPEDFATYYCAQAGTHPTTFGQGTKVEIKR
>DMS0186
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SRPIGTMLSWYQQKPGKAPKLLILFGSRLQSGVPSRFSGSGSGTDFTLT
ISSLQPEDFATYYCAQAGTHPTTFGQGTKVEIKRAAAEQKLISEEDLN
>DMS0186-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SRPIGTMLSWYQQKPGKAPKLLILFGSRLQSGVPSRFSGSGSGTDFTLT
ISSLQPEDFATYYCAQAGTHPTTFGQGTKVEIKR
>DMS0188
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASRPIGTMLSWYQQKPGKAPKLLILFGSRLQSGVPSRFSGSGSGTD
FTLTISSLQPEDFATYYCAQAGTHPTTFGQGTKVEIKRAAAEQKLISEE
DLN
>DMS0188-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASRPIGTMLSWYQQKPGKAPKLLILFGSRLQSGVPSRFSGSGSGTD
FTLTISSLQPEDFATYYCAQAGTHPTTFGQGTKVEIKR
>DMS0189
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKRAAAEQKLISEEDLN
>DMS0189-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SRPIGTTLSWYQQKPGKAPKLLILWNSRLQSGVPSRFSGSGSGTDFTLT
ISSLQPEDFATYYCAQAGTHPTTFGQGTKVEIKR
>DMS0190
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASRPIGTTLSWYQQKPGKAPKLLILWNSRLQSGVPSRFSGSGSGTD
FTLTISSLQPEDFATYYCAQAGTHPTTFGQGTKVEIKRAAAEQKLISEE
DLN
>DMS0190-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASRPIGTTLSWYQQKPGKAPKLLILWNSRLQSGVPSRFSGSGSGTD
FTLTISSLQPEDFATYYCAQAGTHPTTFGQGTKVEIKR
>DMS0191
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SQSIIKHLKWYQQKPGKAPKLLIYGASRLQSGVPSRFSGSGSGTDFTLT
ISSLQPEDFATYYCQQGTRWPQTFGQGTKVEIKRAAAEQKLISEEDLN
>DMS0191-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTDIQMTQSPSSLSASVGDRVTITCRA
SQSIIKHLKWYQQKPGKAPKLLIYGASRLQSGVPSRFSGSGSGTDFTLT
ISSLQPEDFATYYCQQGTRWPQTFGQGTKVEIKR
>DMS0192
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASQSIIKHLKWYQQKPGKAPKLLIYGASRLQSGVPSRFSGSGSGTD
FTLTISSLQPEDFATYYCQQGARWPQTFGQGTKVEIKRAAAEQKLISEE
DLN
>DMS0192-no tag
EVQLLESGGGLVQPGGSLRLSCAASGFTFVKYSMGWVRQAPGKGPEWVS
QISNTGDRTYYADSVKGRFTISRDNSKNTLYLQMNSLRAEDTAVYYCAI
YTGRWEPFDYWGQGTLVTVSSASTSGPSDIQMTQSPSSLSASVGDRVTI
TCRASQSIIKHLKWYQQKPGKAPKLLIYGASRLQSGVPSRFSGSGSGTD
FTLTISSLQPEDFATYYCQQGARWPQTFGQGTKVEIKR
>DMS5519
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASRPIGTMLSWYQQKPGKAPKLLILAFSRLQSGVPSRFSGSGSGTD
FTLTISSLQPEDFATYYCAQAGTHPTTFGQGTKVEIKR
>DMS5520
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAK
YTGHWEPFDYWGQGTLVTVSSASTSGPSDIQMTQSPSSLSASVGDRVTI
TCRASRPIGTTLSWYQQKPGKAPKLLILWNSRLQSGVPSRFSGSGSGTD
FTLTISSLQPEDFATYYCAQAGTHPTTFGQGTKVEIKR
>DMS5521
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTMLSWYQQKPGKAPKLLILAFSRLQSGVPSRFSGSGSGTDFTLT
ISSLQPEDFATYYCAQAGTHPTTFGQGTKVEIKR
>DMS5522
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTMLSWYQQKPGKAPKLLILAFSRLQSGVPSRFSGSGSGTDFTLT
ISSLQPEDFATYYCAQAGTHPTTFGQGTKVEIKRAAAEQKLISEEDLN
>DMS5522-no tag
EVQLLESGGGLVQPGGSLRLSCAASGFTFVKYSMGWVRQAPGKGLEWVS
QISNTADRTYYAHSVKGRFTISRDNSKNTLYLQMNSLRAEDTAVYYCAI
YTGRWVPFEYWGQGTLVTVSSASTDIQMTQSPSSLSASVGDRVTITCRA
SRPIGTMLSWYQQKPGKAPKLLILAFSRLQSGVPSRFSGSGSGTDFTLT
ISSLQPEDFATYYCAQAGTHPTTFGQGTKVEIKR
>DMS5525
EVQLLESGGGLVQPGGSLRLSCAASGFTFVKYSMGWVRQAPGKGLEWVS
QISNTGGHTYYADSVKGRFTISRDNSKNTLYLQMNSLRAEDTAVYYCAK
YTGHWEPFDYWGQGTLVTVSSASTSGPSDIQMTQSPSSLSASVGDRVTI
TCRASRPIGTMLSWYQQKPGKAPKLLILAFSRLQSGVPSRFSGSGSGTD
FTLTISSLQPEDFATYYCAQAGTHPTTFGQGTKVEIKR
>DMS5527
EVQLLESGGGLVQPGGSLRLSCAASGFTFFKYSMGWVRQAPGKGLEWVS
QISDTADRTYYAHSVKGRFTISRDNSKNTLYLQMNSLRAEDTAVYYCAI
YTGRWVPFEYWGQGTLVTVSSASTSGPSDIQMTQSPSSLSASVGDRVTI
TCRASRPIGTMLSWYQQKPGKAPKLLILFGSRLQSGVPSRFSGSGSGTD
FTLTISSLQPEDFATYYCAQAGTHPTTFGQGTKVEIKR
>DOM7h-11
DIQMTQSPSSLSASVGDRVTITCRASRPIGTTLSWYQQKPGKAPKLLIW
FGSRLQSGVPSRFSGSGSGTDFTLTISSLQPEDFATYYCAQAGTHPTTF
GQGTKVEIKR
>DOM7h-11-3
DIQMTQSPSSLSASVGDRVTITCRASRPIGTTLSWYQQKPGKAPKLLIL
WNSRLQSGVPSRFSGSGSGTDFTLTISSLQPEDFATYYCAQAGTHPTTF
GQGTKVEIKR
>DOM7h-11-12
DIQMTQSPSSLSASVGDRVTITCRASRPIGTMLSWYQQKPGKAPKLLIL
FGSRLQSGVPSRFSGSGSGTDFTLTISSLQPEDFATYYCAQAGTHPTTF
GQGTKVEIKR
>DOM7h-11-15
DIQMTQSPSSLSASVGDRVTITCRASRPIGTMLSWYQQKPGKAPKLLIL
AFSRLQSGVPSRFSGSGSGTDFTLTISSLQPEDFATYYCAQAGTHPTTF
GQGTKVEIKR
>DOM7h-14
DIQMTQSPSSLSASVGDRVTITCRASQWIGSQLSWYQQKPGKAPKLLIM
WRSSLQSGVPSRFSGSGSGTDFTLTISSLQPEDFATYYCAQGAALPRTF
GQGTKVEIKR
>DOM7h-14-10
DIQMTQSPSSLSASVGDRVTITCRASQWIGSQLSWYQQKPGKAPKLLIM
WRSSLQSGVPSRFSGSGSGTDFTLTISSLQPEDFATYYCAQGLRHPKTF
GQGTKVEIKR
>DOM7h-14-18
DIQMTQSPSSLSASVGDRVTITCRASQWIGSQLSWYQQKPGKAPKLLIM
WRSSLQSGVPSRFSGSGSGTDFTLTISSLQPEDFATYYCAQGLMKPMTF
GQGTKVEIKR
>DOM7m-16
DIQMTQSPSSLSASVGDRVTITCRASQSIIKHLKWYQQKPGKAPKLLIY
GASRLQSGVPSRFSGSGSGTDFTLTISSLQPEDFATYYCQQGARWPQTF
GQGTKVEIKR
>DOM1h-131-511
EVQLLESGGGLVQPGGSLRLSCAASGFTFAHETMVWVRQAPGKGLEWVS
HIPPVGQDPFYADSVKGRFTISRDNSKNTLYLQMNSLRAEDTAVYYCAL
LPKRGPWFDYWGQGTLVTVSS
>DOM1m-21-23
EVQLLESGGGLVQPGGSLRLSCAASGFTFNRYSMGWLRQAPGKGLEWVS
RIDSYGRGTYYEDPVKGRFSISRDNSKNTLYLQMNSLRAEDTAVYYCAK
ISQFGSNAFDYWGQGTQVTVSS
>DOM1m-15-12
DIQMTQSPSSLSASVGDRVTITCRASQYIHTSVQWYQQKPGKAPKLLIY
GSSRLHSGVPSRFSGSGSGTDFTLTISSLQPEDFATYYCQQNHYSPFTY
GQGTKVEIKR

TABLE 12
Nucleotide Sequences

>DOM1h-509
GAGGTGCAGCTGTTGGAGTCTGGGGGAGGCTTGGTACAGCCTGGGGGG
TCCCTGCGTCTCTCCTGTGCAGCCTCCGGATTCACCTTTAGTCAGTATAG
GATGCATTGGGTCCGCCAGGCTCCAGGGAAGAGTCTAGAGTGGGTCTCA
AGTATTGATACTAGGGGTTCGTCTACATACTACGCAGACCCCGTGAAGGG
CCGGTTCACCATCTCCCGCGACAATTCCAAGAACACGCTGTATCTGCAAA
TGAACAGCCTGCGTGCCGAGGACACCGCGGTATATTACTGTGCGAAAGCT
GTGACGATGTTTTCTCCTTTTTTTGACTACTGGGGTCAGGGAACCCTGG
TCACCGTCTCGAGC
>DOM1h-510
GAGGTGCAGCTGTTGGAGTCTGGGGGAGGCTTGGTACAGCCTGGGGGG
TCCCTGCGTCTCTCCTGTGCAGCCTCCGGATTCACCTTTGCTGATTATGG
GATGCGTTGGGTCCGCCAGGCTCCAGGGAAGGGTCTAGAGTGGGTCTCA
TCTATTACGCGGACTGGTCGTGTTACATACTACGCAGACTCCGTGAAGGG
CCGGTTCACCATCTCCCGCGACAATTCCAAGAACACGCTGTATCTGCAAA
TGAACAGCCTGCGTGCCGAGGACACCGCGGTATATTACTGTGCGAAATGG
CGGAATCGGCATGGTGAGTATCTTGCTGATTTTGACTACTGGGGTCAGG
GAACCCTGGTCACCGTCTCGAGC
>DOM1h-543
GAGGTGCAGCTGTTGGAGTCTGGGGGAGGCTTGGTACAGCCTGGGGGG
TCCCTGCGTCTCTCCTGTGCAGCCTCCGGATTCACCTTTATGAGGTATAG
GATGCATTGGGTCCGCCAGGCTCCAGGGAAGGGTCTAGAGTGGGTCTCA
TCGATTGATTCTAATGGTTCTAGTACATACTACGCAGACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGAAA
GATCGTACGGAGCGTTCGCCGGTTTTTGACTACTGGGGTCAGGGAACCC
TGGTCACCGTCTCGAGC
>DOM1h-549
GAGGTGCAGCTGTTGGAGTCTGGGGGAGGCTTGGTGCAGCCTGGGGGG
TCCCTGCGTCTCTCCTGTGCAGCCTCCGGATTCACCTTTGTTGATTATGA
GATGCATTGGGTCCGCCAGGCTCCAGGGAAGGGTCTAGAGTGGGTCTCAT
CTATTAGTGAGAGTGGTACGACGACATACTACGCAGACTCCGTGAAGGG
CCGGTTCACCATCTCCCGCGACAATTCCAAGAACACGCTGTATCTGCAAA
TGAACAGCCTGCGTGCCGAGGACACCGCGGTATATTACTGTGCGAAACGT
CGTTTTTCTGCTTCTACGTTTGACTACTGGGGTCAGGGAACCCTGGTCA
CCGTCTCGAGC
>DOM1h-574
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGGTCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAA
ATATACGGGTCATTGGGAGCCTTTTGACTACTGGGGTCAGGGAACCCTGG
TCACCGTCTCGAGC
>DOM1h-574-1
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGGTCATACATACTACGCAGACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGAAA
TATACGGGTCGTTGGGAGCCTTATGACTACTGGGGTCAGGGAACCCTGGT
CACCGTCTCGAGC
>DOM1h-574-2
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGGTCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAA
ATATACGGGTCGTTGGGAGCCTTTTGACTACTGGGGTCAGGGAACCCTGG
TCACCGTCTCGAGC
>DOM1h-574-4
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGGTCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAA
ATATACGGGTCGTTGGGAGCCTTTTGAGTACTGGGGTCAGGGAACCCTGG
TCACCGTCTCGAGC
>DOM1h-574-7
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGGTCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACCGTCTCGAGC
>DOM1h-574-8
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGATGGGTCCGCCAGGCTCCAGGGAAAGGTCCAGAGTGGGTCTC
ACAGATTTCGAATACGGGTGGTCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACAGTCTCGAGC
>DOM1h-574-9
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGGTCATACATACTACGCAGACTCCGTGAAG
GGCCGGTTCACCATATCCCGCGACAATTCCAAGAACACGCTGTATATGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACCGTCTCGAGC
>DOM1h-574-10
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAAGTATTC
GATGGGGTGGGTCCGCCAGGCTCCAGGGAAGGATCTAGAGTGGGTCTC
ACAGATTTCGAATACGGGTGGTCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACCGTCTCGAGC
>DOM1h-574-11
GAGGTGCAGCTGTTGGAGTCAGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGGTCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAA
ATATACGGGTCGTTGGGAGCCTTTTGACCACTGGGGTCAGGGGACCCTGG
TCACCGTCTCGAGC
>DOM1h-574-12
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAA
ATATACGGGTCGTTGGGAGCCTTTTGACTACTGGGGTCAGGGAACCCTGG
TCACCGTCTCGAGC
>DOM1h-574-13
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAA
ATATACGGGTCGTTGGGAGCCTTTTGACTACTGGGGTCAGGGAACCCTGG
TCACCGTCTCGAGC
>DOM1h-574-14
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACCGTCTCGAGC
>DOM1h-574-15
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACCGTCTCGAGC
>DOM1h-574-16
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGATGGGTCCGCCAGGCTCCAGGGAAAGGTCCAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACAGTCTCGAGC
>DOM1h-574-17
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGATGGGTCCGCCAGGCTCCAGGGAAAGGTCCAGAGTGGGTCTC
ACAGATTTCGAATACGGGTGATCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACAGTCTCGAGC
>DOM1h-574-18
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAAGTATTC
GATGGGGTGGGTCCGCCAGGCTCCAGGGAAGGATCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTGG
TCACCGTCTCGAGC
>DOM1h-574-19
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAAGTATTC
GATGGGGTGGGTCCGCCAGGCTCCAGGGAAGGATCTAGAGTGGGTCTC
ACAGATTTCGAATACGGGTGATCATACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTG
GTCACCGTCTCGAGC
>DOM1h-574-25
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGTCTACTGGGGTCAGGGAACCCTGG
TCACCGTCTCGAGC
>DOM1h-574-26
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCTGAGGACACCGCGGTATATTACTGTGCGATA
TATACGGGTCGTTGGGAGCCTTTTGAGTACTGGGGTCAGGGAACCCTGG
TCACCGTCTCGAGC
>DOM1h-574-27
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCGGACTCCGTGAAG
GGCCGGTTCACCATCTCCCGCGACAATTCCAAGAACACGCTGTATCTGC
AAATGAACAGCCTGCGTGCTGAGGACACCGCGGTATATTACTGTGCGATA
TATACGGGTCGTTGGAAGCCTTTTGAGTACTGGGGTCAGGGAACCCTGG
TCACCGTCTCGAGC
>DOM1h-574-28
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCTGAGGACACCGCGGTATATTACTGTGCGATA
TATACTGGGCGTTGGGTGCCTTTTGAGTACTGGGGTCAGGGAACCCTGGT
CACCGTCTCGAGC
>DOM1h-574-29
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGATA
TATACGGGTCGTTGGAGGCCTTTTGAGTACTGGGGTCAGGGAACCCTGG
TCACCGTCTCGAGC
>DOM1h-574-30
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTGCGAATACGGGTGATCGTAGATACTACGCAGACTCTGTGAAG
GGCCGGTTCACCATCTCCCGCGACAATTCCAAGAACACGCTGTATCTGC
AAATGAACAGCCTGCGTGCCGAGGACACCGCGGCATATTACTGTGCGAT
ATATACGGGTCGTTGGGAGCCTTTTGACTACTGGGGTCAGGGAACCCTG
GTCACCGTCTCGAGC
>DOM1h-574-31
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACTGCTGATCGTACATACTACGCACACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTAACTACTGGGGTCAGGGAACCCTGG
TCACCGTCTCGAGC
>DOM1h-574-32
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGGTGGGCGCCTTTTGAGTACTGGGGTCAGGGAACCCTG
GTCACCGTCTCGAGC
>DOM1h-574-33
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACTCGCTGTATCTGC
AAATGAACAGCCTGCGTGCCGAGGACACCGCGGTATATTACTGTGCGAT
ATATACGGGTCGTTGGGTGCCTTTTGACAACTGGGGTCAGGGAACCCTG
GTCACCGTCTCGAGC
>DOM1h-574-35
GAGGTGCAGCTGTTGGAGTCTGGGGGAGGCTTGGTACAGCCTGGGGGG
TCCCTGCGTCTCTCCTGTGCAGCCTCCGGATTCACCTTTATTACGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTCAGTACTGGGGTCAGGGAACCCTG
GTCACCGTCTCGAGC
>DOM1h-574-36
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAAGTATTC
GATGGGGTGGGTCCGCCAGGCTCCAGGGAAGGGTCTAGAGTGGGTCTC
ACAGATTTCGAATACGGGTGATCGTACATACTACGCGGACTCCGTGAAG
GGCCGGTTCACCATCTCCCGCGACAATTCCAAGAACACGCTGTATCTGC
AAATGAACAGCCTGCGTGCCGAGGACACCGCGGTATATTACTGTGCGAT
ATATACGGGTCGTTGGGAGCCTTTTGACTACTGGGGTCAGGGAACCCTG
GTCACCGTCTCGAGC
>DOM1h-574-37
GAGGTGCAGCTGTTGGAGTCTGGGGGAGGCTTGGTACAGCCTGGGGGG
TCCCTGCGTCTCTCCTGTGCAGCCTCCGGATTCACCTTTTTTAAGTATTC
GATGGGGTGGGTCCGCCAGGCTCCAGGGAAGGGTCTAGAGTGGGTCTC
ACAGATTTCGAATACGGGTGATCGTACATACTACGCAGACTCCGTGAAG
GGCCGGTTCACCATCTCCCGCGACAATTCCAAGAACACGCTGTATCTGC
AAATGAACAGCCTGCGTGCCGAAGACACCGCGGTATATTACTGTGCGAT
ATATACGGGTCGTTGGGAGCCTTTTGACTACTGGGGTCAGGGAACCCTG
GTCACCGTCTCGAGC
>DOM1h-574-38
GAGGTGCAGCTGTTGGAGTCTGGGGGAGGCTTGGTACAGCCTGGGGGG
TCCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTC
ACAGATTTCGGATACGGGTGATCGTAGATACTACGATGACTCTGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGACTACTGGGGTCAGGGAACCCTGG
TCACCGTCTCGAGC
>DOM1h-574-39
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCG
TCTCGAGC
>DOM1h-574-40
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACG
GGTCGTTGGGAGCCTTTTAAGTACTGGGGTCAGGGAACCCTGGTCACCGT
CTCGAGC
>DOM1h-574-53
GAGGTGCAGCTGTTGGAGTCTGGGGGAGGCTTGGTACAGCCTGGGGGGTC
CCTGCGTCTCTCCTGTGCAGCCTCCGGATTCACCTTTAGTAAGTATTCGA
TGGGGTGGGTCCGCCAGGCTCCAGGGAAGGGTCTAGAGTGGGTCTCACAG
ATTTCGAATACGGGTGAGCGTAGATACTACGCAGACTCAGTGAAGGGCCG
GTTCACCATCTCCCGCGACAATCCCAAGAACACGCTGTATCTGCAAATGA
ACAGCCTGCGTGCCGAGGACACCGCGGTATATTACTGTGCGATATATACG
GGTCGGTGGGAGCCTTTTGAATACTGGGGTCAGGGAACCCTGGTCACCGT
CTCGAGC
>DOM1h-574-54
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAACTATTCGA
TGGGGTGGGTCCGCCAGGCTCCAGGGAAGGGTCTAGAGTGGGTCTCACAG
ATTTCGAATACGGGTGATCGTACATACTACGCGGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTATGAGTACTGGGGTCAGGGAACCCTGGTCACCGT
CACGAGC
>DOM1h-574-65
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGATAATTCCAAGAACACACTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-66
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGAAGCCTTTTGAGTACTGGGGTCAGGGAACCCTGGTCACCGT
CTCGAGC
>DOM1h-574-67
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-68
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGAGGCCTTTTGAGTACTGGGGTCAGGGAACCCTGGTCACCGT
CTCGAGC
>DOM1h-574-69
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-70
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGGTATATACG
GGTCGTTGGGAGCCTTTTGTCTACTGGGGTCAGGGAACCCTGGTCACCGT
CTCGAGC
>DOM1h-574-71
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACG
GGTCGTTGGAAGCCTTTTGAGTACTGGGGTCAGGGAACCCTGGTCACCGT
CTCGAGC
>DOM1h-574-72
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-73
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGAGGCCTTTTGAGTACTGGGGTCAGGGAACCCTGGTCACCGT
CTCGAGC
>DOM1h-574-74
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-75
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-76
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCCCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACG
GGTCGTTGGAAGCCTTTTGAGTACTGGGGTCAGGGAACCCTGGTCACCGT
CTCGAGC
>DOM1h-574-77
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-78
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGAGGCCTTTTGAGTACTGGGGTCAGGGAACCCTGGTCACCGT
CTCGAGC
>DOM1h-574-79
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-84
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-85
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCTGAGGACACCGCGGTATATTACTGTGCGATATATACG
GGTCGTTGGAAGCCTTTTGAGTACTGGGGTCAGGGAACCCTGGTCACCGT
CTCGAGC
>DOM1h-574-86
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCCCCAGGGAAGGGTCTAGAGTGGGTCTCACAG
ATTTCGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAAGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-87
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGAGGCCTTTTGAGTACTGGGGTCAGGGAACCCTGGTCACCGT
CTCGAGC
>DOM1h-574-88
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-90
GAGGTGCAGCTGTTGGAGTCTGGGGGAGGCTTGGTACAGCCTGGGGGGTC
CCTGCGTCTCTCCTGTGCAGCCTCCGGATTCACCTTTTTGAAGTTTTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-91
GAGGTGCAGCTGTTGGAGTCTGGGGGAGGCTTGGTACAGCCTGGGGGGTC
CCTGCGTCTCTCCTGTGCAGCCTCCGGATTCACCTTTTTGAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-92
GAGGTGCAGCTGTTGGAGTCTGGGGGAGGCTTGGTACAGCCTGGGGGGTC
CCTGCGTCTCTCCTGTGCAGCCTCCGGATTCACCTTTTTCAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-93
GAGGTGCAGCTGTTGGAGTCTGGGGGAGGCTTGGTACAGCCTGGGGGGTC
CCTGCGTCTCTCCTGTGCAGCCTCCGGATTCACCTTTTTGAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-94
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGACAATTCCAAGAACACGCTGTATCTGCAAATGA
ACAGCCTGCGTGCCGAGGACACCGCGGCATATTACTGTGCGATATATACG
GGTCGGTGGCCCGACTTTGACTACTGGGGTCAGGGAACCCTGGTCACCGT
CTCGAGC
>DOM1h-574-95
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACTCTGTGAAGGGCCG
GTTCACCATCTCCCGCGACAATTCCAAGAACACGCTGTATCTGCAAATGA
ACAGCCTGCGTGCCGAGGACACCGCGGCATATTACTGTGCGATATATACG
GGTCGGTGGCCCGACTTTGAGTACTGGGGTCAGGGAACCCTGGTCACCGT
CTCGAGC
>DOM1h-574-96
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGCCCGACTTTGACTACTGGGGTCAGGGAACCCTGGTCACCGT
CTCGAGC
>DOM1h-574-97
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGCCCGACTTTGAGTACTGGGGTCAGGGAACCCTGGTCACCGT
CTCGAGC
>DOM1h-574-98
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGCCCGACTTTGACTACTGGGGTCAGGGAACCCTGGTCACCGT
CTCGAGC
>DOM1h-574-99
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACGGGTGATCGTAGATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGCCCGACTTTGAGTACTGGGGTCAGGGAACCCTGGTCACCGT
CTCGAGC
>DOM1h-574-100
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGGCCTGGGGTGACAGGACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGC
>DOM1h-574-101
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAAGGTCCAGAGTGGGTCTCACAG
ATTTCGGACGGCGGTCAGAGGACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGC
>DOM1h-574-102
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGGACTCCGGTTACCGCACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGC
>DOM1h-574-103
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCCAGAGTGGGTCTCACAG
ATTTCGGACGGGGGTACGCGGACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGC
>DOM1h-574-104
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGGACAAGGGTACGCGCACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGC
>DOM1h-574-105
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGGAGACCGGTCGCAGGACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGC
>DOM1h-574-106
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTAACAATACGGGTTCGACCACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGC
>DOM1h-574-107
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCCAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-108
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCCAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-109
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-110
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-111
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGATGACTCTGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGAGGCCTTTTGAGTACTGGGGTCAGGGAACCCTGGTCACCGT
CTCGAGC
>DOM1h-574-112
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACACACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-113
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGCAGATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-114
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTTGAATACTGCTGATCGTACATACTACGATCACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-115
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGATCACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-116
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTAGATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-117
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTAGATACTACGATCACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-118
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGGTATATACT
GGGCGTTGGGTGTCTTTTGAGTACTGGGGTCAGGGAACCCTGGTCACCGT
CTCGAGC
>DOM1h-574-119
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGCTATATACT
GGGCGTTGGGTGTCTTTTGAGTACTGGGGTCAGGGAACCCTGGTCACCGT
CTCGAGC
>DOM1h-574-120
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTTACCATCTCCCGCGACAATTCCAAGAACACGCTGTATCTGCAAATGA
ACAGCCTGCGTGCCGAGGACACCGCGGTATATTACTGTGCGGTATATACT
GGGCGTTGGGTGCCTTTTGAGTACTGGGGTCAGGGAACCCTGGTCACCGT
CTCGAGC
>DOM1h-574-121
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGCTATATACT
GGGCGTTGGGTGCCTTTTGAGTACTGGGGTCAGGGAACCCTGGTCACCGT
CTCGAGC
>DOM1h-574-122
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACTGCTGATCGTAGATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-123
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-124
GAGGTGCAGCTGTTGGAGTCTGGGGGAGGCTTGGTACAGCCTGGGGGGTC
CCTGCGTCTCTCCTGTGCGGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGCGATCGTAGATACTACGCACACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-125
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACTGCTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-126
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCACACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-127
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTAGATACTACGCACACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-128
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGCTGATCGTAGATACTACGCACACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-129
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGTGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-130
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGAATACGGGTGATCGTAGATACTACGCAGACGCGGTGAAGGGGCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-131
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGATCACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-132
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGATCACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGAGGCCTTTTGAGTACTGGGGTCAGGGAACCCTGGTCACCGT
CTCGAGC
>DOM1h-574-133
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGATCACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-134
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACTCACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-135
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACACACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-137
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACACAGACGCGGTGAAGG
GGCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-138
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGGTGGGCGCCTTTTGAGTACTGGGGTCAGGGAACCCTGG
TCACCGTCTCGAGC
>DOM1h-574-139
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGGTGGGCGCCTTTTGAGTACTGGGGTCAGGGAACCCTGG
TCACCGTCTCGAGC
>DOM1h-574-140
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTGCGGATACGGGTGATCGTAGATACTACGATGACTCTGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-141
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTAGATACTACGATGACTCTGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-142
GAGGTGCAGCTGTTGGAGTCTGGGGGAGGCTTGGTACAGCCTGGGGGGT
CCCTGCGCCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACGGGTGATCGTAGATACTACGATCACTCTGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAACCTTTTGTCTACTGGGGTCAGGGAACCCTGG
TCACCGTCTCGAGC
>DOM1h-574-143
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACGGGTGATCGTAGATACTACGATGACGCGGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-144
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTGCGGATACTGCTGATCGTAGATACTACGATGACTCTGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-145
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTGCGGATACGGGTGATCGTAGATACTACGATCACTCTGTGAAGG
GCCGGTTCACTATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-146
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTGCGGATACGGGTGATCGTAGATACTACGATGACGCGGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-147
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGGGCCTTTTGTCTACTGGGGTCAGGGAACCCTGG
TCACCGTCTCGAGC
>DOM1h-574-148
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGTGCCTTTTGCCTACTGGGGTCAGGGAACCCTGG
TCACCGTCTCGAGC
>DOM1h-574-149
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGGACCTTTTCAGTACTGGGGTCAGGGAACCCTGG
TCACCGTCTCGAGC
>DOM1h-574-150
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTCAGTACTGGGGTCAGGGAACTCTGG
TCACCGTCTCGAGC
>DOM1h-574-151
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGCGCCTTTTGAGTACTGGGGTCAGGGAACCCTGG
TCACCGTCTCGAGC
>DOM1h-574-152
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGCGCCTTTTCAGTACTGGGGTCAGGGAACTCTGG
TCACCGTCTCGAGC
>DOM1h-574-153
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGTGCCTTTTCAGTACTGGGGTCAGGGCACCCTGG
TCACCGTCTCGAGC
>DOM1h-574-154
GAGGTGCAGCTGTTGGAGTCTGGGGGAGGCTTGGTACAGCCTGGGGGGT
CCCTGCGTCTCTCCTGTGCAGCCTCCGGATTCACCTTTGTTAAGTATTC
GATGGGGTGGGTCCGCCAGGCTCCAGGGAAGGGTCTAGAGTGGGTCTCA
CAGATTTCGGATACCGGTGATCGTAGATACTACGATCACTCTGTGAAGG
GCCGGTTCACTATCTCCCGCGACAATTCCAAGAACACGCTGTATCTGCA
AATGAACAGCCTGCGTGCCGAGGACACCGCGGTATATTACTGTGCGATA
TATACGGGTCGGTGGGCGCCTTTTGAGTACTGGGGTCAGGGAACCCTGG
TCACCGTCTCGAGC
>DOM1h-574-155
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-156
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-157
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACTGCTGATCGTACATACTACGATCACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGAGGCCTTTTGAGTACTGGGGTCAGGGAACCCTGG
TCACCGTCTCGAGC
>DOM1h-574-158
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTACATACTACGATCACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGAGGCCTTTTGAGTACTGGGGTCAGGGAACCCTGG
TCACCGTCTCGAGC
>DOM1h-574-159
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTACATACTACGATCACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-160
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACTGCTGATCGTACATACTACGATCACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGAGCCTTTTGTCTACTGGGGTCAGGGAACCCTGG
TCACCGTCTCGAGC
>DOM1h-574-161
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACTGCTGATCGTACATACTACTCACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-162
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTACATACTACTCACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-163
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTACATACTACACACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-164
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACTGCTGATCGTACATACTACACACACTCCGTGAAGG
GCCGGTTCACCATCTCCCGCGACAATTCCAAGAACACGCTGTATCTGCA
AATGAACAGCCTGCGTGCTGAGGACACCGCGGTATATTACTGTGCGATA
TATACTGGGCGTTGGGTGCCTTTTGAGTACTGGGGTCAGGGAACCCTGG
TCACCGTCTCGAGC
>DOM1h-574-165
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGCGCCTTTTGAGTACTGGGGTCAGGGAACCCTGG
TCACCGTCTCGAGC
>DOM1h-574-166
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACTGCTGATCGTACATACTACGCACACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGATA
TATACGGGTCGTTGGGCGCCTTTTGAGTACTGGGGTCAGGGAACCCTGG
TCACCGTCTCGAGC
>DOM1h-574-167
GAGGTGCAGCTGTTGGAGTCTGGGGGAGGCTTGGTACAGCCTGGGGGGT
CCCTGCGTCTCTCCTGTGCAGCCTCCGGATTCACCTTTTTGAAGTATTC
GATGGGGTGGGTCCGCCAGGCTCCAGGGAAGGGTCTAGAGTGGGTCTCA
CAGATTTCGGATACCGGTGATCGTAGATACTACGATCACTCTGTGAAGG
GCCGGTTCACTATCTCCCGCGACAATTCCAAGAACACGCTGTATCTGCA
AATGAACAGCCTGCGTGCCGAGGACACCGCGGTATATTACTGTGCGATA
TATACGGGTCGGTGGGCGCCTTTTGAGTACTGGGGTCAGGGAACCCTGG
TCACCGTCTCGAGC
>DOM1h-574-168
GAGGTGCAGCTGTTGGAGTCTGGGGGAGGCTTGGTACAGCCTGGGGGGT
CCCTGCGTCTCTCCTGTGCAGCCTCCGGATTCACCTTTTTCAAGTATTC
GATGGGGTGGGTCCGCCAGGCTCCAGGGAAGGGTCTAGAGTGGGTCTCA
CAGATTTCGGATACCGGTGATCGTAGATACTACGATCACTCTGTGAAGG
GCCGGTTCACTATCTCCCGCGACAATTCCAAGAACACGCTGTATCTGCA
AATGAACAGCCTGCGTGCCGAGGACACCGCGGTATATTACTGTGCGATA
TATACGGGTCGGTGGGCGCCTTTTGAGTACTGGGGTCAGGGAACCCTGG
TCACCGTCTCGAGC
>DOM1h-574-169
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGGATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGCGCGATATATACT
GGGCGTTGGGTGCCTTTTGAGTACTGGGGTCAGGGAACCCTGGTCACCGT
CTCGAGC
>DOM1h-574-170
GAGGTGCAGCTGTTGGAGTCTGGGGGAGGCTTGGTACAGCCTGGGGGGTC
CCTGCGTCTCTCCTGTGCAGCCTCCGGATTCACCTTTTTTAAGTATTCGA
TGGGGTGGGTCCGCCAGGCTCCAGGGAAGGGTCTAGAGTGGGTCTCACAG
ATTTCGGATACTGCTGATCGTACATACTACGCACACGCGGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-171
GAGGTGCAGCTGTTGGAGTCTGGGGGAGGCTTGGTGCAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGGATACTGCTGATCGTACATACTACGATCACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-172
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGGATACTGCTGATCGTACATACTACGATCACGCGGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DOM1h-574-173
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGGATACTGCTGATCGTAGATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-174
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTAGATACTACGCACACGCGGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-175
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGGATACTGCTGATCGTAGATACTACGCACACGCGGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-176
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTAGATACTACGATCACGCGGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-177
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGGATACTGCTGATCGTAGATACTACGATCACGCGGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGGACCCTGGTCACCGT
CTCGAGC
>DOM1h-574-178
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTGCGGATACTGCTGATCGTAGATACTACGATCACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGC
>DOM1h-574-179
GAGGTGCAGCTGCTGGAGTCTGGGGGAGGCTTGGTACAGCCTGGGGGGTC
CCTGCGTCTCTCCTGTGCAGCCTCCGGATTCACCTTTTTCAAGTATTCGA
TGGGGTGGGTCCGCCAGGCTCCAGGGAAGGGTCTAGAGTGGGTCTCACAG
ATTTCGGATACTGCTGATCGTAGATACTACGATGACGCGGTGAAGGGCCG
GTTCACCATCACCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGTCTACTGGGGTCAGGGAACCCTGGTCACCGT
CTCGAGC
>DOM1h-574-180
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGCACACGCGGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGC
>DMS0111
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCAT
CTGTAGGAGACCGTGTCACCATCACTTGCCGGGCAAGTCGTCCGATTGGG
ACGACGTTAAGTTGGTACCAGCAGAAACCAGGGAAAGCCCCTAAGCTCCT
GATCCTTTGGAATTCCCGTTTGCAAAGTGGGGTCCCATCACGTTTCAGTG
GCAGTGGATCTGGGACAGATTTCACTCTCACCATCAGCAGTCTGCAACCT
GAAGATTTTGCTACGTACTACTGTGCGCAGGCTGGGACGCATCCTACGAC
GTTCGGCCAAGGGACCAAGGTGGAAATCAAACGG
>DMS0112
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGATGTTAAGTTGGTACCAGCAGAAACCAGGGAAAGCCCCTAA
GCTCCTGATCTTGTTTGGTTCCCGGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0113
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCAGTGG
ATTGGGTCTCAGTTATCTTGGTACCAGCAGAAACCAGGGAAAGCCCCTAA
GCTCCTGATCATGTGGCGTTCCTCGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCTCAGGGTTTGAGGCATCC
TAAGACGTTCGGCCAAGGGACCAAGGTGGAAATCAAACGG
>DMS0114
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCAGTGG
ATTGGGTCTCAGTTATCTTGGTACCAGCAGAAACCAGGGAAAGCCCCTAA
GCTCCTGATCATGTGGCGTTCCTCGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCTCAGGGTCTTATGAAGCC
TATGACGTTCGGCCAAGGGACCAAGGTGGAAATCAAACGG
>DMS0115
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGACGTTAAGTTGGTACCAGCAGAAACCAGG
GAAAGCCCCTAAGCTCCTGATCCTTTGGAATTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DMS0116
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCTTGTTTGGTTCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DMS0117
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCAGTGGATTGGGTCTCAGTTATCTTGGTACCAGCAGAAACCAGG
GAAAGCCCCTAAGCTCCTGATCATGTGGCGTTCCTCGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCTCAGGG
TTTGAGGCATCCTAAGACGTTCGGCCAAGGGACCAAGGTGGAAATCAAAC
GG
>DMS0118
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCAGTGGATTGGGTCTCAGTTATCTTGGTACCAGCAGAAACCAGG
GAAAGCCCCTAAGCTCCTGATCATGTGGCGTTCCTCGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCTCAGGG
TCTTATGAAGCCTATGACGTTCGGCCAAGGGACCAAGGTGGAAATCAAAC
GG
>DMS0121
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCGGTGGAGGCGGTTCAGGCGGAGGTGGCAGCGGCGGTG
GCGGATCCGACATCCAGATGACCCAGTCTCCATCCTCCCTGTCTGCATCT
GTAGGAGACCGTGTCACCATCACTTGCCGGGCAAGTCGTCCGATTGGGAC
GACGTTAAGTTGGTACCAGCAGAAACCAGGGAAAGCCCCTAAGCTCCTGA
TCCTTTGGAATTCCCGTTTGCAAAGTGGGGTCCCATCACGTTTCAGTGGC
AGTGGATCTGGGACAGATTTCACTCTCACCATCAGCAGTCTGCAACCTGA
AGATTTTGCTACGTACTACTGTGCGCAGGCTGGGACGCATCCTACGACGT
TCGGCCAAGGGACCAAGGTGGAAATCAAACGG
>DMS0122
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCGGTGGAGGCGGTTCAGGCGGAGGTGGCAGCGGCGGTG
GCGGATCCGACATCCAGATGACCCAGTCTCCATCCTCCCTGTCTGCATCT
GTAGGAGACCGTGTCACCATCACTTGCCGGGCAAGTCGTCCGATTGGGAC
GATGTTAAGTTGGTACCAGCAGAAACCAGGGAAAGCCCCTAAGCTCCTGA
TCTTGTTTGGTTCCCGGTTGCAAAGTGGGGTCCCATCACGTTTCAGTGGC
AGTGGATCTGGGACAGATTTCACTCTCACCATCAGCAGTCTGCAACCTGA
AGATTTTGCTACGTACTACTGTGCGCAGGCTGGGACGCATCCTACGACGT
TCGGCCAAGGGACCAAGGTGGAAATCAAACGG
>DMS0123
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCGGTGGAGGCGGTTCAGGCGGAGGTGGCAGCGGCGGTG
GCGGATCCGACATCCAGATGACCCAGTCTCCATCCTCCCTGTCTGCATCT
GTAGGAGACCGTGTCACCATCACTTGCCGGGCAAGTCAGTGGATTGGGTC
TCAGTTATCTTGGTACCAGCAGAAACCAGGGAAAGCCCCTAAGCTCCTGA
TCATGTGGCGTTCCTCGTTGCAAAGTGGGGTCCCATCACGTTTCAGTGGC
AGTGGATCTGGGACAGATTTCACTCTCACCATCAGCAGTCTGCAACCTGA
AGATTTTGCTACGTACTACTGTGCTCAGGGTTTGAGGCATCCTAAGACGT
TCGGCCAAGGGACCAAGGTGGAAATCAAACGG
>DMS0124
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCGGTGGAGGCGGTTCAGGCGGAGGTGGCAGCGGCGGTG
GCGGATCCGACATCCAGATGACCCAGTCTCCATCCTCCCTGTCTGCATCT
GTAGGAGACCGTGTCACCATCACTTGCCGGGCAAGTCAGTGGATTGGGTC
TCAGTTATCTTGGTACCAGCAGAAACCAGGGAAAGCCCCTAAGCTCCTGA
TCATGTGGCGTTCCTCGTTGCAAAGTGGGGTCCCATCACGTTTCAGTGGC
AGTGGATCTGGGACAGATTTCACTCTCACCATCAGCAGTCTGCAACCTGA
AGATTTTGCTACGTACTACTGTGCTCAGGGTCTTATGAAGCCTATGACGT
TCGGCCAAGGGACCAAGGTGGAAATCAAACGG
>DMS0132
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCCTTTGGAATTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0133
GAGGTGCAGCTGTTGGAGTCTGGGGGAGGCTTGGTACAGCCTGGGGGGTC
CCTGCGTCTCTCCTGTGCAGCCTCCGGATTCACCTTTTTCAAGTATTCGA
TGGGGTGGGTCCGCCAGGCTCCAGGGAAGGGTCTAGAGTGGGTCTCACAG
ATTTCGGATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGGTGGGCGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCCTTTGGAATTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0134
GAGGTGCAGCTGTTGGAGTCTGGGGGAGGCTTGGTACAGCCTGGGGGGTC
CCTGCGTCTCTCCTGTGCAGCCTCCGGATTCACCTTTTTCAAGTATTCGA
TGGGGTGGGTCCGCCAGGCTCCAGGGAAGGGTCTAGAGTGGGTCTCACAG
ATTTCGGATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCCTTTGGAATTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0135
GAGGTGCAGCTGTTGGAGTCTGGGGGAGGCTTGGTACAGCCTGGGGGGTC
CCTGCGTCTCTCCTGTGCAGCCTCCGGATTCACCTTTTTCAAGTATTCGA
TGGGGTGGGTCCGCCAGGCTCCAGGGAAGGGTCTAGAGTGGGTCTCACAG
ATTTCGGATACTGCTGATCGTACATACTACTCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCCTTTGGAATTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0136
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGGATACTGCTGATCGTACATACTACGCACACGCGGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCCTTTGGAATTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0162
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGTGT
>DMS0163
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCCTTGCTTTTTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGCGCGCAGGC
TGGGACGCATCCTACGACGTTCGGCCAAGGGACCAAGGTGGAAATCAAAC
GGGCGGCCGCAGAACAAAAACTCATCTCAGAAGAGGATCTGAAT
>DMS0163-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCCTTGCTTTTTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGCGCGCAGGC
TGGGACGCATCCTACGACGTTCGGCCAAGGGACCAAGGTGGAAATCAAAC
GG
>DMS0168
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCAGAGCATTATTAAGCATTTAAAGTGGTACCAGCAGAAACCAGG
GAAAGCCCCTAAGCTCCTGATCTATGGTGCATCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTCAACAGGG
GGCTCGGTGGCCTCAGACGTTCGGCCAAGGGACCAAGGTGGAAATCAAAC
GGGCGGCCGCAGAACAAAAACTCATCTCAGAAGAGGATCTGAAT
>DMS0168-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCAGAGCATTATTAAGCATTTAAAGTGGTACCAGCAGAAACCAGG
GAAAGCCCCTAAGCTCCTGATCTATGGTGCATCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTCAACAGGG
GGCTCGGTGGCCTCAGACGTTCGGCCAAGGGACCAAGGTGGAAATCAAAC
GG
>DMS0169
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCTTGTTTGGTTCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GGGCGGCCGCAGAACAAAAACTCATCTCAGAAGAGGATCTGAAT
>DMS0169-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCTTGTTTGGTTCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DMS0176
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGACATCCAGATGACCCAGTCTCCATCCTCCCTGTCTGCATCTG
TAGGAGACCGTGTCACCATCACTTGCCGGGCAAGTCGTCCGATTGGGACG
ACGTTAAGTTGGTACCAGCAGAAACCAGGGAAAGCCCCTAAGCTCCTGAT
CTGGTTTGGTTCCCGGTTGCAAAGTGGGGTCCCATCACGTTTCAGTGGCA
GTGGATCTGGGACAGATTTCACTCTCACCATCAGCAGTCTGCAACCTGAA
GATTTTGCTACGTACTACTGTGCGCAGGCTGGGACGCATCCTACGACGTT
CGGCCAAGGGACCAAGGTGGAAATCAAACGG
>DMS0177
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGACATCCAGATGACCCAGTCTCCATCCTCCCTGTCTGCATCTG
TAGGAGACCGTGTCACCATCACTTGCCGGGCAAGTCAGTGGATTGGGTCT
CAGTTATCTTGGTACCAGCAGAAACCAGGGAAAGCCCCTAAGCTCCTGAT
CATGTGGCGTTCCTCGTTGCAAAGTGGGGTCCCATCACGTTTCAGTGGCA
GTGGATCTGGGACAGATTTCACTCTCACCATCAGCAGTCTGCAACCTGAA
GATTTTGCTACGTACTACTGTGCTCAGGGTGCGGCGTTGCCTAGGACGTT
CGGCCAAGGGACCAAGGTGGAAATCAAACGG
>DMS0182
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCTGGTTTGGTTCCCGGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGGGCGGCCGCAG
AACAAAAACTCATCTCAGAAGAGGATCTGAAT
>DMS0182-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCTGGTTTGGTTCCCGGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0184
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGACGTTAAGTTGGTACCAGCAGAAACCAGG
GAAAGCCCCTAAGCTCCTGATCTGGTTTGGTTCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DMS0186
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGATGTTAAGTTGGTACCAGCAGAAACCAGGGAAAGCCCCTAA
GCTCCTGATCTTGTTTGGTTCCCGGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGGGCGGCCGCAG
AACAAAAACTCATCTCAGAAGAGGATCTGAAT
>DMS0186-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGATGTTAAGTTGGTACCAGCAGAAACCAGGGAAAGCCCCTAA
GCTCCTGATCTTGTTTGGTTCCCGGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0188
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCTTGTTTGGTTCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GGGCGGCCGCAGAACAAAAACTCATCTCAGAAGAGGATCTGAAT
>DMS0188-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCTTGTTTGGTTCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DMS0189
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCCTTTGGAATTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGGGCGGCCGCAG
AACAAAAACTCATCTCAGAAGAGGATCTGAAT
>DMS0189-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGACGTTAAGTTGGTACCAGCAGAAACCAGGGAAAGCCCCTAA
GCTCCTGATCCTTTGGAATTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTGCGCAGGCTGGGACGCATCC
TACGACGTTCGGCCAAGGGACCAAGGTGGAAATCAAACGG
>DMS0190
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGACGTTAAGTTGGTACCAGCAGAAACCAGG
GAAAGCCCCTAAGCTCCTGATCCTTTGGAATTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GGGCGGCCGCAGAACAAAAACTCATCTCAGAAGAGGATCTGAAT
>DMS0190-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACAGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGACGTTAAGTTGGTACCAGCAGAAACCAGG
GAAAGCCCCTAAGCTCCTGATCCTTTGGAATTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DMS0191
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCAGAGC
ATTATTAAGCATTTAAAGTGGTACCAGCAGAAACCAGGGAAAGCCCCTAA
GCTCCTGATCTATGGTGCATCCCGGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTCAACAGGGGACTCGGTGGCC
TCAGACGTTCGGCCAAGGGACCAAGGTGGAAATCAAACGGGCGGCCGCAG
AACAAAAACTCATCTCAGAAGAGGATCTGAAT
>DMS0191-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCAGAGC
ATTATTAAGCATTTAAAGTGGTACCAGCAGAAACCAGGGAAAGCCCCTAA
GCTCCTGATCTATGGTGCATCCCGGTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGTCAACAGGGGACTCGGTGGCC
TCAGACGTTCGGCCAAGGGACCAAGGTGGAAATCAAACGG
>DMS0192
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGTGACCGTGTCACCATCACTTGCCGG
GCAAGTCAGAGCATTATTAAGCATTTAAAGTGGTACCAGCAGAAACCAGG
GAAAGCCCCTAAGCTCCTGATCTATGGTGCATCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTCAACAGGG
GGCTCGGTGGCCTCAGACGTTCGGCCAAGGGACCAAGGTGGAAATCAAAC
GGGCGGCCGCAGAACAAAAACTCATCTCAGAAGAGGATCTGAAT
>DMS0192-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGATGGGTCCGCCAGGCTCCAGGGAAAGGTCCAGAGTGGGTCTCACAG
ATTTCGAATACGGGTGATCGTACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGATATATACG
GGTCGTTGGGAGCCTTTTGACTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGTGACCGTGTCACCATCACTTGCCGG
GCAAGTCAGAGCATTATTAAGCATTTAAAGTGGTACCAGCAGAAACCAGG
GAAAGCCCCTAAGCTCCTGATCTATGGTGCATCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTCAACAGGG
GGCTCGGTGGCCTCAGACGTTCGGCCAAGGGACCAAGGTGGAAATCAAAC
GG
>DMS5519
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCCTTGCTTTTTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGCGCGCAGGC
TGGGACGCATCCTACGACGTTCGGCCAAGGGACCAAGGTGGAAATCAAAC
GG
>DMS5520
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGGTCATACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGAAATATACG
GGTCATTGGGAGCCTTTTGACTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGACGTTAAGTTGGTACCAGCAGAAACCAGG
GAAAGCCCCTAAGCTCCTGATCCTTTGGAATTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DMS5521
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGATGTTAAGTTGGTACCAGCAGAAACCAGGGAAAGCCCCTAA
GCTCCTGATCCTTGCTTTTTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGCGCGCAGGCTGGGACGCATCC
TACGACGTTCGGCCAAGGGACCAAGGTGGAAATCAAACGG
>DMS5522
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGATGTTAAGTTGGTACCAGCAGAAACCAGGGAAAGCCCCTAA
GCTCCTGATCCTTGCTTTTTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGCGCGCAGGCTGGGACGCATCC
TACGACGTTCGGCCAAGGGACCAAGGTGGAAATCAAACGGGCGGCCGCAG
AACAAAAACTCATCTCAGAAGAGGATCTGAAT
>DMS5522-no tag
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCGACATCCAGATGACCCAGTCTCCATCCTCCCTGT
CTGCATCTGTAGGAGACCGTGTCACCATCACTTGCCGGGCAAGTCGTCCG
ATTGGGACGATGTTAAGTTGGTACCAGCAGAAACCAGGGAAAGCCCCTAA
GCTCCTGATCCTTGCTTTTTCCCGTTTGCAAAGTGGGGTCCCATCACGTT
TCAGTGGCAGTGGATCTGGGACAGATTTCACTCTCACCATCAGCAGTCTG
CAACCTGAAGATTTTGCTACGTACTACTGCGCGCAGGCTGGGACGCATCC
TACGACGTTCGGCCAAGGGACCAAGGTGGAAATCAAACGG
>DMS5525
GAGGTGCAGCTGTTGGAGTCTGGGGGAGGCTTGGTACAGCCTGGGGGGTC
CCTGCGTCTCTCCTGTGCAGCCTCCGGATTCACCTTTGTTAAGTATTCGA
TGGGGTGGGTCCGCCAGGCTCCAGGGAAGGGTCTAGAGTGGGTCTCACAG
ATTTCGAATACGGGTGGTCATACATACTACGCAGACTCCGTGAAGGGCCG
GTTCACCATCTCCCGCGACAATTCCAAGAACACGCTGTATCTGCAAATGA
ACAGCCTGCGTGCCGAGGACACCGCGGTATATTACTGTGCGAAATATACG
GGTCATTGGGAGCCTTTTGACTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCCTTGCTTTTTCCCGTTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGCGCGCAGGC
TGGGACGCATCCTACGACGTTCGGCCAAGGGACCAAGGTGGAAATCAAAC
GG
>DMS5527
GAGGTGCAGCTGTTGGAGTCTGGGGGAGGCTTGGTACAGCCTGGGGGGTC
CCTGCGTCTCTCCTGTGCAGCCTCCGGATTCACCTTTTTCAAGTATTCGA
TGGGGTGGGTCCGCCAGGCTCCAGGGAAGGGTCTAGAGTGGGTCTCACAG
ATTTCGGATACTGCTGATCGTACATACTACGCACACTCCGTGAAGGGCCG
GTTCACCATCTCCCGCGACAATTCCAAGAACACGCTGTATCTGCAAATGA
ACAGCCTGCGTGCTGAGGACACCGCGGTATATTACTGTGCGATATATACT
GGGCGTTGGGTGCCTTTTGAGTACTGGGGTCAGGGAACCCTGGTCACCGT
CTCGAGCGCTAGCACCAGTGGTCCATCGGACATCCAGATGACCCAGTCTC
CATCCTCCCTGTCTGCATCTGTAGGAGACCGTGTCACCATCACTTGCCGG
GCAAGTCGTCCGATTGGGACGATGTTAAGTTGGTACCAGCAGAAACCAGG
GAAAGCCCCTAAGCTCCTGATCTTGTTTGGTTCCCGGTTGCAAAGTGGGG
TCCCATCACGTTTCAGTGGCAGTGGATCTGGGACAGATTTCACTCTCACC
ATCAGCAGTCTGCAACCTGAAGATTTTGCTACGTACTACTGTGCGCAGGC
TGGGACGCATCCTACGACGTTCGGCCAAGGGACCAAGGTGGAAATCAAAC
GG
>DOM7h-11
GACATCCAGATGACCCAGTCTCCATCCTCCCTGTCTGCATCTGTAGGAGA
CCGTGTCACCATCACTTGCCGGGCAAGTCGTCCGATTGGGACGACGTTAA
GTTGGTACCAGCAGAAACCAGGGAAAGCCCCTAAGCTCCTGATCTGGTTT
GGTTCCCGGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCGCAGGCTGGGACGCATCCTACGACGTTCGGCCAA
GGGACCAAGGTGGAAATCAAACGG
>DOM7h-11-3
GACATCCAGATGACCCAGTCTCCATCCTCCCTGTCTGCATCTGTAGGAGA
CCGTGTCACCATCACTTGCCGGGCAAGTCGTCCGATTGGGACGACGTTAA
GTTGGTACCAGCAGAAACCAGGGAAAGCCCCTAAGCTCCTGATCCTTTGG
AATTCCCGTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCGCAGGCTGGGACGCATCCTACGACGTTCGGCCAA
GGGACCAAGGTGGAAATCAAACGG
>DOM7h-11-12
GACATCCAGATGACCCAGTCTCCATCCTCCCTGTCTGCATCTGTAGGAGA
CCGTGTCACCATCACTTGCCGGGCAAGTCGTCCGATTGGGACGATGTTAA
GTTGGTACCAGCAGAAACCAGGGAAAGCCCCTAAGCTCCTGATCTTGTTT
GGTTCCCGGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCGCAGGCTGGGACGCATCCTACGACGTTCGGCCAA
GGGACCAAGGTGGAAATCAAACGG
>DOM7h-11-15
GACATCCAGATGACCCAGTCTCCATCCTCCCTGTCTGCATCTGTAGGAGA
CCGTGTCACCATCACTTGCCGGGCAAGTCGTCCGATTGGGACGATGTTAA
GTTGGTACCAGCAGAAACCAGGGAAAGCCCCTAAGCTCCTGATCCTTGCT
TTTTCCCGTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGCGCGCAGGCTGGGACGCATCCTACGACGTTCGGCCAA
GGGACCAAGGTGGAAATCAAACGG
>DOM7h-14
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTGCGGCGTTGCCTAGGACGTTCGGCCAA
GGGACCAAGGTGGAAATCAAACGG
>DOM7h-14-10
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTTCGGCCAA
GGGACCAAGGTGGAAATCAAACGG
>DOM7h-14-18
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTCTTATGAAGCCTATGACGTTCGGCCAA
GGGACCAAGGTGGAAATCAAACGG
>DOM7m-16
GACATCCAGATGACCCAGTCTCCATCCTCCCTGTCTGCATCTGTAGGAGA
CCGTGTCACCATCACTTGCCGGGCAAGTCAGAGCATTATTAAGCATTTAA
AGTGGTACCAGCAGAAACCAGGGAAAGCCCCTAAGCTCCTGATCTATGGT
GCATCCCGGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGGGGCTCGGTGGCCTCAGACGTTCGGCCAA
GGGACCAAGGTGGAAATCAAACGG
>DOM1h-131-511
GAGGTGCAGCTGTTGGAGTCTGGGGGAGGCTTGGTACAGCCTGGGGGGTC
CCTGCGTCTCTCCTGTGCAGCCTCCGGATTCACCTTTGCGCATGAGACGA
TGGTGTGGGTCCGCCAGGCTCCAGGGAAGGGTCTAGAGTGGGTCTCACAT
ATTCCCCCGGTTGGTCAGGATCCCTTCTACGCAGACTCCGTGAAGGGCCG
GTTCACCATCTCCCGCGACAATTCCAAGAACACGCTATATCTGCAAATGA
ACAGCCTGCGTGCCGAGGACACAGCGGTATATTACTGTGCGCTGCTTCCT
AAGAGGGGGCCTTGGTTTGACTACTGGGGTCAGGGAACCCTGGTCACCGT
CTCGAGC
>DOM1m-21-23
GAGGTGCAGCTGTTGGAGTCTGGGGGAGGCTTGGTACAGCCTGGGGGGTC
CCTGCGTCTCTCCTGTGCAGCCTCCGGATTCACCTTTAATAGGTATAGTA
TGGGGTGGCTCCGCCAGGCTCCAGGGAAGGGTCTAGAGTGGGTCTCACGG
ATTGATTCTTATGGTCGTGGTACATACTACGAAGACCCCGTGAAGGGCCG
GTTCAGCATCTCCCGCGACAATTCCAAGAACACGCTGTATCTGCAAATGA
ACAGCCTGCGTGCCGAGGACACCGCCGTATATTACTGTGCGAAAATTTCT
CAGTTTGGGTCAAATGCGTTTGACTACTGGGGTCAGGGAACCCAGGTCAC
CGTCTCGAGC
>DOM1m-15-12
GACATCCAGATGACCCAGTCTCCATCCTCCCTGTCTGCATCTGTAGGAGA
CCGTGTCACCATCACTTGCCGGGCAAGTCAGTATATTCATACGAGTGTAC
AGTGGTACCAGCAGAAACCAGGGAAAGCCCCTAAACTCCTGATCTATGGG
TCGTCCAGGTTGCATAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGAATCATTATAGTCCTTTTACGTACGGCCAA
GGGACCAAGGTGGAAATCAAACGG

Oligonucleotide sequences

AS9: CAGGAAACAGCTATGACCATG
AS65: TTGTAAAACGACGGCCAGTG
AS339: TTCAGGCTGCGCAACTGTTG
AS639: CGCCAAGCTTGCATGCAAATTC
AS1029: CCTGTGCAGCCTCCGGATTCACCTTTgtTaagtaTtcGatg
ggGTGGGTCCGCCAGG
AS1030: TCCAGGGAAGGGTCTAGAGTGGGTCTCAcagatttcgaata
cgggtgatcgtacataCta CgcagactccgtgaagggcCGGTTCACCAT
CTCCC
AS1031: GAGGACACCGCGGTATATTACTGTGCGatAtaTacgggtcgt
tgGgagccttttgactaCT GGGGTCAGGGAACCCTGGTC
AS1031′: AAAGGTGAATCCGGAGGCTGCACAGG
AS1032: TGAGACCCACTCTAGACCCTTCCCTGGA
AS1033: CGCACAGTAATATACCGCGGTGTCCTC

1. An anti-TNFα receptor type 1 (TNFR1; p55) antagonist for administration to a patient suffering from a TNFR1-mediated disease or condition, wherein the antagonist is a non-competitive inhibitor of TNFR1,
wherein the antagonist at a concentration of 100 nM inhibits TNFR1 by
(i) >50% in a standard MRC5 cell assay in the presence of human TNFα at a TNFα concentration in the assay of 100 pg/ml as determined by an immuno-sandwich method, and
(ii) ≦50% in a standard MRC5 cell assay in the presence of human TNFα at a TNFα concentration in the assay of 2 ng/ml or more as determined by said immuno-sandwich method,
for treating and/or preventing said TNFR1-mediated disease or condition by partially inhibiting TNFR1-mediated signaling in the patient,
wherein the antagonist inhibits binding of human TNFR1 to an immunoglobulin single variable domain selected from DOM1h-574-156, DOM1h-574-72, DOM1h-574-109, DOM1h-574-138, DOM1h-574-162 and DOM1h-574-180.

2. (canceled)

3. A method of treating and/or preventing a TNFR1-mediated disease or condition in a patient, the method comprising partially inhibiting TNFR1-mediated signaling in the patient by administering an effective amount of an anti-TNFα receptor type 1 (TNFR1; p55) antagonist to the patient, wherein the antagonist is a non-competitive inhibitor of TNFR1, and
wherein the antagonist at a concentration of 100 nM inhibits TNFR1 by
(i) >50% in a standard MRC5 cell assay in the presence of human TNFα at a concentration in the assay of 100 pg/ml as determined by an immuno-sandwich method, and
(ii) ≦50% in a standard MRC5 cell assay in the presence of human TNFα at a concentration in the assay of 2 ng/ml or more as determined by said immuno-sandwich method,
wherein the antagonist inhibits binding of human TNFR1 to an immunoglobulin single variable domain selected from DOM1h-574-156, DOM1h-574-72, DOM1h-574-109, DOM1h-574-138, DOM1h-574-162 and DOM1h-574-180.

4. The antagonist of claim 1, wherein the antagonist comprises an immunoglobulin variable domain having an amino acid sequence that is at least 80% identical to the amino acid sequence of DOM1h-574-156, DOM1h-574-72, DOM1h-574-109, DOM1h-574-138, DOM1h-574-162 or DOM1h-574-180.

5. An anti-TNFα receptor type 1 (TNFR1; p55) antagonist for administration to a patient suffering from a TNFR1-mediated disease or condition, wherein the antagonist is a non-competitive inhibitor of TNFR1,
wherein the antagonist at a concentration of 100 nM inhibits TNFR1 by
(i) >50% in a standard L929 cell assay in the presence of murine TNFα at a concentration in the assay of 20 pg/ml, and
(ii) ≦50% in a standard L929 cell assay in the presence of murine TNFα at a concentration in the assay of 100 pg/ml or more,
for treating and/or preventing said TNFR1-mediated disease or condition by partially inhibiting TNFR1-mediated signaling in the patient,
wherein the antagonist inhibits binding of murine TNFR1 to an immunoglobulin single variable domain selected from DOM1h-574-156; DOM1m-21-23, DOM1h-574-156, DOM1h-574-72, DOM1h-574-109, DOM1h-574-138, DOM1h-574-162 and DOM1h-574-180.

6. (canceled)

7. A method of treating and/or preventing a TNFR1-mediated disease or condition in a patient, the method comprising partially inhibiting TNFR1-mediated signaling in the patient by administering an effective amount of an anti-TNFα receptor type 1 (TNFR1; p55) antagonist to the patient, wherein the antagonist is a non-competitive inhibitor of TNFR1, and
wherein the antagonist at a concentration of 100 nM inhibits TNFR1 by
(i) >50% in a standard L929 cell assay in the presence of murine TNFα at a concentration in the assay of 20 pg/ml and
(ii) ≦50% in a standard L929 cell assay in the presence of murine TNFα at a concentration in the assay of 100 pg/ml or more,
for treating and/or preventing said TNFR1-mediated disease or condition by partially inhibiting TNFR1-mediated signaling in the patient,
wherein the antagonist inhibits binding of murine TNFR1 to an immunoglobulin single variable domain selected from DOM1h-574-156; DOM1m-21-23, DOM1h-574-156, DOM1h-574-72, DOM1h-574-109, DOM1h-574-138, DOM1h-574-162 and DOM1h-574-180.

8. The antagonist of claim 5, wherein the antagonist comprises an immunoglobulin variable domain having an amino acid sequence that is at least 80% identical to the amino acid sequence of DOM1h-574-156 or DOM1m-21-23.

9. (canceled)

10. The antagonist of claim 1, wherein said condition is selected from the group consisting of an inflammatory condition, arthritis, ankylosing spondylitis, osteoarthritis, inflammatory bowel disease and psoriasis.

11. An isolated or recombinant nucleic acid comprising a nucleotide sequence encoding for an antagonist according to claim 1.

12. A vector comprising the nucleic acid of claim 11.

13. A host cell comprising the nucleic acid of claim 11.

14. A pharmaceutical composition comprising an anti-TNFR1 antagonist of claim 1 and a pharmaceutically acceptable carrier, excipient or diluent.

15. The method of claim 3, wherein the antagonist comprises an immunoglobulin variable domain having an amino acid sequence that is at least 80% identical to the amino acid sequence of DOM1h-574-156, DOM1h-574-72, DOM1h-574-109, DOM1h-574-138, DOM1h-574-162 or DOM1h-574-180.

16. The method of claim 7, wherein the antagonist comprises an immunoglobulin variable domain having an amino acid sequence that is at least 80% identical to the amino acid sequence of DOM1h-574-156 or DOM1m-21-23.

17. The method of claim 3, wherein said condition is selected from the group consisting of an inflammatory condition, arthritis, ankylosing spondylitis, osteoarthritis, inflammatory bowel disease and psoriasis.

18. The antagonist of claim 5, wherein said condition is selected from the group consisting of an inflammatory condition, arthritis, ankylosing spondylitis, osteoarthritis, inflammatory bowel disease and psoriasis.

19. The method of claim 7, wherein said condition is selected from the group consisting of an inflammatory condition, arthritis, ankylosing spondylitis, osteoarthritis, inflammatory bowel disease and psoriasis.

20. A pharmaceutical composition comprising an anti-TNFR1 antagonist of claim 5 and a pharmaceutically acceptable carrier, excipient or diluent.

21. A host cell comprising the vector of claim 12.
